(19) United States
(12) Patent Application Publication
(10) Pub. No.: US 2022/0040218 A1
(43) Pub. Date: Feb. 10, 2022

(54) Therapeutic RNA for Advanced Stage Solid Tumor Cancers

(71) Applicant: SANOFI, Paris (FR)

(72) Inventors: Serena Masciari, Saugus, MA (US); Semra Yoruk, Istanbul (TR); Karl Hsu, Lexington, MA (US); Timothy R. Wagenaar, Sudbury, MA (US); Nicolas Acquavella, Newton, MA (US); Marie Bernardo, Arlington, MA (US); Robert Jabulowsky, Offenbach (DE); Ugur Sahin, Mainz (DE); Friederike Gieseke, Mainz (DE); Zuzana Jirakova Trnkova, Mainz (DE)

(73) Assignee: SANOFI, Paris (FR)

(21) Appl. No.: 17/380,249

(22) Filed: Jul. 20, 2021

Related U.S. Application Data
(63) Continuation of application No. PCT/US2020/014019, filed on Jan. 17, 2020.
(60) Provisional application No. 62/926,384, filed on Oct. 25, 2019.
(60) Provisional application No. 62/794,889, filed on Jan. 21, 2019.

(30) Foreign Application Priority Data
Nov. 12, 2019 (EP) 19306461.5

(51) Int. Cl. A61K 31/7115; A61P 35/00; A61K 9/00; A61K 38/20; A61K 38/19; A61K 38/21; A61P 35/04

(52) U.S. Cl. CPC A61K 31/7115; A61P 35/00; A61K 9/0019; A61P 35/04; A61K 38/208; A61K 38/193; A61K 38/212; A61K 38/2086

(57) ABSTRACT

This disclosure relates to the field of therapeutic RNAs for treatment of subjects that have failed, or become intolerant, resistant, or refractory to an anti-programmed cell death 1 (PD-1) or anti-programmed cell death 1 ligand 1 (PD-L1) therapy, including innate and acquired PD-1 and/or PD-L1 therapy, as well as in subjects with advanced-stage, unresectable, or metastatic solid tumor cancers with or without failure, intolerance, resistance, or refraction to an anti-programmed cell death 1 (PD-1) or anti-programmed cell death 1 ligand 1 (PD-L1) therapy.

This application is a Continuation of International Application No. PCT/US2020/014019, filed Jan. 17, 2020, which claims the benefit of priority to U.S. Provisional Application No. 62/794,889, filed Jan. 21, 2019, U.S. Provisional Application No. 62/926,384, filed Oct. 25, 2019, and European Patent Application No. 19306461.5, filed Nov. 12, 2019, the contents of each of which are incorporated by reference in their entireties for all purposes.

The instant application contains a Sequence Listing which has been submitted electronically in ASCII format and is hereby incorporated by reference in its entirety. Said ASCII copy, created on Jul. 19, 2021, is named 01183-0021-00US_ST25.txt and is 30,607 bytes in size.

This disclosure relates to the field of therapeutic RNA to treat solid tumor cancers, including, for example, in subjects that have failed, or become intolerant, resistant, or refractory to an anti-programmed cell death 1 (PD-1) or anti-programmed cell death 1 ligand (PD-L1) therapy, including subjects with acquired or innate resistance to an anti-programmed cell death 1 (PD-1) or anti-programmed cell death 1 ligand (PD-L1) therapy, and subjects with advanced-stage or metastatic solid tumors.

The National Cancer Institute defines solid tumors as abnormal masses of tissue that do not normally contain cysts or liquid areas. Solid tumors can be physically located in any tissue or organ including the ovary, breast, colon, and other tissues, and include melanoma, cutaneous squamous cell cancer (CSCC), squamous cell carcinoma of the head and neck (HNSCC), non-small cell lung cancer, kidney cancer, head and neck cancer, thyroid cancer, colon cancer, liver cancer, ovarian cancer, breast cancer.

Immune checkpoint blockade, such as with anti-PD-1 and anti-PD-L1 therapy elicits anticancer responses in the clinic, however a large proportion of patients do not benefit from treatment. Several mechanisms of innate and acquired resistance to checkpoint blockade have been defined and include mutations of MHC I and IFNγ signaling pathways. See, e.g., Sade-Feldman et al. (2017) Nature Communications 8: 1136; see, also, Sharma et al. (2017) Cell 168: 707-723.

Advanced stage solid tumor cancers are particularly difficult to treat. Current treatments include surgery, radiotherapy, immunotherapy and chemotherapy. Surgery alone may be an appropriate treatment for small localized tumors, but large invasive tumors may be unresectable by surgery. Other common treatments such as radiotherapy and chemotherapy are associated with undesirable side effects and damage to healthy cells.

While surgery and current therapies sometimes are able to kill the bulk of the solid tumor, additional cells (including potentially cancer stem cells) may survive therapy. These cells, over time, can form a new tumor leading to cancer recurrence. In spite of multimodal conventional therapies, disease-free survival is less than 25% for many types of solid tumors. Solid tumors that are resistant to multi-modal therapy or that have recurred following therapy are even more difficult to treat, and long-term survival is less than 10%. In particular, there is high need for patients who failed immunotherapies, for example, using monoclonal antibodies against anti-programmed cell death protein 1 or its ligand (anti-PD-1 or anti-PD-L1 therapy).

Disclosed herein are compositions, uses, and methods that can overcome present shortcomings in treatment of solid tumors, such as advanced-stage, unresectable, or metastatic solid tumor cancers, including in subjects that have failed, or become intolerant, resistant, or refractory to an anti-programmed cell death 1 (PD-1) or anti-programmed cell death 1 ligand (PD-L1) therapy. Administration of therapeutic RNAs as disclosed herein can reduce tumor size, prolong time to progressive disease, and/or protect against metastasis and/or recurrence of the tumor and ultimately extend survival time.

### SUMMARY

Provided herein, inter alia, are methods of treating a subject having a solid tumor cancer, comprising administering an effective amount of RNAs comprising RNA encoding an IL-12sc protein, RNA encoding an IL-15 sushi protein. RNA encoding an IFNα protein, and RNA encoding a GM-CSF protein, wherein the subject has failed, or become intolerant, resistant, or refractory to an anti-programmed cell death 1 (PD-1) or anti-programmed cell death 1 ligand (PD-L1) therapy.

In some embodiments, methods of treating a solid tumor cancer in a subject that has failed, or become intolerant, resistant, or refractory to an anti-programmed cell death 1 (PD-1) or anti-programmed cell death 1 ligand (PD-L1) therapy are provided, comprising administering an effective amount of RNAs comprising RNA encoding an IL-12sc protein, RNA encoding an IL-15 sushi protein, RNA encoding an IFNα protein, and RNA encoding a GM-CSF protein to a subject that has failed, or become intolerant, resistant, or refractory to an anti-programmed cell death 1 (PD-1) or anti-programmed cell death 1 ligand (PD-L1) therapy.

Methods of treating a subject having anti-PD-1 and/or anti-PD-L1 resistant solid tumor cancer are provided, comprising administering an effective amount of RNAs comprising RNA encoding an IL-12sc protein, RNA encoding an IL-15 sushi protein, RNA encoding an IFNα protein, and RNA encoding a GM-CSF protein to a subject that has an anti-PD-1 and/or anti-PD-L1 resistant solid tumor cancer.

Encompassed herein are methods of treating a subject having a solid tumor cancer with acquired resistance to anti-PD-1 and/or anti-PD-L1 therapy comprising administering an effective amount of RNAs comprising RNA encoding an IL-12sc protein, RNA encoding an IL-15 sushi protein, RNA encoding an IFNα protein, and RNA encoding a GM-CSF protein to a subject that has a solid tumor cancer with acquired resistance to anti-PD-1 and/or anti-PD-L1 therapy.

In some embodiments, methods of treating a subject having a solid tumor cancer with innate resistance to anti-PD-1 and/or anti-PD-L1 therapy are provided, comprising administering an effective amount of RNAs comprising RNA encoding an IL-12sc protein, RNA encoding an IL-15 sushi protein, RNA encoding an IFNα protein, and RNA encoding a GM-CSF protein to a subject that has a solid tumor cancer with innate resistance to anti-PD-1 and/or anti-PD-L1 therapy.

Embodiments provided herein are not limited by any scientific theory regarding intolerance, resistance, or refraction.

In some embodiments, the intolerance, resistance, refraction (including acquired and innate resistance) to an anti-PD-1 and/or anti-PD-1 therapy results from a cancer cell comprising a partial or total loss of beta-2-microglobulin (B2M) function. In some embodiments, a subject has a cancer cell comprising a partial or total loss of beta-2-microglobulin (B2M) function. In some embodiments, the cancer cell has a partial loss of B2M function. In some embodiments, the cancer cell has a total loss of B2M function. In some embodiments, the partial or total loss of B2M function is assessed by comparing a cancer cell to a non-cancer cell from the same subject, optionally wherein the non-cancer cell is from the same tissue from which the cancer cell was derived. In some embodiments, the cancer cell is in a solid tumor that comprises cancer cells with normal B2M function. In some embodiments, the cancer cell is in a solid tumor in which 25% or more of the cancer cells have a partial or total loss in B2M function. In some embodiments, the cancer cell is in a solid tumor in which 50% or more of the cancer cells have a partial or total loss in B2M function. In some embodiments, the cancer cell is in a solid tumor in which 75% or more of the cancer cells have a partial or total loss in B2M function. In some embodiments, the cancer cell is in a solid tumor in which 95% or more of the cancer cells have a partial or total loss in B2M function. In some embodiments, the solid tumor as a whole (e.g., as assessed in a biopsy taken from the solid tumor) has a partial or total loss of B2M function compared to normal cells or tissue from which the solid tumor is derived. In some embodiments, the subject comprises (e.g., the partial or total loss of function results from) a mutation in the B2M gene. The mutation may be a substitution, insertion, or deletion. In some embodiments, the B2M gene comprises a loss of heterozygosity (LOH).

In some embodiments, the mutation is a frameshift mutation. In some embodiments, the frameshift mutation is in exon 1 of B2M. In some embodiments, the frameshift mutation comprises p.Leu13fs and/or p.Ser14fs. In some embodiments, the subject has a reduced level of B2M protein as compared to a subject without a partial or total loss of B2M function.

In some instances, the solid tumor (e.g., cancer cells within the solid tumor) have a reduced level of cell-surface expressed (also referred to herein as “surface expressed”) major histocompatibility complex class I (MHC I). In some embodiments, a solid tumor sample (e.g., a biopsy comprising cancer cells of the solid tumor) has a reduced level of cell-surface expressed MHC I as compared to a control, optionally wherein the control is a corresponding non-cancerous sample from the same subject. In some embodiments, the level of MHC I expressed on the surface of cancer cells in the solid tumor is reduced as a result of a mutation in a B2M gene. In some embodiments, a subject has a cancer cell comprising a reduced level of surface expressed MHC I. In some embodiments, the cancer cell has no surface expressed MHC I. In some embodiments, the reduced level of surface expressed MHC I is assessed by comparing a cancer cell to a non-cancer cell from the same subject, optionally wherein the non-cancer cell is from the same tissue from which the cancer cell was derived. In some embodiments, the cancer cell is in a solid tumor that comprises cancer cells with a normal level of surface expressed MHC 1. In some embodiments, the cancer cell is in a solid tumor in which 25% or more of the cancer cells have a reduced level of surface expressed MHC I. In some embodiments, the cancer cell is in a solid tumor in which 50% or more of the cancer cells have a reduced level of surface expressed MHC I. In some embodiments, the cancer cell is in a solid tumor in which 75% or more of the cancer cells have a reduced level of surface expressed MHC I. In some embodiments, the cancer cell is in a solid tumor in which 95% or more of the cancer cells have a reduced level of surface expressed MHC I. In some embodiments, the solid tumor as a whole (e.g., as assessed in a biopsy taken from the solid tumor) has a reduced level of surface expressed MHC I compared to normal cells or tissue from which the solid tumor is derived.

In some embodiments, methods for treating a subject having an advanced-stage, unresectable, or metastatic solid tumor cancer are provided comprising administering an effective amount of RNAs comprising RNA encoding an IL-12sc protein, RNA encoding an IL-15 sushi protein, RNA encoding an IFNα protein, and RNA encoding a GM-CSF protein to a subject that has an advanced-stage, unresectable, or metastatic solid tumor cancer.

In some embodiments, the subject has failed, or become intolerant, resistant, or refractory to an anti-programmed cell death 1 (PD-1) therapy. In some embodiments, the subject has failed, or become intolerant, resistant, or refractory to an anti-programmed cell death 1 ligand (PD-L1) therapy.

In some embodiments, the subject has failed an anti-programmed cell death 1 (PD-1) therapy or anti-programmed cell death 1 ligand (PD-L1) therapy.

In some embodiments, the subject has become intolerant to an anti-programmed cell death 1 (PD-1) or anti-programmed cell death 1 ligand (PD-L1) therapy.

In some embodiments, the subject has become resistant to an anti-programmed cell death 1 (PD-1) and/or anti-programmed cell death 1 ligand (PD-L1) therapy.

In some embodiments, the subject has become refractory to an anti-programmed cell death 1 (PD-1) or anti-programmed cell death 1 ligand (PD-L1) therapy. In some embodiments, the refractory or resistant cancer is one that does not respond to a specified treatment. In some embodiments, the refraction occurs from the very beginning of treatment. In some embodiments, the refraction occurs during treatment.

In some embodiments, the cancer is resistant before treatment begins.

In some embodiments, the subject has a cancer that does not respond to the anti-programmed cell death 1 (PD-1) and/or anti-programmed cell death 1 ligand (PD-L1) therapy. In some embodiments, the subject has a cancer that is becoming refractory or resistant to a specified treatment. In some embodiments, the specified treatment is as an anti-PD1 therapy. In some embodiments, the specified treatment is as an anti-PD-L1 therapy. In some embodiments, the subject has become less responsive to the therapy since first receiving it. In some embodiments, the subject has not received the therapy, but has a type of cancer that does not typically respond to the therapy.

In some embodiments, the subject is human.

In some embodiments, the subject has not been treated previously with an anti-PD-1 or anti-PD-L1 therapy. In some embodiments, the solid tumor cancer is one in which an anti-PD-1 or anti-PD-L1 therapy is not routinely used.

In some embodiments, the subject has a metastatic solid tumor. In some embodiments, the subject has a non-metastatic solid tumor. In some embodiments, the subject has an unresectable solid tumor. In some embodiments, the subject has a metastatic and unresectable solid tumor. In some embodiments, the subject has a non-metastatic and unresectable solid tumor.

In some embodiments, the solid tumor is an epithelial tumor, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, melanoma tumor, small cell lung tumor, neuroblastoma tumor, testicular tumor, carcinoma tumor, adenocarcinoma tumor, seminoma tumor, retinoblastoma, cutaneous squamous cell carcinoma (CSCC), squamous cell carcinoma for the head and neck (HNSCC), head and neck cancer, osteosarcoma tumor, cutaneous squamous cell cancer (CSCC), non-small cell lung cancer, kidney tumor, thyroid tumor, liver tumor, or other solid tumors amenable to intratumoral injection.

In some embodiments, the solid tumor is a lymphoma, including Non-Hodgkin lymphoma or Hodgkin lymphoma.

In some embodiments, the solid tumor cancer is melanoma. In some embodiments, the melanoma is uveal melanoma or mucosal melanoma. In some embodiments, the solid tumor cancer is melanoma, optionally uveal melanoma or mucosal melanoma, and comprises superficial, subcutaneous and/or lymph node metastases amenable for intratumoral injection.

In some embodiments, intratumoral injection comprises injection into a solid tumor metastasis within a lymph node. In some embodiments, intratumoral injection comprises injection into a lymphoma tumor within a lymph node. In some embodiments, intratumoral injection comprises injection into a primary or secondary solid tumor that is within 10 cm of the subject's skin surface. In some embodiments, intratumoral injection comprises injection into a primary or secondary solid tumor that is within 5 cm of the subject's skin surface. In some embodiments, intratumoral injection comprises injection into a cutaneous solid tumor. In some embodiments, the cutaneous solid tumor is a metastasis. In some embodiments, the cutaneous solid tumor is a skin cancer. In some embodiments, the cutaneous solid tumor is not a skin cancer. In some embodiments, intratumoral injection comprises injection into a subcutaneous solid tumor. In some embodiments, the subcutaneous solid tumor is a metastasis. In some embodiments, the subcutaneous solid tumor is a skin cancer. In some embodiments, the subcutaneous solid tumor is not a skin cancer.

In some embodiments, the solid tumor is an epithelial tumor. In some embodiments, the solid tumor is a prostate tumor. In some embodiments, the solid tumor is an ovarian tumor. In some embodiments, the solid tumor is a renal cell tumor. In some embodiments, the solid tumor is a gastrointestinal tract tumor. In some embodiments, the solid tumor is a hepatic tumor. In some embodiments, the solid tumor is a colorectal tumor. In some embodiments, the solid tumor is a tumor with vasculature. In some embodiments, the solid tumor is a mesothelioma tumor. In some embodiments, the solid tumor is a pancreatic tumor. In some embodiments, the solid tumor is a breast tumor. In some embodiments, the solid tumor is a sarcoma tumor. In some embodiments, the solid tumor is a lung tumor. In some embodiments, the solid tumor is a colon tumor. In some embodiments, the solid tumor is a melanoma tumor. In some embodiments, the solid tumor is a small cell lung tumor. In some embodiments, the solid tumor is non-small cell lung cancer tumor. In some embodiments, the solid tumor is a neuroblastoma tumor. In some embodiments, the solid tumor is a testicular tumor. In some embodiments, the solid tumor is a carcinoma tumor. In some embodiments, the solid tumor is an adenocarcinoma tumor. In some embodiments, the solid tumor is a seminoma tumor. In some embodiments, the solid tumor is a retinoblastoma. In some embodiments, the solid tumor is a cutaneous squamous cell carcinoma (CSCC). In some embodiments, the solid tumor is a squamous cell carcinoma for the head and neck (HNSCC). In some embodiments, the solid tumor is HNSCC. In some embodiments, the solid tumor is head and neck cancer. In some embodiments, the solid tumor is an osteosarcoma tumor. In some embodiments, the solid tumor is kidney cancer. In some embodiments, the solid tumor is thyroid cancer. In some embodiments, the solid tumor is anaplastic thyroid cancer (ATC). In some embodiments, the solid tumor is liver cancer. In some embodiments, the solid tumor is a colon tumor. In some embodiments, the solid tumor is any two of the above. In some embodiments, the solid tumor is any two or more of the above.

In some embodiments, the solid tumor is lymphoma. In some embodiments, the solid tumor is Non-Hodgkin lymphoma. In some embodiments, the solid tumor is Hodgkin lymphoma. In some embodiments, the solid tumor lymphoma is not a central nervous system lymphoma.

In some embodiments, the solid tumor cancer is HNSCC. In some embodiments, the solid tumor cancer is mucosal melanoma with only mucosal sites. In some embodiments, the solid tumor cancer is HNSCC and mucosal melanoma with only mucosal sites.

In some embodiments, the solid tumor cancer is uveal melanoma or mucosal melanoma. In some embodiments, the solid tumor cancer is breast cancer. In some embodiments, the solid tumor cancer is breast sarcoma or triple negative breast cancer.

In some embodiments, the RNAs are administered as monotherapy.

In some embodiments, the subject has more than one solid tumor. In some instances, at least one tumor is resistant, refractory, or intolerant to PD-1 or PD-L1 therapy. In some embodiments, at least one tumor is resistant, refractory, or intolerant to PD-1 or PD-L1 therapy and at least one tumor is not. In some embodiments, where more than one solid tumor is present, both resistant and non-resistant tumors, if present, are successfully treated.

In some embodiments, the solid tumor cancer is stage III, subsets of stage III, stage IV, or subsets of stage IV. In some embodiments, the solid tumor cancer is stage IIIB, stage IIIC, or stage IV cancer.

In some embodiments, the solid tumor cancer is advanced-stage. In some embodiments, the solid tumor cancer is unresectable. In some embodiments, the solid tumor cancer is advanced-stage and unresectable.

In some embodiments, the solid tumor has spread from its origin to another site in the subject.

In some embodiments, the solid tumor cancer has one or more cutaneous or subcutaneous lesions. In some embodiments, the solid tumor cancer has metastasized. In some embodiments, the solid tumor cancer has metastasized, but is not a skin cancer.

In some embodiments, the subject is without other treatment options.

In some embodiments, the solid tumor cancer is one for which an anti-PD1 or anti-PD-L1 therapy is routinely used, but which has not been treated with the therapy yet.

In some embodiments, the solid tumor cancer is stage IIIB, IIIC, or unresectable stage IV melanoma that is resistant and/or refractory to anti-PD-1 or anti-PD-L1 therapy. In some embodiments, the solid tumor cancer comprises superficial or subcutaneous lesions and/or metastases.

In some embodiments, the subject has measurable disease according to the Response Evaluation Criteria in Solid Tumors (RECIST) 1.1 criteria. In some embodiments, the subject has a life expectancy of more than 3 months. In some embodiments, the subject is at least 18 years of age.

In some embodiments, the RNAs are injected intratumorally.

In some embodiments, the RNAs are injected intratumorally only at mucosal sites of the solid tumor cancer.

In some embodiments, the RNAs are administered for about 5 months. In some embodiments, the RNAs are administered once every week. In some embodiments, the RNAs are administered for a maximum of 52 weeks.

In some embodiments, the IFNα protein is an IFNα2b protein.

In some embodiments, the RNA encoding an IL-12sc protein comprises the nucleotide sequence of SEQ ID NO: 17 or 18, or a nucleotide sequence having at least 99%, 98%, 97%, 96%, 95%, 90%, 85%, or 80% identity to the nucleotide sequence of SEQ ID NO: 17 or 18; and/or the IL-12sc protein comprises the amino acid sequence of SEQ ID NO: 14, or an amino acid sequence having at least 99%, 98%, 97%, 96%, 95%, 90%, 85%, or 80% identity to the amino acid sequence of SEQ ID NO:14; and/or the RNA encoding an IL-12sc protein comprises a nucleotide sequence having at least 99%, 98%, 97%, 96%, 95%, 90%, 85%, or 80% identity to the p40 portion of IL-12sc (nucleotides 1-984 of SEQ ID NO: 17 or 18) and at least 99%, 98%, 97%, 96%, 95%, 90%, 85%, or 80% identity to the p30 portion of IL-12sc (nucleotides 1027-1623 of SEQ ID NO: 17 or 18) and further comprises nucleotides between the p40 and p35 portions encoding a linker polypeptide.

In some embodiments, the RNA encoding an IL-15 sushi protein comprises the nucleotide sequence of SEQ ID NO: 26, or a nucleotide sequence having at least 9′9%, 98%, 97%, 96%, 95%, 90%, 85%, or 80% identity to the nucleotide sequence of SEQ ID NO: 26; and/or the IL-15 sushi protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24; and/or the RNA encoding an IL-15 sushi protein comprises a nucleotide sequence having at least 99%, 98%, 97%, 96%, 95%, 90%, 85%, or 80% identity to the sushi domain of IL-15 receptor alpha (nucleotides 1-321 of SEQ ID NO: 26) and at least 99%, 98%, 97%, 96%, 95%, 90%, 85%, or 80% identity to mature IL-15 (nucleotides 382-729 of SEQ ID NO: 26) and optionally further comprises nucleotides between the sushi domain of IL-15 and the mature IL-15 encoding a linker polypeptide.

In some embodiments, the RNA encoding an IFNα protein comprises the nucleotide sequence of SEQ ID NO: 22 or 23, or a nucleotide sequence having at least 99%, 98%, 97%, 96%, 95%, 90%, 85%, or 80% identity to the nucleotide sequence of SEQ ID NO: 22 or 23 and/or the IFNα protein comprises the amino acid sequence of SEQ ID NO: 19, or an amino acid sequence having at least 99%, 98%, 97%, 96%, 95%, 90%, 85%, or 80% identity to the amino acid sequence of SEQ ID NO: 19.

In some embodiments, the RNA encoding a GM-CSF protein comprises the nucleotide sequence of SEQ ID NO: 29, or a nucleotide sequence having at least 99%, 98%, 97%, 96%, 95%, 90%, 85%, or 80% identity to the nucleotide sequence of SEQ ID NO: 29 and/or the GM-CSF protein comprises the amino acid sequence of SEQ ID NO: 27, or an amino acid sequence having at least 99%, 98%, 97%, 96%, 95%, 90%, 85%, or 80% identity to the amino acid sequence of SEQ ID NO: 27.

In some embodiments, at least one RNA comprises a modified nucleoside in place of at least one uridine. In some embodiments, at least one RNA comprises a modified nucleoside in place of each uridine. In some embodiments, each RNA comprises a modified nucleoside in place of at least one uridine. In some embodiments, each RNA comprises a modified nucleoside in place of each uridine. In some embodiments, the modified nucleoside is independently selected from pseudouridine (Ψ). N1-methyl-pseudouridine (m1Ψ), and 5-methyl-uridine (m5U). In some embodiments, at least one RNA comprises more than one type of modified nucleoside, wherein the modified nucleosides are independently selected from pseudouridine (p), N1-methyl-pseudouridine (m1Ψ), and 5-methyl-uridine (m5U). In some embodiments, the modified nucleoside is N1-methyl-pseudouridine (m1Ψ).

In some embodiments, at least one RNA comprises the 5′ cap m^{2,3′-O}-Gppp(m₁^{2′-O})ApG (also sometimes referred to as m₂^{7,3′-O}G(5′)ppp(5′)m^{2′-O}ApG). In some embodiments, each RNA comprises the 5′ cap m₂^{7,3′-O}Gppp(m₁^{2′-O})ApG (also sometimes referred to as m₂^{7,3′O}G(5′)ppp(5′)m^{2′-O} ApG).

In some embodiments, at least one RNA comprises a 5′ UTR comprising a nucleotide sequence selected from the group consisting of SEQ ID NOs: 4 and 6, or a nucleotide sequence having at least 99%, 98%, 97%, 96%, 95%, 90%, 85%, or 80% identity to a nucleotide sequence selected from the group consisting of SEQ ID NOs: 4 and 6. In some embodiments, each RNA comprises a 5′ UTR comprising a nucleotide sequence selected from the group consisting of SEQ ID NOs: 4 and 6, or a nucleotide sequence having at least 99%, 98%, 97%, 96%, 95%, 90%, 85%, or 80% identity to a nucleotide sequence selected from the group consisting of SEQ ID NOs: 4 and 6.

In some embodiments, at least one RNA comprises a 3′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8. In some embodiments, each RNA comprises a 3′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.

In some embodiments, at least one RNA comprises a poly-A tail. In some embodiments, each RNA comprises a poly-A tail. In some embodiments, the poly-A tail comprises at least 100 nucleotides. In some embodiments, the poly-A tail comprises or consists of the poly-A tail shown in SEQ ID NO: 30.

In some embodiments, one or more RNA comprises:
i. a 5′ cap comprising m27,3′-OGppp(m12′-O)ApG or 3-O-Me-m7G(5′)ppp(5′)G;
ii. a 5′ UTR comprising (i) a nucleotide sequence selected from the group consisting of SEQ ID NOs: 4 and 6, or (ii) a nucleotide sequence having at least 99%, 98%, 97%, 96%, 95%, 90%, 85%, or 80% identity to a nucleotide sequence selected from the group consisting of SEQ ID NOs: 4 and 6;
iii. a 3′ UTR comprising (i) the nucleotide sequence of SEQ ID NO: 8, or (ii) a nucleotide sequence having at least 99%, 98%, 97%, 96%, 95%, 90%, 85%, or 80% identity to the nucleotide sequence of SEQ ID NO:8; and
iv. a poly-A tail comprising at least 100 nucleotides.

In some embodiments, the poly-A tail comprises or consists of SEQ ID NO: 30.

In some embodiments, treating the solid tumor comprises reducing the size of a tumor or preventing cancer metastasis in a subject.

In some embodiments, the RNAs are administered at the same time. In some embodiments, the RNAs are administered via injection. In some embodiments, the RNAs are mixed together in liquid solution prior to injection.

Further embodiments of the present application are as follows:
Embodiment A 1. A composition comprising RNA encoding an IL-12sc protein, RNA encoding an IL-15 sushi protein, RNA encoding an IFNα protein, and RNA encoding a GM-CSF protein for use in treating a subject having a solid tumor cancer, wherein the subject has failed, or become intolerant, resistant, or refractory to an anti-programmed cell death 1 (PD-1) or anti-programmed cell death 1 ligand (PD-L1) therapy.
Embodiment A 2. A composition comprising RNA encoding an IL-12sc protein for use in treating a subject that has failed, or become intolerant, resistant, or refractory to an anti-programmed cell death 1 (PD-1) or anti-programmed cell death 1 ligand (PD-L1) therapy, wherein the RNA is co-administered with RNA encoding an IL-15 sushi, RNA encoding an IFNα protein, and RNA encoding a GM-CSF protein.
Embodiment A 3. A composition comprising RNA encoding an IL-15 sushi protein for use in treating a subject that has failed, or become intolerant, resistant, or refractory to an anti-programmed cell death 1 (PD-1) or anti-programmed cell death 1 ligand (PD-L1) therapy, wherein the RNA is co-administered with RNA encoding an IL-12sc protein, RNA encoding an IFNα protein, and RNA encoding a GM-CSF protein.
Embodiment A 4. A composition comprising RNA encoding an IFNα protein for use in treating a subject that has failed, or become intolerant, resistant, or refractory to an anti-programmed cell death 1 (PD-1) or anti-programmed cell death 1 ligand (PD-L1) therapy, wherein the RNA is co-administered with RNA encoding an IL-12sc protein, RNA encoding an IL-15 sushi protein, and RNA encoding a GM-CSF protein.
Embodiment A 5. A composition comprising RNA encoding a GM-CSF protein for use in treating a subject that has failed, or become intolerant, resistant, or refractory to an anti-programmed cell death 1 (PD-1) or anti-programmed cell death 1 ligand (PD-L1) therapy, wherein the RNA is co-administered with RNA encoding an IL-12sc protein, RNA encoding an IL-15 sushi protein, and RNA encoding an IFNα protein.
Embodiment A 6. The composition of any one of embodiments A 1-5, wherein the subject has failed, or become intolerant, resistant, or refractory to an anti-programmed cell death 1 (PD-1) therapy.
Embodiment A 7. The composition of any one of embodiments A 1-6, wherein the subject has failed, or become intolerant, resistant, or refractory to an anti-programmed cell death 1 ligand (PD-L1) therapy.
Embodiment A 8. The composition of any one of embodiments A 1-7, wherein the subject has failed anti-programmed cell death 1 (PD-1) therapy or anti-programmed cell death 1 ligand (PD-L1) therapy.
Embodiment A 9. The composition of any one of embodiments A 1-8, wherein the subject has become intolerant to an anti-programmed cell death 1 (PD-1) or anti-programmed cell death 1 ligand (PD-L1) therapy.
Embodiment A 10. The composition of any one of embodiments A 1-9, wherein the subject has become resistant to an anti-programmed cell death 1 (PD-1) or anti-programmed cell death 1 ligand (PD-L1) therapy.
Embodiment A 11. The composition of any one of embodiments A 1-10, wherein the subject has become refractory to an anti-programmed cell death 1 (PD-1) or anti-programmed cell death 1 ligand (PD-L i) therapy.
Embodiment A 12. The composition of any one of embodiments A 1-11, wherein the refractory or resistant cancer is one that does not respond to a specified treatment.
Embodiment A 13. The composition of any one of embodiments A 1-12, wherein the refraction occurs from the very beginning of treatment.
Embodiment A 14. The composition of any one of embodiments A 1-13, wherein the refraction occurs during treatment.
Embodiment A 15. The composition of any one of embodiments A 1-14, wherein the cancer is resistant before treatment begins.
Embodiment A 16. The composition of any one of embodiments A 1-15, wherein the subject has a cancer that does not respond to the anti-programmed cell death 1 (PD-1) and/or anti-programmed cell death 1 ligand (PD-L1) therapy.
Embodiment A 17. The composition of any one of embodiments A 1-16, wherein the subject has a cancer that is becoming refractory or resistant to a specified treatment.
Embodiment A 18. The composition of embodiment A 17, wherein the specified treatment is as an anti-PD1 or anti-PD-L1 therapy.
Embodiment A 19. The composition of any one of embodiments A 1-18, wherein the subject has become less responsive to the therapy since first receiving it.
Embodiment A 20. The composition of any one of embodiments A 1-19, wherein the subject has not received the therapy, but has a type of cancer that does not typically respond to the therapy.
Embodiment A 21. The composition of any one of embodiments A 1-20, wherein the subject has anti-PD-1 and/or anti-PD-L1 resistant solid tumor cancer.
Embodiment A 22. The composition of any one of embodiments A 1-21, wherein the subject has a solid tumor cancer with acquired resistance to anti-PD-1 and/or anti-PD-L1 therapy.
Embodiment A 23. The composition of any one of embodiments A 1-22, wherein the subject has a solid tumor cancer with innate resistance to anti-PD-1 and/or anti-PD-L1 therapy.
Embodiment A 24. The composition of any one of embodiments A 1-23, wherein the subject has an advanced-stage, unresectable, or metastatic solid tumor cancer.
Embodiment A 25. The composition of any one of embodiments A 1-24, further comprising the initial step of selecting a subject that has failed, or become intolerant, resistant, or refractory to an anti-programmed cell death 1 (PD-1) or anti-programmed cell death 1 ligand (PD-L1) therapy.
Embodiment A 26. The composition of any one of embodiments A 1-25, wherein the subject is human.
Embodiment A 27. The composition of any one of embodiments A 1-26, wherein the subject has a metastatic solid tumor.
Embodiment A 28. The composition of any one of embodiments A 1-27, wherein the subject has an unresectable solid tumor.
Embodiment A 29. The composition of any one of embodiments A 1-28, wherein the subject has a cancer cell comprising a partial or total loss of beta-2-microglobulin (B2M) function.
Embodiment A 30. The composition of embodiments A 29, wherein the cancer cell has a partial loss of B2M function.
Embodiment A 31. The composition embodiments A 29, wherein the cancer cell has a total loss of B2M function.
Embodiment A 32. The composition of any one of embodiments A 1-31, wherein the partial or total loss of B2M function is assessed by comparing a cancer cell to a non-cancer cell from the same subject, optionally wherein the non-cancer cell is from the same tissue from which the cancer cell was derived.
Embodiment A 33. The composition of any one of embodiments A 1-32, wherein the subject comprises a mutation in the B2M gene.
Embodiment A 34. The composition of any one of embodiments A 1-33, wherein the mutation is a substitution, insertion, or deletion.
Embodiment A 35. The composition of any one of embodiments A 1-34, wherein the B2M gene comprises a loss of heterozygosity (LOH).
Embodiment A 36. The composition of any one of embodiments A 1-35, wherein the subject comprises a frameshift mutation.
Embodiment A 37. The composition of any one of embodiments A 1-36, wherein the subject comprises a frameshift mutation in exon 1 of B2M.
Embodiment A 38. The composition of any one of embodiments A 1-37, wherein the subject comprises a frameshift mutation comprising p.Leu13fs and/or p.Ser14fs Embodiment A 39. The composition of any one of embodiments A 1-38, wherein the subject has a reduced level of B2M protein as compared to a subject without a partial or total loss of B2M function.
Embodiment A 40. The composition of any one of embodiments A 1-39, wherein the subject has a reduced level of surface expressed major histocompatibility complex class I (MHC I) as compared to a control, optionally wherein the control is a non-cancerous sample from the same subject.
Embodiment A 41. The composition of any one of embodiments A 1-40, wherein the solid tumor cancer is an epithelial tumor, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, melanoma tumor, small cell lung tumor, non-small cell lung cancer, neuroblastoma tumor, testicular tumor, carcinoma tumor, adenocarcinoma tumor, seminoma tumor, retinoblastoma, cutaneous squamous cell carcinoma (CSCC), squamous cell carcinoma for the head and neck (HNSCC), head and neck cancer, osteosarcoma tumor, kidney tumor, thyroid tumor, anaplastic thyroid cancer (ATC), liver tumor, colon tumor, or other solid tumors amenable to intratumoral injection.
Embodiment A 42. The composition of any one of embodiments A 1-41, wherein the solid tumor cancer is melanoma.
Embodiment A 43. The composition of any one of embodiments A 1-42, wherein the solid tumor cancer is not melanoma.
Embodiment A 44. The composition of any one of embodiments A 1-42, wherein the solid tumor cancer is melanoma, and wherein the melanoma is uveal melanoma or mucosal melanoma.
Embodiment A 45. The composition of any one of embodiments A 1-43, wherein the solid tumor cancer is melanoma comprising superficial, subcutaneous and/or lymph node metastases amenable for intratumoral injection.
Embodiment A 46. The composition of embodiment 15, wherein the solid tumor cancer is HNSCC and/or mucosal melanoma with only mucosal sites.
Embodiment A 47. The composition of any one of embodiments A 1-46, wherein the RNAs are administered as monotherapy.
Embodiment A 48. The composition of any one of embodiments A 1-47, wherein the subject has more than one solid tumor.
Embodiment A 49. The composition of any one of embodiments A 1-48, wherein at least one tumor is resistant, refractory, or intolerant to an anti-PD-1 or anti-PD-L1 therapy and at least one tumor is not.
Embodiment A 50. The composition of embodiment A 49, wherein both resistant and non-resistant tumors are successfully treated.
Embodiment A 51. The composition of any one of embodiments A 1-50, wherein the solid tumor cancer is stage III, subsets of stage III, stage IV, or subsets of stage IV.
Embodiment A 52. The composition of any one of embodiments A 1-51, wherein the solid tumor cancer is advanced-stage and unresectable.
Embodiment A 53. The composition of any one of embodiments A 1-52, wherein the solid tumor has spread from its origin to another site in the subject.
Embodiment A 54. The composition of any one of embodiments A 1-53, wherein the solid tumor cancer has one or more cutaneous or subcutaneous lesions, optionally wherein the cancer is not a skin cancer.
Embodiment A 55. The composition of any one of embodiments A 1-54, wherein the solid tumor cancer is stage IIIB, stage RIC, or stage IV melanoma.
Embodiment A 56. The composition of any one of embodiments A 1-55, wherein the subject has not been treated previously with an anti-PD-1 or anti-PD-L1 therapy.
Embodiment A 57. The composition of any one of embodiments A 1-56, wherein the solid tumor cancer is one in which an anti-PD-1 or anti-PD-L1 therapy is not routinely used.
Embodiment A 58. The composition of any one of embodiments A 1-57, wherein the solid tumor cancer is not melanoma, non-small cell lung cancer, kidney cancer, head and neck cancer, breast cancer, or CSCC.
Embodiment A 59. The composition of any one of embodiments A 1-58, wherein the subject is without other treatment options.
Embodiment A 60. The composition of any one of embodiments A 1-59, wherein
   a. the solid tumor cancer is not melanoma, CSCC, or HNSCC; and
   b. an anti-PD-1 or anti-PD-L1 therapy is not routinely used; and
   c. there are no other suitable treatment options.
Embodiment A 61. The composition of any one of embodiments A 1-60, wherein the solid tumor cancer is one for which an anti-PD1 or anti-PD-L1 therapy is routinely used, but which has not been treated with the therapy yet.
Embodiment A 62. The composition of any one of embodiments A 1-61, wherein the solid tumor cancer is stage IIIB, IIIC, or unresectable stage IV melanoma that is resistant and/or refractory to anti-PD-1 or anti-PD-L1 therapy.
Embodiment A 63. The composition of any one of embodiments A 1-62, wherein the solid tumor cancer comprises superficial or subcutaneous lesions and/or metastases.
Embodiment A 64. The composition of any one of embodiments A 1-63, wherein the subject has two or three tumor lesions.
Embodiment A 65. The composition of any one of embodiments A 1-64, wherein the subject has measurable disease according to the Response Evaluation Criteria in Solid Tumors (RECIST) 1.1 criteria.
Embodiment A 66. The composition of any one of embodiments A 1-65, wherein the subject has a life expectancy of more than 3 months.
Embodiment A 67. The composition of any one of embodiments A 1-66, wherein the subject is at least 18 years of age.
Embodiment A 68. The composition of any one of the embodiments A 1-67, wherein
   a. the RNA encoding an IL-12sc protein comprises the nucleotide sequence of SEQ ID NO: 17 or 18, or a nucleotide sequence having at least 99%, 98%, 97%, 96%, 95%, 90%, 85%, or 80% identity to the nucleotide sequence of SEQ ID NO: 17 or 18; and/or
   b. the IL-12sc protein comprises the amino acid sequence of SEQ ID NO: 14, or an amino acid sequence having at least 99%, 98%, 97%, 96%, 95%, 90%, 85%, or 80% identity to the amino acid sequence of SEQ ID NO:14, and/or
   c. the RNA encoding an IL-12sc protein comprises a nucleotide sequence having at least 99%, 98%, 97%, 96%, 95%, 90%, 85%, or 80% identity to the p40 portion of IL-12sc (nucleotides 1-984 of SEQ ID NO: 17 or 18) and at least 99%, 98%, 97%, 96%, 95%, 90%, 85%, or 80% identity to the p30 portion of IL-12sc (nucleotides 1027-1623 of SEQ ID NO: 17 or 18) and further comprises nucleotides between the p40 and p35 portions encoding a linker polypeptide.
Embodiment A 69. The composition of any one of the embodiments A 1-68, wherein
   a. the RNA encoding an IL-15 sushi protein comprises the nucleotide sequence of SEQ ID NO: 26, or a nucleotide sequence having at least 99%, 98%, 97%, 96%, 95%, 90%, 85%, or 80% identity to the nucleotide sequence of SEQ ID NO: 26; and/or
   b. the IL-15 sushi protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24; and/or
   c. the RNA encoding an IL-15 sushi protein comprises a nucleotide sequence having at least 99%, 98%, 97%, 96%, 95%, 90%, 85%, or 80% identity to the sushi domain of IL-15 receptor alpha (nucleotides 1-321 of SEQ ID NO: 26) and at least 99%, 98%, 97%, 96%, 95%, 90%, 85%, or 80% identity to mature IL-15 (nucleotides 382-729 of SEQ ID NO: 26) and optionally further comprises nucleotides between the sushi domain of IL-15 and the mature IL-15 encoding a linker polypeptide.
Embodiment A 70. The composition of any one of the embodiments A 1-69, wherein
   a. the RNA encoding an IFNα protein comprises the nucleotide sequence of SEQ ID NO: 22 or 23, or a nucleotide sequence having at least 99%, 98%, 97%, 96%, 95%, 90%, 85%, or 80% identity to the nucleotide sequence of SEQ ID NO; 22 or 23 and/or
   b. the IFNα protein comprises the amino acid sequence of SEQ ID NO: 19, or an amino acid sequence having at least 99%, 98%, 97%, 96%, 95%, 90%, 85%, or 80% identity to the amino acid sequence of SEQ ID NO: 19.
Embodiment A 71. The composition of any one of embodiments A 1-70, wherein
   a. the RNA encoding a GM-CSF protein comprises the nucleotide sequence of SEQ ID NO: 29, or a nucleotide sequence having at least 99%, 98%, 97%, 96%, 95%, 90%, 85%, or 80% identity to the nucleotide sequence of SEQ ID NO; 29 and/or
   b. the GM-CSF protein comprises the amino acid sequence of SEQ ID NO: 27, or an amino acid sequence having at least 99%, 98%, 97%, 96%, 95%, 90%, 85%, or 80% identity to the amino acid sequence of SEQ ID NO: 27.
Embodiment A 72. The composition of any one of embodiments A 1-71, wherein at least one RNA comprises a modified nucleoside in place of at least one uridine.
Embodiment A 73. The composition of any one of the preceding embodiments A 1-72, wherein at least one RNA comprises a modified nucleoside in place of each uridine.
Embodiment A 74. The composition of any one of embodiments A 1-73, wherein each RNA comprises a modified nucleoside in place of at least one uridine.
Embodiment A 75. The composition of any one of embodiments A 1-74, wherein each RNA comprises a modified nucleoside in place of each uridine.
Embodiment A 76. The composition of any one of embodiments 72-75, wherein the modified nucleoside is independently selected from pseudouridine (ψ), N1-methyl-pseudouridine (m¹ψ), and 5-methyl-uridine (m⁵U).
Embodiment A 77. The composition of any one of embodiments A 1-76, wherein at least one RNA comprises more than one type of modified nucleoside, wherein the modified nucleosides are independently selected from pseudouridine (ψ), N1-methyl-pseudouridine (m¹ψ), and 5-methyl-uridine (m⁵U).
Embodiment A 78. The composition of embodiment A 77, wherein the modified nucleoside is N1-methyl-pseudouridine (m¹ψ).
Embodiment A 79. The composition of any one of embodiments A 1-78, wherein at least one RNA comprises the 5′ cap m₂^{7,3′-O}Gppp(m₁^{2′-O})ApG or 3′-O-Me-m⁷G(5′)ppp(5′)G.
Embodiment A 80. The composition of any one of embodiments A 1-79, wherein each RNA comprises the 5′ cap m₂^{7,3′-O}Gppp(m₁^{2′-O})ApG or 3′-O-Me-m⁷G(5′)ppp(5′)G.
Embodiment A 81. The composition of any one of embodiments A 1-80, wherein at least one RNA comprises a 5′ UTR comprising a nucleotide sequence selected from the group consisting of SEQ ID NOs: 4 and 6, or a nucleotide sequence having at least 99%, 98%, 97%, 96%, 95%, 90%, 85%, or 80% identity to a nucleotide sequence selected from the group consisting of SEQ ID NOs: 4 and 6.
Embodiment A 82. The composition of any one of embodiments A 1-81, wherein each RNA comprises a 5′ UTR comprising a nucleotide sequence selected from the group consisting of SEQ ID NOs: 4 and 6, or a nucleotide sequence having at least 99%, 98%, 97%, 96%, 95%, 90%, 85%, or 80% identity to a nucleotide sequence selected from the group consisting of SEQ ID NOs: 4 and 6.
Embodiment A 83. The composition of any one of embodiments A 1-82, wherein at least one RNA comprises a 3′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.
Embodiment A 84. The composition of any one of embodiments A 1-83, wherein each RNA comprises a 3′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.
Embodiment A 85. The composition of any one of embodiments A 1-84, wherein at least one RNA comprises a poly-A tail.
Embodiment A 86. The composition of any one of embodiments A 1-85, wherein each RNA comprises a poly-A tail.
Embodiment A 87. The composition of embodiment A 84 or A 85, wherein the poly-A tail comprises at least 100 nucleotides.
Embodiment A 88. The composition of any one of embodiments A 85-87, wherein the poly-A tail comprises the poly-A tail shown in SEQ ID NO: 30.
Embodiment A 89. The composition of any one of embodiments A 1-88, wherein one or more RNA comprises:
   a. a 5′ cap comprising m₂ 7 Gppp(m₁^{2′-O})ApG or 3-O-Me-m⁷G(5′)ppp(5′)G;
   b. a 5′ UTR comprising (i) a nucleotide sequence selected from the group consisting of SEQ ID NOs: 4 and 6, or (ii) a nucleotide sequence having at least 99%, 98%, 97%, 96%, 95%, 90%, 85%, or 80% identity to a nucleotide sequence selected from the group consisting of SEQ ID NOs: 4 and 6;
   c. a 3′ UTR comprising (i) the nucleotide sequence of SEQ ID NO: 8, or (ii) a nucleotide sequence having at least 99%, 98%, 97%, 96%, 95%, 90%, 85%, or 80% identity to the nucleotide sequence of SEQ ID NO:8; and
   d. a poly-A tail comprising at least 100 nucleotides.
Embodiment A 90. The composition of embodiment A 89, wherein the poly-A tail comprises SEQ ID NO: 30.
Embodiment A 91. The composition of any one of embodiments A 1-90, wherein the composition is used in treating an advanced-stage, unresectable, or metastatic solid tumor cancer in a human.
Embodiment A 92. The composition of any one of embodiments A 1-91, wherein treating the solid tumor comprises reducing the size of a tumor or preventing cancer metastasis in a subject.
Embodiment A 93. The composition of any one of embodiments A 1-92, wherein the RNAs are administered at the same time.
Embodiment A 94. The composition of any one of embodiments A 1-93, wherein the RNAs are administered via injection.
Embodiment A 95. The composition of embodiments A 93 or A 94, wherein the RNAs are mixed together in liquid solution prior to injection.
Embodiment A 96. The composition of any one of embodiments A1-A96, wherein the solid tumor cancer comprises lymphoma.
Embodiment A 97. The composition of any one of embodiments A1-A96, wherein the solid tumor cancer comprises Hodgkin lymphoma.
Embodiment A 98. The composition of any one of embodiments A1-A96, wherein the solid tumor cancer comprises Non-Hodgkin lymphoma.

Further embodiments of the present application are as follows:
Embodiment B 1. A method of treating advanced-stage, unresectable, or metastatic solid tumor cancer in a subject comprising administering an RNA encoding an IL-12sc protein, wherein the RNA is co-administered with RNA encoding an IL-15 sushi, RNA encoding an IFNα protein, and RNA encoding a GM-CSF protein.
Embodiment B 2. A method of treating advanced-stage, unresectable, or metastatic solid tumor cancer in a subject comprising administering an RNA encoding an IL-15 sushi protein, wherein the RNA is co-administered with RNA encoding an IL-12sc protein, RNA encoding an IFNα protein, and RNA encoding a GM-CSF protein.
Embodiment B 3. A method of treating advanced-stage, unresectable, or metastatic solid tumor cancer in a subject comprising administering RNA encoding an IFNα protein, wherein the RNA is co-administered with RNA encoding an IL-12sc protein, RNA encoding an IL-15 sushi protein, and RNA encoding a GM-CSF protein.
Embodiment B 4. A method of treating advanced-stage, unresectable, or metastatic solid tumor cancer in a subject comprising administering RNA encoding a GM-CSF protein, wherein the RNA is co-administered with RNA encoding an IL-12sc protein, RNA encoding an IL-15 sushi protein, and RNA encoding an IFNα protein.
Embodiment B 5. The method of any one of embodiments B 1-4, wherein the solid tumor cancer is stage III, subsets of stage III, stage IV, or subsets of stage IV.
Embodiment B 6. The method of any one of embodiments B 1-5, wherein the solid tumor cancer is advanced-stage and unresectable.
Embodiment B 7. The method of any one of embodiments B 1-6, wherein the solid tumor has spread from its origin to another site in the subject.
Embodiment B 8. The method of any one of embodiments B 1-7, wherein the solid tumor cancer is stage IIIB, stage IIIC, or stage IV cancer.
Embodiment B 9. The method of embodiment B 8, wherein the stage IV cancer is unresectable.
Embodiment B 10. The method of any one of embodiments B 1-9, w % herein the subject has failed, or become intolerant, resistant, or refractory to an anti-programmed cell death 1 (PD-1) or anti-programmed cell death 1 ligand (PD-L1) therapy.
Embodiment B 11. The method of any one of embodiments B 1-10, wherein the solid tumor cancer is melanoma, cutaneous squamous cell cancer (CSCC), squamous cell carcinoma of the head and neck (HNSCC), non-small cell lung cancer, kidney cancer, head and neck cancer, thyroid cancer, colon cancer, liver cancer, ovarian cancer, or breast cancer, or other solid tumors amenable to intratumoral injection.
Embodiment B 12. The method of any one of embodiments B 1-11, wherein the solid tumor cancer is melanoma
Embodiment B 13. The method of any one of embodiments B 1-11, wherein the solid tumor cancer is breast cancer (e.g., breast sarcoma, triple negative breast cancer).
Embodiment B 14. The method of any one of embodiments B 1-11, wherein the solid tumor cancer is ovarian cancer.
Embodiment B 15. The method of embodiment B 14, wherein the ovarian cancer is resistant to platinum-based chemotherapy.
Embodiment B 16. The method of any one of embodiments B 1-11, wherein the solid tumor cancer is thyroid cancer.
Embodiment B 17. The method of embodiment B 16, wherein the thyroid cancer is anaplastic thyroid cancer (ATC).
Embodiment B 18. The method of any one of embodiments B 1-11, wherein the solid tumor cancer has one or more cutaneous or subcutaneous lesions (e.g., metastasis), but is not a skin cancer.
Embodiment B 19. The method of any one of embodiments B 1-12, the solid tumor cancer is stage IIIB, stage IIIC, or stage IV melanoma.
Embodiment B 20. The method of any one of embodiments B 1-19, wherein the subject has not been treated previously with an anti-PD-1 or anti-PD-L1 therapy.
Embodiment B 21. The method of any one of embodiments B 1-20, wherein the solid tumor cancer is one in which an anti-PD-1 or anti-PD-L1 therapy is not routinely used.
Embodiment B 22. The method of any one of embodiments B 1-21, wherein the solid tumor cancer is not melanoma, non-small cell lung cancer, kidney cancer, head and neck cancer, breast cancer, or CSCC.
Embodiment B 23. The method of any one of embodiments B 1-22, wherein the subject is without other treatment options.
Embodiment B 24. The method of any one of embodiments B 1-23, wherein
   a. the solid tumor cancer is not melanoma, CSCC, or HNSCC; and
   b. an anti-PD-1 or anti-PD-L1 therapy is not routinely used; and
   c. there are no other suitable treatment options.
Embodiment B 25. The method of any one of embodiments B 1-24, wherein the solid tumor cancer is one for which an anti-PD1 or anti-PD-L1 therapy is routinely used, but which has not been treated with the therapy yet.
Embodiment B 26. The method of any one of embodiments B 1-25, wherein the solid tumor cancer is stage IIIB, IIIC, or unresectable stage IV melanoma that is resistant and/or refractory to anti-PD-1 or anti-PD-L1 therapy.
Embodiment B 27. The method of any one of embodiments B 1-26, wherein the solid tumor cancer comprises superficial or subcutaneous lesions and/or metastases.
Embodiment B 28. The method of any one of embodiments B 1-27, wherein the solid tumor cancer is an epithelial tumor, HNSCC, CSCC, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, melanoma, small cell lung tumor, neuroblastoma tumor, testicular tumor, carcinoma tumor, adenocarcinoma tumor, seminoma tumor, retinoblastoma, or osteosarcoma tumor.
Embodiment B 29. The method of any one of embodiments B 1-28, wherein the subject has two or three tumor lesions.
Embodiment B 30. The method of any one of embodiments B 1-29, wherein the subject has measurable disease according to the Response Evaluation Criteria in Solid Tumors (RECIST) 1.1 criteria.
Embodiment B 31. The method of any one of embodiments B 1-30, w % herein the subject has a life expectancy of more than 3 months.
Embodiment B 32. The method of any one of embodiments B 1-31, wherein the subject is at least 18 years of age.
Embodiment B 33. The method of any one of embodiments B 1-32, wherein the RNAs are injected intratumorally.
Embodiment B 34. The method of any one of embodiments B 1-33, wherein the RNAs are administered as monotherapy.
Embodiment B 35. The method of any one of embodiments B 1-34, wherein the RNAs are administered for about 5 months.
Embodiment B 36. The method of any one of embodiments B 1-35, wherein the RNAs are administered once every week.
Embodiment B 37. The method of any one of embodiments B 1-36, wherein the RNAs are administered for a maximum of 52 weeks.
Embodiment B 38. The method of any one of embodiments B 1-37, w % herein the IFNα protein is an IFNα2b protein.
Embodiment B 39. The method of any one of embodiments B 1-38, wherein
   a. the RNA encoding an IL-12sc protein comprises the nucleotide sequence of SEQ ID NO: 17 or 18, or a nucleotide sequence having at least 99%, 98%, 97%, 96%, 95%, 90%, 85%, or 80% identity to the nucleotide sequence of SEQ ID NO: 17 or 18; and/or
   b. the IL-12sc protein comprises the amino acid sequence of SEQ ID NO: 14, or an amino acid sequence having at least 99%, 98%, 97%, 96%, 95%, 90%, 85%, or 80% identity to the amino acid sequence of SEQ ID NO:14; and/or
   c. the RNA encoding an IL-12sc protein comprises a nucleotide sequence having at least 99%, 98%, 97%, 96%, 95%, 90%, 85%, or 80% identity to the p40 portion of IL-12sc (nucleotides 1-984 of SEQ ID NO: 17 or 18) and at least 99%, 98%, 97%, 96%, 95%, 90%, 85%, or 80% identity to the p30 portion of IL-12sc (nucleotides 1027-1623 of SEQ ID NO: 17 or 18) and further comprises nucleotides between the p40 and p35 portions encoding a linker polypeptide.
Embodiment B 40. The method of any one of embodiments B 1-39, wherein
   d. the RNA encoding an IL-15 sushi protein comprises the nucleotide sequence of SEQ ID NO: 26, or a nucleotide sequence having at least 99%, 98%, 97%, 96%, 95%, 90%, 85%, or 80% identity to the nucleotide sequence of SEQ ID NO: 26; and/or
   e. the IL-15 sushi protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24; and/or
   f. the RNA encoding an IL-15 sushi protein comprises a nucleotide sequence having at least 99%, 98%, 97%, 96%, 95%, 90%, 85%, or 80% identity to the sushi domain of IL-15 receptor alpha (nucleotides 1-321 of SEQ ID NO: 26) and at least 99%, 98%, 97%, 96%, 95%, 90%, 85%, or 80% identity to mature IL-15 (nucleotides 382-729 of SEQ ID NO: 26) and optionally further comprises nucleotides between the sushi domain of IL-15 and the mature IL-15 encoding a linker polypeptide.
Embodiment B 41. The method of any one of embodiments B 1-40, wherein
   c. the RNA encoding an IFNα protein comprises the nucleotide sequence of SEQ ID NO: 22 or 23, or a nucleotide sequence having at least 99%, 98%, 97%. %6%, 95%, 90%, 85%, or 80% identity to the nucleotide sequence of SEQ ID NO; 22 or 23 and/or
   d. the IFNα protein comprises the amino acid sequence of SEQ ID NO: 19, or an amino acid sequence having at least 99%, 98%, 97%, 96%, 95%, 90%, 85%, or 80% identity to the amino acid sequence of SEQ ID NO: 19.
Embodiment B 42. The method of any one of embodiments B 1-41, wherein
   c. the RNA encoding a GM-CSF protein comprises the nucleotide sequence of SEQ ID NO: 29, or a nucleotide sequence having at least 99%, 98%, 97%, 96%, 95%, 90%, 85%, or 80% identity to the nucleotide sequence of SEQ ID NO; 29 and/or
   d. the GM-CSF protein comprises the amino acid sequence of SEQ ID NO: 27, or an amino acid sequence having at least 99%, 98%, 97%, 96%, 95%, 90%, 85%, or 80% identity to the amino acid sequence of SEQ ID NO: 27.
Embodiment B 43. The method of any one of embodiments B 1-42, wherein at least one RNA comprises a modified nucleoside in place of at least one uridine.
Embodiment B 44. The method of any one of embodiments B 1-43, wherein at least one RNA comprises a modified nucleoside in place of each uridine.
Embodiment B 45. The method of any one of embodiments B 1-44, w % herein each RNA comprises a modified nucleoside in place of at least one uridine.
Embodiment B 46. The method of any one of embodiments B 1-45, wherein each RNA comprises a modified nucleoside in place of each uridine.
Embodiment B 47. The method of any one of embodiments B 1-46, wherein the modified nucleoside is independently selected from pseudouridine (ψ), N1-methyl-pseudouridine (m¹ψ), and 5-methyl-uridine (m⁵U).
Embodiment B 48. The method of any one of embodiments B 1-47, wherein at least one RNA comprises more than one type of modified nucleoside, wherein the modified nucleosides are independently selected from pseudouridine (ψ), N1-methyl-pseudouridine (m¹ψ), and 5-methyl-uridine (m⁵U).
Embodiment B 49. The method of embodiment B 48, wherein the modified nucleoside is N1-methyl-pseudouridine (m¹ψ).
Embodiment B 50. The method of any one of embodiments B 1-49, wherein at least one RNA comprises the 5′ cap m₂^{7,3′-O}Gppp(m₁^{2′-O})ApG or 3′-O-Me-m⁷G(5′)ppp(5′)G.
Embodiment B 51. The method of any one of embodiments B 1-50, wherein each RNA comprises the 5′ cap m₂^{7,3′-O}Gppp(m₁^{2′-O})ApG or 3′-O-Me-m⁷G(5′)ppp(5′)G.
Embodiment B 52. The method of any one of embodiments B 1-51, wherein at least one RNA comprises a 5′ UTR comprising a nucleotide sequence selected from the group consisting of SEQ ID NOs: 4 and 6, or a nucleotide sequence having at least 99%, 98%, 97%, 96%, 95%, 90)%, 85%, or 80% identity to a nucleotide sequence selected from the group consisting of SEQ ID NOs: 4 and 6.
Embodiment B 53. The method of any one of embodiments B 1-52, wherein each RNA comprises a 5′ UTR comprising a nucleotide sequence selected from the group consisting of SEQ ID NOs: 4 and 6, or a nucleotide sequence having at least 99%, 98%, 97%, 96%, 95%, 90%, 85%, or 80% identity to a nucleotide sequence selected from the group consisting of SEQ ID NOs: 4 and 6.
Embodiment B 54. The method of any one of embodiments B 1-53, wherein at least one RNA comprises a 3′ UTR comprising the nucleotide sequence of SEQ ID NO: 8, or a nucleotide sequence having at least 99%, 98%, 97%. %%, 95%, 90%, 85%, or 80% identity to the nucleotide sequence of SEQ ID NO: 8.
Embodiment B 55. The method of any one of embodiments B 1-54, wherein each RNA comprises a 3′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.
Embodiment B 56. The method of any one of embodiments B 1-55, wherein at least one RNA comprises a poly-A tail.
Embodiment B 57. The method of any one of embodiments B 1-56, wherein each RNA comprises a poly-A tail.
Embodiment B 58. The method of embodiment B 56 or B 57, wherein the poly-A tail comprises at least 100 nucleotides.
Embodiment B 59. The method of any one of embodiments B 56-58, wherein the poly-A tail comprises the poly-A tail shown in SEQ ID NO: 30.
Embodiment B 60. The method of any one of embodiments B 1-59, wherein one or more RNA comprises:
   a. a 5′ cap comprising m₂^{7,3′-O}Gppp(m₁^{2′-O})ApG or 3′-O-Me-m⁷G(5′)ppp(5′)G;
   b. a 5′ UTR comprising (i) a nucleotide sequence selected from the group consisting of SEQ ID NOs: 4 and 6, or (ii) a nucleotide sequence having at least 99%, 98%, 97%, 96%, 95%, 90%, 85%, or 80% identity to a nucleotide sequence selected from the group consisting of SEQ ID NOs: 4 and 6;
   c. a 3′ UTR comprising (i) the nucleotide sequence of SEQ ID NO: 8, or (ii) a nucleotide sequence having at least 99%, 98%, 97%, 96%, 95%, 90%, 85%, or 80% identity to the nucleotide sequence of SEQ ID NO:8; and
   d. a poly-A tail comprising at least 100 nucleotides.
Embodiment B 61. The method of embodiment B 60, wherein the poly-A tail comprises SEQ ID NO: 30.
Embodiment B 62. The method of any one of embodiments B 1-61, wherein the subject is human.
Embodiment B 63. The method of any one of embodiments B 1-62, wherein treating the solid tumor comprises reducing the size of a tumor or preventing cancer metastasis in a subject.
Embodiment B 64. The method of any one of embodiments B 1-63, wherein the RNAs are administered at the same time.
Embodiment B 65. The method of any one of embodiments B 1-64, wherein the RNAs are administered via injection.
Embodiment B 66. The method of any one of embodiments B 1-65, wherein the RNAs are mixed together in liquid solution prior to injection.

### FIGURE LEGENDS

FIG. 1A shows an exemplary overall design of treatment.

FIG. 1B shows an exemplary treatment schedule for administration of the cytokine RNA mixture, for treating a patient having an advanced stage solid tumor cancer, including dose escalation and dose expansion of the cytokine RNA mixture. The cytokine RNA mixture is administered intratumorally as monotherapy.

FIGS. 2A-2I show the creation and characterization of a murine model of acquired resistance to anti-PD-1 therapy. FIGS. 2A-2B show the generation of a PD-1 resistant tumor line. FIG. 2A is a diagram of in vivo passaging approach. Briefly, C57BL6 mice bearing MC38 tumors were treated with anti-PD-1 antibody (clone RMP1-14), growing tumors were excised, and cells from the tumors were cultured ex vivo prior to implantation into naïve mice. FIG. 2B shows tumor growth curves for MC38 and MC38-resistant tumor cell lines implanted in C57BL6/J mice treated with 10 mg/kg anti-PD-1 antibody (n=5/group). Antibody treatments were administered as indicated by arrows. FIGS. 2C-2E show that MC38-resistant cells do not exhibit known molecular mechanisms of PD-1 resistance. MC38 and MC38-resistant cells were cultured in vitro and expression of different proteins was assayed by flow cytometry. FIG. 2C is a series of graphs showing surface expression of PD-L1, B2M and IFNGR1 and IFNGR2. Line, unstained; filled, stained sample. FIG. 2D is a graph showing PD-L1 expression following IFNγ treatment in vitro. FIG. 2E is a graph showing expression of SIINFEKL-MHC I complex in OVA-transduced cells. Cells were transduced to express ovalbumin and assayed for presentation of SIINFEKL in MHC I. FIGS. 2F-2I show subcutaneous tumors excised and profiled by RNA-sequencing. FIG. 2F shows global gene expression of many genes dysregulated in resistant tumors (n=13) compared to parental MC38 (n=16). FIG. 2G shows expression of IFNγ target genes is reduced in MC38-resistant tumors. FIG. 2H shows MCPCounter analysis estimating relative immune abundance, revealing significantly reduced T, NK, B cell lineage and monocytic lineage cells. *, p<0.05. FIG. 2I shows immune infiltration by flow cytometry in CD8⁺ T cells (CD45⁺CD3⁺CD4-CD8), CD4⁺ T cells (CD45⁺CD3⁺CD4⁺CD8⁻), macrophages (CD45⁺CD11b⁺F4/80⁺) and natural killer cells (CD45⁺CD3⁻CD49b⁺NK1.1⁺). Results are representative of two independent experiments, n=9 per group. * indicates p<0.05, **p<0.01, ***<0.001 and ****p<0.0001.

FIG. 3 shows that MC38-resistant cells do not express PD-L2. MC38 and MC38-resistant cells were cultured in vitro and expression of different proteins was assayed by flow cytometry. PD-L2 expression following IFNγ treatment is shown.

FIGS. 4A-4B show reduced frequency of immune cells in resistant tumors by immunohistochemical staining. Paraffin embedded MC38 and MC38-resistant tumors were analyzed by immunohistochemical staining for infiltration of CD45⁺ cells (dark color) Results are representative of two independent experiments; n=10 tumors per group. FIG. 4A shows representative images. FIG. 4B shows quantification.

FIGS. 5A-5B show reduced immunogenicity of resistant tumors. Cytotoxic T lymphocyte (CTL) cultures were generated from 5 individual C57BL6 mice bearing parental MC38 tumors that exhibited complete regression in response to PD-1 blockade. CTLs were co-cultured with MC38 and resistant tumor cells, and killing (FIG. 4A) and IFNγ release (FIG. 5B) were measured.

FIGS. 6A-6D show that C57BL6/J mice bearing subcutaneous MC38 or MC38-resistant tumors were successfully treated with intratumoral injection of cytokine RNA mixture (FIGS. 6B and 6D) as measured by tumor burden. mRNA treatments were administered every four days (as indicated by arrows) at a dose of 40 μg total mRNA. “Luc” (FIGS. 6A and 6C) indicates luciferase control mRNA.

FIG. 7 shows that C57BL6/J mice bearing subcutaneous MC38 or MC38-resistant tumors were successfully treated with intratumoral injection of cytokine RNA mixture as measured by overall survival. mRNA treatments were administered every four days (as indicated by arrows) at a dose of 40 μg total mRNA. “Luc” indicates luciferase control mRNA.

FIGS. 8A-8B shows flow cytometry analysis of beta-2 microglobulin (B2M) surface expression in MC38 (FIG. 8A) and MC38 with deletion of B2M (FIG. 8B).

FIGS. 9A-9D show that a combination of the cytokine RNA mixture with anti-PD-1 antibody enhanced survival in a dual flank B16F10 cancer model (FIG. 9A) and MC38 tumor model (FIG. 9B). Overall survival in single flank MC38-B2M knockout treated with cytokine RNA mixture (FIG. 9C) or a heterologous dual flank model with MC38-B2M knockout/MC38-WT tumors (FIG. 9D).

FIG. 10 shows changes in tumor volume after cytokine mRNA mixture, anti-PD-1, or a combination of cytokine mRNA mixture and anti-PD-1 therapy in various in vivo solid tumor cancer models. Numerical values correspond to tumor volume changes from baseline (ΔT/ΔC, %). Changes in tumor volume for each treated (T) and vehicle control (C) group are calculated for each animal by subtracting the tumor volume on the day of first treatment from the tumor volume on the last day when all the control mice were still alive. The median ΔT is calculated for the treated group, and the median ΔC is calculated for the vehicle control group. The ratio ΔT/AC is calculated and expressed as percentage.

FIG. 11 shows a “peri-tumorally.” or “peri-tumoral,” area that is about 2-mm wide and is adjacent to the invasive front of the tumor periphery. The peri-tumoral area comprises host tissue.

### DESCRIPTION OF THE SEQUENCES

Table 1 provides a listing of certain sequences referenced herein.

TABLE 1
DESCRIPTION OF THE SEQUENCES

| SEQ | | |
|---|---|---|
| ID | | |
| NO: | Description | SEQUENCE |
| 5′ UTR | | |
| 1 | Not used | |
| 2 | Not used | |
| 3 | 5′ UTR | GGAATAAACTAGTCTCAACACAACATATACAAAACAAACGAATCTCAAGCAATCAAGCATTCTACTTCTATTGCAGCAATTTAAATCA |
| | (DNA) | TTTCTTTTAAAGCAAAAGCAATTTTCTGAAAATTTTCACCATTTACGAACGATAGCC |
| 4 | 5′ UTR | GGAAUAAACUAGUCUCAACACAACAUAUACAAAACAAACGAAUCUCAAGCAAUCAAGCAUUCUACUUCUAUUGCAGCAAUUUAAAUCA |
| | (RNA) | UUUCUUUUAAAGCAAAAGCAAUUUUCUGAAAAUUUUCACCAUUUACGAACGAUAGCC |
| 5 | Alternative | AGACGAACTAGTATTCTTCTGGTCCCCACAGACTCAGAGAGAACCCGCCACC |
| | Mod 5′ UTR | |
| | (DNA) | |
| 6 | Alternative | AGACGAACUAGUAUUCUUCUGGUCCCCACAGACUCAGAGAGAACCCGCCACC |
| | Mod 5′ UTR | |
| | (RNA) | |
| 3′ UTR | | |
| 7 | 3′ UTR | CTCGAGCTGGTACTGCATGCACGCAATGCTAGCTGCCCCTTTCCCGTCCTGGGTACCCCGAGTCTCCCCCGACCTCGGGTCCCAGGTA |
| | (DNA) | TGCTCCCACCTCCACCTGCCCCACTCACCACCTCTGCTAGTTCCAGACACCTCCCAAGCACGCAGCAATGCAGCTCAAAACGCTTAGC |
| | | CTAGCCACACCCCCACGGGAAACAGCAGTGATTAACCTTTAGCAATAAACGAAAGTTTAACTAAGCTATACTAACCCCAGGGTTGGTC |
| | | AATTTCGTGCCAGCCACACCGAGACCTGGTCCAGAGTCGCTAGCCGCGTCGCT |
| 8 | 3′ UTR | CUCGAGCUGGUACUGCAUGCACGCAAUGCUAGCUGCCCCUUUCCCGUCCUGGGUACCCCGAGUCUCCCCCGACCUCGGGUCCCAGGUA |
| | (RNA) | UGCUCCCACCUCCACCUGCCCCACUCACCACCUCUGCUAGUUCCAGACACCUCCCAAGCACGCAGCAAUGCAGCUCAAAACGCUUAGC |
| | | CUAGCCACACCCCCACGGGAAACAGCAGUGAUUAACCUUUAGCAAUAAACGAAAGUUUAACUAAGCUAUACUAACCCCAGGGUUGGUC |
| | | AAUUUCGUGCCAGCCACACCGAGACCUGGUCCAGAGUCGCUAGCCGCGUCGCU |
| 9-13 | Not used | |
| IL-12sc | | |
| 14 | Human IL- | MCHQQLVISWFSLVFLASPLVAINELKRDVYVVELDWYPDAPGEMVVLTCDTPEEDGITWTLDQSSEVLGSGKTLTIQVREFGDAGQY |
| | 12sc (amino | TCHKGGEVLSHSLLLLHKKEDGIWSTDILKDQKEPENKTFLRCEAKNYSGRFTCWWLTTISTDLTFSVESSRGSSDPQGVTCGAATLS |
| | acid) | AERVRGDNKEYEYSVECQEDSACPAAEESLPIEVMVDAVHKLKYENYTSSFFIRDIIKPDPPKNLQLKPLKNSRQVEVSWEYPDTWST |
| | | PHSYFSLTFCVQVQGKSKREEKDRVETDKTSATVICRENASISVRAQDRYYSSSWSEWASVPCSGSSGGGGSPGGGSSRNLPVATPDP |
| | | GMFPCLHHSQNLLRAVSNMLQKARQTLEFYPCTSEEIDHEDITKDKTSTVEACLPLELTKNESCLNSRETSFITNGSCLASRRTSEMM |
| | | ALCLSSIYEDLKMYQVEFKTMNAKLLMDPKRQIFLDQNMLAVIDELMQALNFNSETVPQKSSLEEPDFYKTKIKLCILLHAFRIRAVT |
| | | IDRVMSYLNAS |
| 15 | Human non- | ATGTGTCACCAGCAGTTGGTCATCTCTTGGTTTTCCCTGGTTTTTCTGGCATCTCCCCTCGTGGCCATATGGGAACTGAAGAAAGATG |
| | optimized | TTTATGTCGTAGAATTGGATTGGTATCCGGATGCCCCTGGAGAAATGGTGGTCCTCACCTGTGACACCCCTGAAGAAGATGGTATCAC |
| | IL-12sc | CTGGACCTTGGACCAGAGCAGTGAGGTCTTAGGCTCTGGCAAAACCCTGACCATCCAAGTCAAAGAGTTTGGAGATGCTGGCCAGTAC |
| | (CDS DNA) | ACCTGTCACAAAGGAGGCGAGGTTCTAAGCCATTCGCTCCTGCTGCTTCACAAAAAGGAAGATGGAATTTGGTCCACTGATATTTTAA |
| | Seauence | AGGACCAGAAAGAACCCAAAAATAAGACCTTTCTAAGATGCGAGGCCAAGAATTATTCTGGACGTTTCACCTGCTGGTGGCTGACGAC |
| | annotation | AATCAGTACTGATTTGACATTCAGTGTCAAAAGCAGCAGAGGGTCTTCTGACCCCCAAGGGGTGACGTGCGGAGCTGCTACACTCTCT |
| | CAPS: p40 | GCAGAGAGAGTCAGAGGGGACAACAAGGAGTATGAGTACTCAGTGGAGTGCCAGGAGGACAGTGCCTGCCCAGCTGCTGAGGAGAGTC |
| | domain; | TGCCCATTGAGGTCATGGTGGATGCCGTTCACAAGCTCAAGTATGAAAACTACACCAGCAGCTTCTTCATCAGGGACATCATCAAACC |
| | CAPS: | TGACCCACCCAAGAACTTGCAGCTGAAGCCATTAAAGAATTCTCGGCAGGTGGAGGTCAGCTGGGAGTACCCTGACACCTGGAGTACT |
| | linker; | CCACATTCCTACTTCTCCCTGACATTCTGCGTTCAGGTCCAGGGCAAGAGCAAGAGAGAAAAGAAAGATAGAGTCTTCACGGACAAGA |
| | CAPS: p35. | CCTCAGCCACGGTCATCTGCCGCAAAAATGCCAGCATTAGCGTGCGGGCCCAGGACCGCTACTATAGCTCATCTTGGAGCGAATGGGC |
| | | ATCTGTGCCCTGCAGTGGCTCTAGCGGAGGGGGAGGCTCTCCTGGCGGGGGATCTAGCAGAAACCTCCCCGTGGCCACTCCAGACCCA |
| | | GGAATGTTCCCATGCCTTCACCACTCCCAAAACCTGCTGAGGGCCGTCAGCAACATGCTCCAGAAGGCCAGACAAACTCTAGAATTTT |
| | | ACCCTTGCACTTCTGAGGAAATTGATCATGAAGATATCACAAAAGATAAAACCAGCACAGTGGAGGCCTGTTTACCATTGGAATTAAC |
| | | CAAGAATGAGAGTTGCCTAAATTCCAGAGAGACCTCTTTCATAACTAATGGGAGTTGCCTGGCCTCCAGAAAGACCTCTTTTATGATG |
| | | GCCCTGTGCCTTAGTAGTATTTATGAAGACTTGAAGATGTACCAGGTGGAGTTCAAGACCATGAATGCAAAGCTTCTGATGGATCCTA |
| | | AGAGGCAGATCTTTCTAGATCAAAACATGCTGGCAGTTATTGATGAGCTGATGCAGGCCCTGAATTTCAACAGTGAGACTGTGCCACA |
| | | AAAATCCTCCCTTGAAGAACCGGATTTTTATAAAACTAAAATCAAGCTCTGCATACTTCTTCATGCTTTCAGAATTCGGGCAGTGACT |
| | | ATTGATAGAGTGATGAGCTATCTGAATGCTTCCTGATGA |
| 16 | Human | ATGTGTCACCAGCAGCTGGTGATCTCATGGTTCTCCCTGGTATTTCTGGCATCTCCTCTTGTCGCAATCTGGGAACTGAAGAAAGACG |
| | optimized | TGTATGTCGTTGAGCTCGACTGGTATCCGGATGCGCCTGGCGAGATGGTGGTGCTGACCTGTGACACCCCAGAGGAGGATGGGATCAC |
| | IL-12sc | TTGGACCCTTGATCAATCCTCCGAAGTGCTCGGGTCTGGCAAGACTCTGACCATACAAGTGAAAGAGTTTGGCGATGCCGGGCAGTAC |
| | (CDS DNA) | ACTTGCCATAAGGGCGGAGAAGTTCTGTCCCACTCACTGCTGCTGCTGCACAAGAAAGAGGACGGAATTTGGAGTACCGATATCCTGA |
| | Sequence | AATGATCAGAAAGAGCCCAAAACAAAACCTTCTTGCGGTGCGAAGCCAAGAACTACTCAGGGAGATTTACTTGTTGGTGGCTGACGAC |
| | annotation | GATCAGCACCGATCTGACTTTCTCCGTGAAATCAAGTAGGGGATCATCTGACCCTCAAGGAGTCACATGTGGAGCGGCTACTCTGAGC |
| | CAPS: p40 | GCTGAACGCGTAAGAGGGGACAATAAGGAGTACGAGTATAGCGTTGAGTGCCAAGAGGATAGCGCATGCCCCGCCGCCGAAGAATCAT |
| | domain; | TGCCCATTGAAGTGATGGTGGATGCTGTACACAAGCTGAAGTATGAGAACTACACAAGCTCCTTCTTCATCCGTGACATCATCAAACC |
| | CAPS: | AGATCCTCCTAAGAACCTCCAGCTTAAACCTCTGAAGAACTCTAGACAGGTGGAAGTGTCTTGGGAGTATCCCGACACCTGGTCTACA |
| | linker; | CCACATTCCTACTTCAGTCTCACATTCTGCGTTCAGGTACAGGGCAAGTCCAAAAGGGAGAAGAAGGATCGGGTCTTTACAGATAAAA |
| | CAPS: p35. | CAAGTGCCACCGTTATATGCCGGAAGAATGCCTCTATTTCTGTGCGTGCGCAGGACAGATACTATAGCAGCTCTTGGAGTGAATGGGC |
| | | CAGTGTCCCATGTTCAGGGTCATCCGGTGGTGGCGGCAGCCCCGGAGGCGGTAGCTCCAGAAATCTCCCTGTGGCTACACCTGATCCA |
| | | GGCATGTTTCCCTGTTTGCACCATAGCCAAAACCTCCTGAGAGCAGTCAGCAACATGCTCCAGAAAGCTAGACAAACACTGGAATTCT |
| | | ACCCATGCACCTCCGAGGAAATAGATCACGAGGATATCACTAAGGACAAAACAAGCACTGTCGAAGCATGCCTTCCCTTGGAACTGAC |
| | | AAAGAACGAGAGTTGCCTTAATTCAAGAGAAACATCTTTCATTACAAACGGTAGCTGCTTGGCAAGCAGAAAAACATCTTTTATGATG |
| | | GCCCTTTGTCTGAGCAGTATTTATGAGGATCTCAAAATGTACCAGGTGGAGTTTAAGACCATGAATGCCAAGCTGCTGATGGACCCAA |
| | | AGAGACAGATTTTCCTCGATCAGAATATGCTGGCTGTGATTGATGAACTGATGCAGGCCTTGAATTTCAACAGCGAAACCGTTCCCCA |
| | | GAAAAGCAGTCTTGAAGAACCTGACTTTTATAAGACCAAGATCAAACTGTGTATTCTCCTGCATGCCTTTAGAATCAGAGCAGTCACT |
| | | ATAGATAGAGTGATGTCCTACCTGAATGCTTCCTGATGA |
| 17 | Human non- | AUGUGUCACCAGCAGUUGGUCAUCUCUUGGUUUUCCCUGGUVUUUCUGGCAUCUCCCCUCGUGGCCAUAUGGGAACUGAAGAAAGAUG |
| | optimized | UUUAUGUCGUAGAAUUGGAUUGGUAUCCGGAUGCCCCUGGAGAAAUGGUGGUCCUCACCUGUGACACCCCUGAAGAAGAUGGUAUCAC |
| | IL-12sc | CUGGACCUUGGACCAGAGCAGUGAGGUCUUAGGCUCUGGCAAAACCCUGACCAUCCAAGUCAAAGAGUUUGGAGAUGCUGGCCAGUAC |
| | (RNA | ACCUGUCACAAAGGAGGCGAGGUUCUAAGCCAUUCGCUCCUGCUGCUUCACAAAAAGGAAGAUGGAAUUUGGUCCACUGAUAUUUUAA |
| | encoding | AGGACCAGAAAGAACCCAAAAAUAAGACCUUUCUAAGAUGCGAGGCCAAGAAUUAUUCUGGACGUUUCACCUGCUGGUGGCUGACGAC |
| | CDS) | AAUCAGUACUGAUUUGACAUUCAGUGUCAAAAGCAGCAGAGGGUCUUCUGACCCCCAAGGGGUGACGUGCGGAGCUGCUACACUCUCU |
| | | GCAGAGAGAGUCAGAGGGGACAACAAGGAGUAUGAGUACUCAGUGGAGUGCCAGGAGGACAGUGCCUGCCCAGCUGCUGAGGAGAGUC |
| | | UGCCCAUUGAGGUCAUGGUGGAUGCCGUUCACAAGCUCAAGUAUGAAAACUACACCAGCAGCUUCUUCAUCAGGGACAUCAUCAAACC |
| | | UGACCCACCCAAGAACUUGCAGCUGAAGCCAUUAAAGAAUUCUCGGCAGGUGGAGGUCAGCUGGGAGUACCCUGACACCUGGAGUACU |
| | | CCACAUUCCUACUUCUCCCUGACAUUCUGCGUUCAGGUCCAGGGCAAGAGCAAGAGAGAAAAGAAAGAUAGAGUCUUCACGGACAAGA |
| | | CCUCAGCCACGGUCAUCUGCCGCAAAAAUGCCAGCAUUAGCGUGCGGGCCCAGGACCGCUACUAUAGCUCAUCUUGGAGCGAAUGGGC |
| | | AUCUGUGCCCUGCAGUGGCUCUAGCGGAGGGGGAGGCUCUCCUGGCGGGGGAUCUAGCAGAAACCUCCCCGUGGCCACUCCAGACCCA |
| | | GGAAUGUUCCCAUGCCUUCACCACUCCCAAAACCUGCUGAGGGCCGUCAGCAACAUGCUCCAGAAGGCCAGACAAACUCUAGAAUUUU |
| | | ACCCUUGCACUUCUGAGGAAAUUGAUCAUGAAGAUAUCACAAAAGAUAAAACCAGCACAGUGGAGGCCUGUUUACCAUUGGAAUUAAC |
| | | CAAGAAUGAGAGUUGCCUAAAUUCCAGAGAGACCUCUUUCAUAACUAAUGGGAGUGGCCUGGCCUCCAGAAAGACCUCUUUUAUGAUG |
| | | GCCCUGUGCCUUAGUAGUAUUUAUGAAGACUGGAAGAUGUACCAGGUGGAGUUCAAGACCAUGAAUGCAAAGCUUCUGAUGGAUCCUA |
| | | AGAGGCAGAUCUUUCUAGAUCAAAACAUGCUGGCAGUUAUUGAUGAGCUGAUGCAGGCCCUGAAUUUCAACAGUGAGACUGUGCCACA |
| | | AAAAUCCUCCCUUGAAGAACCGGAUUUUUAUAAAACUAAAAUCAAGCUCUGCAUACUUCUUCAUGCUUUCAGAAUUCGGGCAGUGACU |
| | | AUUGAUAGAGUGAUGAGCUAUCUGAAUGCUUCCUGAUGA |
| 18 | Human | AUGUGUCACCAGCAGCUGGUGAUCUCAUGGUUCUCCCUGGUAUUUCUGGCAUCUCCUCUUGUCGCAAUCUGGGAACUGAAGAAAGACG |
| | optimized | UGUAUGUCGUUGAGCUCGACUGGUAUCCGGAUGCGCCUGGCGAGAUGGUGGUGCUGACCUGUGACACCCCAGAGGAGGAUGGGAUCAC |
| | IL-12sc | UUGGACCCUUGAUCAAUCCUCCGAAGUGCUCGGGUCUGGCAAGACUCUGACCAUACAAGUGAAAGAGUUUGGCGAUGCCGGGCAGUAC |
| | (RNA | ACUUGCCAUAAGGGCGGAGAAGUUCUGUCCCACUCACUGCUGCUGCUGCACAAGAAAGAGGACGGAAUUUGGAGUACCGAUAUCCUGA |
| | encoding | AAGAUCAGAAAGAGCCCAAGAACAAAACCUUCUUGCGGUGCGAAGCCAAGAACUACUCAGGGAGAUUUACUUGUUGGUGGCUGACGAC |
| | CDS) | GAUCAGCACCGAUCUGACUUUCUCCGUGAAAUCAAGUAGGGGAUCAUCUGACCCUCAAGGAGUCACAUGUGGAGCGGCUACUCUGAGC |
| | | GCUGAACGCGUAAGAGGGGACAAUAAGGAGUACGAGUAUAGCGUUGAGUGCCAAGAGGAUAGCGCAUGCCCCGCCGCCGAAGAAUCAU |
| | | UGCCCAUUGAAGUGAUGGUGGAUGCUGUACACAAGCUGAAGUAUGAGAACUACACAAGCUCCUUCUUCAUCCGUGACAUCAUCAAACC |
| | | AGAUCCUCCUAAGAACCUCCAGCUUAAACCUCUGAAGAACUCUAGACAGGUGGAAGUGUCUUGGGAGUAUCCCGACACCUGGUCUACA |
| | | CCACAUUCCUACUUCAGUCUCACAUUCUGCGUUCAGGUACAGGGCAAGUCCAAAAGGGAGAAGAAGGAUCGGGUCUUUACAGAUAAAA |
| | | CAAGUGCCACCGUUAUAUGCCGGAAGAAUGCCUCUAUUUCUGUGCGUGCGCAGGACAGAUACUAUAGCAGCUCUUGGAGUGAAUGGGC |
| | | CAGUGUCCCAUGUUCAGGGUCAUCCGGUGGUGGCGGCAGCCCCGGAGGCGGUAGCUCCAGAAAUCUCCCUGUGGCUACACCUGAUCCA |
| | | GGCAUGUUUCCCUGUUUGCACCAUAGCCAAAACCUCCUGAGAGCAGUCAGCAACAUGCUCCAGAAAGCUAGACAAACACUGGAAUUCU |
| | | ACCCAUGCACCUCCGAGGAAAUAGAUCACGAGGAUAUCACUAAGGACAAAACAAGCACUGUCGAAGCAUGCCUUCCCUUGGAACUGAC |
| | | AAAGAACGAGAGUUGCCUUAAUUCAAGAGAAACAUCUUUCAUUACAAACGGUAGCUGCUUGGCAAGCAGAAAAACAUCUUUUAUGAUG |
| | | GCCCUUUGUCUGAGCAGUAUUUAUGAGGAUCUCAAAAUGUACCAGGUGGAGUUUAAGACCAUGAAUGCCAAGCUGCUGAUGGACCCAA |
| | | AGAGACAGAUUUUCCUCGAUCAGAAUAUGCUGGCUGUGAUUGAUGAACUGAUGCAGGCCUUGAAUUUCAACAGCGAAACCGUUCCCCA |
| | | GAAAAGCAGUCUUGAAGAACCUGACUUUUAUAAGACCAAGAUCAAACUGUGUAUUCUCCUGCAUGCCUUUAGAAUCAGAGCAGUCACU |
| | | AUAGAUAGAGUGAUGUCCUACCUGAAUGCUUCCUGAUGA |
| IFNalpha2b | | |
| (IFNα2b) | | |
| 19 | Human | MALTFALLVALLVLSCKSSCSVGCDLPQTHSLGSRRTLMLLAQMRRISLFSCLKDRHDFGFPQEEFGNQFQKAETIPVLHEMIQQIFN |
| | IFNα2b | LFSTKDSSAAWDETLLDKFYTELYQQLNDLEACVIQGVGVTETPLMKEDSILAVRKYFQRITLYLKEKKYSPCAWEVVRAEIMRSFSL |
| | (amino | STNLQESLRSKE |
| | acid) | |
| 20 | Human non- | ATGGCCTTGACCTTTGCTTTACTGGTGGCCCTCCTGGTGCTCAGCTGCAAGTCAAGCTGCTCTGTGGGCTGTGATCTGCCTCAAACCC |
| | optimized | ACAGCCTGGGTAGCAGGAGGACCTTGATGCTCCTGGCACAGATGAGGAGAATCTCTCTTTTCTCCTGCTTGAAGGACAGACATGACTT |
| | IFNα2b (CDS | TGGATTTCCCCAGGAGGAGTTTGGCAACCAGTTCCAAAAGGCTGAAACCATCCCTGTCCTCCATGAGATGATCCAGCAGATCTTCAAC |
| | DNA) | CTTTTCAGCACAAAGGACTCATCTGCTGCTTGGGATGAGACCCTCCTAGACAAATTCTACACTGAACTCTACCAGCAGCTGAATGACC |
| | | TGGAAGCCTGTGTGATACAGGGGGTGGGGGTGACAGAGACTCCCCTGATGAAGGAGGACTCCATTCTGGCTGTGAGGAAATACTTCCA |
| | | AAGAATCACTCTCTATCTGAAAGAGAAGAAATACAGCCCTTGTGCCTGGGAGGTTGTCAGAGCAGAAATCATGAGATCTTTTTCTTTG |
| | | TCAACAAACTTGCAAGAAAGTTTAAGAAGTAAGGAATGATGA |
| 21 | Human | ATGGCCCTGACTTTTGCCCTTCTCGTGGCTTTGTTGGTGCTGAGTTGCAAATCTTCCTGTAGTGTCGGATGTGATCTGCCTCAAACCC |
| | optimized | ACAGTCTGGGATCTAGGAGAACACTGATGCTGTTGGCACAGATGAGGAGAATTAGCCTCTTTTCCTGCCTGAAGGATAGACATGACTT |
| | IFNα2b (CDS | CGGCTTTCCCCAAGAGGAGTTTGGCAATCAGTTCCAGAAAGCGGAAACGATTCCCGTTCTGCACGAGATGATCCAGCAGATCTTCAAC |
| | DNA) | CTCTTTTCAACCAAAGACAGCTCAGCAGCCTGGGATGAGACACTGCTGGACAAATTCTACACAGAACTGTATCAGCAGCTTAACGATC |
| | | TGGAGGCATGCGTGATCCAAGGGGTTGGTGTGACTGAAACTCCGCTTATGAAGGAGGACTCCATTCTGGCTGTACGGAAGTACTTCCA |
| | | GAGAATAACCCCCTATCTGAAGGAGAAGAAGTACTCACCATGTGCTTGGGAAGTCGTGAGAGCCGAAATCATGAGATCCTTCAGCCTT |
| | | AGCACCAATCTCCAGGAATCTCTGAGAAGCAAAGAGTGATGA |
| 22 | Human non- | AUGGCCUUGACCUUUGCUUUACUGGUGGCCCUCCUGGUGCUCAGCUGCAAGUCAAGCUGCUCUGUGGGCUGUGAUCUGCCUCAAACCC |
| | optimized | ACAGCCUGGGUAGCAGGAGGACCUUGAUGCUCCUGGCACAGAUGAGGAGAAUCUCUCUUUUCUCCUGCUUGAAGGACAGACAUGACUU |
| | IFNα2b (RNA | UGGAUUUCCCCAGGAGGAGUUUGGCAACCAGUUCCAAAAGGCUGAAACCAUCCCUGUCCUCCAUGAGAUGAUCCAGCAGAUCUUCAAC |
| | encoding | CUUUUCAGCACAAAGGACUCAUCUGCUGCUUGGGAUGAGACCCUCCUAGACAAAUUCUACACUGAACUCUACCAGCAGCUGAAUGACC |
| | CDS) | UGGAAGCCUGUGUGAUACAGGGGGUGGGGGUGACAGAGACUCCCCUGAUGAAGGAGGACUCCAUUCUGGCUGUGAGGAAAUACUUCCA |
| | | AAGAAUCACUCUCUAUCUGAAAGAGAAGAAAUACAGCCCUUGUGCCUGGGAGGUUGUCAGAGCAGAAAUCAUGAGAUCUUUUUCUUUG |
| | | UCAACAAACUUGCAAGAAAGUUUAAGAAGUAAGGAAUGAUGA |
| 23 | Human | AUGGCCCUGACUUUUGCCCUUCUCGUGGCUUUGUUGGUGCUGAGUUGCAAAUCUUCCUGUAGUGUCGGAUGUGAUCUGCCUCAAACCC |
| | optimized | ACAGUCUGGGAUCUAGGAGAACACUGAUGCUGUUGGCACAGAUGAGGAGAAUUAGCCUCUUUUCCUGCCUGAAGGAUAGACAUGACUU |
| | IFNα2b (RNA | CGGCUUUCCCCAAGAGGAGUUUGGCAAUCAGUUCCAGAAAGCGGAAACGAUUCCCGUUCUGCACGAGAUGAUCCAGCAGAUCUUCAAC |
| | encoding | CUCUUUUCAACCAAAGACAGCUCAGCAGCCUGGGAUGAGACACUGCUGGACAAAUUCUACACAGAACUGUAUCAGCAGCUUAACGAUC |
| | CDS) | UGGAGGCAUGCGUGAUCCAAGGGGUUGGUGUGACUGAAACUCCGCUUAUGAAGGAGGACUCCAUUCUGGCUGUACGGAAGUACUUCCA |
| | | GAGAAUAACCCUCUAUCUGAAGGAGAAGAAGUACUCACCAUGUGCUUGGGAAGUCGUGAGAGCCGAAAUCAUGAGAUCCUUCAGCCUU |
| | | AGCACCAAUCUCCAGGAAUCUCUGAGAAGCAAAGAGUGAUGA |
| IL-15 sushi | | |
| 24 | Human 1L-15 | MAPRRARGCRTLGLPALLLLLLLRPPATRGITCPPPMSVEHADIWVKSYSLYSRERYICNSGFKRKAGTSSLTECVLNKATNVAHWTT |
| | sushi | PSLKCIRDPALVHQRPAPPGGGSGGGGSGGGSGGGGSLQNWVNVISDLKKIEDLIQSMHIDATLYTESDVHPSCKVTAMKCFLLELQV |
| | (amino | ISLESGDASIHDTVENLIILANNSLSSNGNVTESGCKECEELEEKNIKEFLQSFVHIVQMFINTS |
| | acid) | |
| 25 | Human IL-15 | ATGGCCCCGCGGCGGGCGCGCGGCTGCCGGACCCTCGGTCTCCCGGCGCTGCTACTGCTGCTGCTGCTCCGGCCGCCGGCGACGCGGG |
| | sushi (CDS | GCATCACGTGCCCTCCCCCCATGTCCGTGGAACACGCAGACATCTGGGTCAAGAGCTACAGCTTGTACTCCAGGGAGCGGTACATTTG |
| | DNA) | TAACTCTGGTTTCAAGCGTAAAGCCGGCACGTCCAGCCTGACGGAGTGCGTGTTGAACAAGGCCACGAATGTCGCCCACTGGACAACC |
| | Sequence | CCCAGTCTCAAATGCATTAGAGACCCTGCCCTGGTTCACCAAAGGCCAGCGCCACCCGGGGGAGGATCTGGCGGCGGTCCCTCTGGCG |
| | annotations | GGGGATCTGGCGGAGGAGGAAGCTTACAGAACTGGGTGAATGTAATAAGTGATTTGAAAAAAATTGAAGATCTTATTCAATCTATGCA |
| | CAPS: IL-1b | TATTGATGCTACTTTATATACGGAAAGTGATGTTCACCCCAGTTGCAAAGTAACAGCAATGAAGTGCTTTCTCTTGGAGTTACAAGTT |
| | sushi; | ATTTCACTTGAGTCCGGAGATGCAAGTATTCATGATACAGTAGAAAATCTGATCATCCTAGCAAACAACAGTTTGTCTTCTAATGGGA |
| | CAPS: | ATGTAACAGAATCTGGATGCAAAGAATGTGAGGAACTGGAGGAAAAAAATATTAAAGAATTTTTGCAGAGTTTTGTACATATTGTCCA |
| | linker; | AATGTTCATCAACACTTCTTGATGA |
| | CAPS: | |
| | mature IL- | |
| | 15 | |
| 26 | Human IL-15 | AUGGCCCCGCGGCGGGCGCGCGGCUGCCGGACCCUCGGUCUCCCGGCGCUGCUACUGCUGCUGCUGCUCCGGCCGCCGGCGACGCGGG |
| | sushi (RNA | GCAUCACGUGCCCUCCCCCCAUGUCCGUGGAACACGCAGACAUCUGGGUCAAGAGCUACAGCUUGUACUCCAGGGAGCGGUACAUUUG |
| | encoding | UAACUCUGGUUUCAAGCGUAAAGCCGGCACGUCCAGCCUGACGGAGUGCGUGUUGAACAAGGCCACGAAUGUCGCCCACUGGACAACC |
| | CDS) | CCCAGUCUCAAAUGCAUUAGAGACCCUGCCCUGGUUCACCAAAGGCCAGCGCCACCCGGGGGAGGAUCUGGCGGCGGUGGGUCUGGCG |
| | | GGGGAUCUGGCGGAGGAGGAAGCUUACAGAACUGGGUGAAUGUAAUAAGUGAUUUGAAAAAAAUUGAAGAUCUUAUUCAAUCUAUGCA |
| | | UAUUGAUGCUACUUUAUAUACGGAAAGUGAUGUUCACCCCAGUUGCAAAGUAACAGCAAUGAAGUGCUUUCUCUUGGAGUUACAAGUU |
| | | AUUUCACUUGAGUCCGGAGAUGCAAGUAUUCAUGAUACAGUAGAAAAUCUGAUCAUCCUAGCAAACAACAGUUUGUCUUCUAAUGGGA |
| | | AUGUAACAGAAUCUGGAUGCAAAGAAUGUGAGGAACUGGAGGAAAAAAAUAUUAAAGAAUUGUUGCAGAGUUUUGUACAUAUUGUCCA |
| | | AAUGUUCAUCAACACUUCUUGAUGA |
| GM-CSF | | |
| 27 | Human GM- | MWLQSLLLLGTVACSISAPARSPSPSTQPWEHVNAIQEARRLLNLSRDTAAEMNETVEVISEMFDLQEPTCLQTRLELYKQGLRGSLT |
| | CSF (amino | KLKGPLTMMASHYKQHCPPTPETSCATQIITFESFKENLKDFLLVIPFDCWEPVQE |
| | acid) | |
| 28 | Human GM- | ATGTGGCTCCAGAGCCTGCTGCTCTTGGGCACTGTGGCCTGCTCCATCTCTGCACCCGCCCGCTCGCCCAGCCCCAGCACGCAGCCCT |
| | CSF (CDS | GGGAGCATGTGAATGCCATCCAGGAGGCCCGGCGTCTGCTGAACCTGAGTAGAGACACTGCTGCTGAGATGAATGAAACAGTAGAAGT |
| | DNA) | CATCTCAGAAATGTTTGACCTCCAGGAGCCGACCTGCCTACAGACCCGCCTGGAGCTGTACAAGCAGGGCCTGCGGGGCAGCCTCACC |
| | | AAGCTCAAGGGCCCCTTGACCATGATGGCCAGCCACTACAAGCAGCACTGCCCTCCAACCCCGGAAACTTCCTGTGCAACCCAGATTA |
| | | TCACCTTTGAAAGTTTCAAAGAGAACCTGAAGGACTTTCTGCTTGTCATCCCCTTTGACTGCTGGGAGCCAGICCAGGAGTGATGA |
| 29 | Human GM- | AUGUGGCUCCAGAGCCUGCUGCUCUUGGGCACUGUGGCCUGCUCCAUCUCUGCACCCGCCCGCUCGCCCAGCCCCAGCACGCAGCCCU |
| | CSF (RNA | GGGAGCAUGUGAAUGCCAUCCAGGAGGCCCGGCGUCUGCUGAACCUGAGUAGAGACACUGCUGCUGAGAUGAAUGAAACAGUAGAAGU |
| | encoding | CAUCUCAGAAAUGUUUGACCUCCAGGAGCCGACCUGCCUACAGACCCGCCUGGAGCUGUACAAGCAGGGCCUGCGGGGCAGCCUCACC |
| | CDS) | AAGCUCAAGGGCCCCUUGACCAUGAUGGCCAGCCACUACAAGCAGCACUGCCCUCCAACCCCGGAAACUUCCUGUGCAACCCAGAUUA |
| | | UCACCUUUGAAAGUUUCAAAGAGAACCUGAAGGACUUUCUGCUUGUCAUCCCCUUUGACUGCUGGGAGCCAGUCCAGGAGUGAUGA |
| 30 | Exemplary | AAAAAAAAAAAAAAAAAAAAAAAAAAAAAAAAAAAAAAAAAAAAAAAAAAAAAAAAAAAAAAAAAAAAAAAAAAAAAAAAAAAAAAAA |
| | Poly-A | AAAAAAAAAAAAAAAAAAAAAA |
| 31 | sgRNA in | GGCGTATGTATCAGTCTCAG |
| | Example 3 | |

### DETAILED DESCRIPTION

### Definitions

As used herein, a “cytokine RNA mixture,” also sometimes referred to as “cytokine mRNA mixture,” “mRNA cytokine mixture,” or “RNA cytokine mixture” comprises RNA encoding IFNα, RNA encoding IL-15 sushi, RNA encoding IL-12sc, and RNA encoding GM-CSF, as described herein.

“PD-1” may also be referred to as “programmed cell death 1” or “programmed cell death-.” “PD-L1” may also be referred to as “programmed cell death 1 ligand,” “programmed cell death-1 ligand 1,” or “programmed cell death-ligand I.”

As used herein, an “advanced stage solid tumor cancer,” sometimes referred to herein as “advanced solid tumor,” or “advanced solid tumor cancer,” comprises a solid tumor cancer whose stage is identified as stage III, subsets of stage III, stage IV, or subsets of stage IV, assessed by a known system, e.g., the tumor, node, and metastasis (TNM) staging system developed by the American Joint Committee on Cancer (AJCC) (see AJCC Cancer Staging Manual, 8^{th} Edition). In some embodiments, the TNM staging system is used for solid tumor cancers other than melanoma. In some embodiments, the cancer is melanoma or advanced melanoma, which comprises stage 111B, stage IIIC, or stage V as assessed by the AJCC melanoma staging (edition 8, 2018). Non-limiting descriptions relating to AJCC melanoma staging are provided in Gershenwald J E, Scolyer R A, Hess K R, et al. Melanoma of the skin. In: Amin M B, ed. AJCC Cancer Staging Manual. 8th ed. Chicago, Ill.: AJCC-Springer; 2017:563-585, the entire contents of which are incorporated herein by reference. In some embodiments, the cancer is cutaneous squamous cell carcinoma (CSCC), or squamous cell carcinoma of the head and neck (HNSCC), both of which may be advanced. Similar staging systems exists for all major cancers and are generally based on the clinical and/or pathological details of the tumor and how these factors have been shown to impact survival.

“Tumor” may also be referred to herein as “neoplasm”. For instance, the terms “solid tumor” and “solid neoplasm” are interchangeable.

An “unresectable” (e.g., advanced-stage unresectable) cancer typically cannot be removed with surgery.

RECIST (Response Evaluation Criteria for Solid Tumours (also Tumors)) provides a methodology to evaluate the activity and efficacy of cancer therapeutics in solid tumors. RECIST guidelines were created by the RECIST Working Group comprising representatives from the European Organization for Research and Treatment of Cancer, National Cancer Institute of the United States and Canadian Cancer Trials Group, as well as several pharmaceutical companies, and published in Eisenhauer E A, Therasse P, Bogaerts J et al. New response evaluation criteria in solid tumours: Revised RECIST guideline (version 1.1) Eur J Cancer. 45 (2009) 228-247, the entire contents of which are incorporated herein by reference. Section 4.3.1 of the guidelines (page 232-233 of Eisenhauer) provides the following regarding evaluation of target lesions:
Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

Section 4.3.3 of the guidelines (page 233 of Eisenhauer) provides the following regarding evaluation of non-target lesions:

While some non-target lesions may actually be measurable, they need not be measured and instead should be assessed only qualitatively at the time points specified in the protocol.
Complete Response (CR): Disappearance of all non-target lesions and normalization of tumour marker level. All lymph nodes must be non-pathological in size (<10 mm short axis).
Non-CR/Non-PD: Persistence of one or more non-target lesion(s) and/or maintenance of tumour marker level above the normal limits.
Progressive Disease (PD): Unequivocal progression of existing non-target lesions.

(Note: the appearance of one or more new lesions is also considered progression).

A subject having “innate” or “primary” resistance to an anti-PD-1 or anti-PD-L1 therapy, does not initially respond to anti-PD-1 or anti-PD-L1 therapy. A subject having innate or primary resistance never demonstrated a clinical response to PD-1/PD-L1 blockade. See. e.g., Sharma et al. (2017) Cell 168:707-723 at 709; see also, Hugo et al. (2016) Cell 165 (1) 35-44; see also, Nowicki et al. (2018) Cancer J. 24(1): 47-53, the entire contents of which are incorporated herein by reference. In some embodiments, a subject with innate resistance to an anti-PD-1 or anti-PD-L1 therapy is characterized after treatment with anti-PD-1 or anti-PD-L1 therapy (any length of time) as having Progressive Disease or Stable Disease according to RECIST criteria (version 1.1). In some embodiments, a subject with innate resistance to an anti-PD-1 or anti-PD-L1 therapy is characterized after treatment with anti-PD-1 or anti-PD-L1 therapy (any length of time) as having non-CR/Non-PD for non-target lesions comprising viable cancer cells. In some embodiments, a subject with innate resistance to an anti-PD-1 therapy is characterized after treatment with anti-PD-1 therapy (any length of time) as having Progressive Disease according to RECIST criteria (version 1.1). In some embodiments, a subject with innate resistance to an anti-PD-L1 therapy is characterized after treatment with anti-PD-L1 therapy (any length of time) as having Progressive Disease according to RECIST criteria (version 1.1). In some embodiments, a subject with innate resistance to an anti-PD-1 therapy is characterized after treatment with anti-PD-1 therapy (any length of time) as having Stable Disease according to RECIST criteria (version 1.1). In some embodiments, a subject with innate resistance to an anti-PD-L1 therapy is characterized after treatment with anti-PD-L1 therapy (any length of time) as having Stable Disease according to RECIST criteria (version 1.1). In some embodiments, a subject with innate resistance to an anti-PD-1 or anti-PD-L1 therapy is characterized after treatment with anti-PD-1 or anti-PD-L1 therapy (any length of time) as having at least a 20% increase in the longest diameter of a solid tumor and/or the appearance of one or more new solid tumors. In some embodiments, a subject with innate resistance to an anti-PD-1 is characterized after treatment with anti-PD-1 therapy (any length of time) as having at least a 20% increase in the longest diameter of solid tumors and/or the appearance of one or more new solid tumors. In some embodiments, a subject with innate resistance to an anti-PD-L1 therapy is characterized after treatment with anti-PD-L1 therapy (any length of time) as having at least a 20% increase in the longest diameter of solid tumors and/or the appearance of one or more new solid tumors. In some embodiments, the increase in the longest diameter is an increase of at least 5 mm. In some embodiments, the length of time is about 6 weeks, about 8 weeks, or at least 6 or 8 weeks. In some embodiments, the length of time is 2, 3, 6, 12, or more months. In some embodiments, the solid tumor is a primary tumor. In some embodiments, the solid tumor is an injectable tumor. In some embodiments, the solid tumor has been injected with the cytokine mRNA mixture. In some embodiments, the solid tumor has been selected for injection with the cytokine mRNA mixture. In some embodiments, the solid tumor is a subcutaneous lesion ≥0.5 cm in longest diameter. In some embodiments, the solid tumor is within a group of multiple injectable merging lesions that are confluent. In some embodiments, the solid tumor is within a group of multiple injectable merging lesions that are confluent and have the longest diameter (sum of diameters of all involved target lesions) of ≥0.5 cm. In some embodiments, the solid tumor is not bleeding or weeping. In some embodiments, the longest diameter of the solid tumor is at least 10 mm (e.g., as measured by Computed Tomography (CT) scan or caliper). In some embodiments, the solid tumor is in the chest of a subject and longest diameter of the solid tumor is at least 20 mm (e.g., as measured by chest X-ray). In some embodiments, the solid tumor is in a lymph node. In some embodiments, the lymph node is at least 15 mm in short axis (e.g., when assessed by CT scan). In some embodiments, the solid tumor is a lymphoma. In some embodiments, a subject with innate resistance to an anti-PD-1 or anti-PD-L1 therapy is characterized after treatment with anti-PD-1 or anti-PD-L1 therapy (any length of time) as having no response or stable disease according to the Lugano Classification. The version of the Lugano Classification referred to herein is described in Cheson et al. 2014 J Clin Oncol. 32(27):3059-68, the entire content of which is incorporated herein by reference. In some embodiments, a subject with innate resistance to an anti-PD-1 or anti-PD-L1 therapy is characterized after treatment with anti-PD-1 or anti-PD-L1 therapy (any length of time) as having progressive disease according to the Lugano Classification. In some embodiments, a subject with innate resistance to an anti-PD-1 or anti-PD-L1 therapy is characterized after treatment with anti-PD-1 or anti-PD-L1 therapy (any length of time) as having a lymphoma tumor within a lymph node. In some embodiments, a subject with innate resistance to an anti-PD-1 or anti-PD-L1 therapy is characterized after treatment with anti-PD-1 or anti-PD-L1 therapy (any length of time) as having a lymphoma tumor within a lymph node, wherein the lymph node has (i) a longest diameter of greater than 1.5 cm, and (ii) an increase of at least 50% from the product of the perpendicular diameters (PPDs) nadir. In some embodiments, the increase in the longest diameter is an increase of at least 5 mm. In some embodiments, the length of time is about 6 weeks, about 8 weeks, or at least 6 or 8 weeks. In some embodiments, the length of time is 2, 3, 6, 12, or more months.

A subject having “acquired” or “adaptive” resistance to an anti-PD-1 or anti-PD-L1 therapy initially responds to therapy (e.g., any level of response), but after a period of time relapses and progresses. In some embodiments, response to therapy is assessed as per RECIST criteria (version 1.1). In some embodiments, acquired or adaptive resistance to an anti-PD-1 or anti-PD-L1 therapy is seen in subjects who eventually progresses while on therapy despite an initial Complete Response or Partial Response, all according to RECIST criteria (version 1.1). In some embodiments, acquired or adaptive resistance to an anti-PD-1 or anti-PD-L1 therapy is seen in subjects who are unresponsive to re-initiation of an anti-PD-1 or anti-PD-L1 therapy. See, Sharma et al. (2017) Cell 168:707-723 at 708; see also, Nowicki et al. (2018) Cancer J. 24(1): 47-53, the entire contents of which are incorporated herein by reference. In some embodiments, a subject with adaptive resistance to an anti-PD-1 therapy comprises a solid tumor whose volume (i) decreased for a period of time after anti-PD-1 therapy began; and then (ii) increased after the period of time despite continued anti-PD-1 therapy. In some embodiments, a subject with adaptive resistance to an anti-PD-L1 therapy comprises a solid tumor whose volume (i) decreased for a period of time after anti-PD-L1 therapy began; and then (ii) increased after the period of time despite continued anti-PD-L1 therapy. In some embodiments, the adaptive resistance is associated with an acquired underlying mechanism of resistance. In embodiments, the adaptive resistance is associated with a mutation or an epigenetic change. In some embodiments, the adaptive resistance is associated with a mutation in a B2M gene. In some embodiments, the period of time is from 6 to 12 months. In some embodiments, the period of time is from 6 to 18 months. In some embodiments, the period of time is from 6 to 36 months. In some embodiments, the period of time is from 3 to 9 months. In some embodiments, the period of time is from 3 to 24 months. In some embodiments, the period of time is from 12 to 24 months. In some embodiments, the period of time is at least about 3, about 4, about 5, about 6, about 7, about 8, about 9, about 10, about 11, about 12, about 13, about 14, about 15, about 16, about 17, about 18, about 19, about 20, about 21, about 22, about 23, or about 24 months. In some embodiments, the period of time is at least about 4 months. In some embodiments, the period of time is at least about 6 months. In some embodiments, the period of time is at least about 12 months. In some embodiments, the period of time is at least about 24 months. In some embodiments, the period of time is at least about 30 months. In some embodiments, the period of time is at least about 36 months. In some embodiments, a subject with adaptive resistance to an anti-PD-1 or anti-PD-L1 therapy was characterized at any point during treatment as having a Complete Response and thereafter (and during treatment) was characterized as having Progressive Disease according to RECIST criteria (version 1.1). In some embodiments, a subject with adaptive resistance to an anti-PD-1 or anti-PD-L1 therapy was characterized at any point during treatment as having a Partial Response and thereafter (and during treatment) was characterized as having a Progressive Disease or Stable Disease, all according to RECIST criteria (version 1.1). In some embodiments, a subject with adaptive resistance to an anti-PD-1 or anti-PD-L1 therapy was characterized at any point during treatment as having a Partial Response and thereafter (and during treatment) was characterized as having Progressive Disease according to RECIST criteria (version 1.1). In some embodiments, a subject with adaptive resistance to an anti-PD-1 or anti-PD-L1 therapy was characterized at any point during treatment as having a Partial Response and thereafter (and during treatment) was characterized as having Stable Disease according to RECIST criteria (version 1.1). In some embodiments, the longest diameter of solid tumors in the subject decreased by at least 30% after the anti-PD-1 or anti-PD-L1 therapy began and then increased. In some embodiments, the longest diameter of solid tumors in the subject decreased by at least 30% after the anti-PD-1 or anti-PD-L1 therapy began and then increased by at least 20%. In some embodiments, the longest diameter of solid tumors in the subject decreased by at least 30% after the anti-PD-1 or anti-PD-L1 therapy began and then one or more new solid tumors appeared. In some embodiments, a subject with adaptive resistance to an anti-PD-1 or anti-PD-L1 therapy was characterized at any point during treatment as having least a 30% decrease in the longest diameter of solid tumors and thereafter (and during treatment) was characterized as having at least a 20% increase in the longest diameter of a solid tumors and/or the appearance of one or more new solid tumors. In some embodiment, the increase in the longest diameter is an increase of at least 5 mm. In some embodiments, a subject with adaptive resistance to an anti-PD-1 or anti-PD-L1 therapy was characterized at any point during treatment as having a disappearance of a solid tumor (e.g., every solid tumor that was present if more than one solid tumor was present) and thereafter (and during treatment) was characterized as having the reappearance of the solid tumor (e.g., in the same location as a solid tumor that disappeared) and/or the appearance of one or more new solid tumors. In some embodiments, the solid tumor is a primary tumor. In some embodiments, the solid tumor is an injectable tumor. In some embodiments, the tumor has been injected with the cytokine mRNA mixture. In some embodiments, the tumor has been selected for injection with the cytokine mRNA mixture. In some embodiments, the solid tumor is a subcutaneous lesion ≥0.5 cm in longest diameter. In some embodiments, the solid tumor is within a group of multiple injectable merging lesions that are confluent. In some embodiments, the solid tumor is within a group of multiple injectable merging lesions that are confluent and have the longest diameter (sum of diameters of all involved target lesions) of ≥0.5 cm. In some embodiments, the solid tumor is not bleeding or weeping. In some embodiments, the longest diameter of the solid tumor is at least 10 mm (e.g., as measured by Computed Tomography (CT) scan or caliper). In some embodiments, the solid tumor is in the chest of a subject and longest diameter of the solid tumor is at least 20 mm (e.g., as measured by chest X-ray). In some embodiments, the solid tumor is in a lymph node. In some embodiments, the lymph node is at least 15 mm in short axis (e.g., when assessed by CT scan). In some embodiments, the solid tumor is a lymphoma. In some embodiments, a subject with adaptive resistance to an anti-PD-1 or anti-PD-L1 therapy was characterized at any point during treatment as having a complete response and thereafter (and during treatment) was characterized as having progressive disease according to the Lugano Classification. In some embodiments, a subject with adaptive resistance to an anti-PD-1 or anti-PD-L1 therapy was characterized at any point during treatment as having at least a 50% decrease in the sum of the product of the perpendicular diameters (PPDs) for multiple lesions (e.g. for 1, 2, 3, 4, 5, or 6 lymph node or extranodal sites) and thereafter (and during treatment) was characterized as having a lymphoma tumor within a lymph node, wherein the lymph node has (i) a longest diameter of greater than 1.5 cm, and (ii) an increase of at least 50% from the PPD nadir.

A “refractory” or “resistant” cancer is one that does not respond to a specified treatment. In some embodiments, refraction occurs from the very beginning of treatment. In some embodiments, refraction occurs during treatment. In some embodiments, a cancer is resistant before treatment begins. In some embodiments, a cancer is refractory or resistant to anti-PD-1 therapy (i.e., the cancer does not respond to the therapy). In some embodiments, a cancer is refractory or resistant to anti-PD-L1 therapy (i.e., the cancer does not respond to the therapy). In some embodiments, a subject has a cancer that is becoming refractory or resistant to a specified treatment (such as an anti-PD1 or anti-PD-L1 therapy), e.g., the subject has become less responsive to the treatment since first receiving it. In some embodiments, the subject has not received the treatment, but has a type of cancer that does not typically respond to the treatment.

A “superficial” (also sometimes referred to as “cutaneous”) lesion or metastasis is a lesion or metastasis that is within the skin or is at the surface of skin. In some embodiments, a superficial lesion or metastasis is within the cutis. In some embodiments, a superficial lesion or metastasis is within the dermis. In some embodiments, a superficial lesion or metastasis is within the epidermis.

A “subcutaneous” lesion or metastasis is under the skin. In some embodiments, a subcutaneous lesion or metastasis is with the subcutis.

In some embodiments, and in the context of a solid tumor cancer, a “tumor lesion” or “lesion” is a solid tumor, e.g., a primary solid tumor or a solid tumor that has arisen from a metastasis from another solid tumor.

The term “squamous cell” refers to any thin flat cells found, for example, in the surface of the skin, eyes, various internal organs, and the lining of hollow organs and ducts of some glands.

The term “cutaneous squamous cell carcinoma” (or “CSCC”) refers to all stages and all forms of cancer that begin in cells that form the epidermis (outer layer of the skin). The term “cutaneous squamous cell carcinoma” is used interchangeably with the term “squamous cell carcinoma” of the skin.

The term “squamous cell carcinoma of the head and neck” (or “head and neck squamous cell carcinoma” or “HNSCC” or “squamous cell carcinoma for the head and neck”) refers to all stages and all forms of cancer of the head and neck that begin in squamous cells. Squamous cell carcinoma of the head and neck includes (but is not limited to) cancers of the nasal cavity, sinuses, lips, mouth, salivary glands, throat, and larynx (voice box).

The term “melanoma” refers to all stages and all forms of cancer that begins in melanocytes. Melanoma typically begins in a mole (skin melanoma), but can also begin in other pigmented tissues, such as in the eye or in the intestines.

A “tumor-involved regional lymph node” or “tumor-involved node” refers to metastasis-containing regional lymph node. In some embodiments, a tumor-involved regional lymph node is a clinically occult tumor-involved regional lymph node. In some embodiments, a tumor-involved regional lymph node is a clinically detectable tumor-involved regional lymph node. A “clinically occult” tumor-involved regional lymph node describes microscopically identified regional node metastasis without clinical or radiographic evidence of regional node metastasis. In some embodiments, a clinically occult tumor-involved regional lymph node is detected by sentinel lymph node (SLN) biopsy and without clinical or radiographic evidence of regional node metastasis. In some embodiments, “clinically detectable” nodal metastasis describes patients with regional node metastasis identifiable by clinical, radiographic, or ultrasound examination and usually (but not necessarily) confirmed by biopsy.

“Non-nodal locoregional sites” refer to metastases that are a consequence of intralymphatic or angiotrophic tumor spread and include microsatellite, satellite, and in-transit metastases. “Satellite” metastases refer to clinically evident cutaneous and/or subcutaneous metastases occurring within 2 cm of a primary melanoma.

“Microsatellite” metastases refer to microscopic cutaneous and/or subcutaneous metastases found adjacent or deep to a primary melanoma on pathological examination of the primary site. In some embodiments, microsatellite metastases are completely discontinuous from a primary melanoma with unaffected stroma occupying the space between.

“In-transit” metastases refer to clinically evident cutaneous and/or subcutaneous metastases identified at a distance more than 2 cm from a primary melanoma in the region between the primary and the first echelon of regional lymph nodes. In some embodiments, satellite or in-transmit metastases may occur distal to a primary melanoma.

“Matted nodes” refer to two or more nodes adherent to one another through involvement by metastatic disease. In some embodiments, matted nodes are identified at the time a specimen is examined macroscopically in a pathology laboratory.

A “distant metastasis” refers to cancer that has spread from the primary tumor to a distant organ or a distant lymph node. In some embodiments, the distant metastasis is detectable in skin, subcutaneous tissue, muscle, or distant lymph nodes. In some embodiments, the distant metastasis is detectable in a lung. In some embodiments, the distant metastasis is detectable in central nerve system (CNS). In some embodiments, the distant metastasis is detectable in any other visceral site other than CNS, including the lungs, the heart, or an organ of the digestive, excretory, reproductive, or circulatory system. In some embodiments, a distant metastasis is in a tissue or organ that is not in direct contact (e.g., touching or directly connected to) the tissue or organ containing the primary tumor.

In some embodiments, a metastasis (e.g., a distant metastasis) is in (e.g., is detectable in) the liver.

“Extranodal extension” (ENE) refers to the extension of metastatic cells through the nodal capsule into the perinodal tissue during nodal metastasis. Cystic metastasis that stretches, but does not breach, the lymph node capsule may be classified as ENE-negative. In some embodiments, the ENE-positive includes large extranodal vessels. In some embodiments, the ENE-positive extends less than 2 mm from the node capsule. In some embodiments, the ENE-positive extends more than 2 mm from the lymph node capsule or is apparent to the naked eye at dissection.

“Deep invasion” refers to as thickness greater than 6 mm or invasion deeper than subcutaneous fat. In some embodiments, invasion is present in nerves greater than 0.1 mm, deeper than the dermis.

The term “effective amount” refers to an amount of an agent (such as a mixture of RNAs) that provides a desired biological, therapeutic, and/or prophylactic result. That result can be reduction, amelioration, palliation, lessening, delaying, prevention, and/or alleviation of one or more of the signs, symptoms, or causes of a disease (such as advanced stage solid tumor cancer). In some embodiments, an effective amount comprises an amount sufficient to cause a solid tumor/lesion to shrink. In some embodiments, an effective amount is an amount sufficient to decrease the growth rate of a solid tumor (such as to suppress tumor growth). In some embodiments, an effective amount is an amount sufficient to delay tumor development. In some embodiments, an effective amount is an amount sufficient to prevent or delay tumor recurrence. In some embodiments, an effective amount is an amount sufficient to increase a subject's immune response to a tumor, such that tumor growth and/or size and/or metastasis is reduced, delayed, ameliorated, and/or prevented. An effective amount can be administered in one or more administrations. In some embodiments, administration of an effective amount (e.g., of a composition comprising mRNAs) may: (i) reduce the number of cancer cells; (ii) reduce tumor size: (iii) inhibit, retard, slow to some extent and may stop cancer cell infiltration into peripheral organs, (iv) inhibit (e.g., slow to some extent and/or block or prevent) metastasis; (v) inhibit tumor growth: (vi) prevent or delay occurrence and/or recurrence of tumor; and/or (vii) relieve to some extent one or more of the symptoms associated with the cancer. In some embodiments. Inhibit, inhibitory, and the like refer to a complete or partial block of an interaction, or a reduction in a biological effect, for example, inhibiting tumor growth or metastasis includes reduction or complete cessation.

The term “co-administered” or “co-administration” or the like as used herein refers to administration of two or more agents concurrently, simultaneously, or essentially at the same time, either as part of a single formulation or as multiple formulations that are administered by the same or different routes. “Essentially at the same time” as used herein means within about 1 minute, 5 minutes, 10 minutes, 15 minutes, 30 minutes, 1 hour, 2 hours, or 6 hours period of each other.

In some embodiments, the RNA comprises a modified nucleobase in place of at least one (e.g., every) uridine. In some embodiments, the RNA comprises a Cap1 structure at the 5′ end of the RNA. In some embodiments, the RNA comprises a modified nucleobase in place of at least one (e.g., every) uridine and a Cap1 structure at the 5′ end of the RNA. In some embodiments, the 5′ UTR comprises SEQ ID NOs: 4 or 6. In some embodiments, the RNA has been processed to reduce double-stranded RNA (dsRNA), such as, for example, by purification on cellulose (as described in the Examples and as known in the art), or via high performance liquid chromatography (HPLC). The “Cap1” structure may be generated after in-vitro transcription by enzymatic capping or during in-vitro transcription (co-transcriptional capping).

In some embodiments, the building block cap for modified RNA is as follows, which is used when co-transcriptionally capping: m₂^{7,3′-O}Gppp(m₁^{2′-O})ApG (also sometimes referred to as m₂^{7,3′-O}G(5′)ppp(5′)m^{2′-O}ApG), which has the following structure:

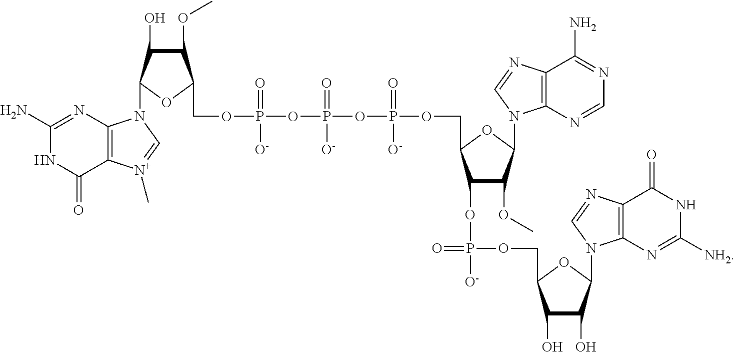

Below is an exemplary Cap1 RNA after co-transcriptional capping, which comprises RNA and m₂^{7,3′-O}G(5′)ppp(5′)m^{2′-O}ApG:

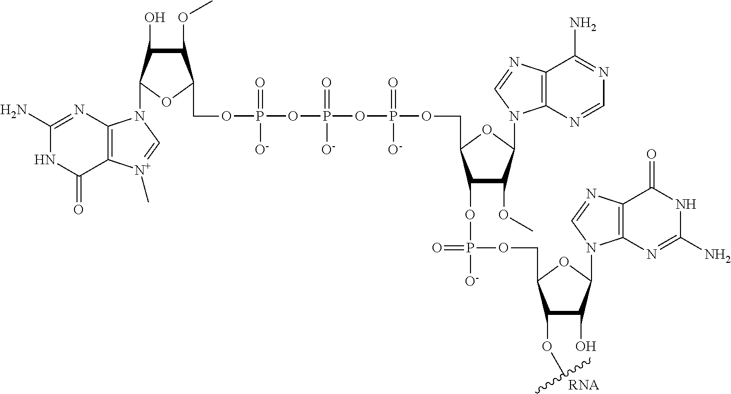

Below is another exemplary Cap1 RNA after enzymatic capping (no cap analog):

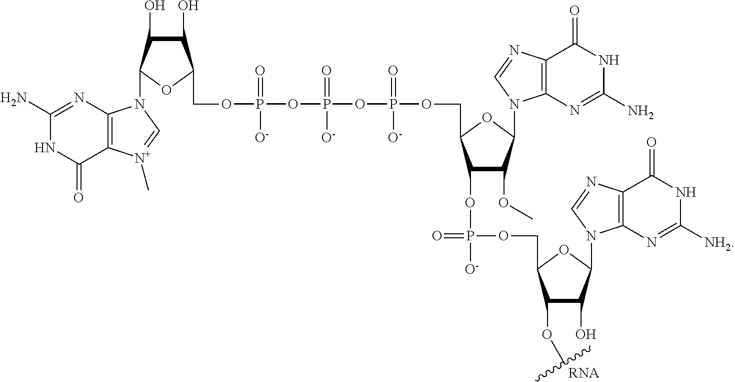

In some embodiments, the RNA is modified with “Cap0” structures generated during in-vitro transcription (co-transcriptional capping) using, in one embodiment, the cap analog anti-reverse cap (ARCA Cap (m₂^{7,3′-O}G(5′)ppp(5′)G)) with the structure:

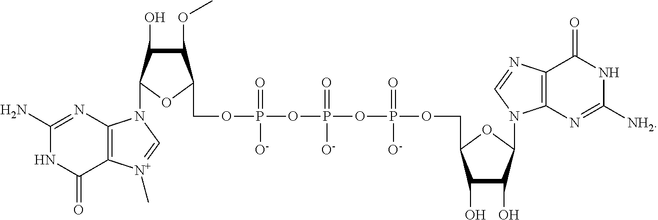

Below is an exemplary CapO RNA comprising RNA and m₂^{7,3′-O}G(5′)ppp(5′)G:

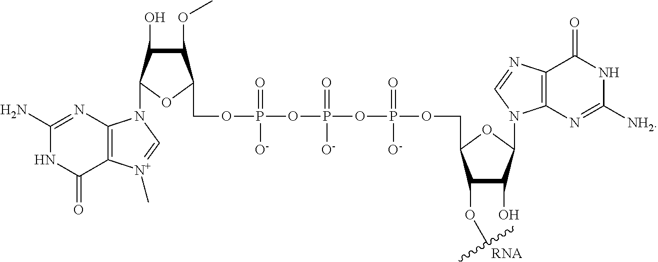

In some embodiments, the “Cap0” structures are generated during in-vitro transcription (co-transcriptional capping) using the cap analog Beta-S-ARCA (m₂^{7,3′-O}G(5′)ppSp(5′)G) with the structure:

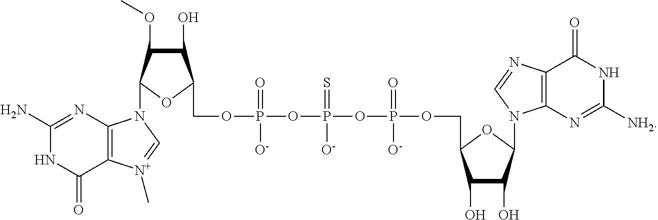

Below is an exemplary Cap0 RNA comprising Beta-S-ARCA (m₂^{7,3′-O}G(5′)ppSp(5′)G) and RNA.

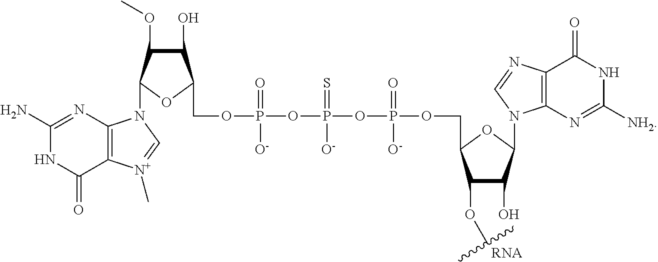

The term “uracil,” as used herein, describes one of the nucleobases that can occur in the nucleic acid of RNA. The structure of uracil is:

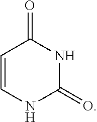

The term “uridine,” as used herein, describes one of the nucleosides that can occur in RNA. The structure of uridine is:

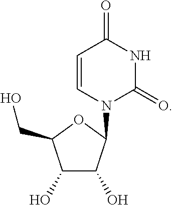 [SMILES: O=c1ccn([C@@H]2O[C@H](CO)[C@@H](O)[C@H]2O)c(=O)[nH]1]

UTP (uridine 5′-triphos hate has the following structure:

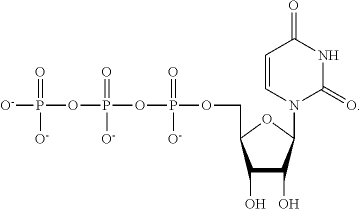

Pseudo-UTP (pseudouridine 5′-triphosphate) has the following structure:

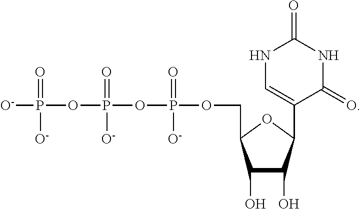

“Pseudouridine” is one example of a modified nucleoside that is an isomer of uridine, where the uracil is attached to the pentose ring via a carbon-carbon bond instead of a nitrogen-carbon glycosidic bond. Pseudouridine is described, for example, in Charette and Gray, Life; 49:341-351 (2000).

Another exemplary modified nucleoside is N1-methyl-pseudouridine (m1Ψ), which has the structure:

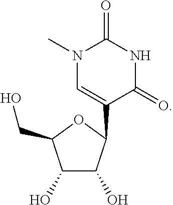 [SMILES: Cn1cc([C@@H]2O[C@H](CO)[C@@H](O)[C@H]2O)c(=O)[nH]c1=O]

N1-methyl-pseudo-UTP has the following structure:

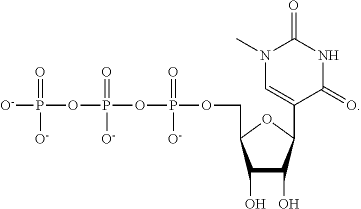

Another exemplary modified nucleoside is 5-methyl-uridine (m5U), which has the structure:

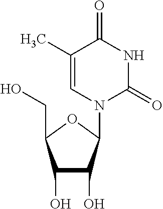

As used herein, the term “poly-A tail” or “poly-A sequence” refers to an uninterrupted or interrupted sequence of adenylate residues which is typically located at the 3′ end of an RNA molecule. Poly-A tails or poly-A sequences are known to those of skill in the art and may follow the 3′ UTR in the RNAs described herein. An uninterrupted poly-A tail is characterized by consecutive adenylate residues. In nature, an uninterrupted poly-A tail is typical. RNAs disclosed herein can have a poly-A tail attached to the free 3′ end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holikamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g. 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′ end, i.e., the poly-A tail is not masked or followed at its 3′ end by a nucleotide other than A.

In some embodiments, a poly-A tail comprises the sequence: AAAAAAAAAAAAAAAAAAAAAAAAAAAAAAGCAUAUGACUAAAAAAAAAAAAAA AAAAAAAAAAAAAAAAAAAAAAAAAAAAAAAAAAAAAAAAAAAAAAAAAAAAAA AA (SEQ ID NO. 30), which is also shown in Table 1.

In general, “RNA” and “mRNA” are used interchangeably, except where the context makes clear that one or the other is appropriate, such as where “mRNA” is appropriate to use to distinguish from other types of RNA (rRNA or tRNA) and where “RNA” is appropriate to refer to the structure of the transcription product prior to the 5′ capping to form a mRNA.

“IFNα” is used generically herein to describe any interferon alpha Type I cytokine, including IFNα2b and IFNα4.

The term “treatment,” as used herein, covers any administration or application of a therapeutic for disease in a subject, and includes inhibiting the disease, arresting its development, relieving one or more symptoms of the disease, curing the disease, or preventing reoccurrence of the disease. For example, treatment of a solid tumor may comprise alleviating symptoms of the solid tumor, decreasing the size of the solid tumor, eliminating the solid tumor, reducing further growth of the tumor, or reducing or eliminating recurrence of a solid tumor after treatment. Treatment may also be measured as a change in a biomarker of effectiveness or in an imaging or radiographic measure.

The term “monotherapy,” as used herein, means a therapy that uses one type of treatment, such as, e.g., RNA therapy alone, radiation therapy alone, or surgery alone, to treat a certain disease or condition (such as cancer). In drug therapy, monotherapy refers to the use of a single drug (which may include multiple active agents, such as, e.g., a mixture of RNAs) to treat a disease or condition. In some embodiments, the monotherapy is a therapy that is administered to treat cancer, without any other therapy that is used to treat the cancer. In some embodiments, a monotherapy for treating a cancer may optionally be combined with another treatment to ameliorate a symptom of the cancer but not treat the cancer per se (e.g., the treatment is not intended or expected to impact the growth or size of a solid tumor), but may not be combined with any other therapy directed against the cancer, such as, e.g., a chemotherapeutic agent or radiation therapy. In such embodiments, administering a mixture of RNAs as a monotherapy means administering the mixture of RNAs without, e.g., radiation therapy or any chemotherapeutic agent. However, in such embodiments, administering a mixture of RNAs as a monotherapy does not preclude administering concurrently or simultaneously with the mixture of RNAs, agents that are not directed against the cancer, such as, e.g., agents that reduce pain.

The term “prevention,” as used herein, means inhibiting or arresting development of cancer, including solid tumors, in a subject deemed to be cancer free.

“Metastasis” means the process by which cancer spreads from the place at which it first arose as a primary tumor to other locations in the body.

The term “intratumorally,” or “intratumoral” as used herein, means into the tumor. For example, intra-tumoral injection means injecting the therapeutic at any location that touches the tumor.

As used herein, “lymphoma” is a solid tumor cancer derived from lymphocytes. Lymphoma includes Hodgkin and Non-Hodgkin lymphoma. Lymphoma forms solid tumors/neoplasms within lymph nodes, and can also be found in non-lymph node tissues when metastasized.

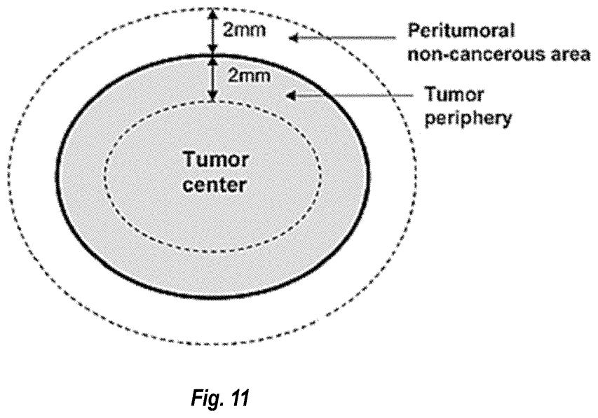

The term “peri-tumorally,” or “peri-tumoral,” or “peritumoral.” or “peritumorally” as used herein, is an area that is about 2-mm wide and is adjacent to the invasive front of the tumor periphery. The peri-tumoral area comprises host tissue. See, for example, FIG. 11.

“Administering” means providing a pharmaceutical agent or composition to a subject, and includes, but is not limited to, administering by a medical professional and self-administering.

The disclosure describes nucleic acid sequences and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes the ability to act as a cytokine, in particular when administered to a subject. In some embodiments, a nucleic acid sequence or amino acid sequence having a particular degree of identity to a given nucleic acid sequence or amino acid sequence is functionally equivalent to the given sequence.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). As used in a clause of a claim, the transitional term “comprising”, which is synonymous with “including,” “containing,” or “characterized by,” is inclusive or open-ended and does not exclude additional, unrecited elements or method steps. As used in a clause of a claim, the transitional phrase “consisting of” excludes any element, step, or component not specified in the claim, and the transitional phrase “consisting essentially of” limits the scope of the claim term to the recited components and those that do not materially affect the basic and novel characteristics of the claimed term, as understood from the specification.

### Administered RNAs

In some embodiments, methods for treating advanced-stage solid tumor cancers are encompassed comprising administering to a subject having an advanced-stage solid tumor cancer RNA encoding an IL-12sc protein, RNA encoding an IL-15 sushi protein, RNA encoding an IFNα protein, and RNA encoding a GM-CSF protein. Details of the administered RNA follow.

In some embodiments, administering RNAs comprises administering RNA encoding IFNα. RNA encoding IL-15 sushi, RNA encoding IL-12sc, and RNA encoding GM-CSF, optionally modified to have a modified nucleobase in place of each uridine and a Cap1 structure at the 5′ end of the RNA.

In some embodiments, administering RNAs comprises administering RNA encoding IL-12sc and further administering an RNA encoding IFNα, IL-15 sushi, and GM-CSF.

In some embodiments, administering RNAs comprises administering RNA encoding IFNα and further administering an RNA encoding IL-12sc, IL-15 sushi, and GM-CSF.

In some embodiments, administering RNAs comprises administering RNA encoding IL-15 sushi and further administering an RNA encoding IL-12sc, IFNα, and GM-CSF.

In some embodiments administering RNAs comprises administering RNA encoding GM-CSF sushi and further administering an RNA encoding IL-12sc, IFNα, and IL-15 sushi.

In some embodiments, the IFNα protein in the cytokine RNA mixture is an IFNα2b protein, and the method comprises administering RNA encoding an IFNα2b protein.

In some embodiments, (i) the RNA encoding an IL-12sc protein comprises the nucleotide sequence of SEQ ID NO: 17 or 18, or a nucleotide sequence having at least 99%, 98%, 97%, 96%, 95%, 90%, 85%, or 80% identity to the nucleotide sequence of SEQ ID NO: 17 or 18 and/or (ii) the IL-12sc protein comprises the amino acid sequence of SEQ ID NO: 14, or an amino acid sequence having at least 99%, 98%, 97%, 96%, 95%, 90%, 85%, or 80% identity to the amino acid sequence of SEQ ID NO: 14.

In some embodiments, (i) the RNA encoding an IL-15 sushi protein comprises the nucleotide sequence of SEQ ID NO: 26, or a nucleotide sequence having at least 99%, 98%, 97%, 96%, 95%, 90%, 85%, or 80% identity to the nucleotide sequence of SEQ ID NO: 26 and/or (ii) the IL-15 sushi protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.

In some embodiments, (i) the RNA encoding an IFNα protein comprises the nucleotide sequence of SEQ ID NO: 22 or 23, or a nucleotide sequence having at least 99%, 98%, 97%, 96%, 95%, 90%, 85%, or 80% identity to the nucleotide sequence of SEQ ID NO: 22 or 23 and/or (ii) the IFNα protein comprises the amino acid sequence of SEQ ID NO: 19, or an amino acid sequence having at least 99%, 98%, 97%, 96%, 95%, 90%, 85%, or 80% identity to the amino acid sequence of SEQ ID NO: 19.

In some embodiments, (i) the RNA encoding a GM-CSF protein comprises the nucleotide sequence of SEQ ID NO: 29, or a nucleotide sequence having at least 99%, 98%, 97%, 96%, 95%, 90%, 85%, or 80% identity to the nucleotide sequence of SEQ ID NO: 29 and/or (ii) the GM-CSF protein comprises the amino acid sequence of SEQ ID NO: 27, or an amino acid sequence having at least 99%, 98%, 97%, 96%, 95%, 90%, 85%, or 80% identity to the amino acid sequence of SEQ ID NO: 27.

### Interleukin-12 Single-Chain (IL-12sc)

In some embodiments, an RNA that encodes interleukin-12 single-chain (IL-12sc) is provided. In some embodiments, the interleukin-12 single-chain (IL-12sc) RNA is encoded by a DNA sequence encoding interleukin-12 single-chain (IL-12sc) (e.g., SEQ ID NO: 14), which comprises IL-12 p40 (sometimes referred to as IL-12B; encoded by nucleotides 1-984 of SEQ ID NO: 15), a linker, such as a GS linker, and IL-12 p35 (sometimes referred to as IL-12A; encoded by nucleotides 1027-1623 of SEQ ID NO: 15). In some embodiments, the IL-12p40, linker, and IL-12p35 are consecutive with no intervening nucleotides. An exemplary DNA sequence encoding IL-12sc is provided in SEQ ID NO: 15. In some embodiments, the interleukin-12 single-chain (IL-12sc) RNA is provided at SEQ ID NO: 17 or 18, both of which encode the protein of SEQ ID NO: 14. The RNA sequence of IL-12 p40 is shown at nucleotides 1-984 of SEQ ID NO: 17 or 18 and the RNA sequence of IL-12 p35 is shown at nucleotides 1027-1623 of SEQ ID NO: 17 or 18.

In some embodiments, the IL-12sc RNA is encoded by a codon-optimized DNA sequence encoding IL-12sc. In some embodiments, the IL-12sc RNA is encoded by a codon-optimized DNA sequence encoding IL-12 p40. In some embodiments, the IL-12sc RNA is encoded by a codon-optimized DNA sequence encoding IL-12 p35. In some embodiments, the codon-optimized DNA sequence comprises or consists of SEQ ID NO: 16. In some embodiments, the DNA sequence comprises a codon-optimized DNA sequence with 70%, 75%, 80%, 85%, 90%, 95%, 96%, 97%, 98%, or 99% identity to SEQ ID NO: 16. In some embodiments, the codon-optimized DNA sequence encoding IL-12 p40 comprises the nucleotides encoding the IL-12sc-p40 (nucleotides 1-984 of SEQ ID NO: 16). In some embodiments, the codon-optimized DNA sequence encoding IL-12 p35 comprises the nucleotides encoding the IL-12sc-p35 (nucleotides 1027-1623 of SEQ ID NO: 16). In some embodiments, the codon-optimized DNA sequence encoding IL-12sc comprises the nucleotides encoding the IL-12sc-p40 (nucleotides 1-984 of SEQ ID NO: 16) and -p35 (nucleotides 1027-1623 of SEQ ID NO: 16) portions of SEQ ID NO: 16 and further comprises nucleotides between the p40 and p35 portions (e.g., nucleotides 985-1026 of SEQ ID NO: 16) encoding a linker polypeptide connecting the p40 and p35 portions. Any linker known to those of skill in the art may be used. The p40 portion may be 5′ or 3′ to the p35 portion.

In some embodiments, the IL-12sc RNA comprises an RNA sequence that is, for example, transcribed from a DNA sequence encoding IL-12sc. The RNA may also be recombinantly produced. In some embodiments, the RNA sequence is transcribed from a nucleotide sequence comprising SEQ ID NOs: 15 or 16. In some embodiments, the RNA sequence comprises or consists of SEQ ID NOs: 17 or 18. In some embodiments, the RNA sequence comprises or consists of an RNA sequence with 70%, 75%, 80%, 85%, 90%, 95%, 96%, 97%, 98%, or 99% identity to SEQ ID NOs: 17 or 18. In some embodiments, the RNA sequence comprises the nucleotides encoding the IL-12sc-p40 (nucleotides 1-984 of SEQ ID NOs: 17 or 18) and -p35 (nucleotides 1027-1623 of SEQ ID NOs: 17 or 18) portions of SEQ ID NOs: 17 or 18. In some embodiments, the codon-optimized RNA sequence encoding IL-12sc comprises the nucleotides encoding the IL-12sc-p40 (nucleotides 1-984 of SEQ ID NO: 18) and −p35 (nucleotides 1027-1623 of SEQ ID NO: 18) portions of SEQ ID NO: 18 and further comprises nucleotides between the p40 and p35 portions encoding a linker polypeptide connecting the p40 and p35 portions. Any linker known to those of skill in the art may be used.

In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-12sc RNA comprises an altered nucleotide at the 5′ end. In some embodiments, the RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-O or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′O-mG(5′)ppsp(5′)G, m7G(5′)ppp(5′)2′O-mG, and m7,3′O-mG(5′)ppp(5′)2′O-mA.

In some embodiments, the IL-12sc RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from SEQ ID NOs: 3 or 5. In some embodiments, the 5′ UTR RNA sequence comprises or consists of SEQ ID NOs: 4 or 6. In some embodiments, the 5′ UTR RNA sequence is at least 70%, 75%, 80%, 85%, 90%, 95%, 96%, 97%, 98%, or 99% identical to SEQ ID NOs: 4 or 6.

In some embodiments, the IL-12sc RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the IL-12sc RNA comprises both a 5′ UTR and a 3′ UTR. In some embodiments, the IL-12sc RNA comprises only a 5′ UTR. In some embodiments, the IL-12sc RNA comprises only a 3′ UTR.

In some embodiments, the IL-12sc RNA comprises a poly-A tail. In some embodiments, the RNA comprises a poly-A tail of at least about 25, at least about 30, at least about 50 nucleotides, at least about 70 nucleotides, or at least about 100 nucleotides. In some embodiments, the poly-A tail comprises 200 or more nucleotides. In some embodiments, the poly-A tail comprises or consists of SEQ ID NO: 30.

In some embodiments, the RNA comprises a 5′ cap, a 5′ UTR, a nucleic acid encoding IL-12sc, a 3′ UTR, and a poly-A tail, in that order.

In some embodiments, the IL-12sc RNA is encoded by a DNA sequence comprising or consisting of a nucleic acid sequence that is at least 70%, 75%, 80%, 85%, 90%, 95%, 96%, 97%, 98%, 99%, or 100% identical to SEQ ID NOs: 15 or 16 and at least 70%, 75%, 80%, 85%, 90%, 95%, 96%, 97%, 98%, 99%, or 100% identical to SEQ ID NOs: 3 or 5.

In some embodiments, the IL-12sc RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16 and at least 70%, 75%, 80%, 85%, 90%, 95%, 96%, 97%, 98%, 99%, or 100% identical to SEQ ID NOs: 3 or 5. The RNA may also be recombinantly produced. In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-12sc RNA is encoded by a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16 and at least 70%, 75%, 80%, 85%, 90%, 95%, 96%, 97%, 98%, 99%, or 100% identical to SEQ ID NO: 7.

In some embodiments, the IL-12sc RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16 and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-12sc RNA is encoded by a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16; at least 70%, 75%, 80%, 85%, 90%, 95%, 96%, 97%, 98%, 99%, or 100% identical to SEQ ID NOs: 3 or 5; and at least 70%, 75%, 80%, 85%, 90%, 95%, 96%, 97%, 98%, 99%, or 100% identical to SEQ ID NO: 7.

In some embodiments, the IL-12sc RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 15 or 16; at least 70%, 75%, 80%, 85%, 90%, 95%, 96%, 97%, 98%, 99%, or 100% identical to SEQ ID NOs: 3 or 5; and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-12sc RNA comprises an RNA sequence comprising or consisting of a nucleic acid sequence at least 70%, 75%, 80%, 85%, 90%, 95%, 96%, 97%, 98%, 99%, or 100% identical to SEQ ID NOs: 17 or 18; at least 70%, 75%, 80%, 85%, 90%, 95%, 96%, 97%, 98%, 99%, or 100% identical to SEQ ID NOs: 4 or 6, and at least 70%, 75%, 80%, 85%, 90%, 95%, 96%, 97%, 98%, 99%, or 100% identical to SEQ ID NO: 8. In some embodiments, one or more uridine in the IL-12sc RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U).

Interferon Alpha (IFNα)

In some embodiments, the interferon alpha (IFNα) RNA is encoded by a DNA sequence encoding interferon alpha (IFNα) (e.g., SEQ ID NO: 19). An exemplary DNA sequence encoding this IFNα is provided in SEQ ID NO: 20.

In some embodiments, the IFNα RNA is encoded by a codon-optimized DNA sequence encoding IFNα. In some embodiments, the codon-optimized DNA sequence comprises or consists of the nucleotides of SEQ ID NO: 21. In some embodiments, the DNA sequence comprises or consists of a codon-optimized DNA sequence with 70%, 75%, 80%, 85%, 90%, 95%, 96%, 97%, 98%, or 99% identity to SEQ ID NO: 21.

In some embodiments, the IFNα RNA comprises an RNA sequence that is, for example, transcribed from a DNA sequence encoding IFNα. The RNA may also be recombinantly produced. In some embodiments, the RNA sequence is transcribed from a nucleotide sequence comprising SEQ ID NOs: 20 or 21. In some embodiments, the RNA sequence comprises or consists of SEQ ID NOs: 22 or 23. In some embodiments, the RNA sequence comprises or consists of an RNA sequence with 70%, 75%, 80%, 85%, 90%, 95%, 96%, 97%, 98%, or 99% identity to SEQ ID NOs: 22 or 23.

In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, each uridine in the RNA is modified. In some embodiments, each uridine in the RNA is modified with N1-methyl-pseudouridine (m¹ψ).

In some embodiments, the IFNα RNA comprises an altered nucleotide at the 5′ end. In some embodiments, the IFNα RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-O or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′O-mG(5′)ppsp(5′)G, m7G(5′)ppp(5′)2′O-mGand m7,3′O-mG(5′)ppp(5′)2′O-mA.

In some embodiments, the IFNα RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from a nucleotide sequence comprising SEQ ID NOs: 3 or 5. In some embodiments, the 5′ UTR RNA sequence comprises or consists of SEQ ID NOs: 4 or 6. In some embodiments, the 5′ UTR RNA sequence is at least 70%, 75%, 80%, 85%, 90%, 95%, 96%, 97%, 98%, or 99% identical to SEQ ID NOs: 4 or 6.

In some embodiments, the IFNα RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from a nucleotide sequence comprising SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the IFNα RNA comprises both a 5′ UTR and a 3′ UTR In some embodiments, the composition comprises only a 5′ UTR. In some embodiments, the composition comprises only a 3′ UTR.

In some embodiments, the IFNα RNA comprises a poly-A tail. In some embodiments, the IFNα RNA comprises a poly-A tail of at least about 25, at least about 30, at least about 50 nucleotides, at least about 70 nucleotides, or at least about 100 nucleotides. In some embodiments, the poly-A tail comprises 200 or more nucleotides. In some embodiments, the poly-A tail comprises or consists of SEQ ID NO: 30.

In some embodiments, the RNA comprises a 5′ cap, a 5′ UTR, a nucleic acid encoding IFNα, a 3′ UTR, and a poly-A tail, in that order.

In some embodiments, the IFNu RNA is encoded by a DNA sequence comprising or consisting of a nucleic acid sequence that is at least 70%, 75%, 80%, 85%, 90%, 95%, 96%, 97%, 98%, 99%, or 100% identical to SEQ ID NOs: 20 or 21 and at least 70%, 75%, 80%, 85%, 90%, 95%. %%, 97%, 98%, 99%, or 100% identical to SEQ ID NOs: 3 or 5.

In some embodiments, the IFNα RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21 and at least 70%, 75%, 80%, 85%, 90%, 95%, %%, 97%, 98%, 99%, or 100% identical to SEQ ID NOs: 3 or 5. The RNA may also be recombinantly produced. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IFNα RNA is encoded by a DNA sequence comprising or consisting of a nucleic acid sequence at least 70%, 75%, 80%, 85%, 90%, 95%. %%, 97%, 98%, 99%, or 100% identical to SEQ ID NOs: 20 or 21 and at least 70%, 75%, 80%, 85%, 90%, 95%, 96%, 97%, 98%, 99%, or 100% identical to SEQ ID NO: 7.

In some embodiments, the IFNα RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21 and at least 70%, 75%, 80%, 85%, 90%, 95%, %%, 97%, 98%, 99%, or 100% identical to SEQ ID NO: 7. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IFNα RNA is encoded by a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21; at least 70%, 75%, 80%, 85%, 90%, 95%, 96%, 97%, 98%, 99%, or 100% identical to SEQ ID NOs: 3 or 5; and at least 70%, 75%, 80%, 85%, 90%, 95%, 96%, 97%, 98%, 99%, or 100% identical to SEQ ID NO: 7.

In some embodiments, the IFNα RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NOs: 20 or 21; at least 70%, 75%, 80%, 85%, 90%, 95%, 96%, 97%, 98%, 99%, or 100% identical to SEQ ID NOs: 3 or 5; and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).In some embodiments, the composition comprises an RNA sequence comprising or consisting of a nucleic acid sequence at least 70%, 75%, 80%, 85%, 90%, 95%, 96%, 97%, 98%, 99%, or 100% identical to SEQ ID NOs: 22 or 23; at least 70%, 75%, 80%, 85%, 90%, 95%, 96%, 97%, 98%, 99%, or 100% identical to SEQ ID NOs: 4 or 6; and at least 70%, 75%, 80%, 85%, 90%, 95%, 96%, 97%, 98%, 99%, or 100% identical to SEQ ID NO: 8. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m¹ψ).

Interleukin 15 (IL-15) Sushi

In some embodiments, an RNA that encodes an interleukin-15 (IL-15) sushi is administered. As used herein, the term “IL-15 sushi” describes a construct comprising the soluble interleukin 15 (IL-15) receptor alpha sushi domain and mature interleukin alpha (IL-15) as a fusion protein. In some embodiments, the IL-15 sushi RNA is encoded by a DNA sequence encoding IL-15 sushi (SEQ ID NO: 24), which comprises the soluble IL-15 receptor alpha chain (sushi) followed by a glycine-serine (GS) linker followed by the mature sequence of IL-15. The DNA sequence encoding this IL-15 sushi is provided in SEQ ID NO: 25.

In some embodiments, the IL-15 sushi RNA is an RNA sequence that is, for example, transcribed from a DNA sequence encoding IL-15 sushi. The RNA may also be recombinantly produced. In some embodiments, the RNA sequence is transcribed from a nucleotide sequence comprising SEQ ID NO: 25. In some embodiments, the nucleotides encoding the linker may be completely absent or replaced in part or in whole with any nucleotides encoding a suitable linker. In some embodiments, the RNA sequence comprises or consists of SEQ ID NO: 26. In some embodiments, the RNA sequence comprises an RNA sequence with 70%, 75%, 80%, 85%, 90%, 95%, 96%, 97%, 98%, or 99% identity to SEQ ID NO: 26. In some embodiments, the DNA or RNA sequence encoding IL-15 sushi comprises the nucleotides encoding the sushi domain of IL-15 receptor alpha (e.g., nucleotide 1-321 of SEQ ID NOs: 25 or 26) and mature IL-15 (e.g., nucleotide 382-729 of SEQ ID NO: 25 or 26). In some embodiments, the DNA or RNA sequence encoding IL-15 sushi comprises the nucleotides encoding the sushi domain of IL-15 receptor alpha (e.g., nucleotide 1-321 of SEQ ID NOs: 25 or 26) and mature IL-15 (e.g., nucleotide 382-729 of SEQ ID NOs: 25 or 26) and further comprises nucleotides between these portions encoding a linker polypeptide connecting the portions. In some embodiments, the linker comprises nucleotides 322-381 of SEQ ID Nos: 25 or 26. Any linker known to those of skill in the art may be used.

In some embodiments, one or more uridine in the IL-15 sushi RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-15 sushi RNA comprises an altered nucleotide at the 5′ end. In some embodiments, the IL-15 sushi RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-O or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′O-mG(5′)ppsp(5′)G, m7G(5′)ppp(5′)2′O-mG and m7,3′O-mG(5′)ppp(5′)2′-mA.

In some embodiments, the IL-15 sushi RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from SEQ ID NOs: 3 or 5. In some embodiments, the 5′ UTR RNA sequence comprises or consists of SEQ ID NOs: 4 or 6. In some embodiments, the 5′ UTR RNA sequence is at least 70%, 75%, 80%, 85%, 90%, 95%, 96%, 97%, 98%, or 99% identical to SEQ ID NOs: 4 or 6.

In some embodiments, the IL-15 sushi RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the IL-15 sushi RNA comprises both a 5′ UTR and a 3′ UTR. In some embodiments, the IL-15 sushi RNA comprises only a 5′ UTR. In some embodiments, the IL-15 sushi RNA comprises only a 3′ UTR.

In some embodiments, the IL-15 sushi RNA comprises a poly-A tail. In some embodiments, the RNA comprises a poly-A tail of at least about 25, at least about 30, at least about 50 nucleotides, at least about 70 nucleotides, or at least about 100 nucleotides. In some embodiments, the poly-A tail comprises 200 or more nucleotides. In some embodiments, the poly-A tail comprises or consists of SEQ ID NO: 30.

In some embodiments, the RNA comprises a 5′ cap, a 5′ UTR, a nucleic acid encoding IL-15 sushi, a 3′ UTR, and a poly-A tail, in that order.

In some embodiments, the IL-15 sushi RNA is encoded by a DNA sequence comprising or consisting of a nucleic acid sequence that is at least 70%, 75%, 80%, 85%, 90%, 95%, 96%, 97%, 98%, 99%, or 100% identical to SEQ ID NO: 25 and at least 70%, 75%, 80%, 85%, 90%, 95%, 96%, 97%, 98%, 99%, or 100% identical to SEQ ID NOs: 3 or 5.

In some embodiments, the IL-15 sushi RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 25 and at least 70%, 75%, 80%, 85%, 90%, 95%, 96%, 97%, 98%, 99%, or 100% identical to SEQ ID NOs: 3 or 5. The RNA may also be recombinantly produced. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-15 sushi RNA comprises a DNA sequence comprising or consisting of a nucleic acid sequence at least 70%, 75%, 80%, 85%, 90%, 95%, 96%, 97%, 98%, 99%, or 100% identical to SEQ ID NO: 25 and at least 70%, 75%, 80%, 85%, 90%, 95%, 96%, 97%, 98%, 99%, or 100% identical to SEQ ID NO: 7.

In some embodiments, the IL-15 sushi RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 25 and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-15 sushi RNA comprises a DNA sequence comprising or consisting of a nucleic acid sequence at least 70%, 75%, 80%, 85%, 90%, 95%, 96%, 97%, 98%, 99%, or 100% identical to SEQ ID NO: 25; at least 70%, 75%, 80%, 85%, 90%, 95%, 96%, 97%, 98%, 99%, or 100% identical to SEQ ID NOs: 3 or 5; and at least 70%, 75%, 80%, 85%, 90%, 95%, 96%, 97%, 98%, 99%, or 100% identical to SEQ ID NO: 7.

In some embodiments, the IL-15 sushi RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 25; at least 70%, 75%, 80%, 85%, 90%, 95%, 96%, 97%, 98%, 99%, or 100% identical to SEQ ID NOs: 3 or 5; and at least 70%, 75%, 80%, 85%, 90%, 95%, %%, 97%, 98%, 99%, or 100% identical to SEQ ID NO: 7. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the IL-15 sushi RNA comprises an RNA sequence comprising or consisting of a nucleic acid sequence at least 70%, 75%, 80%, 85%, 90%, 95%, 96%, 97%, 98%, 99%, or 100% identical to SEQ ID NO: 26; at least 70%, 75%, 80%, 85%, 90%, 95%, 96%, 97%, 98%, 99%, or 100% identical to SEQ ID NOs: 4 or 6; and at least 70%, 75%, 80%, 85%, 90° %, 95%, 96%, 97%, 98%, 99%, or 100% identical to SEQ ID NO: 8. In some embodiments, one or more uridine in the IFNα RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U).

Granulocyte-Macrophage Colony-Stimulating Factor (GM-CSF)

In some embodiments, an RNA that encodes granulocyte-macrophage colony-stimulating factor (GM-CSF) is administered. In some embodiments, the GM-CSF RNA is encoded by a DNA sequence encoding granulocyte-macrophage colony-stimulating factor (GM-CSF) (e.g., SEQ ID NO: 27). In some embodiments, the DNA sequence encoding GM-CSF is provided in SEQ ID NO: 28.

In some embodiments, the GM-CSF RNA comprises an RNA sequence that is, for example, transcribed from a DNA sequence encoding GM-CSF. In some embodiments, the RNA sequence is transcribed from SEQ ID NO: 28. The RNA may also be recombinantly produced. In some embodiments, the RNA sequence comprises or consists of SEQ ID NO: 29. In some embodiments, the RNA sequence comprises an RNA sequence with 70%, 75%, 80%, 85%, 90%, 95%, 96%, 97%, 98%, or 99% identity to SEQ ID NOs: 29.

In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ). In some embodiments, the GM-CSF RNA comprises an altered nucleotide at the 5′ end. In some embodiments, the RNA comprises a 5′ cap. Any 5′ cap known in the art may be used. In some embodiments, the 5′ cap comprises a 5′ to 5′ triphosphate linkage. In some embodiments, the 5′ cap comprises a 5′ to 5′ triphosphate linkage including thiophosphate modification. In some embodiments, the 5′ cap comprises a 2′-0 or 3′-O-ribose-methylated nucleotide. In some embodiments, the 5′ cap comprises a modified guanosine nucleotide or modified adenosine nucleotide. In some embodiments, the 5′ cap comprises 7-methylguanylate. In some embodiments, the 5′ cap is Cap0 or Cap1. Exemplary cap structures include m7G(5′)ppp(5′)G, m7,2′O-mG(5′)ppsp(5′)G, m7G(5′)ppp(5′)2′O-mG and m7,3′O-mG(5′)ppp(5′)2′O-mA.

In some embodiments, the GM-CSF RNA comprises a 5′ untranslated region (UTR). In some embodiments, the 5′ UTR is upstream of the initiation codon. In some embodiments, the 5′ UTR regulates translation of the RNA. In some embodiments, the 5′ UTR is a stabilizing sequence. In some embodiments, the 5′ UTR increases the half-life of RNA. Any 5′ UTR known in the art may be used. In some embodiments, the 5′ UTR RNA sequence is transcribed from SEQ ID NOs: 3 or 5. In some embodiments, the 5′ UTR RNA sequence comprises or consists of SEQ ID NOs: 4 or 6. In some embodiments, the 5′ UTR RNA sequence is at least 70%, 75%, 80%, 85%, 90%, 95%, 96%, 97%, 98%, or 99% identical to SEQ ID NOs: 4 or 6.

In some embodiments, the GM-CSF RNA comprises a 3′ UTR. In some embodiments, the 3′ UTR follows the translation termination codon. In some embodiments, the 3′ UTR regulates polyadenylation, translation efficiency, localization, or stability of the RNA. In some embodiments, the 3′ UTR RNA sequence is transcribed from SEQ ID NO: 7. In some embodiments, the 3′ UTR RNA sequence comprises or consists of SEQ ID NO: 8. In some embodiments, the 3′ UTR RNA sequence is at least 70%, 75%, 80%, 85%, 90%, 95%, 96%, 97%, 98%, or 99% identical to SEQ ID NO: 8.

In some embodiments, the GM-CSF RNA comprises both a 5′ UTR and a 3′ UTR. In some embodiments, the RNA comprises only a 5′ UTR. In some embodiments, the composition comprises only a 3′ UTR.

In some embodiments, the GM-CSF RNA comprises a poly-A tail. In some embodiments, the RNA comprises a poly-A tail of at least about 25, at least about 30, at least about 50 nucleotides, at least about 70 nucleotides, or at least about 100 nucleotides. In some embodiments, the poly-A tail comprises 200 or more nucleotides. In some embodiments, the poly-A tail comprises or consists of SEQ ID NO: 30.

In some embodiments, the GM-CSF RNA comprises a 5′ cap, a 5′ UTR, nucleotides encoding GM-CSF, a 3′ UTR, and a poly-A tail, in that order.

In some embodiments, the GM-CSF RNA is encoded by a DNA sequence comprising or consisting of a nucleic acid sequence that is at least 70%, 75%, 80%, 85%, 90%, 95%, 96%, 97%, 98%, 99%, or 100% identical to SEQ ID NO: 28 and at least 70%, 75%, 80%, 85%, 90%, 95%, 96%, 97%, 98%, 99%, or 100% identical to SEQ ID NOs: 3 or 5.

In some embodiments, the GM-CSF RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 28 and at least 70%, 75%, 80%, 85%, 90%, 95%, 96%, 97%, 98%, 99%, or 100% identical to SEQ ID NOs: 3 or 5. The RNA may also be recombinantly produced. In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the GM-CSF RNA is encoded by a DNA sequence comprising or consisting of a nucleic acid sequence at least 70%, 75%, 80%, 85%, 90%, 95%, 96%, 97%, 98%, 99%, or 100% identical to SEQ ID NO: 28 and at least 70%, 75%, 80%, 85%, 90%, 95%, 96%, 97%, 98%, 99%, or 100/o identical to SEQ ID NO: 7.

In some embodiments, the GM-CSF RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 28 and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the GM-CSF RNA is replaced by a modified nucleoside as described herein. In some embodiments, the modified nucleoside replacing uridine is pseudouridine (ψ). N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the GM-CSF RNA comprises a DNA sequence comprising or consisting of a nucleic acid sequence at least 70%, 75%, 80%, 85%, 90%, 95%, 96%, 97%, 98%, 99%, or 100% identical to SEQ ID NO: 28; at least 70%, 75%, 80%, 85%, 90%, 95%, 96%, 97%, 98%, 99%, or 100% identical to SEQ ID NOs: 3 or 5; and at least 70%, 75%, 80%, 85%, 90%, 95%, 96%, 97%, 98%, 99%, or 100% identical to SEQ ID NO: 7.

In some embodiments, the GM-CSF RNA comprises an RNA sequence that is, for example, transcribed from a DNA sequence comprising or consisting of a nucleic acid sequence at least 70%, 75%, 80%, 85%, 90%, 95%, 96%, 97%, 98%, 99%, or 100% identical to SEQ ID NO: 28; at least 70%, 75%, 80%, 85%, 90%, 95%, %*%, 97%, 98%, 99%, or 100% identical to SEQ ID NOs: 3 or 5; and at least 70%, 75%, 80%, 85%, 90%, 95%, 96%, 97%, 98%, 99%, or 100% identical to SEQ ID NO: 7. The RNA may also be recombinantly produced. In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U). In some embodiments, the RNA comprises a modified nucleoside in place of each uridine. In some embodiments, the modified nucleoside is N1-methyl-pseudouridine (m¹ψ).

In some embodiments, the GM-CSF RNA comprises an RNA sequence comprising or consisting of a nucleic acid sequence at least 0%, 75%, 80%, 85%, 90%, 95%, 96%, 97%, 98%, 99%, or 100% identical to SEQ ID NO: 29; at least 70%, 75%, 80%, 85%, 90%, 95%, %*%, 97%, 98%, 99%, or 100% identical to SEQ ID NOs: 4 or 6; and at least 70%, 75%, 80%, 85%, 90%, 95%, 96%, 97%, 98%, 99%, or 100% identical to SEQ ID NO: 8. In some embodiments, one or more uridine in the GM-CSF RNA is replaced by a modified nucleoside as described herein. In some embodiments, the modified nucleoside replacing uridine is pseudouridine (ψ), N1-methyl-pseudouridine (m¹ψ) or 5-methyl-uridine (m⁵U).

Modifications

Each of the RNAs described herein may be modified in any way known to those of skill in the art. In some embodiments, each RNA is modified as follows:
a modified nucleobase in place of each uridine;
a Cap1 structure at the 5′ end of the RNA.

In some embodiments, the 5′ UTR comprises SEQ ID NOs: 4 or 6. In some embodiments, the RNA has been processed to reduce double-stranded RNA (dsRNA) as described above. The “Cap1” structure may be generated after in-vitro transcription by enzymatic capping or during in-vitro transcription (co-transcriptional capping).

In some embodiments, one or more uridine in the RNA is replaced by a modified nucleoside. In some embodiments, the modified nucleoside is a modified uridine.

In some embodiments, the modified uridine replacing uridine is pseudouridine (ψ), N1-methyl-pseudouridine (m1ψ), or 5-methyl-uridine (m5U).

In some embodiments, one or more cytosine, adenine or guanine in the RNA is replaced by modified nucleobase(s). In one embodiment, the modified nucleobase replacing cytosine is 5-methylcytosine (m⁵C). In another embodiment, the modified nucleobase replacing adenine is N⁶-methyladenine (m⁶A). In another embodiment, any other modified nucleobase known in the art for reducing the immunogenicity of the molecule can be used.

In some embodiments, the modified nucleoside replacing one or more uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-undineor 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonvlmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm³U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (Wm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, at least one RNA comprises a modified nucleoside in place of at least one uridine. In some embodiments, at least one RNA comprises a modified nucleoside in place of each uridine. In some embodiments, each RNA comprises a modified nucleoside in place of at least one uridine. In some embodiments, each RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, at least one RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, at least one RNA used in the method comprises the 5′ cap m₂^{7,3′-O}Gppp(m₁^{2′-O})ApG or 3′-O-Me-m⁷G(5′)ppp(5′)G. In some embodiments, each RNA used in the method comprises the 5′ cap m₂^{7,3′-O}Gppp(m₁^{2′-O})ApG or 3′-O-Me-m⁷G(5′)ppp(5′)G. In some embodiments, each RNA used in the method comprises the 5′ cap m₂^{7,3′-O}Gppp(m₁^{2′-O})ApG. In some embodiments, each RNA used in the method comprises the 3′-O-Me-m⁷G(5′)ppp(5′)G. In some embodiments, each RNA used in the method comprises the 5′ cap m₂^{7,3′-O}Gppp(m₁^{2′-O})ApG and 3′-O-Me-m⁷G(5′)ppp(5′)G.

In some embodiments, at least one RNA comprises a 5′ UTR comprising a nucleotide sequence selected from the group consisting of SEQ ID NOs: 4 and 6, or a nucleotide sequence having at least 99%, 98%, 97%, 96%, 95%, 90%, 85%, or 80% identity to a nucleotide sequence selected from the group consisting of SEQ ID NOs: 4 and 6. In some embodiments, each RNA comprises a 5′ UTR comprising a nucleotide sequence selected from the group consisting of SEQ ID NOs: 4 and 6, or a nucleotide sequence having at least 99%, 98%, 97%, 96%, 95%, 90%, 85%, or 80% identity to a nucleotide sequence selected from the group consisting of SEQ ID NOs: 4 and 6.

In some embodiments, at least one RNA comprises a 3′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8. In some embodiments, each RNA comprises a 3′ UTR comprising the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.

In some embodiments, at least one RNA comprises a poly-A tail. In some embodiments, each RNA comprises a poly-A tail. In some embodiments, the poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 A nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail may comprise the poly-A tail shown in SEQ ID NO: 30. In some embodiments, the poly-A tail comprises at least 100 nucleotides. In some embodiments, the poly-A tail comprises about 150 nucleotides. In some embodiments, the poly-A tail comprises about 120 nucleotides.

In some embodiments, one or more RNA comprises: (1) a 5′ cap comprising m₂^{7,3′-O}Gppp(m₁^{2′-O})ApG or 3′-O-Me-m⁷G(5′)ppp(5′)G; (2) a 5′ UTR comprising (i) a nucleotide sequence selected from the group consisting of SEQ ID NOs: 4 and 6, or (ii) a nucleotide sequence having at least 99%, 98%, 97%, 96%, 95%, 90%, 85%, or 80% identity to a nucleotide sequence selected from the group consisting of SEQ ID NOs: 4 and 6; (3) a 3′ UTR comprising (i) the nucleotide sequence of SEQ ID NO: 8, or (ii) a nucleotide sequence having at least 99%, 98%, 97%, 96%, 95%, 90%, 85%, or 80% identity to the nucleotide sequence of SEQ ID NO:8; and (4) a poly-A tail comprising at least 100 nucleotides.

### Therapeutic Methods

The cytokine RNA mixture provided herein may be used in methods, e.g., therapeutic methods. In some embodiments, methods for treating advanced-stage, unresectable, or metastatic solid tumor cancers are encompassed, comprising administering the cytokine RNA mixture, wherein the advanced-stage solid tumor cancer comprises an epithelial tumor, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, melanoma tumor, small cell lung tumor, neuroblastoma tumor, testicular tumor, carcinoma tumor, adenocarcinoma tumor, seminoma tumor, retinoblastoma, cutaneous squamous cell carcinoma (CSCC), lymphoma, including Non-Hodgkin lymphoma and Hodgkin lymphoma, squamous cell carcinoma for the head and neck (HNSCC), head and neck cancer, osteosarcoma tumor, non-small cell lung cancer, kidney tumor, thyroid tumor, liver tumor, other solid tumors amenable to intratumoral injection, or combinations thereof

In some embodiments, the advanced-stage solid tumor cancer comprises an epithelial tumor, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, melanoma tumor, small cell lung tumor, neuroblastoma tumor, testicular tumor, carcinoma tumor, adenocarcinoma tumor, seminoma tumor, retinoblastoma, cutaneous squamous cell carcinoma (CSCC), squamous cell carcinoma for the head and neck (HNSCC), head and neck cancer, osteosarcoma tumor, non-small cell lung cancer, kidney tumor, thyroid tumor, liver tumor, other solid tumors amenable to intratumoral injection, or combinations thereof.

In some embodiments, the advanced-stage solid tumor cancer comprises lymphoma, such as Non-Hodgkin lymphoma or Hodgkin lymphoma.

In some embodiments, the solid tumor cancer is melanoma. In some embodiments, the melanoma is uveal melanoma or mucosal melanoma. In some embodiments, the solid tumor cancer is melanoma, optionally uveal melanoma or mucosal melanoma, and comprises superficial, subcutaneous and/or lymph node metastases amenable for intratumoral injection.

In some embodiments, intratumoral injection comprises injection into a solid tumor metastasis within a lymph node. In some embodiments, intratumoral injection comprises injection into a lymphoma tumor within a lymph node. In some embodiments, intratumoral injection comprises injection into a primary or secondary solid tumor that is within 10 cm of the subject's skin surface. In some embodiments, intratumoral injection comprises injection into a primary or secondary solid tumor that is within 5 cm of the subject's skin surface. In some embodiments, intratumoral injection comprises injection into a cutaneous solid tumor. In some embodiments, the cutaneous solid tumor is a metastasis. In some embodiments, the cutaneous solid tumor is a skin cancer. In some embodiments, the cutaneous solid tumor is not a skin cancer. In some embodiments, intratumoral injection comprises injection into a subcutaneous solid tumor. In some embodiments, the subcutaneous solid tumor is a metastasis. In some embodiments, the subcutaneous solid tumor is a skin cancer. In some embodiments, the subcutaneous solid tumor is not a skin cancer.

In some embodiments, the solid tumor is an epithelial tumor. In some embodiments, the solid tumor is a prostate tumor. In some embodiments, the solid tumor is an ovarian tumor. In some embodiments, the solid tumor is a renal cell tumor. In some embodiments, the solid tumor is a gastrointestinal tract tumor. In some embodiments, the solid tumor is a hepatic tumor. In some embodiments, the solid tumor is a colorectal tumor. In some embodiments, the solid tumor is a tumor with vasculature. In some embodiments, the solid tumor is a mesothelioma tumor. In some embodiments, the solid tumor is a pancreatic tumor. In some embodiments, the solid tumor is a breast tumor. In some embodiments, the solid tumor is a sarcoma tumor. In some embodiments, the solid tumor is a lung tumor. In some embodiments, the solid tumor is a colon tumor. In some embodiments, the solid tumor is a melanoma tumor. In some embodiments, the solid tumor is a small cell lung tumor. In some embodiments, the solid tumor is non-small cell lung cancer tumor. In some embodiments, the solid tumor is a neuroblastoma tumor. In some embodiments, the solid tumor is a testicular tumor. In some embodiments, the solid tumor is a carcinoma tumor. In some embodiments, the solid tumor is an adenocarcinoma tumor. In some embodiments, the solid tumor is a seminoma tumor. In some embodiments, the solid tumor is a retinoblastoma. In some embodiments, the solid tumor is a cutaneous squamous cell carcinoma (CSCC). In some embodiments, the solid tumor is a squamous cell carcinoma for the head and neck (HNSCC). In some embodiments, the solid tumor is HNSCC. In some embodiments, the solid tumor is head and neck cancer. In some embodiments, the solid tumor is an osteosarcoma tumor. In some embodiments, the solid tumor is kidney cancer. In some embodiments, the solid tumor is thyroid cancer. In some embodiments, the solid tumor is anaplastic thyroid cancer (ATC). In some embodiments, the solid tumor is liver cancer. In some embodiments, the solid tumor is a colon tumor. In some embodiments, the solid tumor is any two of the above. In some embodiments, the solid tumor is any two or more of the above.

In some embodiments, the solid tumor is lymphoma. In some embodiments, the solid tumor is Non-Hodgkin lymphoma. In some embodiments, the solid tumor is Hodgkin lymphoma.

In some embodiments, the method comprises the use of a cytokine RNA mixture comprising RNA encoding IFNα, RNA encoding IL-15 sushi, RNA encoding IL-12sc, and RNA encoding GM-CSF, optionally modified to have a modified nucleobase in place of each uridine and a Cap1 structure at the 5′ end of the RNA.

In some embodiments, a method for treating an advanced-stage, unresectable, or metastatic solid tumor cancer is provided comprising administering to a subject having an advanced-stage, unresectable, or metastatic solid tumor cancer RNA encoding an IL-12sc protein, RNA encoding an IL-15 sushi protein, RNA encoding an IFNα protein, and RNA encoding a GM-CSF protein.

In some embodiments, methods for treating advanced-stage, unresectable, or metastatic solid tumor cancers are encompassed comprising administering to a subject having an advanced-stage solid tumor cancer a therapeutically effective amount of RNA comprising RNA encoding an IL-12sc protein, RNA encoding an IL-15 sushi protein, RNA encoding an IFNα protein, and RNA encoding a GM-CSF protein.

In some embodiments, a composition for use in treating advanced-stage, unresectable, or metastatic solid tumor cancers is encompassed comprising administering RNA encoding IL-12sc and further administering an RNA encoding IFNα, IL-15 sushi, and GM-CSF.

In some embodiments, a composition for use in treating advanced-stage, unresectable, or metastatic solid tumor cancers is encompassed comprising administering RNA encoding IFNα and further administering an RNA encoding IL-12sc, IL-15 sushi, and GM-CSF.

In some embodiments, a composition for use in treating advanced-stage, unresectable, or metastatic solid tumor cancers is encompassed comprising administering RNA encoding IL-15 sushi and further administering an RNA encoding IL-12sc, IFNα, and GM-CSF.

In some embodiments, a composition for use in treating advanced-stage, unresectable, or metastatic solid tumor cancers is encompassed comprising administering RNA encoding GM-CSF sushi and further administering an RNA encoding IL-12sc, IFNα, and IL-15 sushi.

### A. Administration Routes and Timing

In some embodiments, the RNAs are co-administered. In some embodiments, the RNAs are administered concurrently or sequentially. If sequential, administration can be in any order and at any appropriate time intervals known to those of skill in the art. In some embodiments, the RNAs are administered via injection into the tumor (e.g., intratumorally), or near the tumor (peri-tumorally). In some embodiments, the RNAs are mixed together in liquid solution prior to injection. In some embodiments, the RNAs are administered via direct intratumoral injection.

In some embodiments, the RNAs are injected intratumorally or peri-tumorally. In some embodiments, the RNAs are injected intratumorally.

In some embodiments, the RNAs are administered in a neoadjuvant setting. “Neoadjuvant setting” refers to a clinical setting in which the method is carried out before the primary/definitive therapy (e.g., before surgical resection of a tumor).

In some embodiments, the RNAs are administered as monotherapy. In some embodiments, the RNAs are administered as part of a combined therapy with one or more other treatment options (e.g., radiation and/or one or more chemotherapeutic agents).

In some embodiments, the cytokine RNA mixture is administered intratumorally once per week in a 3- or 4-week cycle (i.e., three doses every 21 or four doses every 28 days). In some embodiments, the cytokine RNA mixture is administered intratumorally or peri-tumorally once per week. In some embodiments, intratumoral injection continues weekly until the second tumor assessment, at which time a change of the dose interval of the cytokine RNA mixture to every three weeks may be made.

In some embodiments, the cytokine RNA mixture is administered on a 3- or 4-week cycle, wherein the cytokine RNA mixture is administered once every week. In some embodiments, the cytokine RNA mixture is administered on a 3- or 4-week cycle, wherein the cytokine RNA mixture is administered once every 2 weeks. In some embodiments, the cytokine RNA mixture is administered on a 3- or 4-week cycle, wherein the cytokine RNA mixture is administered once every 3 weeks. In some embodiments, the cytokine RNA mixture is administered on a 3- or 4-week cycle, wherein the cytokine RNA mixture is administered once every 4 weeks.

In some embodiments, the RNAs are administered for about 5, 6, 7, 8, 9, 10, 11, or 12 months. In some embodiments, the RNAs are administered for about 5 months. In some embodiments, the RNAs are administered for a maximum of 52 weeks.

In some embodiments, combinations of RNA are administered as a 1:1:1:1 ratio based on equal RNA mass (i.e., 1:1:1:1% (w/w/w/w)).

In some embodiments, the RNAs are administered in a therapeutically effective amount.

### B. Indications and Patient Populations

In some embodiments, the cytokine RNA mixture provided herein is used in a method of treating a subject having a solid tumor, wherein the subject:
i. has failed, or become intolerant, resistant, or refractory to an anti-programmed cell death 1 (PD-1) or anti-programmed cell death 1 ligand (PD-L1) therapy; and/or
ii. has a PD-1 and/or PD-L1 resistant solid tumor; and/or
iii. has acquired resistance to an anti-PD-1 and/or anti-PD-L1 therapy: and/or
iv. has innate resistance to anti-PD-1 and/or anti-PD-L1 therapy.

In some embodiments, the cytokine RNA mixture provided herein is used in a method of treating a solid tumor in a subject that has failed an anti-programmed cell death 1 (PD-1) or anti-programmed cell death 1 ligand (PD-L1) therapy.

In some embodiments, the cytokine RNA mixture provided herein is used in a method of treating a solid tumor in a subject that has become intolerant to an anti-programmed cell death 1 (PD-1) or anti-programmed cell death 1 ligand (PD-L1) therapy.

In some embodiments, the cytokine RNA mixture provided herein is used in a method of treating a solid tumor in a subject that has become resistant an anti-programmed cell death 1 (PD-1) or anti-programmed cell death 1 ligand (PD-L1) therapy.

In some embodiments, the cytokine RNA mixture provided herein is used in a method of treating a solid tumor in a subject that has become intolerant an anti-programmed cell death 1 (PD-1) or anti-programmed cell death 1 ligand (PD-L1) therapy.

In some embodiments, the cytokine RNA mixture provided herein is used in a method of treating a solid tumor in a subject that has a PD-1 and/or PD-L1 resistant solid tumor.

In some embodiments, the cytokine RNA mixture provided herein is used in a method of treating a solid tumor in a subject, wherein the subject has acquired resistance to an anti-PD-1 and/or anti-PD-L1 therapy.

In some embodiments, the cytokine RNA mixture provided herein is used in a method of treating a solid tumor in a subject, wherein the subject has innate resistance to an anti-PD-1 and/or anti-PD-L1 therapy.

In some embodiments, the subject has a metastatic solid tumor. In some embodiments, the subject has an unresectable solid tumor. In some embodiments, the subject has an advanced-stage solid tumor. In some embodiments, the subject has a metastatic solid tumor cancer. In some embodiments, the subject has an advanced stage, unresectable, and metastatic solid tumor. In some embodiments, the subject has an advanced stage and unresectable solid tumor. In some embodiments, the subject has an advanced stage and metastatic solid tumor. In some embodiments, the subject has an unresectable and metastatic solid tumor.

In some embodiments, the subject has a cancer cell comprising a partial or total loss of beta-2-microglobulin (B2M) function. In some embodiments, the subject has a cancer cell with a partial loss of B2M function. In some embodiments, the subject has a cancer cell has a total loss of B2M function. In some embodiments, the partial or total loss of B2M function is assessed by comparing a cancer cell to a non-cancer cell from the same subject, wherein the non-cancer cell is from the same tissue from which the cancer cell was derived. In some embodiments, the partial or total loss of B2M function is assessed by comparing a cancer cell to a non-cancer cell from the same subject, wherein the non-cancer cell is not from the same tissue from which the cancer cell was derived. In some embodiments, the partial or total loss of B2M function is assessed by comparing a cancer cell to a non-cancer cell from a different subject. In some embodiments, the partial or total loss of B2M function is assessed by comparing a cancer cell to a non-cancer cell control.

In some embodiments, the cancer cell is in a solid tumor that comprises cancer cells with normal B2M function. In some embodiments, the cancer cell is in a solid tumor in which 25% or more of the cancer cells have a partial or total loss in B2M function. In some embodiments, the cancer cell is in a solid tumor in which 50% or more of the cancer cells have a partial or total loss in B2M function. In some embodiments, the cancer cell is in a solid tumor in which 75% or more of the cancer cells have a partial or total loss in B2M function. In some embodiments, the cancer cell is in a solid tumor in which 95% or more of the cancer cells have a partial or total loss in B2M function.

In some embodiments, the subject comprises a cell comprising a mutation in the B2M gene.

In some embodiments, the mutation is a substitution, insertion, or deletion. In some embodiments, the B2M gene comprises a loss of heterozygosity (LOH). In some embodiments, the mutation is a frameshift mutation. In some embodiments, the mutation is a deletion mutation. In some embodiments, the frameshift mutation is in exon 1 of B2M. In some embodiments, the frameshift mutation results in a truncation of B2M. In some embodiments, the mutation is a complete or partial deletion (e.g., truncation) of B2M. In some embodiments, a deletion mutation is in exon 1 of B2M. In some embodiments, the frameshift mutation comprises p.Leu13fs and/or p.Ser14fs. In some embodiments, the frameshift mutation comprises V69Wfs*34, L15fs*41, L13P, L15fs*41, and/or p. S31* according to Middha et al. (2019) JCO Precis Oncol. (doi: 10.1200/PO.18.00321). In some embodiments, the mutation comprises a frameshift and/or deletion (e.g., truncation) mutation upstream of a kinase domain for JAK1 and/or JAK2.

In some embodiments, the subject has a reduced level of B2M protein as compared to a subject without a partial or total loss of B2M function.

In some embodiments, the subject comprises a partial or total loss of beta-2-microglobulin (B2M) function. In some embodiments, the subject comprises a partial loss of B2M function. In some embodiments, the subject comprises a total loss of B2M function. The partial or total loss of B2M function may be assessed by comparing to a tissue sample from the same subject. The partial or total loss of B2M function may be assessed by comparing a tissue sample from the tumor to a tissue sample from the same tissue from which the tumor sample was derived.

In some embodiments, the solid tumor as a whole (e.g., as assessed in a biopsy taken from the solid tumor) has a partial or total loss of B2M function compared to normal cells or tissue from which the solid tumor is derived. In some embodiments, the subject comprises (e.g. the partial or total loss of function results from) a mutation in the B2M gene.

In some embodiments, certain cells within the tumor have a B2M loss of function. In some embodiment, certain cells within the tumor have a partial or total loss of B2M function while other cells in the tumor do not.

In some embodiments, subject has a reduced level of surface expressed major histocompatibility complex class I (MHC I) as compared to a control, optionally wherein the control is a non-cancerous sample from the same subject. In some embodiments, a subject has a cancer cell comprising a reduced level of surface expressed MHC I. In some embodiments, the cancer cell has no surface expressed MHC I. In some embodiments, the reduced level of surface expressed MHC I is assessed by comparing a cancer cell to a non-cancer cell from the same subject, optionally wherein the non-cancer cell is from the same tissue from which the cancer cell was derived. In some embodiments, the cancer cell is in a solid tumor that comprises cancer cells with a normal level of surface expressed MHC I. In some embodiments, the cancer cell is in a solid tumor in which 25% or more of the cancer cells have a reduced level of surface expressed MHC I. In some embodiments, the cancer cell is in a solid tumor in which 50% or more of the cancer cells have a reduced level of surface expressed MHC I. In some embodiments, the cancer cell is in a solid tumor in which 75% or more of the cancer cells have a reduced level of surface expressed MHC I. In some embodiments, the cancer cell is in a solid tumor in which 95% or more of the cancer cells have a reduced level of surface expressed MHC I.

In some embodiments, the solid tumor as a whole (e.g., as assessed in a biopsy taken from the solid tumor) has a reduced level of surface expressed MHC I compared to normal cells or tissue from which the solid tumor is derived.

In some embodiments, the cytokine RNA mixture provided herein is used in a method of treating an advanced-stage solid tumor cancer.

In some embodiments, the cytokine RNA mixture provided herein is used in a method of treating an unresectable solid tumor cancer.

In some embodiments, the cytokine RNA mixture provided herein is used in a method of treating a metastatic solid tumor cancer.

In some embodiments, the cytokine RNA mixture is injected into one or more a solid tumor cancer within a lymph node.

In some embodiments, the advanced-stage solid tumor cancer comprises a tumor that is suitable for direct intratumoral injection. In some embodiments, the advanced-stage solid tumor cancer is stage III, subsets of stage III, stage IV, or subsets of stage IV. In some embodiments, the cancer is melanoma. In some embodiments, the melanoma is stage IIIB, stage IIIC, or stage IV. In some embodiments, the cancer is cutaneous squamous cell carcinoma (CSCC). In some embodiments, the cancer is head and neck squamous cell carcinoma (HNSCC). In some embodiments, the CSCC or HNSCC is stage III or stage IV. In some embodiments, the solid tumor cancer is melanoma, optionally wherein the melanoma is uveal melanoma or mucosal melanoma; and comprises superficial, subcutaneous and/or lymph node metastases amenable for intratumoral injection. In some embodiments, the solid tumor cancer is HNSCC and/or mucosal melanoma with only mucosal sites. In some embodiments, the solid tumor cancer is HNSCC. In some embodiments, the solid tumor cancer is uveal melanoma or mucosal melanoma. In some embodiments, the solid tumor cancer is uveal melanoma. In some embodiments, the solid tumor cancer is mucosal melanoma. In some embodiments, the RNAs are injected intratumorally only at mucosal sites of the solid tumor cancer, wherein the solid tumor cancer is HNSCC or mucosal melanoma.

In some embodiments, the subject has failed a prior anti-programmed cell death 1 (PD-1) or anti-programmed cell death 1 ligand (PD-L1) therapy. In other embodiments, the subject has not been treated previously with an anti-PD-1 or anti-PD-L1 therapy. In some embodiments, the subject is without other treatment options.

In some embodiments, the method may comprise reducing the size of a tumor or preventing cancer metastasis in a subject.

In some embodiments, the subject has at least two tumor lesions or at least three tumor lesions. In some embodiments, the subject has two tumor lesions. In some embodiments, the subject has three tumor lesions.

In some embodiments, the subject has measurable disease according to the Response Evaluation Criteria in Solid Tumors (RECIST) 1.1 criteria as described herein.

In some embodiments, the subject has a tumor that is suitable for direct intratumoral injection. In some embodiments, whether a tumor is suitable for direct intratumoral injection may be based on the dose volume. In some embodiments, a tumor is suitable for direct intratumoral injection of a cytokine RNA mixture if it includes a cutaneous or subcutaneous lesion ≥0.5 cm in longest diameter or multiple injectable merging lesions which become confluent and have the longest diameter (sum of diameters of all involved target lesions) of ≥0.5 cm suitable for injection (i.e., not bleeding or weeping). In some embodiments, lymph nodes ≥1.5 cm that are suitable for ultrasonography (USG)-guided intratumoral injection and confirmed as metastatic disease are also suitable. In some embodiments, the tumor is uveal melanoma or mucosal melanoma. In some embodiments, the tumor is uveal melanoma or mucosal melanoma: and comprises superficial, subcutaneous and/or lymph node metastases amenable for intratumoral injection.

In some embodiments, the subject is human. In some embodiments, the subject may have a life expectancy of more than 3 months, 4 months, 5 months or 6 months. In some embodiments, the subject has a life expectancy of more than 3 months. In some embodiments, the subject is at least 18 years of age.

In some embodiments, methods for treating an advanced-stage melanoma, cutaneous squamous cell carcinoma (CSCC) or head and neck squamous cell carcinoma (HNSCC) are provided, comprising administering to a subject having an advanced-stage melanoma RNA encoding an IL-12sc protein, RNA encoding an IL-15 sushi protein, RNA encoding an IFNα protein, and RNA encoding a GM-CSF protein. In some embodiments, (a) the subject is at least 18 years of age; (b) the subject has failed prior anti-PD1 or anti-PD-L1 therapies; (c) the subject has a minimum of 2 lesions; and (d) the melanoma, CSCC, or HNSCC comprises a tumor that is suitable for direct intratumoral injection.

In some embodiments, the subject has measurable disease according to the Response Evaluation Criteria in Solid Tumors (RECIST) 1.1 criteria. In some embodiments, the subject has a life expectancy of more than 3 months.

In some embodiments, the solid tumor is an epithelial tumor, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, melanoma tumor, small cell lung tumor, neuroblastoma tumor, testicular tumor, carcinoma tumor, adenocarcinoma tumor, seminoma tumor, retinoblastoma, cutaneous squamous cell carcinoma (CSCC), squamous cell carcinoma for the head and neck (HNSCC), head and neck cancer, or osteosarcoma tumor.

In some embodiments, the solid tumor comprises a primary tumor of any size. In some embodiments, tumor thickness measurements are reported rounded to the nearest 0.1 mm. In some embodiments, the solid tumor comprises a primary tumor having ≤1.0 mm in thickness. In some embodiments, the solid tumor comprises a primary tumor having 0, 1, 0.2, 0.3, 0.4, 0.5, 0.6, 0.7, 0.8, 0.9, or 1.0 mm in thickness. In some embodiments, the solid tumor comprises a primary tumor having <0.8 mm (or less than 0.8 mm) in thickness without ulceration. In some embodiments, the solid tumor comprises a primary tumor having <0.8 mm (or less than 0.8 mm) in thickness with ulceration. In some embodiments, the solid tumor comprises a primary tumor having from 0.8 to 1.0 mm in thickness. In some embodiments, the solid tumor comprises a primary tumor having 0.8, 0.9, or 1.0 mm in thickness. In some embodiments, the solid tumor comprises a primary tumor having from 0.8 to 1.0 mm in thickness without or with ulceration. In some embodiments, the solid tumor comprises a primary tumor having >1.0-2.0 mm in thickness. In some embodiments, the solid tumor comprises a primary tumor having 1.1, 1.2, 1.3, 1.4, 1.5, 1.6, 1.7, 1.8, 1.9, or 2.0 mm in thickness. In some embodiments, the solid tumor comprises a primary tumor having >1.0-2.0 mm in thickness without or with ulceration. In some embodiments, the solid tumor comprises a primary tumor having >2.0-4.0 mm in thickness. In some embodiments, the solid tumor comprises a primary tumor having 3.0-4.0 mm in thickness. In some embodiments, the solid tumor comprises a primary tumor having 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mm in thickness. In some embodiments, the solid tumor comprises a primary tumor having >2.0-4.0 mm in thickness without or with ulceration. In some embodiments, the solid tumor comprises a primary tumor having >4.0 mm in thickness. In some embodiments, the solid tumor comprises a primary tumor having 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0 or 10.0 mm in thickness. In some embodiments, the solid tumor comprises a primary tumor having >4.0 mm in thickness without or with ulceration. In some embodiments, the thickness is at the thickest (i.e., greatest) dimension of the tumor. In some embodiments, the tumor is a skin cancer tumor and the thickness is from the skin surface to the deepest part of the tumor (e.g., the thickness is not the lateral spread of the tumor). In some embodiments, the tumor is a skin metastasis of a cancer other than a skin cancer, and the thickness of the tumor is from the skin surface to the deepest part of the tumor (e.g., the thickness is not the lateral spread of the tumor).

In some embodiments, the solid tumor is a melanoma solid tumor. In some embodiments, the melanoma comprises a primary tumor of any size. In some embodiments, tumor thickness measurements are reported rounded to the nearest 0.1 mm. In some embodiments, the melanoma comprises a primary tumor having ≤1.0 mm in thickness. In some embodiments, the melanoma comprises a primary tumor having 0.1, 0.2, 0.3, 0.4, 0.5, 0.6, 0.7, 0.8, 0.9, or 1.0 mm in thickness. In some embodiments, the melanoma comprises a primary tumor having <0.8 mm (or less than 0.8 mm) in thickness without ulceration. In some embodiments, the melanoma comprises a primary tumor having <0.8 mm (or less than 0.8 mm) in thickness with ulceration. In some embodiments, the melanoma comprises a primary tumor having from 0.8 to 1.0 mm in thickness. In some embodiments, the melanoma comprises a primary tumor having 0.8, 0.9, or 1.0 mm in thickness. In some embodiments, the melanoma comprises a primary tumor having from 0.8 to 1.0 mm in thickness without or with ulceration. In some embodiments, the melanoma comprises a primary tumor having >1.0-2.0 mm in thickness. In some embodiments, the melanoma comprises a primary tumor having 1.1, 1.2, 1.3, 1.4, 1.5, 1.6, 1.7, 1.8, 1.9, or 2.0 mm in thickness. In some embodiments, the melanoma comprises a primary tumor having >1.0-2.0 mm in thickness without or with ulceration. In some embodiments, the melanoma comprises a primary tumor having >2.0-4.0 mm in thickness. In some embodiments, the melanoma comprises a primary tumor having 3.0-4.0 mm in thickness. In some embodiments, the melanoma comprises a primary tumor having 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mm in thickness. In some embodiments, the melanoma comprises a primary tumor having >2.0-4.0 mm in thickness without or with ulceration. In some embodiments, the melanoma comprises a primary tumor having >4.0 mm in thickness. In some embodiments, the melanoma comprises a primary tumor having 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0 or 10.0 mm in thickness. In some embodiments, the melanoma comprises a primary tumor having >4.0 mm in thickness without or with ulceration. In some embodiments, the thickness is from the skin surface to the deepest part of the tumor (the thickness is not the lateral spread of the tumor).

In some embodiments, the melanoma comprises one tumor-involved regional lymph node or any number of in-transit, satellite, and/or microsatellite metastases with no tumor-involved nodes. In some embodiments, the melanoma comprises one clinically occult tumor-involved regional lymph node. In some embodiments, the melanoma comprises one clinically detectable tumor-involved regional lymph node. In some embodiments, the melanoma comprises any number of in-transit, satellite, and/or microsatellite metastases with no tumor-involved nodes. In some embodiments, the melanoma comprises two or three tumor-involved regional lymph nodes or any number of in-transit, satellite, and/or microsatellite metastases with no tumor-involved nodes. In some embodiments, the melanoma comprises two or three clinically occult tumor-involved regional lymph nodes. In some embodiments, the melanoma comprises two or three tumor-involved regional lymph nodes, at least one of which is clinically detectable. In some embodiments, the melanoma comprises two or three tumor-involved regional lymph nodes, one of which is clinically occult or clinically detectable and with presence of in-transit, satellite, and/or microsatellite metastases. In some embodiments, the melanoma comprises any number of in-transit, satellite, and/or microsatellite metastases with one tumor-involved node. In some embodiments, the melanoma comprises four or more tumor-involved regional lymph nodes or any number of in-transit, satellite, and/or microsatellite metastases with two or more tumor-involved nodes or any number of matted nodes without or with in-transit, satellite, and/or microsatellite metastases. In some embodiments, the melanoma comprises four or more clinically occult tumor-involved regional lymph nodes. In some embodiments, the melanoma comprises four or more clinically occult tumor-involved regional lymph nodes, at least one of which is clinically detectable or with presence of any number of matted nodes. In some embodiments, the melanoma comprises two or three tumor-involved regional lymph nodes, one of which is clinically occult or clinically detectable. In some embodiments, the melanoma comprises four or more clinically occult tumor-involved regional lymph nodes, two or more of which are clinically occult or clinically detectable and/or with presence of any number of matted nodes, and with presence of in-transit, satellite, and/or microsatellite metastases.

In some embodiments, the melanoma
a. comprises a primary tumor of any size;
b. comprises one or more tumor-involved regional lymph nodes: or in-transit, satellite, and/or microsatellite metastases with no tumor-involved regional lymph nodes; and
c. comprises no detectable distant metastasis.

In some embodiments, the melanoma has a detectable distant metastasis.

In some embodiments, the melanoma
a. comprises a primary tumor having <0.8 mm in thickness without ulceration; or a primary tumor having from 0.8 to 1.0 mm in thickness and a primary tumor less than 0.8 mm in thickness with ulceration; or a primary tumor having >1.0-2.0 mm in thickness without ulceration;
b. comprises one or two or three clinically occult tumor-involved regional lymph nodes: and
c. comprises no detectable distant metastasis.

In some embodiments, the melanoma
a. comprises a primary tumor having <0.8 mm in thickness without ulceration; or a primary tumor having from 0.8 to 1.0 mm in thickness and a primary tumor less than 0.8 mm in thickness with ulceration; or a primary tumor having >1.0-2.0 mm in thickness without ulceration;
b. comprises one clinically detectable tumor-involved regional lymph node; or no tumor-involved regional lymph node with presence of in-transit, satellite, and/or microsatellite metastases: or two or three tumor-involved regional lymph nodes, at least one of which is clinically detectable; and
c. comprises no detectable distant metastasis.

In some embodiments, the melanoma
a. comprises a primary tumor having >1.0-2.0 mm in thickness with ulceration; or a primary tumor having >2.0-4.0 mm in thickness without ulceration;
b. comprises one clinically detectable or clinically occult tumor-involved regional lymph node: or none or one tumor-involved regional lymph nodes with in-transit, satellite, and/or microsatellite metastases; and
c. comprises no detectable distant metastasis.

In some embodiments, the melanoma
a. comprises one clinically detectable tumor-involved regional lymph node; or no tumor-involved regional lymph nodes with presence of in-transit, satellite, and/or microsatellite metastases; and
b. comprises no detectable distant metastasis.

In some embodiments, the melanoma has no detectable distant metastasis: and comprises
a. two or three tumor-involved regional lymph nodes, at least one of which is clinically detectable;
b. one clinically occult or detectable tumor-involved regional lymph node with presence of in-transit, satellite, and/or microsatellite metastases;
c. four or more tumor-involved regional lymph nodes, at least one of which is clinically detectable, or the presence of one or more matted nodes: or
d. two or more clinically occult or clinically detectable tumor-involved regional lymph nodes and/or presence of one or more matted nodes with presence of in-transit, satellite, and/or microsatellite metastases.

In some embodiments, the melanoma comprises a primary tumor having <0.8 mm or >1.0-2.0 or >2.0-4.0 mm in thickness without ulceration: comprises no detectable distant metastasis: and comprises:
a. one clinically occult or clinically detected tumor-involved regional lymph nodes with presence of in-transit, satellite, and/or microsatellite metastases; or
b. four or more tumor-involved regional lymph nodes; or one or more in-transit, satellite, and/or microsatellite metastases with two or more tumor-involved nodes: or one or more matted nodes without or with in-transit, satellite, and/or microsatellite metastases.

In some embodiments, the melanoma
a. comprises a primary tumor having >2.0-4.0 mm in thickness with ulceration or a primary tumor having >4.0 mm in thickness without ulceration;
b. comprises one or more tumor-involved regional lymph nodes; or one or more in-transit, satellite, and/or microsatellite metastases optionally with one or more tumor-involved regional lymph nodes; or one or more matted nodes without or with in-transit, satellite, and/or microsatellite metastases: and
c. comprises no detectable distant metastasis.

In some embodiments, the melanoma
a. comprises a primary tumor in >4.0 mm in thickness without ulceration;
b. comprises one or two or three tumor-involved regional lymph nodes; or one or more in-transit, satellite, and/or microsatellite metastases with no or one tumor-involved regional lymph nodes; and
c. comprises no detectable distant metastasis.

In some embodiments, the melanoma
a. comprises a primary tumor >4.0 mm in thickness with ulceration;
b. comprises four or more tumor-involved regional lymph nodes; or one or more in-transit, satellite, and/or microsatellite metastases with two or more tumor-involved regional lymph nodes, or one or more matted nodes without or with in-transit, satellite, and/or microsatellite metastases; and
c. comprises no detectable distant metastasis.

In some embodiments, the cutaneous squamous cell carcinoma (CSCC) or squamous cell carcinoma for the head and neck (HNSCC) comprises a tumor of any size. In some embodiments, the CSCC or HNSCC comprises no identified tumor. In some embodiments, the CSCC or HNSCC comprises a tumor that is 2 cm or smaller in its greatest dimension. In some embodiments, the CSCC or HNSCC comprises a tumor larger than 2 cm but not larger than 4 cm in its greatest dimension. In some embodiments, the CSCC or HNSCC comprises a tumor that is larger than 4 cm in greatest dimension or has minimal erosion of the bone or perineural invasion or deep invasion. In some embodiments, the CSCC or HNSCC comprises a tumor with extensive cortical or medullary bone involvement or invasion of the base of the cranium or invasion through the foramen of the base of the cranium.

In some embodiments, the cutaneous squamous cell carcinoma (CSCC) or squamous cell carcinoma for the head and neck (HNSCC) comprises no regional lymph node metastasis. In some embodiments, the CSCC or HNSCC comprises metastasis in a single ipsilateral lymph node, is 3 cm or smaller in greatest dimension, and is ENE-negative. In some embodiments, the CSCC or HNSCC comprises metastasis in a single ipsilateral lymph node larger than 3 cm but not larger than 6 cm in greatest dimension and ENE-negative. In some embodiments, the CSCC or HNSCC comprises metastases in multiple ipsilateral lymph nodes, none larger than 6 cm in their greatest dimension and is ENE-negative. In some embodiments, the CSCC or HNSCC comprises metastasis in bilateral or contralateral lymph nodes, none larger than 6 cm in greatest dimension, and is ENE-negative. In some embodiments, the CSCC or HNSCC comprises metastasis in a lymph node larger than 6 cm in its greatest dimension and is ENE-negative; or metastasis in any lymph nodes and ENE-negative. In some embodiments, the cutaneous squamous cell carcinoma (CSCC) or squamous cell carcinoma for the head and neck (HNSCC):
a. comprises a tumor larger than 4 cm in greatest dimension or has minimal erosion of the bone or perineural invasion or deep invasion; and
b. comprises
   i. no regional lymph node metastasis; or
   ii. metastasis in a single ipsilateral lymph node, 3 cm or smaller in greatest dimension and ENE-negative; and
c. comprises no detectable distant metastasis.

In some embodiments, the cutaneous squamous cell carcinoma (CSCC) or squamous cell carcinoma for the head and neck (HNSCC) comprises:
a. a tumor that is 2 cm or smaller in greatest dimension;
b. metastasis in a single ipsilateral lymph node, 3 cm or smaller in its greatest dimension and is ENE-negative; and
c. no detectable distant metastasis.

In some embodiments, the cutaneous squamous cell carcinoma (CSCC) or squamous cell carcinoma for the head and neck (HNSCC) comprises:
a. a tumor larger than 2 cm but not larger than 4 cm in its greatest dimension;
b. metastasis in a single ipsilateral lymph node, 3 cm or smaller in its greatest dimension and is ENE-negative; and
c. no detectable distant metastasis.

In some embodiments, the cutaneous squamous cell carcinoma (CSCC) or squamous cell carcinoma for the head and neck (HNSCC)
a. comprises:
   i. a tumor that is 2 cm or smaller in its greatest dimension; or
   ii. a tumor larger than 2 cm but not larger than 4 cm in its greatest dimension: or
   iii. a tumor larger than 4 cm in its greatest dimension or minimal erosion of the bone or perineural invasion or deep invasion; and
b. comprises
   i. metastasis in a single ipsilateral lymph node larger than 3 cm but not larger than 6 cm in its greatest dimension and is extranodal extension (ENE)-negative; or
   ii. metastases in multiple ipsilateral lymph nodes, none larger than 6 cm in its greatest dimension and is ENE-negative; or
   iii. metastasis in bilateral or contralateral lymph nodes, none larger than 6 cm in its greatest dimension and is ENE-negative: and
c. comprises no detectable distant metastasis.

In some embodiments, the cutaneous squamous cell carcinoma (CSCC) or squamous cell carcinoma for the head and neck (HNSCC)
a. comprises
   i. a tumor that is 2 cm or smaller in greatest dimension; or
   ii. a tumor larger than 2 cm but not larger than 4 cm in its greatest dimension; or
   iii. a tumor larger than 4 cm in greatest dimension or minimal erosion of the bone or perineural invasion or deep invasion; or
   iv. a tumor with extensive cortical or medullary bone involvement or invasion of the base of the cranium or invasion through the foramen of the base of the cranium; and
b. comprises metastasis in a lymph node larger than 6 cm in its greatest dimension and is ENE-negative; or metastasis in any lymph nodes and is ENE-negative: and
c. comprises no detectable distant metastasis.

In some embodiments, the cutaneous squamous cell carcinoma (CSCC) or squamous cell carcinoma for the head and neck (HNSCC)
a. comprises tumor with extensive cortical or medullary b one involvement or invasion of the base of the cranium or invasion through the foramen of the base of the cranium;
b. comprises
   i. no regional lymph node metastasis; or
   ii. metastasis in a single ipsilateral lymph node, 3 cm or smaller in greatest dimension and ENE-negative; or
   iii. metastasis in a single ipsilateral lymph node larger than 3 cm but not larger than 6 cm in greatest dimension and ENE-negative; or metastases in multiple ipsilateral lymph nodes, none larger than 6 cm in greatest dimension and ENE-negative; or metastasis in bilateral or contralateral lymph nodes, none larger than 6 cm in greatest dimension and ENE-negative: or
   iv. metastasis in a lymph node larger than 6 cm in greatest dimension and ENE-negative; or metastasis in any lymph nodes and ENE-negative and
c. comprises no detectable distant metastasis.

In some embodiments, the cutaneous squamous cell carcinoma (CSCC) or squamous cell carcinoma for the head and neck (HNSCC)
a. comprises tumor with extensive cortical or medullary bone involvement or invasion of the base of the cranium or invasion through the foramen of the base of the cranium, and
b. comprises no detectable distant metastasis.

In some embodiments, the cutaneous squamous cell carcinoma (CSCC) or squamous cell carcinoma for the head and neck (HNSCC)
a. comprises
   i. a tumor 2 cm or smaller in greatest dimension; or
   ii. a tumor larger than 2 cm but not larger than 4 cm in greatest dimension; or
   iii. a tumor larger than 4 cm in greatest dimension or minimal erosion of the bone or perineural invasion or deep invasion, or
   iv. tumor with extensive cortical or medullary bone involvement or invasion of the base of the cranium or invasion through the foramen of the base of the cranium;
b. comprises
   i. no regional lymph node metastasis; or
   ii. metastasis in a single ipsilateral lymph node, 3 cm or smaller in greatest dimension and ENE-negative: or
   iii. metastasis in a single ipsilateral lymph node larger than 3 cm but not larger than 6 cm in greatest dimension and ENE-negative: or metastases in multiple ipsilateral lymph nodes, none larger than 6 cm in greatest dimension and ENE-negative; or metastasis in bilateral or contralateral lymph nodes, none larger than 6 cm in greatest dimension and ENE-negative;
   iv. metastasis in a lymph node larger than 6 cm in greatest dimension and ENE-negative; or metastasis in any lymph nodes and ENE-negative: and
c. comprises detectable distant metastasis.

In some embodiments, the cutaneous squamous cell carcinoma (CSCC) or squamous cell carcinoma for the head and neck (HNSCC) comprises no detectable distant metastasis.

In some embodiments, the therapeutically effective amount of the RNAs results in one or more of: (a) a reduction in the severity or duration of a symptom of cancer; (b) inhibition of tumor growth, or an increase in tumor necrosis, tumor shrinkage and/or tumor disappearance; (c) delay in tumor growth and/or development; (d) inhibited or retarded or stopped tumor metastasis; (e) prevention or delay of recurrence of tumor growth; (f) increase in survival of a subject: and/or (g) a reduction in the use or need for conventional anticancer therapy (e.g., reduced or eliminated use of chemotherapeutic or cytotoxic agents), optionally as compared to an untreated subject or a subject administered only 1, 2, or 3 of the RNAs in the RNA mixture.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include”, and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way. In the Examples discussed below, the cytokine RNA mixture, as defined above, may be also referred as “the mixture,” “the cytokine mixture,” “the composition,” or “the drug” interchangeably.

### Example 1—Dose Escalation and Dose Expansion of the Cytokine RNA Mixture

Overall design: A first in human, open-label, dose escalation and expansion study for the evaluation of the maximum tolerated and administered doses, safety, tolerability, pharmacokinetics, pharmacodynamics, and anti-tumor activity of the cytokine RNA mixture administered intratumorally as a single agent is performed.

Number of participants: Enrollment of up to 72 participants is planned, depending on the investigated dose levels during the escalation phase.

Dose escalation phase: There is no formal sample size calculation in the dose escalation phase. The cytokine RNA mixture is administered to patients with advanced solid tumors who have failed a prior anti-PD-1 or anti-PD-L1 based therapy, and/or patients without other treatment options for those indications in which anti-PD-1 is not routinely used. Up to 38 dose limiting toxicities (DLT)-evaluable participants enroll in the dose escalation phase with expected assessment of about 8 dose levels. The actual sample size varies depending on DLTs observed and number of dose levels actually explored.

Dose expansion phase: A Simon's two-stage design is used in the expansion phase and approximately 34 participants with advanced melanoma who failed prior anti-PD-1/anti-PD-L1 therapies enroll. After the first 16 treated participants, there is an interim analysis, and if response is observed in at least 2 participants, accrual continues to the full sample size of 34 participants.

Intervention groups and duration: The duration of the study for a participant includes a period for screening of up to 28 days. Once successfully screened, participants may receive study intervention until disease progression, unacceptable AE, participant's decision to stop the treatment, or for a maximum of 1 year if no disease progression occurs. Continuation of cytokine RNA mixture will be considered beyond 1 year by the study committee on a case by case basis for those participants that clearly continue to derive clinical benefit in a safe manner with reasonable toxicity. After discontinuing study intervention, participants return to the study site approximately 30 days after the last IMP administration or before the participant receives another anticancer therapy, whichever is earlier, for end-of-treatment assessments. If the participant discontinues study intervention for reasons other than progression, follow-up visits are performed every 3 months until disease progression, initiation of another anticancer treatment, or death (whichever comes first).

The expected duration of treatment for participants who benefit from the cytokine RNA mixture may vary, based on progression date; but median expected duration of study per participant is estimated as 9 months (1 month for screening, 5 months for treatment, and 3 months for end of treatment follow-up).

IMP is administered intratumorally once per week in a 4-week cycle (i.e., four doses every 28 days). After each cycle of treatment, the frequency of intratumoral injection may continue weekly. After the second tumor assessment, change of the dose interval to, e.g., once a month may occur.

Advancement to higher dose levels during the escalation phase occurs based on toxicity; intermediate doses may also be considered. Once early efficacy signals are seen at a dose level that is declared safe, it may be expanded to confirm the efficacy.

Dose omissions or dose delay may occur throughout the study; the occurrence of dose limiting toxicities (DLTs) determines the need for these modifications. Participants who experience a DLT stop the treatment and are followed until resolution to Grade 1 or baseline. After recovery from dose omission that does not exceed two weeks (i.e., 2 dose omissions), the participant may resume therapy with a new cycle of treatment at the same or a lower dose level: no dose re-escalation is allowed for such re-dosed participants at a lower dose level. If the participant experiences the same AE leading to a second dose omission for 2 weeks (i.e., 2 dose omissions), then the participant may be permanently discontinued.

Route of administration: Intratumoral injection

Dose regimen: the cytokine RNA mixture is administered at assigned dose levels once a week, 4 injections within a 28-day cycle.

Noninvestigational medicinal products(s): No pre-defined premedication is administered.

Post-trial access to study medication: All participants are treated for 1 year or until disease progression, whichever is the earliest.

Statistical considerations:

a. Primary Analysis:

Dose escalation: In the dose escalation phase, DLTs are summarized by dose level. Details of DLTs are provided by participant. The treatment-emergent AEs/SAEs and laboratory abnormalities during the on-treatment period are summarized using descriptive statistics by dose level.

Dose expansion: Objective response rate (ORR) per RECIST 1.1 are summarized with descriptive statistics. A 90% two-sided confidence interval is computed using Clopper-Pearson method. The statistical inference is based on the hypothesis and alpha level defined in the sample size calculation section.

b. Analysis of Secondary Endpoints:

Dose escalation: Concentration and PK parameters of the cytokines encoded by the mixture is summarized with descriptive statistics during cycles in which PK is assessed. Anti-drug antibodies (ADAs) against the cytokines encoded by the mixture is descriptively summarized.

Dose expansion: The treatment-emergent AEs/SAEs and laboratory abnormalities during the on-treatment period is summarized using descriptive statistics. DoR and PFS per RECIST 1.1 and iRECIST are summarized using the Kaplan-Meier method. A similar analysis as ORR per RECIST 1.1 is provided for DCR per RECIST 1.1 and iRECIST, and the ORR per iRECIST. PK concentration and parameters of the cytokines encoded by the cytokine mixture are summarized with descriptive statistics during cycles in which PK is assessed. ADAs against the cytokines encoded by the cytokine RNA mixture are descriptively summarized.

The cytokine RNA mixture is a 1:1:1:1 weight ratio (w:w:w:w) of synthetic, chemically modified mRNAs encoding the human cytokines IL-15sushi, IL-12sc. GM-CSF, and IFNα2b. The chosen mixture of cytokines is expected to exhibit superior anti-tumor activity versus individual cytokines.

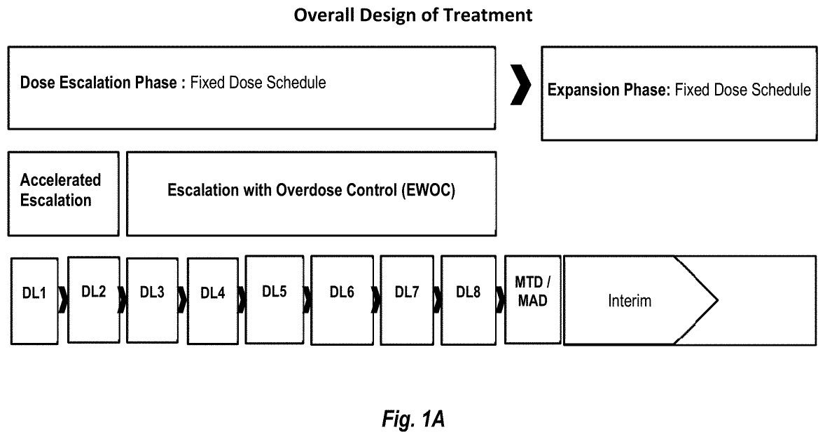

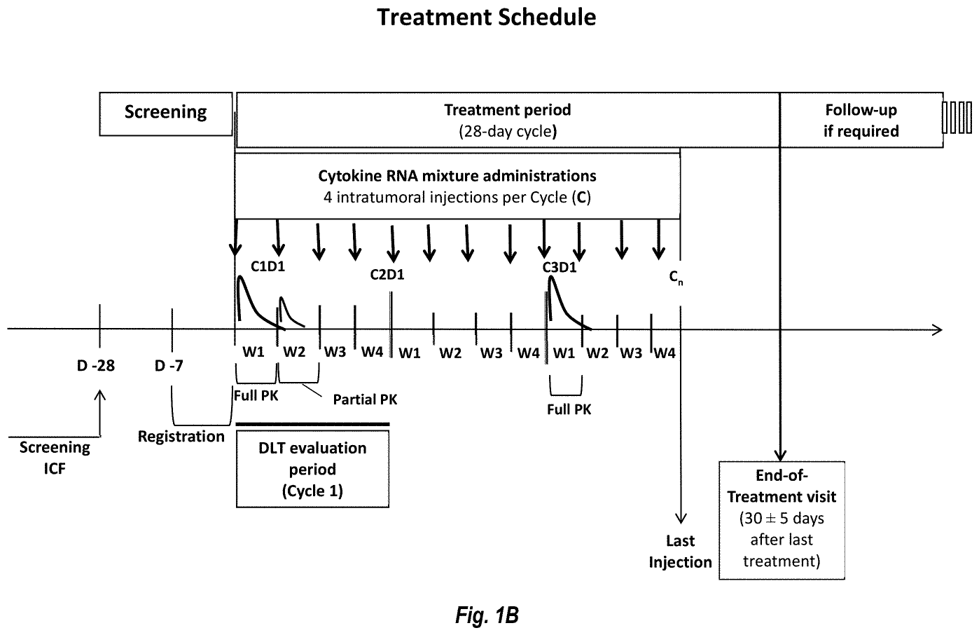

FIG. 1A shows a graphic of the overall design of the study, while FIG. 1B shows a graphic of the treatment scheduling per patient. The dose escalation phase aims to determine the MTD or MAD of the cytokine RNA mixture administered weekly as monotherapy to patients who have failed anti-PD-1 or anti-PD-L1. During the accelerated escalation phase, the occurrence of toxicities observed in Cycle 1 is assessed on one participant. As soon as a related Grade ≥2 AE or DLT occurs in anyone of the Accelerated Escalation DLs (DL I or 2, whichever occurs first), or starting from DL3, a Bayesian Escalation with Overdose Control is initiated with evaluation of at least 3 participants/cohort. When the dose escalation Phase ends, the MTD/MAD to be evaluated in the Expansion Phase is determined based on safety. In the expansion phase, testing of the MTD/MAD of the fixed dose administered weekly in patients with stage IIIB. IIIC or IV melanoma after failure of anti-PD-1 or anti-PD-L1 is planned.

Tables 2 and 3 show the Schedule of Activities (SOA) with Table 2 showing the treatment flowchart and Table 3 showing the PK and PDy flowchart for the dose escalation and expansion phases.

TABLE 2
Schedule of Activities (SOA) and Treatment Flowchart

| | | Treatment^{b} Cycle 1 | | | | | | | | Treatment Cycle 2 mad subsequent | | | | | | | |
|---|---|---|---|---|---|---|---|---|---|---|---|---|---|---|---|---|---|
| | | Cycle length 28 days in monotherapy | | | | | | | | Cycle length 28 days in monotherapy | | | | | | | |
| | | Week 1 | | Week 2 | | Week 3 | | Week 4 | | Week 1 | | Week 2 | Week 3 | Week 4 | | | |
| | | Days prior to initial dose | | | | | | | | | | | | | | EO | |
| Evaluation^{a} | Screening | D1 | D2 | D8 | D9 | D15 | D16 | D22 | D23 | D1 | D2 | D8 | D15 | D22 | D24 | T^{x} | FU^{t,y} |
| Inclusion/Exclusion | ≤28 | | | | | | | | | | | | | | | | |
| criteria/Informed | | | | | | | | | | | | | | | | | |
| Consent | | | | | | | | | | | | | | | | | |
| Demographics and | ≤28 | | | | | | | | | | | | | | | | |
| Medical/Disease | | | | | | | | | | | | | | | | | |
| History^{c} | | | | | | | | | | | | | | | | | |
| ECOG PS, Body | ≤78 | X | | X | | X | | X | | X | X | X | | X | | | |
| Weight^{d}, Height^{e} | | | | | | | | | | | | | | | | | |
| (only baseline) | | | | | | | | | | | | | | | | | |
| Vital signs^{f} | ≤7 | X | X | X | | X | | X | | X | | X | X | X | | X | |
| Physical | ≤7 | X | | X | | X | | X | | X | | X | X | X | | X | |
| examination^{g} | | | | | | | | | | | | | | | | | |
| Digital Photography^{h} | ≤7 | Digital photographs must correspond with radiographic assessment timepoints | | | | | | | | | | | | | | | |
| Serum pregnancy testⁱ | ≤7 | | | | | | | | | X | | | | | | X | |
| HBsAg & HCV | ≤28 | | | | | | | | | | | | | | | | |
| serology (and HIV | | | | | | | | | | | | | | | | | |
| test for participants at | | | | | | | | | | | | | | | | | |
| German study sites | | | | | | | | | | | | | | | | | |
| only) | | | | | | | | | | | | | | | | | |
| Blood Hematology^{j} | ≤7 | X | | X | | X | | X | | X | | X | X | X | | X | |
| Coagulation^{k} | ≤7 | X | | X | | X | | X | | X | | X | X | X | | X | |
| Serum Chemistry^{l} | ≤7 | X | | X | | X | | X | | X | | X | X | X | | X | |
| CRP/ferritin^{m} | ≤28 | X | X | X | X | X | X | X | X | X | X^{m} | X | X | X | | X | |
| Secondary plasma | | X | X | X | X | | | | | X^{m} | X^{m} | | | | | X | |
| cytokines^{m} | | | | | | | | | | | | | | | | | |
| 12-lead ECGⁿ | ≥28 | X | | | | | | | | Xⁿ | | | | | | X | |
| Bone marrow | As per Lugano 2014 Classification (see herein) | | | | | | | | | | | | | | | | |
| biopsy/aspirate^{o} | | | | | | | | | | | | | | | | | |
| Neck, Chest, | | | | | | | | | | | | | | | | | |
| Abdominal, Pelvic | <28 | FDG PET-CT/CT scans approximately every 12 weeks, to confirm CR or | | | | | | | | | | | | | | | |
| FDG -PET/CT, CT^{P} | | PD and as clinically indicated (see herein) | | | | | | | | | | | | | | | |
| Assessment of | <28 | In accordance with disease response assessment | | | | | | | | | | | | | | X | |
| lymphoma B | | | | | | | | | | | | | | | | | |
| symptoms | | | | | | | | | | | | | | | | | |
| Urinalysis^{q} | <28 | X | | X | | X | | X | | X | | X | X | X | | X | |
| Urine biomarker^{r} | | X^{r} | X | X | | | | | | | | | | | | | |
| Ophthalmologic exam^{s} | X | | | | | | | | | | | | | | | | |

| | | | |
|---|---|---|---|
| Adverse Events | X | Continuous throughout study intervention | X^{t} |
| Assessment^{t} | | | |
| Concomitant | | Continuous throughout study intervention | |
| Medications^{u} | | | |

Study Drug Administration^{v}

| | | | | | | | | |
|---|---|---|---|---|---|---|---|---|
| the cytokine RNA | X | X | X | X | X | X | X | X |
| mixture | | | | | | | | |

Tumor Assessment^{w}

| | | |
|---|---|---|
| RECIST1.1 and | ≤28 | X^{w,y} |
| iRECiST | | |

Pharmacokinetics (PK)

| | |
|---|---|
| the cytokine RNA | See details in the monotherapy PK/PDy flowchart (Table 3) |
| mixture PK | |
| assessments | |

Pharmacodynamics

| | |
|---|---|
| Pharmacodynamics | See details in tile monotherapy PK/PDy flowchart (Table 3) |
| (PDy) assessments | |

Immuutogenicity

| | |
|---|---|
| Blood for antibodies | See details in the monotherapy PK/PDy flowchart (Table 3) |
| against cytokines | |
| encoded by the | |
| cytokine RNA | |
| mixture | |

^{a}Evaluation: Assessments are performed prior to administration of study drug unless otherwise indicated. Results are reviewed by the investigator prior to the administration of the next dose. Tumor biopsy is collected for immunohistochemistry, genomic. RNA-sequencing, and neo-antigen analyses
^{b}A cycle is 28 days, with the cytokine RNA mixture administered intratumorally every week as monotherapy.
^{c}Demography: Includes age, gender, race, and ethnicity. Medical/Surgical History: Includes relevant history of previous pathologies and surgeries. Disease History: Includes stage at diagnosis and at study entry, and previous anti-tumor therapy (type, duration, reason for discontinuation and response to the therapy). In addition, specific mutations depending on tumor type.
^{d}Body weight is measured prior to treatment on the first day of each cycle.
^{e}Height is measured during baseline only.
^{f}Vital signs include: temperature, blood pressure, heart rate, respiration rate. Vital signs must be checked every 6 hours during each 24 hour inpatient hospitalization period during C1D1 at each new dose level while participants are monitored to assess for acute toxicities.
^{g}Physical examination includes: examination of major body systems including cardiovascular system, digestive system, central nervous system, respiratory system, and hematopoietic system (hepatomegaly, splenomegaly, lymphadenopathy), and skin. Signs and symptoms are reported in the eCRF as AEs only if they are still present at the time of first IMP administration.
^{h}Given that a modest pharmacological effect (e.g, redness, edema or flattening of a cytokine RNA mixture injected tumor lesion) is expected to occur following DL3, color digital photographs are mandatory starting at DL4 of mono escalation, starting from first DL in combo escalation and during expansion phase. Digital photographs are mandatory at screening prior to first dose of cytokine RNA mixture and at the time of radiographic tumor assessment from superficial and/or visible subcutaneous injected lesions to document overall disease status and to document responses. In addition, ad hoc color digital photographs must be taken in between screening and tumor assessment windows to capture other cytokine RNA mixture potentially induced changes such as skin redness and/or edema, All collected by the clinical site must be systematically shared with the Sponsor for review as per study reference manual.
ⁱSerum pregnancy testing is performed for women of child bearing potential. A seven-day window is acceptable at baseline assessment.
^{j}Blood hematology: Hemoglobin, hematocrit, WBC with differential (including absolute nentrophil count [ANC]), platelet count. These tests are done before each IMP administration (−1 day window is acceptable). If Grade 4 neutropenia, assess ANC every 2-3 days until ANC ≥0.5 × 10⁹/L, then weekly until recovery, The Cycle 1 Day 1 assessment is done within 2 days of IMP administration, if abnormal at baseline,
^{k}Coagulation: activated partial thromboplastin time (aPTT), PT, international normalized ration (INR), fibrinogen (and D-dimer at Screening). The Cycle 1 Day 1 assessment is done within 2 days of IMP administration, if abnormal at baseline.
^{l}Senun chemistry: Liver function tests: AST, ALT, total bilirubin, direct bilirubin, alkaline phosphatase (ALP), Renal function tests: Urea or BUN & creatinine, and determination of estimated CrCL when required (if creatinine between 1.0 and 1.5x ULN). Electrolytes: Sodium, potassium, total calcium, phosphorus, chloride, magnesium and bicarbonate. Others, glucose, lactate dehydrogenase (LDH), albumin, total proteins, and amylase. The liver function tests, renal function tests, electrolytes, glucose, LDH, albumin and total proteins are performed before IMP administration (−1 day window is acceptable), unless clinically indicated, In case of Grade ≥3 liver function abnormal tests, additional tests are repeated every 2-3 days until recovery to baseline value. The Cycle 1 Day 1 serum chemistry assessment is done within 2 days of IMP administration, if abnormal at baseline.
^{m}Serum C-reactive protein (CRP), ferritin, and secondary plasma cytokines (including interleukin-6 and interferon-alpha) are be collected at the specified time points and in case of occurrence of CRS Grade ≥2 symptoms. Serum CRP and Ferritin samples are collected just before each study intetvention (D1) and at 24 hr (D2) during Cycle 1 (for each Week, 1-4) and during Cycle 3 Week 1. On other study intervention days, only pre-dose samples are collected; additional samples are withdrawn whenever the appearance of Grade ≥2 symptoms of CRS. Routine sampling of secondary plasma cytokines occurs only in Cycles 1 and 3, and at EOT. Samples are collected at pre-dose and 6 and 24 hours after the cytokine RNA mixture administration at Cycle 1, Weeks 1 and 2, and Cycle 3 Week 1; at EOT; and in case of Grade ≥2 symptoms of CRS.
ⁿ12-lead ECG: to be done at screening and pretreatment at Cycle 1 Day 1, Cycle 3 Day 1, Cycle 7 Day 1, and EOT, and when clinically indicated.
^{o}Bone marrow aspirate: Only for patient with lymphoma.
^{p}FDG-PET-CT/CT: FDG PET only applicable for patients with lymphoma as per Lugano classification to be performed within 28 days of IMP administration (−7 days), and approximately every 12 weeks (±7 days) to confirm CR and PD and as clinically indicated.
^{q}Urinalysis: Dipstick (qualitative) tests on rooming spot by dipstick are performed at baseline and before each IMP administration and at EOT. Quantitative urinalysis for leukocytes and red blood cells on morning spot urine are performed at baseline, at uneven cycles, at the end of treatment, and in case of abnormality in the dipstick test (qualitative), In case of proteinuria ≥++ (dipstick), proteinuria quantification by-proteinuria/24 hr urine collection is performed.
^{r}Urine biomarker: kidney injury molecule-1 (KIM-1), urinary microalbumin, and urinary creatinine (in spot urine) are assessed at pre-dose on Cycle 1 Day 1 (within 7 days beforehand is acceptable), 24 hr after the first IMP administration, and pre-dose on day 8 after the first IMP administration
^{s}Ophthalmologic exam including Schirmer's test is performed at baseline and in case of ocular symptoms during therapy. Ocular and visual symptoms are assessed on Day 1 of each Cycle.
^{t}Adverse Event assessment: The period of observation for collection of adverse events extends from the signature of the Informed Consent Form (ICF) until 30 days after the last administration of the study drug. Serious adverse events are assessed and reported as described in the protocol. After the EOT visit, ongoing SARs and AESIs, related AEs, and new related AEs are to be followed up to stabilization, recovery, or initiation of further therapy.
^{u}Concomitant Medication assessment: Concomitant medications are recorded from 14 days prior to the initial dose of study drug until 30 days after the last administration of study drug, resolution of ongoing study-drug related adverse events, or when another anticancer therapy is received.
^{v}Study drug administration: Participants may receive premedication(s) as specified herein. At each new dose level at Cycle 1 and Day 1, participants are monitored for at least 24 hr in the hospital to assess acute toxicities. With subsequent administrations, participants undergo observation for 4-6 hrs with optional hospitalization up to 24 hr at Investigator discretion. Cytokine RNA mixture can be administered with a window of +/− 1 days during Cycle 1 and with a window of +/− 3 days starting from Cycle 2.
^{w}Tumor assessment: CT-scan or magnetic resonance imaging (MRI) and any other exams as clinically indicated are performed to assess disease status at baseline (within 28 days of IMP administration +/− 7 days), every 8 weeks following IMP administration (−/+ 7 days) up to Week 24, then every 12 weeks (−/+ 7 days) and at the end of study intervention, except if already done at last cycle. Patients who discontinued study intervention without progressive disease are followed every 12 weeks until the documented progressive disease. Tumor assessment is repeated to confirm a partial or complete response as well as progressive disease (at least 4 weeks after initial documented response). For participants who do not have visceral/deep lymphatic lesions, radiological tumor assessment of abdomen and thorax are performed at 24 weeks, if there is no clinical sign of metastatic disease, and at EOT if not already done at last cycle. Intermittent ultrasonography (USG) or clinically indicated assessment can be considered in case of clinical signs or laboratory abnormalities, mainly liver function tests, to exclude potential metastatic disease.
^{x}End of treatment (30 ±5 days after last treatment): Obtain end of treatment assessments if not performed during the last week of the study.
^{y}Post-study follow-up for disease status: Participants without documented disease progression at the end of a treatment visit who have not yet started treatment with another anticancer therapy proceed with 3 months follow-up visits until initiation of another anticancer therapy, disease progression, death, or study cutoff date (whichever comes first).

TABLE 3
PK and PDy flowchart for Dose Escalation and Expansion phases

Week 1 of
subsequent

| | | |
|---|---|---|
| | odd- | Weeks |
| | numbered | following first |
| | cycles | administration |
| Cycle 1 | beyond | in |

| | | | | |
|---|---|---|---|---|
| Cylce 1 Week 1 and Cycle 3 Week l | Cycle 1 (Week 2) | (Weeks 3,4) | Cycle 3 | Cycle 1 Week 1 |

Day

| | | | | | | | | |
|---|---|---|---|---|---|---|---|---|
| D1 | D2 | D3 | D5/6 | D8 | D9 | D10 | D15, D22 | D1 |

Time (hour/minute) = RNT

| | | 0 H | 1 H | 2 H | 6 H | 24 H | 48 H | 96 H/120 H | 0 H | 2 H | 6 H | 24 H | 48 H | 0 H | 0 H | 6 H | | | | End of | |
|---|---|---|---|---|---|---|---|---|---|---|---|---|---|---|---|---|---|---|---|---|---|
| | Screen- | Indicative clock time | | | | | | | | | | | | | | | | | | treat- | Follow |
| Cycle | ing | 8:00 | 9.00 | 10:00 | 14:00 | 8:00 | 8:00 | 8:00 | 8:00 | 10:00 | 14:00 | 8:00 | 8:00 | 8:00 | 8:00 | 14:00 | 5 Wk | 8 Wk | Cycle 6 | ment | up |

Study treatment administration

| | | | | |
|---|---|---|---|---|
| The cytokine | X | X | X | X |
| RNA mixture | | | | |

Pharmacokinetics

| | | | | | | | | | | | | | | | | | | |
|---|---|---|---|---|---|---|---|---|---|---|---|---|---|---|---|---|---|---|
| Blood for target | X^{b} | X | X | X^{b} | X | X | X | X | X^{a} | X | X | X^{a} | X | X^{b} | X^{b} | X^{a} | X | X^{a} |

expression of the
cytokine RNA
mixture-
encoded
cytokines^{a}

Pharmacodynamics

| | | | | | | | | | | | | | |
|---|---|---|---|---|---|---|---|---|---|---|---|---|---|
| Blood for PDy | | X | X | X | X | X | X | X^{d} | X^{d} | | | X | X^{c} |
| cytokines^{c} | | | | | | | | | | | | | |
| Blood for HLA | X | | | | | | | | | | | | |
| typing and | | | | | | | | | | | | | |
| genetic analysis^{e} | | | | | | | | | | | | | |
| Blood for | X | | | | | | | | | X^{f} | | X | |
| Antigen specific | | | | | | | | | | | | | |
| T-cell^{f} | | | | | | | | | | | | | |
| Tumor biopsy | X | | | | | | | | | X^{g} | X^{g} | | |
| for immune | | | | | | | | | | | | | |
| assessment and | | | | | | | | | | | | | |
| RNA seq | | | | | | | | | | | | | |
| analysis^{g} | | | | | | | | | | | | | |

Immunogenicity

| | | | | | |
|---|---|---|---|---|---|
| Antibodies | | | | | |
| against cytokines | | | | | |
| encoded by the | X | X^{h} | X^{h} | X | X^{h} |
| cytokine RNA | | | | | |
| mixture (ADA)^{h} | | | | | |

^{a}Blood samples for PK are collected for evaluation of target expression of the cytokine RNA mixture -encoded cytokines in all enrolled participants on Cycle 1 Week 1 at pre-dose and 1, 2, 6, 24, 48, and 96 or 120 hours after IMP administration. At Cycle 1 Week 2 in the dose escalation phase, samples are collected at pre-dose and 2, 6, 24, and 48 hours post dosing; in the dose expansion phase, Cycle 1 Week 2 sampling occurs only at pre-dose, 6, and 24 hours post dosing. At Cycle 1 Week 3 and subsequent Cycles, see footnote^{b}. Samples are also collected right before the tumor biopsy, at EOT and the first follow up visit. Further information is detailed in the study laboratoty manual. No PK samples are collected following the second study cut-off date (see herein).
^{b}For PK, for Cycle 3 Week 1 (ie, week 9 front first administration), the schedule for Cycle 1 Week 1 is repeated. Beyond Cycle 3, PK sampling is to occur at 0 and 6 hours at Week 1 of every odd-numbered cycle. Beyond Cycle 3, PK samples of odd-numbered cycles can be omitted by notification of the Sponsor, if available data are considered sufficient.
^{c}Blood sample for immune assessment and circulating factors: Blood samples are collected at pre-dose, 6, and 24 hr of Cycle 1 Weeks 1 and 2, at EOT, and FU in all participants to assess systemic immune modulations including IFNγ and IP10. Further information us be detailed in the study laboratory manual.
^{d}In Cycle 3 Week 1 only, the sampling schedule for Cycle 1 Week 1 is repeated for immune assessment and circulating factors. Beyond Cycle 3, PDy sampling is to occur at 0 and 6 his at Week 1 of every odd-numbered Cycle. No sampling of blood for PDy cytokines occurs during even-numbered cycles during the monotherapy part of the study.
^{e}Blood for genetic analysis is used to establish the germline DNA sapience and HLA typing
^{f}Blood samples (leukapheresis or 80 mL of blood) are collected pre-dose Cycle 1 Week 1, pre-dose Cycle 2 Week 2 (ie, 5 weeks post-dose on Cycle 1), and at EOT for the analysis of antigen specific T-cell. This analysis will occur only for participants with melanoma in the monotherapy escalation phase and for all participants (melanoma) in the monotherapy expansion phase.
^{g}Tumor biopsy for immune assessment: biopsies are collected. during the screening period (before IMP administration on Cycle 1 Day 1), between Weeks 5 and 8, and at Cycle 6 or at disease progression (whichever occurs first), to assess immune modulations. Tumor transcriptomics (RNA sequencing), genomics, neo-antigens, and TIL isolation (expansion only in melanoma patients) may also be performed upon sample availability (see herein). For melanoma patients only, a single tumor core biopsy performed between Weeks 5-8 is dedicated for TILs isolation. This is applied to a limited number (aiming no more than 10 patients with successful TILs isolation) of selected melanoma patients (expansion for monotherapy and only in Cohort A of combination therapy expansion). This will not be an additional biopsy, but instead the sample dedicated for genomic assessment will be used for TILs isolation (handled under special conditions-not formalin fixed). This kind of sample and testing is applied to patients with clinical signs of response to treatment (tumor size reduction and/or redness at the tumor site) as determined by the treating investigator.
^{h}Plasma samples to monitor development of antibodies to the cylokine RNA mixture -encoded cytokines are collected pre-dose Day 1 for Cycles 1, 3, 6, 9, 12 and/or EOT, and at FU (Day 90 after last IMP administration). Additional collections beyond these timepoints are every 3 months if the participant continues on study for follow-up visits. No ADA samples are collected following the second cut-off date.

Objectives and endpoints for the treatment are shown in Table 4.

TABLE 4
Objectives and endpoints

| | |
|---|---|
| Objectives | Endpoints |

Primary

| | |
|---|---|
| Dose Escalation: To determine the maximum | Incidence of DLTs during the first 28 days of |
| tolerated dose (MTD) of maximum | treatment. |
| administered dose (MAD) and the overall safety | MTD, defined as the highest dose level with a true |
| and tolerability profile of the cytokine RNA | DLT rate during the first 28 days of treatment |
| mixture when administered intratumorally as | within tile target range of 16% to 33% and with |
| monotherapy in patients with advanced solid | less than 0.25 probability of a true DLT rate |
| tumors who have failed a prior anti-PD-1 or | greater than 33%. |
| anti-PD-L1 based therapy and/or patients | Doses above DL8 are not tested and if this MAD |
| without other treatment options for those | is safe, it will be considered the MTD. |
| indications in which anti-PD-1 is not routinely | Adverse events (AEs)/setious adverse events |
| used. | (SAEs), and laboratory abnormalities. |
| Dose Expansion: To determine the objective | Objective response rate (ORR) is assessed by |
| response rate of the cytokine RNA mixture | evaluation of anti-tumor response information |
| administered intratumorally in monotherapy in | according to RECIST 1.1 |
| patients with advanced melanoma that have | |
| failed a prior therapy based on anti-PD-1 or | |
| anti-PD-L1. | |

Secondary

| | |
|---|---|
| Dose Escalation and Expansion: To | Cmax and AUC of the cytokines encoded by the |
| characterize the pharmacokinetic (PK) profile | RNA mixture at Cycle 1 Week 1 and Cycle 3 |
| of the cytokines encoded by the mixture | Week 1; Ctrough of the cytokines encoded by |
| administered as monotherapy. | RNA mixture before IMP administration at each |
| | cycle. |
| Dose Escalation and Expansion: To assess the | Human antibodies against the cytokines encoded |
| immunogenicity of cytokines encoded by the | by the mixture up to end of study intervention and |
| mixture. | during follow-up will be evaluated. |
| Dose Expansion: To characterize the safely of | AE/SAEs and laboratory abnormalities. |
| the cytokine RNA mixture when administered | |
| intrattunorally as monotherapy in patients with | |
| advanced melanoma. | |
| Dose Expansion: To determine the disease | DCR, DoR, and PFS assessed according to |
| control rate (DCR), duration of response (DoR) | RECIST 1.1 and iRECIST criteria; ORR |
| and progression free survival (PFS) of the | assessed according to iRECIST criteria. |
| cytokine RNA mixture. | |
| Dose Escalation: To determine the | Recommended dose based on the MTD/MAD |
| recommended dose of the cytokine RNA | defined by the Bayesian model, the overall |
| mixture for the expansion phase.. | safety, activity and PK/PDy data.. |

Tertiary/exploratory

| | |
|---|---|
| Dose Escalation: To assess preliminaty clinical | Preliminary clinical benefit is assessed by |
| benefit by evaluation of anti-tumor activity of the | evaluation of anti-tumor response according |
| cytokine RNA mixture according to RECIST 1.1 | to RECIST 1.1 and iRECIST criteria of the |
| and iRECIST criteria. | cytokine RNA mixture monotherapy. |
| | Categories of response such as complete |
| | response (CR), partial response (PR), stable |
| | disease as best response or progressive |
| | disease is obtained in participants for |
| | assessment of ORR and DCR. |
| Dose Escalation and Expansion: To evaluate the | Blood samples are collected pre and post- |
| immune-modulation related pharmacodynamic | treatment during Cycle 1 and subsequent |
| (PDy) effects of the cytokine RNA mixture in | cycles to assess immune-modulation related |
| peripheral blood by measuring changes of | pharmacodynamic (PDy) effects (e.g., IFNγ |
| circulating factors including cytokines, | and IP10) and measuring a panel of |
| chemokines and other soluble proteins and | circulating cytokines to monitor safety and to |
| correlate with clinical parameters. | correlate with clinical parameters. |
| Dose Escalation and Expansion: To evaluate | Tumor biopsies are collected pre- and |
| treatment related changes in transcriptome profiles | post-treatment to define: |
| and tumor immune-infiltrate by RNA sequencing | Change in gene expression |
| (RNAseq) and immunohistochemistry (IHC), | profiles and tumor antigen |
| respectively. | expression by RNA sequencing |
| | Changes in types, quantities, and |
| | location of immune cells in the tumor |
| | microenvironment by IHC |
| | Changes in expression of expressed |
| | cytokines by ELISPOT |
| Dose Escalation and Expansion: To evaluate the | Blood samples are collected before the first |
| immune response against tumor antigens relevant | IMP administration and after Cycle 1 to |
| for the respective tumor entity by detecting of | evaluate the immune response by detection of |
| antigen-specific T-cell responses from peripheral | antigen-specific T-cell response to shared |
| blood (in melanoma patients). | antigens and tumor specific neo-antigens. |
| Dose Escalation and EKpansion: To explore | Genomic DNA and RNA are extracted from |
| tumor genetic markers at baseline including tumor | peripheral blood and tumor biopsy tissue and |
| mutation burden (TMB; assessed only in | analyzed by whole exome and RNA |
| combinalam therapy), and HLA typing (assessed | sequencing. Tumor mutation burden is |
| in both monotherapy and combination therapy). | calculated by determining the total number of |
| | single nucleotide variants in each sample. |
| | HLA alleles and allele groups are determined |
| | using DNA-based methods. |

### Example 1.2—Dose Escalation and Dose Expansion of the Cytokine RNA Mixture in Escalation Phase and Expansion Phase

A dose escalation and dose expansion study of the cytokine RNA mixture is performed in patients with advanced solid tumors in escalation phase and advanced melanoma in expansion phase, based on clinical, pharmacokinetic [PK], pharmacodynamic [PDy], and biomarker evaluations, to assess the safety and preliminary activity of the cytokine RNA mixture when administered intratumorally as monotherapy, and to define the optimal dose of drug as a single agent.

Screening occurs for up to 28 days before participants receive their first dose of the cytokine RNA mixture, and evaluations occur on a schedule with drug administration intratumorally at days 1, 8, 15, and 22 of a 4-week cycle. Treatment is continued weekly as a 4-week cycle until disease progression or AE leading to permanent discontinuation; otherwise it is continued up to 1 year of treatment. A single-participant dose escalation for the first two dose levels (DLs) is used in the escalation phase, followed by escalation to higher doses using a rational design.

### Example 1.2A. Dose Escalation Phase

During dose escalation, participants with advanced solid tumors amenable for intratumoral injection who failed a prior therapy based on anti-PD-1/PD-L1 are enrolled. Participants with solid tumors (other than melanoma), for which anti-PD-1/PD-L1 therapy is not routinely used, are also eligible if there are no other suitable treatment options, based on the discretion of the Investigator. Participants are treated with intratumoral injection of the cytokine RNA mixture administered weekly as monotherapy.

The starting dose level (DL 1) is determined from the results of various preclinical studies examining the PK of cytokines encoded by the cytokine RNA mixture in human xenograft models, and allometric scaling from mouse to human using modeling and simulation.

The experiments include an accelerated dose escalation design for the first two DLs (DL1 and DL2), where one participant is treated by DL and an escalation between two dose levels is applied until observation of any IMP-related Grade ≥2 AE or dose limiting toxicity (DLT). If an IMP-related Grade ≥2 AE is observed at either of the first two DLs, two additional participants are treated at the same DL and dose escalation will proceed using an adaptive rational design.

If no IMP-related Grade ≥2 AE or DLT occurs in the first 2 DLs, then an adaptive dose escalation starts from DL3. Dose escalation for subsequent cohorts (DL3-DL8) proceeds. Enrollment to the next DL does not proceed before at least three participants treated at the previous DL have been followed for a duration of at least I cycle (i.e., 28 days), and are evaluable for DLT assessment with no DLT. No intra-participant dose escalation is allowed. There is a gap of at least one week between the first and second participants treated at the same dose level.

### Example 1.2B. Lesions to be Injected

All dose levels (DL1-DL8) follow the guidance on lesion size provided in Table 5. Participants have a minimum of one measurable lesion as target lesion according to the Response Evaluation Criteria in Solid Tumors (RECIST 1.1) criteria (see Inclusion criterion I 05), and minimum of one or more cutaneous/subcutaneous lesion(s) for injection and tumor biopsy. Participants are selected based on the size of the tumor lesions which have to be sufficient for the injection volume of that given dose level (Table 5), with the consideration of biopsy of one lesion at baseline as well as between weeks 5th-8th of first administration as on-treatment assessment.

In the escalation phase only, if non-target lesions allow signal of response assessment, participants who have no measurable lesions may be evaluated case by case for eligibility with the agreement of study committee. Enrollment of patients with solely mucosal sites for injection is done only at dose levels in which significant inflammation of superficial, subcutaneous and/or lymph node metastases have not been observed with cytokine RNA mixture to minimize the risk of airway obstruction.

### Example 1.2C. Treatments

For the first treatment, among the 3 minimum lesions, one measurable lesion (cutaneous, visceral or lymph node) is left intact for measurements according to RECIST 1.1 criteria and one lesion is used for biopsy. If the lesion to be injected is large enough to be used for biopsy with no impact on dose administration at planned dose level, then two lesions are sufficient for eligibility. A minimum of one lesion is subject to administration of the cytokine RNA mixture (size of the lesion[s] should be assessed per dose level for participant's eligibility). The largest lesion(s) is injected first with the cytokine RNA mixture. For the remaining lesion(s), rank of injection is based on lesion size until maximum injection volume is used (see Table 5 below).

TABLE 5
Injection Volume

| Lesion size | |
|---|---|
| (longest diameter) | Injection Volume |
| >5 cm | Up to 4 mL |
| >2.5 cm to 5 cm | Up to 2 mL |
| >1.5 cm to 2.5 cm | Up to 1 mL |
| >0.5 cm to 1.5 cm | Up to 0.5 mL |

For all subsequent treatments (weekly), injection of lesion(s) is ranked based on lesion size until maximum injection volume is used or until all injectable lesion(s) are treated.

The volume to be injected is based on the size of the lesion, and the maximum injection volume for each treatment visit should not exceed the volume assigned for that DL for all injected lesions combined. The maximum injection volume allowed for DL8 is 4 mL.

If lesions are clustered together, they are injected as a single lesion according to the table and guidance above.

It is preferable to inject only one lesion per treatment based on the volume and size of the lesion ratio in Table 5. If it is not possible to inject only one lesion, then the volume/dose is divided in multiple lesions. At each visit, lesions for injection are prioritized based on size starting with the largest lesion first. The largest lesion is injected with maximum injection volume based on the lesion size and dose levels. If the volume is not all used, the next lesion is administered with maximum injection volume allowable for lesion size. Administration continues from largest to smallest until the entire dose volume has been administered.

When it is not possible to inject all lesions at each treatment visit or over the full course of treatment, previously injected and/or non-injected lesion(s) are injected at subsequent treatment visits. The administration details per lesions are collected in the electronic case report form (eCRF).

### Example 1.2D. Dose Escalation Decision

Decisions to escalate consider the results of clinical safety. The DLT observation period is the first 4 weeks of treatment (Cycle 1). A participant is considered evaluable for DLT assessment if he/she receives at least 70% of his/her cohort planned dose in the first 28 days of the treatment (i.e., DLT period) and is evaluated for 28 days, or if an earlier DLT occurs. Participants who are not evaluable for DLT assessment in the dose escalation phase (e.g., early progressive disease before Cycle 1 Day 28; any missing DLT assessment parameters) are replaced.

For the escalation of the first two DLs, the second DL begins after the DLT observation period for the first participant is completed without an IMP-related AE Grade 2 or DLT. If an IMP-related AE Grade 2 or any DLT is observed at either of the first two DLs, two additional participants are treated at the same DL and dose escalation proceeds using an adaptive design. If no IMP-related AE Grade ≥2 occurs in the first two DLs, then an adaptive Bayesian EWOC starts from DL3. Enrollment to DL2 or DL3 in the monotherapy part of the study may not proceed until the patient enrolled in DL1 or DL2 has been followed for 28 days, and is evaluable for AE assessment with no IMP-related AE Grade 2.

Dose escalation is stopped as soon as the MTD is determined. If an MTD is not determined, dose escalation continues until the MAD is achieved.

### Example 1.2E. Dose Expansion Phase

Based on the MTD/MAD, the overall safety, activity, and PK/PDy data, the recommended dose for the expansion phase is decided.

Up to 34 participants with advanced melanoma that have failed a prior therapy based on anti-PD-1/PD-L1 are enrolled at the MAD or MTD to further assess safety (especially any cumulative toxicity), anti-tumor activity, PDy, and PK activities.

### Example 1.2F. Duration of Study Period

The duration for each participant includes a period for screening of up to 28 days. The cycle duration is 28 days. After completion of the first cycle, participants may continue to receive additional administrations of the cytokine RNA mixture at the same DL every week, if this dosing regimen is considered safe and the participant is achieving a clinical benefit. The expected treatment period for participants who benefit from the cytokine RNA mixture may vary, based on progression date.

After discontinuation of intervention, participants return 30 days (for end-of-treatment [EOT] assessments) and 90 days (for ADA sample) after the last IMP administration or before the start of another anticancer therapy, whichever is earlier.

After the EOT visit, additional follow-up visits may be required to monitor all ongoing related and new related AEs until resolution or stabilization (i.e., an event ongoing without any change for at least 3 months). After the EOT, during the safety follow-up period, the events to be reported, monitored, and followed-up to resolution or stabilization are as follows: all ongoing AEs, SAEs, or Events of Special Interest regardless of relationship and all new AEs, SAEs, or Events of Special Interest considered related, including deaths due to related events.

In addition, if the participant discontinues intervention for reasons other than progression, follow-up visits are performed every 3 months until progression or initiation of another anti-tumor treatment, or death (whichever comes first) in order to document disease progression.

The total median estimated duration of enrollment is approximately 24 months. The expected duration of treatment for participants who benefit from the cytokine RNA mixture may vary, based on progression date; but median expected duration of treatment per participant is estimated as 9 months (1 month for screening, 5 months for treatment, and 3 months for the EOT and first follow-up visits).

Stopping Rules: in case of any deaths (other than death related to progressive disease (PD)) within 30 days of therapy, or Grade 4 TEAEs in more than one third of patients enrolled at a certain dose level (e.g. 2 out of 3 patients), enrollment in the trial will be paused until an appropriate evaluation of the cause of death and toxicity is conducted by the Study Committee and a correction plan is established.

### Example 1.2G. Choice of Starting Dose

The starting dose is generally established for anticancer compounds based on the results of toxicology studies in rodent and non-rodent species. The cytokine RNA mixture is administered via intratumoral injection, and its biological activity depends on uptake and translation of the administered mRNA. Preclinical toxicology studies were performed in non-tumor bearing rodent and non-rodent species, and surrogate routes of administration may not accurately reflect the intratumoral route of administration. As a result, the conventional procedure for determination of a first-in-human starting dose based on the International Council for Harmonisation (ICH) S9 recommendation of 1/10 the Severely Toxic Dose in 10% of the animals (STD 10) in rodents and no observed-adverse-effect-level (NOAEL) is not relevant for locally administered intratumoral mRNA agents.

To determine a starting dose for human, in vivo experiments are performed in immunocompromised mice bearing human A375 melanoma xenografts. Intratumoral administration of the cytokine RNA mixture in the A375 xenograft leads to translation of each of the cytokine components of the cytokine RNA mixture. While the cytokine mixture is expressed locally within the tumor, the encoded cytokines are secreted and enter into circulation leading to systemic exposure to the cytokines. The PK parameters of the cytokines encoded by the cytokine RNA mixture are assessed. The serum PK parameters of the cytokine RNA mixture encoded cytokines in the A375 xenograft showed a dose-dependent expression relationship.

Assuming a comparable tumor expression potential of the cytokine RNA mixture encoded cytokines between mouse and human, the individual PK models in mouse are scaled to human using allometry, and simulations are performed to predict the human systemic cytokine exposure at different dose levels of the cytokine RNA mixture. Due to the uncertainties of pharmacological activity in humans versus animals and interspecies differences related to cytokines, a wide safety margin is applied, and a human dose is selected.

### Example 1.2H—End of Study Definition

A participant is considered to have completed the study if he/she has completed all phases of the study intervention up to a maximum of 1 year (including End of Treatment), or if treatment is terminated due to another reason and the participant completed follow-up visits until progressive disease.

There are two cut-off dates for the study:

The first trial cut-off date is at the end of the 28 days of the last participant treated in the dose escalation phase in order to have all participants with evaluable DLT data for determination of the MTD/MAD.

The second cut-off date is either when the last participant on treatment in the expansion phase will have had two post-baseline tumor assessments or end of treatment assessment, whichever occurs first, in order to assess tumor response.

If a participant, treated in either the dose escalation phase or the expansion phase, continues to benefit from the treatment after the second study cut-off, the participant can continue study intervention (for up to 1 year of treatment) and will undergo assessments for IMP-related AEs, any SAE, and blood samples for assay of immunogenicity, if applicable.

The end of the study is defined as the date of the last visit of the last participant in the study.

### Example 1.3—Study Population

### Example 1.3A—Inclusion Criteria

Participants are eligible to be included in the study only if all of the following criteria apply as shown in Table 6.

TABLE 6
Inclusion Criteria

| Category | | Criteria |
|---|---|---|
| Age | I 01. | Participants must be 18 years of age inclusive, at the time of |
| | | signing the informed consent. |
| Type of | I 02. | Dose escalation phase: Participants with a histologically |
| participant | | confirmed, advanced unresectable or metastatic solid tumor |
| and disease | | including lymphomas who, according to international treatment |
| characteristics | | guidelines and in the opinion of the Investigator, for whom no |
| | | alternative suitable treatment options exist. |
| | I 03. | Dose expansion phase: Participants with histologically confirmed |
| | | advanced or metastatic melanoma, regardless of BRAF status, |
| | | including stage IIIB-C or unresectable stage IV (M1a-c and M1d if |
| | | criterion E09 is satisfied) as assessed by the American Joint |
| | | Committee on Cancer melanoma staging edition 8, for whom no |
| | | alternative suitable treatment options exist. Eligible patients must |
| | | be ineligible for or decline to receive available standard of care |
| | | (SOC). Investigators must inform prospective patients participants |
| | | of the availability of current SOC treatment options prior to trial |
| | | participation. |
| | I 04. | For melanoma anti-PD-1/PD L1 failure (expansion phase), |
| | | participants previously treated with anti-PD-1/PD-L1 and had |
| | | confirmed progressive disease. |
| | | Only in the monotherapy dose expansion phase: in melanoma, |
| | | patients who become intolerant to anti-PD-1/PD-L1 therapies |
| | | are also eligible. |
| | I 05. | Participants have a minimum of 3 lesions (or 2 for selected cases, |
| | | as described below) at enrollment, with measurable disease defined |
| | | as: |
| | | One lesion as target lesion for measurable disease*, defined as: |
| | | One cutaneous lesion of at least 1 cm as target lesion to be |
| | | measured according to RECIST criteria |
| | | OR |
| | | One or multiple visceral lesion(s) that can be accurately |
| | | and serially measured in at least 2 dimensions, and for |
| | | which the longest diameter is ≥1 cm (as measured by |
| | | contrast enhanced or spiral computed tomography [CT] |
| | | scan) for visceral or soft tissue disease or ≥1.5 cm in the |
| | | short axis for lymph nodes. These visceral lesions will be |
| | | used for RECIST criteria measurements. |
| | | Patients with non-FDG avid lymphomas were required to |
| | | have measurable disease defined as at least one measurable |
| | | node that must have an LDi (longest diameter) >1.5 cm |
| | | and/or measurable extranodal lesion that must an LDi |
| | | >1 cm by a contrast-enhanced CT according to Lugano |
| | | classification (29) (See herein). Patients with FDG-avid |
| | | lymphomas were not required to have measurable disease. |
| | | *Only in the escalation phase, if non-target lesion(s) is suitable |
| | | for surrogate response assessment, participants who do not have |
| | | measurable disease will also be eligible based on case by case |
| | | discussion with sponsor. |
| | | One lesion for biopsy cutaneous, subcutaneous or lymph node |
| | | amenable for biopsy); this lesion can also be used for injection, |
| | | if feasible, in which case, 2 lesions might be sufficient for |
| | | eligibility of participants. |
| | | At least one lesion amenable for intratumoral injection, as |
| | | detailed in I06. |
| | I 06. | Participants have lesions in which an injection can be performed |
| | | (i.e., suitable for direct intratumoral injection based on the dose |
| | | level volume of each cohort and according to the investigator's |
| | | judgement) defined as cutaneous or subcutaneous lesions ≥0.5 cm |
| | | in longest diameter or multiple injectable merging lesions which |
| | | become confluent and have the longest diameter (sum of diameters |
| | | of all involved target lesions) of ≥0.5 cm suitable for injection (i.e., |
| | | not bleeding or weeping) to be treated with the cytokine RNA |
| | | mixture at each visit. Lymph nodes ≥1.5 cm that are suitable for |
| | | ultrasonography (USG)-guided intratumoral injection and |
| | | confirmed as metastatic disease are also acceptable. |
| | I 07. | Participants must be able and willing to provide mandatory tumor |
| | | biopsies prior to and after study intervention. |
| | I 08. | Participants eligible for second line therapy, if not a candidate for |
| | | available treatment options due to tumor characteristics, co- |
| | | morbidities, pre-existing autoimmune disease, drug unavailability |
| | | or not a standard of care at each country as well as if participant |
| | | declined these options that have been transparently disclosed. |
| | I 09. | Participants with life expectancy more than 3 months. |
| | I 10. | Participants with metastatic uveal and mucosal melanoma are |
| | | eligible for the monotherapy dose escalation parts of the study if |
| | | they have superficial, subcutaneous and/or lymph node metastases |
| | | amenable for intratumoral injection. Participants with HNSCC and |
| | | mucosal melanoma with only mucosal sites for intratumoral |
| | | injection are eligible for the monotherapy and combination dose |
| | | escalation parts of the study following a discussion and approval |
| | | by the Sponsor. |
| Sex | I 11. | Male or Female |
| | | Male participants: A male participant must agree to use |
| | | contraception during the intervention period and for at least 6 |
| | | months after the last dose of study intervention and refrain |
| | | from donating sperm during this period. |
| | | Female participants: A female participant is eligible to |
| | | participate if she is not pregnant, not breastfeeding, and at least |
| | | one of the following conditions applies: |
| | | Not a woman of childbearing potential (WOCBP) OR |
| | | A WOCBP who agrees to follow the contraceptive guidance |
| | | during the intervention period and for at least 6 months after |
| | | the last dose of study intervention. |
| | I 12. | Metastatic Uveal and Mucosal Melanoma |
| | | Participants with metastatic uveal and mucosal melanoma are |
| | | eligible for the monotherapy dose escalation parts of the study |
| | | if they have superficial, subcutaneous and/or lymph node |
| | | metastases amenable for intratumoral injection. Participants |
| | | with HNSCC and mucosal melanoma with only mucosal sites |
| | | for intratumoral injection are eligible for the monotherapy dose |
| | | escalation parts of the study following a discussion and |
| | | approval by the Sponsor. |
| | I 13. | Participants must have an adequate cardiac function with left |
| | | ventricular ejection fraction (LVEF) of at least 50%. |
| Informed | I 14. | Capable of giving signed informed consent which includes |
| Consent | | compliance with the requirements and restrictions listed in the |
| | | informed consent form (ICF) and in this protocol. |

### Example 1.3B—Exclusion Criteria

Participants are excluded from the study if any of the following criteria apply as shown in Table 7.

TABLE 7
Exclusion Criteria

| Category | | Criteria |
|---|---|---|
| Medical | E 01. | Eastern Cooperative Oncology Group (ECOG) performance |
| conditions | | status >1. |
| | E 02. | Significant & uncontrolled concomitant illness, including any |
| | | psychiatric condition that, in the opinion of the Investigator or |
| | | Sponsor, would adversely affect the participant's participation |
| | | in the study. |
| | E 03. | Active infections, including unexplained fever (temperature |
| | | >38.1° C.), or antibiotics therapy within 1 week prior to |
| | | enrollment. |
| | E 04. | Any prior organ transplant including those who had received |
| | | allogeneic bone marrow transplant. |
| | E 05. | History within the last 5 years of an invasive malignancy |
| | | other than the one treated in this study, with the exception of |
| | | resected/ablated basal or squamous-cell carcinoma of the skin |
| | | or carcinoma in situ of the cervix, or other local tumors |
| | | considered cured by local treatment. |
| | E 06. | History of known acquired immunodeficiency syndrome |
| | | (AIDS) related illnesses or known HIV disease requiring |
| | | antiretroviral treatment, or active hepatitis A, B (defined as |
| | | either positive HBsAg or negative HBsAg with positive HBc |
| | | antibody), or C (defined as a known positive hepatitis C |
| | | antibody result and known quantitative HCV RNA results |
| | | greater than the lower limits of detection of the assay) |
| | | infection. HIV serology at screening will be conducted only |
| | | for participants at German study sites. |
| | E 07. | Participants who had splenectomy. |
| | E 08. | Tumor lesions to be injected located in mucosal regions or |
| | | close to an airway, major blood vessel or spinal cord that, in |
| | | the opinion of the Investigators, could cause occlusion or |
| | | compression in the case of tumor swelling or erosion into a |
| | | major vessel in the case of necrosis. |
| | E 09. | Untreated brain metastasis(es) that may be considered active. |
| | | Patients with previously treated brain metastases may |
| | | participate provided they are stable (i.e., without evidence of |
| | | progression by imaging for at least 6 weeks prior to the first |
| | | dose of study treatment, and any neurologic symptoms have |
| | | returned to baseline), and there is no evidence of new or |
| | | enlarging brain metastases, and the patient does not require |
| | | any systemic corticosteroids for management of brain |
| | | metastases within 4 weeks prior to the first dose of the |
| | | Cytokine RNA mixture. |
| | E 10. | Symptomatic congestive heart failure (NYHA 3 or 4), history |
| | | of myocarditis, serious uncontrolled cardiac arrhythmia, |
| | | myocardial infarction 6 months prior to study entry, unstable |
| | | angina pectoris, uncontrolled or unresolved acute renal failure, |
| | | or significant pulmonary conditions such as the following: |
| | | Uncontrolled chronic lung disease |
| | | A known history past 5 years) of, or any evidence of, |
| | | interstitial lung disease |
| | | Active, non-infectious pneumonitis. |
| | E 11. | Ongoing or recent (within 5 years) evidence of significant |
| | | autoimmune disease that required treatment with systemic |
| | | immunosuppressive treatments, which may suggest risk for |
| | | immune-related adverse events. The following are not |
| | | exclusionary: vitiligo, childhood asthma that has resolved, |
| | | residual hypothyroidism that required only hormone |
| | | replacement or psoriasis that does not require systemic |
| | | treatment. |
| | E 12. | Non-resolution of any prior treatment related toxicity to Grade |
| | | <2, except for alopecia, vitiligo, fatigue and active |
| | | hypothyroidism according to National Cancer Institute |
| | | common terminology criteria for adverse events (NCI |
| | | CTCAE) version 5.0. |
| | E 13. | Histoly of a systemic hypersensitivity reaction, other than |
| | | localized injection site reaction, to any biologic drug and |
| | | known hypersensitivity to any constituent of the Cytokine |
| | | RNA mixture. |
| | E 14. | Primary uveal melanoma without injectable superficial, |
| | | subcutaneous, or lymph node metastatic disease. |
| Prior/concomitant | E 15. | Concurrent treatment with any other anticancer therapy |
| therapy | | (including radiotherapy or investigational agents) or |
| | | participation in another interventional clinical study. |
| | E 16. | Washout period of less than 3 weeks to prior anticancer |
| | | therapy (including chemotherapy, targeted agents, |
| | | radiotherapy, immunotherapy, vaccination, or any |
| | | investigational agent), or 5 times the half-life, whichever is |
| | | shorter. For participants who received prior immunotherapies |
| | | (including anti-PD-1 therapies), a washout period of at least 4 |
| | | weeks is required before a participant receive the IMP. |
| | E 17. | Immunosuppressive corticosteroid doses (prednisone >7.5 mg |
| | | daily orally [PO] or intravenously [IV], or equivalent) within |
| | | 2 weeks prior to the first dose of IMP and maintenance |
| | | therapy with prednisolone >7.5 mg/day orally or equivalent |
| | | during the study. |
| | E 18. | Prior treatment with other immune modulating agents within |
| | | fewer than 4 weeks or 5 half-lives of the agent (whichever is |
| | | shorter) prior to the first dose of IMP. Examples of immune |
| | | modulating agents include blockers of CTLA-4, 4-1BB |
| | | (CD137), OX-40, therapeutic vaccines, or cytokine |
| | | treatments. |
| | E 19. | Prior treatment with live, attenuated vaccines within 4 weeks |
| | | prior to initiation of study intervention, during treatment, and |
| | | for 3 months after the last dose of the IMP. |
| Prior/concurrent | E 20. | Previous enrollment in this study. |
| clinical study | E 21. | The participant is the Investigator or any sub-investigator, |
| experience | | research assistant, pharmacist, study coordinator, other staff or |
| | | relative thereof directly involved in the conduct of the |
| | | protocol. |
| Diagnostic | E 22. | Inadequate hematologic function including neutrophils <1.5 × |
| assessments | | 10⁹/L; hemoglobin <9.0 g/dL; plately count <100 × 10⁹/L. |
| | E 23. | Inadequate renal function with creatinine ≥1.5x ULN, or |
| | | between 1.0-1.5x ULN with a CrCl <60 mL/min/1.73 m² as |
| | | estimated by using the aMDRD formula. |
| | E 24. | Inadequate liver function or coagulation test: Total bilirubin |
| | | >1.5x ULN unless Gilbert's syndrome (for this situation, total |
| | | bilirubin >2.5x ULN), or ALT, AST, or ALP >2.5x ULN in |
| | | the absence of hepatic metastases. In the presence of hepatic |
| | | metastases, total bilirubin <3x ULN and AST or ALT <5x |
| | | ULN are acceptable. Alkaline phosphatase (ALP) up to Grade |
| | | 2, i.e., <5x ULN, would be acceptable only if related to the |
| | | presence of bone metastases as judged by the Investigator. |
| | | Prothrombin time (PT) or international normalized ratio (INR) |
| | | >1.5x ULN. The participants on anticoagulant therapy will be |
| | | excluded. Participants on low dose aspirin (≤100 mg daily) |
| | | and prophylactic low dose heparin are not excluded. |
| Other exclusions | E 25. | Prisoners or subjects who are legally institutionalized, or those |
| | | unwilling or unable to comply with scheduled visits, drug |
| | | administration plan, laboratory tests, other study procedures, |
| | | and study restrictions. |
| | E 26. | Central nervous system lymphoma |
| | E 27. | Prior allogeneic HSCT for patients with lymphoma. |
| | E 28. | Auto-HSCT less than 90 days prior to initiation of study |
| | | intervention, |

### Example 1.4—Study Intervention

Study intervention is defined as any investigational intervention(s), marketed product(s), placebo, or medical device(s) intended to be administered to a study participant according to the study protocol.

### Example 1.4A—Study Intervention(s) Administered

TABLE 8
Overview of study interventions administered

| Study | |
|---|---|
| intervention | The cytokine |
| name | RNA mixture |
| Dosage | Concentrate for |
| formulation | solution for |
| | injection |
| Route of | Intratumoral |
| administration | injection |
| Dosing | Injection(s) |
| instructions | administered at |
| | assigned dose |
| | level once a |
| | week; 4 doses |
| | within a 28-day |
| | cycle.^{a} |

^{a}No predefined premedication will be administered to all participants, but secondary premedication might be recommended for some participants.

The cytokine RNA mixture is the investigational medicinal product and is a 1:1:1:1 weight ratio of synthetic, chemically modified mRNAs encoding the human cytokines IL-15sushi, IL-12sc, GM-CSF, and IFNα2b.

The cytokine RNA mixture is administered intratumorally once per week in a 4-week cycle (i.e., four doses every 28 days). After each cycle of treatment, the frequency of intratumoral injection continues weekly. However, during the conduct of the study, the dose administration frequency may be reduced to less frequent administration based on tumor burden decrease, which may interfere with administration of the intended dose.

As the route of administration is intratumoral injection, no acute allergic reaction is expected so there is no pre-defined premedication to be administered to all participants; however, premedication may be recommended for some participants. All the drugs used as premedication are entered to the concomitant medication pages.

### Example 1.4A1—Guidelines for the Management of Potential Tumor Lysis Syndrome (TLS)

In case of TLS, study treatment (cytokine mRNA mixture) should be held until all serum chemistries have resolved. To ensure normal hydration, correct laboratory abnormalities, fluid overload, electrolyte or acid-base deviation. Management must be done according to the local site guideline. Use of inhibitors (e.g., allopurinol) or urate oxidase (e.g., rasburicase) is allowed. TLS complications including renal function should be monitored, and study treatment can be reinstituted at full doses after resolution.

The laboratory abnormalities normally associated with TLS, and the possible clinical manifestations which can be associated with TLS are presented in Table 9.

TABLE 9
Laboratory and clinical abnormalities possibly consistent with TLS

| Laboratory | Clinical |
|---|---|
| Uric acid >8 mg/dL (>475.8 μmol/L) | Acute kidney injury: increase in the serum creatinine |
| Potassium >6.0 mmol/L | level of (≥1.5 times the ULN) or the presence of |
| Phosphorus >4.5 mg/dL (1.5 mmol/L) | oliguria, defined as an average urine output of |
| | <0.5 mL/kg/hour for 6 hours. Seizures, cardiac |
| Corrected calcium^{a} <7.0 mg/dL(<1.75 | dysrhythmia, neuromuscular irritability (tetany, |
| mmol/L) or ionized calcium <1.12 mg/dL | paresthesias, muscle twitching, carpopedal spasm, |
| (<0.3 mmol/L) | Trousseau's sign, Chvostek's sign, laryngospasm, |
| Increase in the serum creatinine level | bronchospasm), hypotension, or heart failure |
| (≥1.5 times the upper limit of normal [ULN] | probably or definitely caused by hypocalcemia. |
| | Dysrhythmias probably or definitely caused by |
| | hyperkalemia. |

^{a}The corrected calcium level in milligrams per deciliter = measured calcium level in milligrams per deciliter + 0.8 × (4-albumin in grams per deciliter)

### Example 1.4B—Concomitant Therapy

Any medication (including over-the-counter or prescription medicines, vitamins, and/or herbal supplements) that the participant is receiving at the time of enrollment or receives during the study must be recorded with reason for use, and dates of administration including start and end date.

Concomitant medications are recorded in the eCRF from 14 days prior to the initial dose of study drug until 30 days after the last administration of study drug, resolution of ongoing study-drug related adverse events, or when another anticancer therapy is received.

Concomitant medication may be considered on a case-by-case, in accordance with the following guidelines:
Systemic steroids and TNF-α antagonists may attenuate the potential benefit of the IMP, however in an emergency situation the treating investigator is allowed to apply these drugs. Nevertheless, this should be communicated to the sponsor as soon as possible and a common decision if and how the participants can proceed with study participation.
If feasible, alternatives to corticosteroids should always be considered. Physiological doses of corticosteroids or replacement steroids are allowed after consultation with the Sponsor. The use of inhaled steroids and oral mineralocorticoids (e.g., fludrocortisone for participants with orthostatic hypotension or adrenocortical insufficiency) is allowed.
   A participant may receive corticosteroids acutely for an emergency, allergic reaction, or similar; Participants may not receive maintenance therapy with prednisolone >7.5 mg/day (PO or IV) or equivalent during the study. Equally physiologic doses of corticosteroids given for adrenal insufficiency are allowed.
Concomitant treatment with myelosuppressive chemotherapy is prohibited for all participants during the treatment phase as well as 3 weeks or 5 half-lives, whichever is shorter, before treatment start.
Concomitant treatment with antipyretics (e.g., 1 g acetaminophen/paracetamol when a participant develops a fever >38.5° C.) is allowed.
Palliative radiotherapy may be given for control of pain for palliative intents. Sponsor should be notified to obtain prior approval prior to treatment if palliative radiotherapy is being considered, and prior to resuming therapy on the study. The irradiated area should be as small as possible and should never involve more than 20% of the bone-marrow in any given 3-week period. In all such cases, the possibility of tumor progression should be ruled out by physical and radiological assessments of the tumor. If the only evaluable lesions are to be irradiated, the participant will stop the study intervention. The irradiated area cannot be used as a parameter for response assessment.
Serious events manifested by dyspnea, hypotension, wheezing, bronchospasm, tachycardia, reduced oxygen saturation, or respiratory distress should be managed with supportive therapies as clinically indicated (e.g., supplemental oxygen and 02 adrenergic agonists).
Any background therapy taken by the participant for concomitant illnesses other than cancer (e.g., hormone-replacement therapy, statin, antihypertensive medication) is allowed to be continued at a stable dose.
Premedication may be needed in the select participants.

### Example 1.4C—Prior Therapy

Any prior anticancer therapy participant received before start of this study (medications and therapies including radiotherapies) should be entered in the eCRF.

Any previous treatment should have been finished according to the following timelines:
Any previous anticancer therapy, whether investigational or approved, including chemotherapy, hormonal therapy, and/or radiotherapy, has to be ended at least 3 weeks prior to initiation of study intervention.
If the participant has received investigational therapy before entering in this study at least 3 weeks or 5 half-lives should have passed before entering this study.
Any herbal therapy should be ended at least 1 week before administration of the IMP.
Prior treatment with cytokines is allowed provided that at least 4 weeks or 5 half-lives of the drug, whichever is shorter, have elapsed between the last dose the planned first administration of the IMP.
Prior treatment with immune checkpoint inhibitors, immunomodulatory monoclonal antibody (mAbs), and/or mAb derived therapies is allowed provided that 4 weeks or 5 half-lives (whichever is shorter) have passed until start of the therapy.

If a participant receives maintenance therapy with corticosteroids, the participants is eligible only if the dose can be tapered to <7.5 mg/day by 2 weeks before the first administration of IMP, and the participant should not have the risk of dose increase throughout the study intervention period.

### Example 1.4D—Premedication

As the route of administration is intratumoral injection, no acute allergic reaction is expected so there is no pre-defined premedication to be administered to all participants. However, premedication at and following the second cycle may be recommended depending on whether the participant experienced an inflammatory reaction following the first administration.

If participants had previously experienced drug-induced related allergic reactions (i.e., from mild itching to moderate symptoms that occurred within 24 hours of IMP administration), premedication with a histamine H1 antagonist (diphenhydramine 50 mg orally, or equivalent [e.g., dexchlorpheniramine], given approximately 30-60 minutes before administration of the cytokine RNA mixture) can be considered before administration of the cytokine RNA mixture. If participants had Grade 2 events including hypersensitivity or CRS, premedication might also include oral steroids (dexamethasone 20 mg or equivalent) for future administrations. Corticosteroid usage should be limited to the treatment of severe drug induced allergic reactions or life-threatening conditions.

Premedication with antipyretics is permitted for participants who developed inflammatory symptoms such as fever and shivering after the first administration of the IMP. Local anesthetics can be used based on location of lesion(s) to be injected.

### Example 1.4E—Prohibited Therapy

Use of the following therapies is prohibited during the study:
Concomitant therapy intended for the treatment of cancer (e.g., chemotherapy or immunotherapy), with the exception of palliative radiotherapy, is not allowed during the trial.
   Radiotherapy may be considered for symptom palliation (e.g., treatment of painful bony metastases, obstructing lung lesion) if participants are otherwise deriving benefit.
Live, attenuated vaccines are prohibited within 4 weeks prior to initiation of study intervention, during treatment, and for 3 months after the last dose of the IMP. All other vaccines are to be avoided if these are not the best solution for the participant's condition.
Concomitant treatment with IFN is prohibited for all participants during the participation in the study.
Systemic immunostimulatory agents (including but not limited to IFNs and IL-2) are prohibited within 4 weeks or 5 half-lives of the drug (whichever is longer) prior to initiation of intervention and during intervention because an interaction cannot be excluded and an increased risk for the participant could result.
Immunosuppressive medications, including but not limited to cyclophosphamide, azathioprine, methotrexate, and thalidomide. These agents could potentially alter the activity.
Maintenance therapy with prednisolone >7.5 mg/day (PO or IV) or equivalent is prohibited.
Systemic granulocyte colony stimulating factors (e.g., granulocyte colony stimulating factor, GM-CSF, and/or pegfilgrastim) as this could alter the activity of the IMP.
Participants in this study are not allowed to receive any other IMP concomitantly.

### Example 1.4F—Dose Modification

Dose Modification for the Cytokine RNA Mixture

If necessary or in case of an IMP-related Grade ≥2 AE, the start of the cytokine RNA mixture can be delayed by up to 3 days beyond the anticipated day of treatment at any week, and a delay of 2 or 3 days will be considered as a dose delay. The next dose should be planned 7 days after the last dose to respect a 7 day interval between doses.

If the cytokine RNA mixture dose needs to be delayed ≥4 days beyond the anticipated day of treatment for the weekly dose, then that dose needs to be skipped and will therefore be considered a dose omission. The participant may resume the cytokine RNA mixture if the IMP-related Grade ≥2 AE has resolved to Grade ≤1 (or Grade 2 if controlled with replacement therapies) within an acceptable period. In case of two sequential dose omissions, the patient may be re-treated with the cytokine RNA mixture if the AE is not life-threatening and continuation of treatment is considered best for the patient's condition. In case of more than two sequential dose omissions the cytokine RNA mixture will be terminated definitively.

Participants who experience a DLT in the monotherapy dose escalation part of the study, will have their study intervention stopped and will be followed until the toxicity has resolved.
If the DLT occurs during the first 28 days from the initiation of the cytokine RNA mixture (DLT observation period in the escalation phase), the cytokine RNA mixture will be terminated definitively.
If an AE fulfilling the DLT definition occurs after 28 days from the initiation of the cytokine RNA mixture (DLT observation period), the cytokine RNA mixture can be resumed following a discussion with the Sponsor and potential endorsement by study committee after ensuring that the following criteria are met:
   AE has resolved to Grade <1 (or Grade 2 if controlled with replacement therapies
   The Investigator believes that it is in the patient's best interest to resume the study intervention

Applicable only to dose escalation (not expansion phase), the participant will resume therapy with a new cycle of treatment at the same dose level of the cytokine RNA mixture with prophylactic treatment (if available) or at a lower dose level, based on agreement with the Sponsor. No dose re-escalation is allowed.

In the event of DLTs attributed to the cytokine RNA mixture whose re-occurrence would not necessarily be life-threatening (ie, skin rash not related to CRS, endocrinopathies such as hypothyroidism, fever, fatigue, arthromyalgia, headache) and recovery to CTCAE Grade 1 or baseline values occurs promptly, the situation will be evaluated on a case by case basis and determine if it is safe to resume therapy at the same dose level or at a lower dose level and if it is in accordance with the benefit/risk balance for the participant. On the contrary, in the event of DLTs whose re-occurrence could be potentially life threatening (i.e., cytokine release syndrome, pneumonitis) then participants will be removed from further treatment and will not be replaced.

### Example 1.4G—Discontinuation of Study Intervention and Participant

Discontinuation/Withdrawal. In case the IMP is discontinued, it is determined whether this discontinuation is temporary (i.e., a dose omission or cycle delay); permanent IMP discontinuation before disease progression, unless reaching the end of 1-year treatment period, is a last resort. Any IMP discontinuation must be fully documented in the eCRF. In any case, the participant should remain in the study until the documentation of progressive disease.

Definitive Discontinuation of Study Intervention. Permanent intervention discontinuation is any intervention discontinuation associated with the definitive decision from the Investigator not to re-expose the participant to the IMP at any time during the study, or from the participant not to be re-exposed to the IMP whatever the reason.

Study intervention is discontinued if, in the Investigator's opinion, continuation of the study intervention is detrimental to the participant's wellbeing, such as in any of the following cases:

1. Unacceptable adverse event.
2. Confirmed disease progression.
3. Poor compliance to the study protocol.
4. Completion of the 1-year treatment period.
5. Other conditions, such as concurrent illness, that prevents further administration of study intervention.

If participants are clinically stable, and deriving clinical benefit from therapy with minimal toxicity, they will be maintained on treatment until progressive disease or for a maximum treatment of 1 year, whichever comes first.

Discontinuation of study intervention for abnormal liver function is considered by the Investigator when the increase is not related to the underlying disease and if the Investigator believes that it is in the best interest of participant safety.

Participants may withdraw from treatment with IMPs if they decide to do so, at any time and irrespective of the reason, or this may be done at the discretion of the Investigator. Treatment with the IMP should be discontinued in any of the following cases: At the participant's request, at any time and irrespective of the reason (consent's withdrawal), or at the request of their legally authorized representative.

“Legally authorized representative” is considered to be an individual or judicial or other body authorized under applicable law to consent on behalf of a prospective participant to the participant's participation in the procedure(s) involved in the research. Withdrawal of consent for treatment is distinguished from withdrawal of consent for follow-up visits and from withdrawal of consent for non-participant contact follow-up, e.g., medical records check.

Participants requesting withdrawal are informed that withdrawal of consent for follow-up may jeopardize the public health value of the study. Participants who withdraw are explicitly asked about the contribution of possible AEs to their decision to withdraw consent, and any AE information elicited is documented. Preferably the participant withdraws consent in writing and, if the participant or the participant's representative refuses or is physically unavailable, the site documents and signs the reason for the participant's failure to withdraw consent in writing.

Participants are followed-up according to the study procedures specified in this protocol up to the scheduled date of study completion, or up to recovery or stabilization of any AE to be followed-up as specified in this protocol, whichever comes last.

If possible, and after the permanent discontinuation of intervention, the participants are assessed using the procedure normally planned for the last dosing day with the IMP including a pharmacokinetics sample, if appropriate.

All cases of permanent intervention discontinuation are recorded by the Investigator in the appropriate pages of the eCRF when considered as confirmed.

### Example 1.4H—Lost to Follow Up

A participant is considered lost to follow-up if he or she repeatedly fails to return for scheduled visits and is unable to be contacted by the study site.

The following actions are taken if a participant fails to return to the clinic for a required study visit:
The site attempts to contact the participant and reschedule the missed visit as soon as possible and counsel the participant on the importance of maintaining the assigned visit schedule and ascertain whether or not the participant wishes to and/or should continue in the study.
Before a participant is deemed lost to follow up, the Investigator or designee makes every effort to regain contact with the participant (where possible, 3 telephone calls and, if necessary, a certified letter to the participant's last known mailing address or local equivalent methods). These contact attempts are documented in the participant's medical record.
A participant, whom continues to be unreachable, is considered to have withdrawn from the study.

### Example 1.4I—Study Assessments and Procedures

Procedures conducted as part of the participant's routine clinical management (e.g., blood count) and obtained before signing of the ICF may be utilized for screening or baseline purposes provided the procedures met the protocol-specified criteria and are performed within the time frame defined in the SoA.

Repeat or unscheduled samples may be taken for safety reasons or for technical issues with the samples.

### Example 1.5—Efficacy Assessments

In the escalation phase, the objective response information is obtained based on RECIST 1.1, if there are measurable intact lesions based on RECIST 1.1.

In the expansion phase, the assessment of response to the cytokine RNA mixture is a primary objective. All participants treated in the expansion phase must have at least one measurable intact lesion for inclusion (see above inclusion criterion I 05). Tumor assessment is performed at fixed intervals as described in the Schedule of Activities (SOA) in Tables 2 and 3, and the assessment window is not impacted by dose delay or dose omission.

All tumor assessment data are recorded to related eCRF pages based on RECIST 1.1 criteria. As a requirement of RECIST 1.1 criteria, a partial or complete response must be confirmed on a second examination done at least 4 weeks apart, in order to be documented as a confirmed response to therapy. Based on RECIST for immunotherapies (iRECIST), progressive disease should also be confirmed on a second examination done at least 4 weeks apart to exclude pseudoprogression, in case of no clinically progressive disease.

The RECIST 1.1 criteria are followed for assessment of tumor response, and iRECIST criteria also are followed for reporting response criteria as secondary/exploratory endpoints. In case progressive disease is confirmed on second assessment, the date of progression is recorded based on the initial assessment. If disease progression is not confirmed, participants continue the treatment and unconfirmed progressive disease (iUPD) is recorded.

All measurable lesions (even those below the threshold value of measurability based on RECIST 1.1), are measured for optimization of study intervention. An exploratory analysis, as part of an efficacy assessment in terms of ORR, is performed by assessment of total tumor volume with consideration of the size of the non-target lesions, and analyses of injected versus non-injected lesions will be part of this exploratory assessment. Measurement procedures and documentation in eCRF are detailed in SRM, and statistical analyses plan is detailed in the SAP.

Secondary efficacy variables include disease control rates, duration of response, and progression free survival. All these parameters are detailed in the SAP.

### Example 1.5A—FDG-PET-CT and/or Contrast-Enhanced CT for Lymphoma Patients

ORR is defined as the proportion of participants with CR, and PR based on responses as assessed using the 5-point scale as per Lugano classification 2014 (Cheson B D et al. (2014) J Clin Onc 32(27):3059-68).

Tumor assessment includes FDG-PET-CT scan in case of FDG-avid lymphomas and contrast enhanced CT in case of non-FDG avid lymphomas. Tumor assessments are performed at fixed intervals as described in SoA, and the assessment window is not impacted by dose delay or dose omission.

If CT and/or PET scans at screening are negative for disease involvement in the neck, subsequent CT scans may not include the neck area. If PET and/or CT scans at screening are positive for disease involvement in the neck, subsequent CT scans must include the neck area. Tumor response assessments should occur at Screening (within 28 days 1-7 days] prior to first IMP), and every 12 weeks (+7 days) thereafter. Imaging timing should follow calendar days and should not be adjusted for delays in cycle. For participants who discontinue for reasons other than PD, assessments should continue until the participant has documented PD or start a new anti-cancer therapy. The first assessment may be performed earlier than 12 weeks if in the opinion of the investigator the participant is clinically progressing.

If participants have a PR, or a CR a repeated scan 4 weeks apart is required for confirmation and patients should continue on every 12 week assessment schedule. In the setting where a participant is clinically stable, but imaging shows PD at Week 12, study drug may be continued, at the discretion of the investigator, until the next disease response assessment. However, imaging should occur at any time when there is clinical suspicion of progression.

Assessment of lymphoma B symptoms should occur with each disease response assessment.

In participants with PD at Week 12, who continue study therapy beyond Week 12 a radiological assessment is performed at the time of treatment discontinuation. If previous scan was obtained within 4 weeks prior to the date of discontinuation, then a repeat scan at treatment discontinuation is not mandatory.

### Example 1.5B Bone Marrow Biopsy & Aspirate for Lymphoma Patients

All participants may have bone marrow biopsy/aspirate performed as clinically indicated as per Lugano 2014 criteria (Cheson B D et al. (2014)). FDG-PET-CT is adequate for determination of bone marrow involvement and can be considered highly suggestive for involvement of bone marrow. Bone marrow biopsy confirmation can be considered if necessary at baseline (if the FDG-PET-CT is negative in the bone marrow site then biopsy/aspirate is performed to identify involvement). Subsequent bone marrow assessments will only be performed in participants who have bone marrow involvement at baseline.

### Example 1.5C—Safety Assessments

The major purpose of this FIH study is to establish, based on DLTs, the biologically optimal dose of the cytokine RNA mixture when administered as a weekly intratumoral injection. Safety is thus a primary study endpoint and is assessed continuously. The safety profile is assessed from the findings of physical examination (preferably by the same physician) and laboratory tests and will be based on incidence, severity (as graded by the NCI CTCAE ver. 5.0), and cumulative nature of AEs. Planned time points for all safety assessments are provided in the SOA.

### Example 1.5D—Physical Examinations

A complete physical examination includes, at minimum, assessments of the Central Nervous System and the cardiovascular, respiratory, gastrointestinal, hematopoietic (hepatomegaly, splenomegaly, lymphadenopathy), and dermatological systems. Height (only at baseline) and weight (at pre-dose of each cycle) is measured and recorded in the eCRF.

ECOG performance status is assessed before each IMP administration and recorded in the eCRF. Investigators pay attention to clinical signs related to previous serious illnesses, as well as progress of skin lesions. Any new finding or worsening of previous finding are reported as a new adverse event. The schedule for physical examinations is described in the SOA.

### Example 1.5E—Vital Signs

During treatment phase, vital signs are monitored just before starting infusion of the IMP and at the end of injection. Monitoring is also performed as clinically indicated. Temperature, pulse rate, respiratory rate, and blood pressure are assessed. Blood pressure and pulse measurements should be preceded by at least 5 minutes of rest for the participant in a quiet setting without distractions (e.g., television, cell phones).

### Example 1.5F—Electrocardiograms, Echocardiogram and MUGA Scan

Single 12-lead ECGs are obtained as outlined in the SOA. Clinically significant abnormalities should be reported as AE, developed following signing of the ICF. Preexisting conditions should be recorded in the participant's medical history. Echocardiograms or MUGA scans will be obtained as outlined in the SoA (see herein) only at screening for patients in the combination part of the study.

### Example 1.5G—Pulmonary Function Test

DLCO is performed at baseline for participants with lymphoma previously treated with bleomycin.

### Example 1.5H—Clinical Safety Laboratory Assessments

The Investigator reviews the laboratory report and documents this review. The laboratory reports are filed with the source documents. Laboratory abnormalities are reported as AEs only in the event they:
Lead to investigational medicinal product discontinuation, treatment or dose modification.
Fulfill a serious or AE of special interest (AESI) definition (note: remaining laboratory tests are reported in the eCRF laboratory pages).
Previous mRNA and interleukin-triggering trials have shown transient changes in hematological parameters; these transient changes as part of the mode of action should not be registered as AEs by default. However, the clinical Investigator decides whether in a specific case a laboratory change should be reported as clinically significant and/or as AE.
All laboratory tests with values considered clinically significantly abnormal during participation in the study or within 30 days after the last dose of study intervention (i.e., EOT assessment) are repeated until the values return to normal or baseline or are no longer considered clinically significant by the Investigator or medical monitor.
If such values do not return to normal/baseline within a period of time judged reasonable by the Investigator, the etiology is identified, and the Sponsor notified.

All protocol-required laboratory assessments are conducted in accordance with the laboratory manual and the SoA.

If laboratory values from non-protocol specified laboratory assessments performed at the institution's local laboratory require a change in participant management or are considered clinically significant by the Investigator (e.g., SAE or AE or dose modification), then the results are recorded in the eCRF. All unplanned laboratory tests performed for safety follow-up or for further investigation of AE are reported in the eCRF.

### Example 1.5I—Dose Limiting Toxicities (DLTs)

DLTs are defined as any of the following AEs related to the IMPs in the absence of clear evidence to the contrary, after validation by the Study Committee, and if not related to a disease progression grading using NCI CTCAE ver. 5.0. The duration of the DLT observation period is longer for participants who delay initiation of Cycle 2 due to treatment-related AE for which the duration must be assessed in order to determine if the event is a DLT. The NCI CTCAE ver. 5.0 is used to assess the severity of AEs.

Hematological toxicity:
Grade ≥3 febrile neutropenia or Grade ≥3 neutropenia with documented infection.
Grade ≥3 hematologic toxicity lasting >72 hours.
Grade 4 thrombocytopenia or Grade 3 with hemorrhage or requiring transfusion.

Non-hematological toxicity:
Any Grade ≥3 immune-related AEs, except for Grade 3 skin reactions.
   An irAE can occur shortly after the first dose or several months after the last dose of treatment. All AEs of unknown etiology associated with drug exposure are evaluated to determine possible immune etiology. If an irAE is suspected, efforts are made to rule out neoplastic, infectious, metabolic, toxin or other etiologic causes prior to labeling an AE as an irAE.
Any other Grade ≥3 non-hematological toxicities;
   Excluding Grade 3 nausea, vomiting and diarrhea, if controlled with adequate anti-diarrheal therapy and resolving to Grade ≤1 within 48 hours.
   If a participant with known liver metastases was enrolled with Grade 2 AST or ALT abnormalities at baseline, an increase in AST or ALT is considered a DLT only if the increase was >3 times the baseline and the elevation was confirmed ≥5 days later.
   If a participant with Gilbert's Syndrome was enrolled with a Grade 2 bilirubin abnormality at baseline, an increase in bilirubin is considered a DLT only if the increase was >3 times the baseline and the elevation was confirmed ≥5 days later.
Grade ≥2 uveitis.

Other “non-gradable” toxicities:
A treatment-emergent adverse event that in the opinion of the Study Committee is of potential clinical significance such that further dose escalation would expose participants to unacceptable risk.
Toxicity related to IMP leading to more than 1 dose omission, in the absence of recovery to baseline or Grade ≤1 (except for alopecia, vitiligo, fatigue and hypothyroidism).

The occurrence of DLTs during the first 28 days of treatment for the escalation phase is used to define the MTD or MAD. In Cycle I and in subsequent cycles, the occurrence of DLTs determines the need for dose omissions or reductions (if the DLT occurs during the DLT observation period, study intervention is terminated definitively; beyond the DLT observation period).

Participants who experience a DLT will have their therapy with the cytokine RNA mixture stopped and they will be followed until this toxicity has resolved to CTCAE Grade ≤1 or to the participant's baseline value, if higher. After recovery from the toxicity in question, with a maximum of 2 dose omission and agreement of the Study Committee, and if the Investigator believes that it is in the participant's best interest to resume therapy with the cytokine RNA mixture, the participant may resume therapy with a new cycle of treatment at the same dose level or at a lower dose level, based on agreement with the Sponsor. No dose re-escalation is allowed for such re-dosed participants.

### Example 1.5J—Systemic Reactions

Management of hypersensitivity and anaphylactic reactions, along with associated dose modifications, is detailed below.

Systemic Inflammatory Reaction

Systemic reaction due to inflammatory reactions may occur with the cytokine RNA mixture administration. Antigen-specific T-lymphocyte responses. TLR-mediated signaling, and the transient release of pro-inflammatory cytokines may cause systemic inflammatory reactions. Typical clinical symptoms of systemic inflammatory reactions may include tachycardia, reduced blood pressure, dyspnea, shivers, vomiting, dizziness, and fever.

Possible actions in case of systemic inflammatory reactions are:
evaluation of vital functions (BP, HR, respiration, body temperature)
treatment with paracetamol and/or non-steroidal anti-inflammatory drugs (NSAIDs)
blood sample collection for IL-6, IFNγ, TNFα, IL-2; GM-CSF, IL-10, IL-8, IL-5, CRP, and Ferritin
Hospitalization until recovery upon discretion of the Investigator may be needed, accompanied, e.g., by:
   close monitoring of vital function (BP, HR, respiration, body temperature)
   administration of NSAIDs
   single high dose of intravenous cortisone
   single dose of tocilizumab 8 mg/kg infusion (if not recovering)

Cytokine Release Syndrome

Cytokine-associated toxicity, also known as CRS, is a non-antigen specific toxicity that occurs as a result of potent immune activation. CRS clinically manifests when large numbers of lymphocytes (B cells, T cells, and/or NK cells) and/or myeloid cells (macrophages, dendritic cells, and monocytes) become activated and release inflammatory cytokines. CRS has classically been associated with therapeutic monoclonal antibody infusions, and in these settings symptom onset typically occurs within minutes to hours afler the infusion begins. Though it is not expected that serum cytokine levels following intratumoral injection with the cytokine RNA mixture will approach levels observed in participants following direct injection of recombinant cytokines, there is a possibility that, in the course of sustained intratumoral cytokine levels providing clinical benefit, participants may have sustained levels of systemic cytokine levels which could cause adverse effects. Thus, participants receiving intratumoral injections of the cytokine RNA mixture are monitored closely for signs of cytokine-associated toxicities. In case a participant develops Grade 2 or higher signs and symptoms of CRS he/she needs to be hospitalized. Vital signs monitoring shall be made continuously if CRS Grade ≥2 develops. The participant should be transferred to the intensive care unit (ICU) in case he/she develops hemodynamic or respiratory compromise. The ICU should be staffed by a critical care physician who has experience in treating CRS. In addition, the ICU must have the necessary equipment to commence immediate treatment and monitoring of a participant with CRS Grade ≥2 before he/she is admitted to ICU.

For clinical signs and symptoms associated with CRS, see below.

The timing of symptom onset and CRS severity depends on the inducing agent and the magnitude of immune cell activation. The incidence and severity of the syndrome also appears greater when patients have large tumor burdens, presumably because this leads to higher levels of T-cell activation. As with CRS associated with monoclonal antibody therapy. CRS associated with adoptive T-cell therapies has been associated with elevated IFNγ, IL-6, and TNFα levels; increases in IL-2, GM-CSF, IL-10, IL-8, IL-5, and fracktalkine have also been reported. Emerging evidence implicates IL-6 as a central mediator of toxicity in CRS; IL-6 is a pleiotropic cytokine with anti-inflammatory and proinflammatory properties. However, real time analysis of a broad panel of cytokines does not significantly impact management of individual patients with CRS at the current time and treatment decisions are typically based on clinical parameters.

Assays for serum C-reactive protein (CRP) and ferritin are performed. Plasma levels of cytokines, including IL-6 and IFNγ, are collected and retrospectively analyzed only in case of development of CRS Grade ≥2 symptoms. Sampling is performed following the initial dose and after each dose increase, in order to assess for signs of CRS, and in case of development of CRS Grade ≥2 symptoms. CRP is an acute phase reactant produced by the liver, largely in response to IL-6. Serum CRP levels serve as a surrogate for increases in IL-6 bioactivity. During CRS, serum CRP levels may increase by several logs. The serum CRP assay is rapid, inexpensive, and readily available in most hospitals. In some series, peak CRP levels and fold change in CRP have identified patients at risk for severe CRS. It is important to emphasize, however, that CRP levels are also elevated during infection and cannot be used to distinguish between inflammation caused by infection and inflammation related to CRS. Extreme elevations in serum ferritin have been observed in many patients with CRS after chimeric antigen receptor (CAR) T-cell infusion, which supports a resemblance between CRS and macrophage activation syndrome/hemophagocytic lymphohistiocytosis (HLH).

To assess the severity of CRS in individual participants, the grading system and mitigation strategy for CRS that is based on the 2014 NCI Consensus Guidelines are used. This grading system was modified to define mild, moderate, severe, and life-threatening CRS regardless of the inciting agent and to guide treatment recommendations with corticosteroids and/or anti-human IL-6 monoclonal antibodies such as tocilizumab.

### Example 1.6—Adverse Events and Serious Adverse Events

Adverse Event of Special Interest

An AESI is an AE (serious or nonserious) of scientific and medical concern specific to the Sponsor's product or program, for which ongoing monitoring and immediate notification by the Investigator to the Sponsor is required. Such events may require further investigation in order to characterize and understand them. Adverse events of special interest may be added, modified or removed during a study by protocol amendment.
Pregnancy of a female subject entered in a study as well as pregnancy occurring in a female partner of a male subject entered in a study with IMP is qualified as an SAE only if it fulfills one of the seriousness criteria (see below).
   In the event of pregnancy in a female participant, IMP is discontinued.
      Follow-up of the pregnancy in a female participant or in a female partner of a male participant is mandatory until the outcome has been determined.
Symptomatic overdose (serious or nonserious) with IMP/noninvestigational medicinal product (NIMP)
   An overdose (accidental or intentional) with the IMP/NIMP is an event suspected by the Investigator or spontaneously notified by the participant and defined as at least 30% above the intended administered dose.
   Of note, asymptomatic overdose is reported as a standard AE.
Other project specific AESIs
   All protocol defined potential or IMP related DLTs are considered as AESI, regardless of the cycle of occurrence (i.e., after first 28 days of treatment in both escalation and expansion phases)
   Cytokine release syndrome (any Grade)

AE is reported by the participant (or, when appropriate, by a caregiver, surrogate, or the participant's legally authorized representative).

The Investigator and any qualified designees are responsible for detecting, documenting, and recording events that meet the definition of an AE or SAE and remain responsible for following up AEs that are serious, considered related to the study intervention or study procedures, or that caused the participant to discontinue the Cytokine RNA mixture.

Adverse Event (AE)

An AE is any untoward medical occurrence in a participant or clinical study participant, temporally associated with the use of study intervention, whether or not considered related to the study intervention. AE can therefore be any unfavorable and unintended sign (including an abnormal laboratory finding), symptom, or disease (new or exacerbated) temporally associated with the use of study intervention.

Serious Adverse Event (SAE)

A SAE is any untoward medical occurrence that at any dose:
Results in death,
Is life threatening (note: the term “life-threatening” refers to an event/reaction in which the participant was at risk of death at the time of the event/reaction; it does not refer to an event/reaction which hypothetically might have caused death if it were more severe),
Requires inpatient hospitalization or results in prolongation of existing hospitalization,
Results in persistent or significant disability/incapacity, permanent or significant (if transient), and substantial disruption of his/her ability to carry out normal life functions. Disability is not intended to include experiences of relatively minor significance, such as headache, nausea, vomiting, or accidental minor trauma
Is a congenital anomaly/birth defect, an anomaly of fetus infant, or any anomaly that results in fetal loss
Is a medically important event or reaction. Medical and scientific judgment should be exercised in deciding whether other situations should be considered serious, such as important medical events that might not be immediately life-threatening or result in death or hospitalization, but might jeopardize the participant or might require intervention to prevent one of the other outcomes listed in the definition above. Investigator is responsible to assess Medically Important AEs as Serious AEs (SAEs) Examples: intensive treatment in an emergency room, or at home, for allergic bronchospasm; blood dyscrasias without hospitalization; asymptomatic ALT increase over 10×ULN without hospitalization)

A treatment-emergent adverse event (TEAE) is defined as an AE that is reported during the on-treatment period up to 30 days after last dose of study interventions.

Related Adverse Event: there is a reasonable possibility according to the Investigator Sponsored Studies (ISS) that the product may have caused the event. The causality of the SAE (i.e., its relationship to study intervention) will be assessed by the physician, who is completing the CRF. For regulatory reporting purposes, if the relationship is unknown or unstated, it meets the definition of an adverse drug reaction (suspected association—ADR).

Immune-related Adverse event (ir-AE): a subset of treatment related adverse events, is defined as a clinically significant adverse event of any organ that is associated with immune based therapy (e.g., immune check point inhibitor exposure), of unknown etiology, and is consistent with an immune-mediated mechanism.

Adverse Event of Special Interest (AESI): an adverse event (serious or non-serious) of scientific and medical concern specific to the Sponsor's product or program, for which ongoing monitoring and rapid communication by the Investigator to the Sponsor may be appropriate. Such events may require further investigation in order to characterize and understand them. AESIs may be added or removed during a study by protocol amendment.

New safety finding: any finding other than reportable individual case safety report (ICSR) or safety issue that may impact the known risk-benefit balance or the safety profile of the product.

Expected AE/SAE: The determination of expectedness under an approved indication and regimen of the product is to be determined based on local label (if available) or EU Summary of Product Characteristics (SmPC). When the product is administered in any non-approved combination/regimen, or for a non-approved indication/population, or for a non-approved dosing, the determination of expectedness should be based on the IB (consider the labeling of each specific marketed drug within the combination, based upon reference documents as defined in the study protocol).

Suspected unexpected serious adverse reaction (SUSAR): Causality, seriousness and expectedness are independent criteria. It is a combination, which defines expedited reporting to Health Authorities.

Events Meeting the AE Definition:
Any abnormal laboratory test results (e.g., hematology, clinical chemistry, or urinalysis) or other safety assessments (e.g., ECG, radiological scans, vital signs measurements), including those that worsen from baseline, considered clinically significant in the medical and scientific judgment of the Investigator (i.e., not related to progression of underlying disease).
Exacerbation of a chronic or intermittent pre-existing condition including either an increase in frequency and/or intensity of the condition.
New conditions detected or diagnosed after study intervention administration even though it may have been present before the start of the study.
Signs, symptoms, or the clinical sequelae of a suspected drug-drug interaction.
Signs, symptoms, or the clinical sequelae of a suspected overdose of either study intervention or a concomitant medication.

Events NOT Meeting the AE Definition:
The disease/disorder being studied or expected progression, signs, or symptoms of the disease/disorder being studied, unless more severe than expected for the participant's condition.
Medical or surgical procedure (e.g., endoscopy, appendectomy): the condition that leads to the procedure is the AE.
Situations in which an untoward medical occurrence did not occur (social and/or convenience admission to a hospital).
Anticipated day-to-day fluctuations of pre-existing disease(s) or condition(s) present or detected at the start of the study that do not worsen

If an event is not an AE per definition above, then it cannot be an SAE even if serious conditions are met (e.g., hospitalization for signs/symptoms of the disease under study, death due to progression of disease).

Recording and Follow-Up of AE and/or SAE

AE and SAE Recording

When an AE/SAE occurs, all documentation (e.g., hospital progress notes, laboratory reports, and diagnostics reports) related to the event are reviewed and all relevant AE/SAE information in the eCRF are recorded. There may be instances when copies of medical records for certain cases are requested by the Sponsor. In this case, all participant identifiers, with the exception of the participant number, are redacted on the copies of the medical records before submission to the Sponsor. The Investigator attempts to establish a diagnosis of the event based on signs, symptoms, and/or other clinical information. Whenever possible, the diagnosis (not the individual signs/symptoms) is documented as the AE/SAE.

Assessment of Intensity

Intensity of AE/SAE is assessed based on NCI CTCAE version 5.0.

Assessment of Causality

The Investigator is obligated to assess the relationship between study intervention and each occurrence of each AE/SAE. A “reasonable possibility” of a relationship conveys that there are facts, evidence, and/or arguments to suggest a causal relationship, rather than a relationship cannot be ruled out. The Investigator uses clinical judgment to determine the relationship. Alternative causes, such as underlying disease(s), concomitant therapy, and other risk factors, as well as the temporal relationship of the event to study intervention administration will be considered and investigated. The Investigator also consults the Investigator's Brochure (IB) and/or Product Information, for marketed products, in his/her assessment.

For each AE/SAE, the Investigator must document in the medical notes that he/she has reviewed the AE/SAE and has provided an assessment of causality. There may be situations in which an SAE has occurred, and the Investigator has minimal information to include in the initial report to the Sponsor. However, it is very important that the Investigator always assess causality for every event before the initial transmission of the SAE data to the Sponsor. The Investigator may change his/her opinion of causality in light of follow-up information and send a SAE follow-up report with the updated causality assessment.

Follow-Up of AEs and SAEs

The Investigator is obligated to perform or arrange for the conduct of supplemental measurements and/or evaluations as medically indicated or as requested by the representative of the monitoring team to elucidate the nature and/or causality of the AE or SAE as fully as possible. This may include additional laboratory tests or investigations, histopathological examinations, or consultation with other health care professionals. New or updated information will be recorded in the originally completed eCRF.

Reporting of SAEs

SAE reporting to the Sponsor via an electronic data collection tool. The primary mechanism for reporting an SAE to the Sponsor is the electronic data collection tool. If the electronic system is unavailable for more than 24 hours, then the site uses the paper SAE data collection tool (see herein). The site enters the SAE data into the electronic system as soon as it becomes available. After the study is completed at a given site, the electronic data collection tool is taken off-line to prevent the entry of new data or changes to existing data. If a site receives a report of a new SAE from a study participant or receives updated data on a previously reported SAE after the electronic data collection tool has been taken off-line, then the site can report this information on a paper SAE form (see next herein) or to the Sponsor or representative by facsimile.

SAE Reporting to the Sponsor Via Paper CRF

Facsimile transmission of the SAE paper CRF is the preferred method to transmit this information to the Sponsor or representative. In rare circumstances and in the absence of facsimile equipment, notification by telephone is acceptable with a copy of the SAE data collection tool sent by overnight mail or courier service. Initial notification via telephone does not replace the need for the Investigator to complete and sign the SAE CRF pages within the designated reporting time frames.

### Example 1.6A—Time Period and Frequency for Collecting AE and SAE Information

All AEs (including SAEs) are collected from the signing of the ICF until EOT at the time points specified in the SOA. After EOT, only IMP-related or unexpected events (including those for which the relationship to IMP is unclear) are reported.

All SAEs and AESI are recorded and reported to the Sponsor or designee within 24 hours, as indicated below. The Investigator submits any updated SAE data to the Sponsor within 24 hours of it being available.

Investigators are not obligated to actively seek AE or SAE after conclusion of the study participation. However, if the Investigator learns of any SAE, including a death, at any time after a participant has been discharged from the study, and he/she considers the event to be reasonably related to the study intervention or study participation, the Investigator must promptly notify the Sponsor.

The method of recording, evaluating, and assessing causality of AE and SAE and the procedures for completing and transmitting SAE reports are provided below.

### Example 1.6B3—Method of Detecting AEs and SAEs

Care is taken not to introduce bias when detecting AEs and/or SAEs. Open-ended and non-leading verbal questioning of the participant is the preferred method to inquire about AE occurrences.

### Example 1.6C—Follow-Up of AEs and SAEs

After the initial AE/SAE report, the Investigator is required to proactively follow each participant at subsequent visits. After the EOT, during the safety follow-up period, the events to be reported, monitored, and followed-up to resolution or stabilization are as follows:
All ongoing AEs, SAEs, or Events of Special Interest regardless of relationship
All new AEs, SAEs, or Events of Special Interest considered related, including deaths due to related events

Further information on follow-up procedures is given below.

### Example 1.6D—Disease-Related Events and/or Disease-Related Outcomes not Qualifying as AEs or SAEs

The following disease related events (DREs) are common in participants with cancer and can be serious/life threatening:
Progression of underlying disease, as it is the study endpoint.
Death due to progression of underlying disease, if it occurs after 30 days of the last IMP administration. All death that occurs within the 30 days of last study intervention should be reported as a SAE.

Because these events are typically associated with the disease under study, they are not reported according to the standard process for expedited reporting of SAEs even though the event may meet the definition of a SAE. These events are recorded on the corresponding page in the participant's eCRF within the appropriate time frame.

However, if either of the following conditions applies, then the event must be recorded and reported as an SAE (instead of a DRE): the event is, in the Investigator's opinion, of greater intensity, frequency, or duration than expected for the individual participant; or the Investigator considers that there is a reasonable possibility that the event was related to study intervention.

Pregnancy:

Details of all pregnancies in female participants and, if indicated, female partners of male participants will be collected after the start of study intervention and at least 6 months after the last dose of study intervention.

If a pregnancy is reported, the Investigator informs the Sponsor within 24 hours of learning of the pregnancy. Abnormal pregnancy outcomes (e.g., spontaneous abortion, fetal death, stillbirth, congenital anomalies, ectopic pregnancy) are considered SAEs. Pregnancy follow-up describes the outcome of the pregnancy, including any voluntary or spontaneous termination, details of the birth, the presence or absence of any congenital abnormalities, birth defects, maternal or newborn complications and their presumed relation to the study drug.

### Example 1.7—Pharmacokinetics

### Example 1.7A—Sampling Time

The following blood collection time points are defined to measure concentrations of cytokines encoded by the cytokine RNA mixture in plasma and conduct the PK analysis:

The sampling times for blood collection can be found in the PK/PDy flow chart (Table 3). It is of utmost importance to collect all blood samples at the specified times and according to the specifications.

Samples missed or lost for any reason are recorded. Actual times of blood collection are recorded in the eCRF. The dates and times of sampling and drug administration are also precisely recorded.

### Example 1.7B—Bioanalytical Method

Bioanalytical methods are summarized in Table 10. Briefly, systemic levels of the four target cytokines (IL-12sc, IL-15 sushi, GM-CSF, and IFNα2b) translated from the cytokine RNA mixture in plasma are monitored retrospectively in each participant cohort. These cytokine assays (IL-12sc, GM-CSF, IFNα, and IL-15 sushi) are performed on either the MSD or Quanterix SIMOA platforms based on needs for detection sensitivity.

TABLE 10
Bioanalytical methods for cytokines encoded by the cytokine
RNA mixture pharmacokinetic analysis

| Analyte | IL-15sushi | IL-12sc | CM-CSF | IFNα2b |
|---|---|---|---|---|
| Matrix | Plasma | Plasma | Plasma | Plasma |
| Analytical | Single or | Single or | Single or | Single or |
| technique | multiplexed | multiplexed | multiplexed | multiplexed |
| | cytokine | cytokine | cytokine | cytokine |
| | assay | assay | assay | assay |
| | on MSD or | on MSD or | on MSD or | on MSD or |
| | similar | similar | similar | similar |

### Example 1.7C—PK Parameters

Pharmacokinetic parameters are calculated with PKDMS software (Pharsight), using non compartmental methods, from intensively sampled plasma concentrations of cytokines encoded by the cytokine RNA mixture. The parameters include, but are not be limited to, the following:

TABLE 11
List of pharmacokinetic parameters and definitions

| | Analyte | | | | |
|---|---|---|---|---|---|
| | IL- | IL- | CM- | | |
| Parameters | 15sushi | 12sc | CSF | IFNα2b | Definition |
| Cₘₐₓ | X | X | X | X | Maximum plasma |
| | | | | | concentration observed |
| | | | | | over the dosing interval |
| tₘₐₓ | X | X | X | X | Time to reach Cₘₐₓ |
| Cₗₐₛₜ | X | X | X | X | Last concentration |
| | | | | | observed above the |
| | | | | | lower limit of |
| | | | | | quantification overt |
| | | | | | the dosing interval |
| tₗₐₛₜ | X | X | X | X | Time of Cₗₐₛₜ |
| C_{trough} | X | X | X | X | Plasma concentration |
| | | | | | observed just |
| | | | | | before treatment |
| | | | | | administration |
| | | | | | during repeated dosing |
| AUC_{0-7 d} | X | X | X | X | Area under the plasma |
| | | | | | concentration versus time |
| | | | | | curve calculated using the |
| | | | | | trapezoidal method over the |
| | | | | | dosing interval (7 days) |

Population PK approaches may be used for cytokines encoded by the cytokine RNA mixture. If done, the data generated are reported in a standalone report(s).

### Example 1.8—Pharmacodynamics

Target engagement, PDy, and safety biomarkers of the cytokine RNA mixture are important for dose escalation and PoC trial success. Quantitative or semi-quantitative biomarkers can help establish the correlation of dose level with target expression, PDy, and PK parameters, and aid in determination of the MTD/MAD. The biomarkers for the cytokine RNA mixture program can be broadly classified into circulating target expression, PDy/safety markers, and the tissue derived PDy markers.

When possible, PDy sample collection coincide with scheduled PK sampling.

### Example 1.8A—Circulating Target Engagement and Safety Biomarker Monitoring Plan

Systemic levels of the four target cytokines (IL-12sc, IL-15sushi, GM-CSF, and IFNα2b) translated from the cytokine RNA mixture and their downstream PDy targets (IFNγ and IFNγ-induced protein 10 [IP10]) in plasma are monitored retrospectively in each participant cohort.

The safety biomarkers CRP and ferritin are used along with clinical parameters (e.g., fever, nausea, fatigue, headache, myalgias, malaise, hypoxia, hypotension) to assist in identification of clinical AEs. Plasma samples are collected for monitoring of secondary CRS. A panel of 6 cytokines (IL-1p, IL-2, IL-6, IL-8, IL-10, and TNFα) are assessed retrospectively during the conduct of the study only in case of development of CRS Grade ≥2 symptoms.

### Example 1.8B—Tumor Biopsy for Immune Assessment

Mandatory tumor biopsies are collected before the first IMP administration, between weeks 5 and 8, and at Cycle 6 or upon disease progression (whichever occurs first). For on-treatment biopsy specimens (i.e., the one at week 5-8, and the other one at Cycle 6 or at the time of disease progression), it is required to get biopsy specimens from both injected and un-injected lesions. Preferably, one of the lesions to be biopsied on-treatment should be the one that has been biopsied at baseline. If this is not feasible, tissue specimen from another injected lesion could be considered. If there is a limitation of lesions to be biopsied, then biopsy of only the un-injected lesion could be considered if another sample from the same site has been previously collected or could be collected at the following sampling time point.

Biopsies for all participants undergo hematoxylin and eosin staining and standard IHC for CD3, CD8, and tumor cells will be determined by SOX10 markers (for melanoma) or pancytokeratin ([CK] for patients with epithelial tumors HNSCC and CSCC) and lymphoma markers in respect to the tumor type interrogated; a subset of participant biopsies (from both responders and non-responders) undergo a 12 marker multiplex IHC, which will consist of CD3, CD4, CD8, CD38, CD45, CD45RO, CD56, CD68, FoxP3, PD-1, PD-L1, and SOX10 or PanCK or lymphoma markers. IHC on pre- and post-treatment biopsies is collected and used to assess changes in the tumor microenvironment, specifically assessing the frequency and density of infiltrating T-cells in the tumor and stroma. Increases in T-cells between pre- and post-biopsies are a positive immune correlate used to help define proof of mechanism.

For melanoma patients only during expansion monotherapy, a single tumor core biopsy performed between weeks 5-8 will be dedicated for TILs isolation. This will be applied to a limited number (e.g., no more than ten patients with successful TILs isolation) of selected melanoma patients. This will not be an additional biopsy, but instead the sample dedicated for genomic assessment will be used for TILs isolation (handled under special conditions-not formalin fixed). This kind of sample and testing is applied to patients with clinical signs of response to treatment (tumor size reduction and/or redness at the tumor site) as determined by the treating investigator.

Tumor transcriptomics (RNA Sequencing) genomics, and neo-antigens are also analyzed upon sample availability.

### Example 1.9—Genomics

Several analyses are conducted to analyze genomics in the context of treatment, including somatic mutations and HLA typing on PBMCs, RNA sequencing (RNAseq) on tumors. In addition, tumor RNAseq data (also planned as part of the biomarker analysis) are required to determine gene signatures, neo-antigens within tumor, TMB, and TCR diversity. HLA typing will be performed in blood. Participation in these analyses is mandatory if adequate sample material is available.

Neo-antigens are assessed only in melanoma participants.

In the event of DNA or RNA extraction failure, a replacement sample (tumor or blood) is requested from the participant. Signed informed consent is required to obtain a replacement sample unless it was included in the original consent. In case of feasibility constraints on sample handling and shipment, samples from related clinical sites will not be assessed for these (or some of these) analyses.

### Example 1.9A—Immunogenicity Assessments

Antibodies to the cytokine RNA mixture-encoded cytokines are evaluated in plasma samples collected from all participants according to the SOA. Additionally, plasma samples are also collected at the final visit from participants who discontinued study intervention or were withdrawn from the study. These samples are tested by the Sponsor or Sponsor's designee.

Plasma samples are screened for antibodies binding to each of the four expressed cytokines from the cytokine RNA mixture and the titer of confirmed positive samples is reported. Other analyses are performed to further characterize the immunogenicity of the cytokine RNA mixture.

The detection and characterization of antibodies to the cytokine RNA mixture are performed using a validated assay method by or under the supervision of the Sponsor. Antibodies are further characterized and/or evaluated for their ability to neutralize the activity of the study intervention. Samples are stored for a maximum of 5 years (or according to local regulations) following the last participant's last visit for the study at a facility selected by the Sponsor to enable further analysis of immune responses to the cytokine RNA mixture.

### Example 1.9B—RNA Transcriptome Research

Exploratory transcriptome studies are conducted using microarray, and/or alternative equivalent technologies, which facilitates the simultaneous measurement of the relative abundances of thousands of RNA species resulting in a transcriptome profile for each tissue biopsy sample. Tumor tissue remaining after 1HC is subject to RNA sequencing analysis to assess global gene expression changes within the tumor environment, in particular looking for development of pro-inflammatory and/or IFNγ gene signatures. This enables the evaluation of changes in transcriptome profiles that correlate with an adaptive immune response relating to the action of the cytokine RNA mixture.

The same samples are also used to confirm findings by application of alternative technologies.

### Example 1.10—Statistical Considerations

### Example 1.10A—Statistical Hypotheses

Dose Escalation

There is no formal statistical hypothesis in the dose escalation part of this study. This study aims to establish the MTD or MAD of the cytokine RNA mixture according to DLTs observed. Dose escalation proceeds using a single-participant dose escalation for the first two DLs followed by a rational design.

Dose Expansion

The null hypothesis is that the true ORR per RECIST 1.1 is <10%, and the alternative hypothesis is that the true ORR per RECIST 1.1 is >26%.

### Example 1.10B—Sample Size Determination

Dose Escalation Phase

There is no formal sample size calculation in the dose escalation phase. Approximately 38 DLT-evaluable participants are enrolled in the dose escalation phase with assessment of about 8 DLs. The actual sample size varies depending on DLTs observed and number of dose levels actually explored.

Dose Expansion Phase

A rational design is used in the dose expansion phase. The null hypothesis that the true response rate is 10% is tested against a one-sided alternative. In the first stage, 16 participants are accrued. If there are 1 or fewer responses, according to RECIST 1.1 criteria, in these 16 participants, the study is stopped. Otherwise, 18 additional participants are accrued for a total of 34. The null hypothesis is rejected if 7 or more responses are observed in 34 participants with advanced melanoma that have failed a prior therapy based on anti-PD-1 or anti-PD-L I. This design yields a one-sided type I error rate of 5% and power of 80% when the true response rate is 26%.

### Example 1.10C—Populations for Analyses

For purposes of analysis, the following populations are defined as shown in Table 12:

TABLE 12
Populations for analyses

| Population | Description |
|---|---|
| All treated | For both dose escalation and dose expansion phases of the |
| | study, the all treated population will include all participants |
| | who have given their informed consent and received at least |
| | one dose (even incomplete) of treatment with the cytokine |
| | RNA mixture. |
| | This population is the primary population for the analyses of |
| | efficacy and safety parameters. All analyses using this |
| | population will be based on the dose level actually received |
| | in the first cycle. |
| DLT Evaluable | The DLT evaluable population is defined as participants in |
| (dose escalation phase) | the dose escalation phase receiving at least 70% of the |
| | planned doses of the cytokine RNA mixture in during the |
| | first 28 days of the treatment, and who completed the DLT |
| | observation period after the first IMP administration, unless |
| | they discontinue the study intervention(s) due to DLT. The |
| | dose recommended for dose expansion phase will be |
| | determined based on the DLT evaluable population. |
| PK | The PK population will include all participants from the all |
| | treated population with at least 1 measurable cytokine |
| | encoded by the cytokine RNA mixture concentration after the |
| | first dose of study intervention. |
| PDy | The pharmacodynamic population will include all |
| | participants from the all treated population with at least 1 |
| | pharmacodynamic marker result after the first dose of study |
| | intervention. |
| ADA | The ADA evaluable population includes all participants from |
| Evaluable | the all treated population with at least 1 non missing ADA |
| | result after the first dose of study intervention. |
| Safety | Safety population is the same as all treated population. |

### Example 1.10D—Statistical Analyses

Efficacy Analyses

Objective response rate (ORR) per RECIST 1.1 based on pre-selected lesions, including injected and un-injected lesions, are summarized with descriptive statistics. A 90% two-sided confidence interval is computed using Clopper-Pearson method. The statistical inference is based on the hypothesis and alpha level defined in the sample size calculation section. A similar analysis is provided for the DCR per RECIST 1.1 and iRECIST, and the ORR per iRECIST. In addition, a summary of tumor burden change is provided for injected and un-injected lesions separately as a supportive analysis. DoR and PFS are summarized using the Kaplan-Meier method.

TABLE 13
Efficacy Analysis

| Endpoint | Statistical Analysis Methods |
|---|---|
| Primary | |
| Dose expansion: ORR per | Descriptive statistics and |
| RECIST 1.1 | Clopper-Pearson method |
| Secondary | |
| Dose expansion: DoR and PFS | Kaplan-Meier method |
| per REC1ST 1.1 and iRECIST | |
| Dose expansion: DCR per | Descriptive statistics and |
| REC1ST 1.1 and iRECIST, and | Clopper-Pearson method |
| ORR per iRECIST | |
| Exploratory | Will be described in the statistical |
| | analysis plan finalized before |
| | database lock |

Safety Analyses

All safety analyses will be performed on the all-treated population.

TABLE 14
Safety analyses

| Endpoint | Statistical Analysis Methods |
|---|---|
| Primary | |
| Dose escalation: DLTs | Descriptive statistics |
| Dose escalation: | Descriptive statistics |
| AEs/SAEs and laboratory | |
| abnormalities | |
| Secondary | |
| Dose escalation: | Descriptive statistics |
| Immunogenicity | |
| Dose expansion: | Descriptive statistics |
| Immunogenicity | |
| Dose expansion: | Descriptive statistics |
| AEs/SAEs and laboratory | |
| abnormalities | |

Dose-Limiting Toxicities

In the dose escalation phase, DLTs are summarized by dose level. Details of DLTs are provided by the participant. DLTs are defined using NCI CTCAE version 5.0, as described above.

Analyses of Adverse Events

The observation period is divided into 3 segments: screening, TEAE and post-treatment. The screening period is defined as the time informed consent is signed until the administration of the first dose of study intervention. The treatment-emergent adverse event (TEAE) period is defined as the time from the first dose of study interventions up to 30 days after last dose of study interventions. The post-treatment period is defined as the time starting 31 days after the last dose of study interventions to study closure or death, whichever comes first.

Pre-treatment AEs are defined as any AE during the screening period. Treatment-emergent AEs are defined as AEs that develop, worsen (according to the Investigator opinion) or become serious during the TEAE period. Post-treatment AEs are defined as AEs that are reported during the post-treatment period. The primary focus of AE reporting is on TEAEs. Pre-treatment and post-treatment AEs are described separately.

The TEAEs are coded according to Medical Dictionary for Regulatory Activities (MedDRA). AEs are graded according to the NCI CTCAE version 5.0. The grade is considered in the summary. For participants with multiple occurrences of the same preferred term (PT), the maximum grade is used.

An overall summary of TEAEs is provided. The number and percentage of participants who experience any of the following are provided:
TEAEs.
TEAEs of Grade 23.
TEAEs of Grade 3 or 4.
Grade 5 TEAE (any TEAE with a fatal outcome during the treatment period).
Serious TEAEs.
Serious treatment-related TEAEs.
TEAE leading to treatment discontinuation.
AESIs.
Treatment-related TEAEs.
Treatment-related TEAEs of Grade ≥3.

The number and percentage of participants experiencing TEAEs by primary system organ class (SOC) and PT are summarized by NCI CTCAE grade (all grades and Grade ≥3). Similar tables are prepared for treatment-related TEAEs, AESIs. TEAEs leading to treatment discontinuation, TEAEs leading to dose modification, serious TEAEs, TEAEs with fatal outcome and AEs/SAEs occurring during the post-treatment dosing period.

Clinical Laboratory Evaluations

Clinical laboratory results are graded according to NCI CTCAE version 5.0, when applicable. Number (%) of participants with laboratory abnormalities (i.e., all grades and Grade ≥3) using the worst grade during the TEAE period is provided for the all-treated population.

As explained above, not all transient changes in laboratory values based on mode of action are documented as TEAEs; the Investigator evaluates whether a laboratory change is clinically relevant in order to document it as a TEAE.

When the NCI CTCAE version 5.0 scale is not applicable, the number of participants with laboratory abnormality out-of-normal laboratory range value is displayed.

### Example 1.11—Clinical Laboratory Tests

The tests detailed in Table Tables 14 and 15 are performed and the results are entered into the eCRF. Protocol-specific requirements for inclusion or exclusion of participants are detailed in the protocol. Additional tests are performed at any time during the study as determined necessary by the Investigator or required by local regulations. Investigators must document their review of each laboratory safety report.

TABLE 15
Protocol-required safety laboratory assessments

| Laboratory | | | | |
|---|---|---|---|---|
| assessments | Parameters | | | |
| Hematology | Platelet count | | White blood cell (WBC) count | |
| | Red blood cell (RBC) count | | with differential: | |
| | Hemoglobin | | Neutrophils | |
| | Hematocrit | | Lymphocytes | |
| | | | Monocytes | |
| | | | Eosinophils | |
| | | | Basophils | |
| Clinical | Urea or Blood | Potassium | Bicarbonate | Phosphorus |
| chemistry^{a} | urea nitrogen | | | |
| | (BUN) | | | |
| | Creatinine | Sodium | Magnesium | Chloride |
| | Glucose | Total calcium | Alkaline | Total and |
| | | Uric acid | phosphatase | direct bilirubin |
| | Aspartate | Alanine | Total protein | Albumin |
| | aminotransferase | aminotransferase | | |
| | (AST)/Serum | (ALT)/Serum | | |
| | glutamic- | glutamic | | |
| | oxaloacetic | pyruvic | | |
| | transaminase | transaminase | | |
| | (SGOT) | (SGPT) | | |
| | Lactate | Amylase | | |
| | dehydrogenase | | | |
| | (LDH) | | | |

| | |
|---|---|
| Routine | Dipstick assessment for |
| urinalysis | pH, glucose, protein, blood, ketones by dipstick |
| | Leukocytes and RBCs |
| | Microalbumin |
| | Microscopic examination (if blood or protein is abnormal) |
| Other tests | Serum human chorionic gonadotropin (hCG) pregnancy test (as |
| | needed for women of childbearing potential) |
| | CRP, feiritin |
| | Coagulation: activated partial thromboplastin time (aPTT), PT |
| | international normalized ratio (INR), and fibrinogen |
| Screening tests | Serology (hepatitis B surface antigen [HBsAg], Hepatitis core |
| | antibody [HBcAb] hepatitis C virus antibody, HCV RNA, and, |
| | only for participants at study sites in Germany, HIV antibodies) |
| | Coagulation: D-dimer |

NOTES:
^{a}Details of liver chemistry stopping criteria and required actions and follow-up assessments after liver stopping or monitoring event are given below. All events of ALT ≥3 × upper limit of normal (ULN) and bilirubin ≥2 × ULN (>35% direct bilirubin) or ALT ≥3 × ULN and international normalized ratio (INR) >1.5, if INR measured which may indicate severe liver injury (possible Hy's Law) must be reported as an SAE .

TABLE 16
Protocol-required assessments

| Laboratory | | | | | | |
|---|---|---|---|---|---|---|
| assessments | Parameters | | | | | |
| Secondary | IL-6 | IL-10 | IL-2 | IL-8 | | |
| plasma cytokines | IL-10 | TNFα | | | | |
| PK cytokines | IL-12sc | IFNα | IL-15sushi | GM-CSF | | |
| (Expression of | | | | | | |
| the cytokine | | | | | | |
| RNA mixture- | | | | | | |
| encoded. | | | | | | |
| cytokines) | | | | | | |
| PDy cytokines | IP-10 | IFNγ | | | | |
| PDy antigen | CD4/CD8 T-cell responses against | | HLA phenotype | | | |
| specific T-cell | well-expressed immunogenic | | | | | |
| assessment | melanoma-associated antigens | | | | | |
| Immunogenicity | Antibodies against cytokines encoded by the cytokine RNA mixture | | | | | |
| | (i.e., ADAs against IL-12sc, IFNα, IL-15sushi, and GM-CSF) | | | | | |
| Tumor biopsy | Standard IHC (all participants) | | | Multiplex IHC (subset of participants) | | |
| for immune | CD3 | CD8 | SOX10 | CD3 | CD4 | CD8 |
| assessment | | | or CK | CD38 | CD45 | CD45RO |
| | | | | CD56 | CD68 | FoxP3 |
| | | | | PD-1 | PD-L1 | SOX10 |
| | | | | | | or CK |

| | | | |
|---|---|---|---|
| Urine biomarker | KIM-1 | Urinary creatinine | Urinary microalbumin |

### Example 1.12—Contraceptive Guidance and Collection of Pregnancy

Information

Woman of childbearing potential (WOCBP): A woman of childbearing potential is a woman who:

1. has achieved menarche at some time point,
2. has not undergone a hysterectomy or bilateral oophorectomy, or
3. has not been naturally postmenopausal (amenorrhea following cancer therapy does not rule out childbearing potential) for at least 24 consecutive months (i.e., has had menses at any time in the preceding 24 consecutive months).

Contraception Guidance
Male participants
   Male participants with female partners of childbearing potential are eligible to participate if they agree to ONE of the following during the intervention period and for 6 months after the last dose of study intervention:
      Are abstinent from penile-vaginal intercourse as their usual and preferred lifestyle (abstinent on a long term and persistent basis) and agree to remain abstinent
      Agree to use a male condom plus partner use of a contraceptive method with a failure rate of <1% per year as described below when having penile-vaginal intercourse with a woman of childbearing potential who is not currently pregnant
   In addition, male participants must refrain from donating sperm for the duration of the study and for 6 months after the last dose of study intervention
   Male participants with a pregnant or breastfeeding partner must agree to remain abstinent from penile vaginal intercourse or use a male condom during each episode of penile penetration during the intervention period and for 6 months after the last dose of study intervention.
Female participants
   Female participants of childbearing potential are eligible to participate if they agree to use a highly effective method of contraception consistently and correctly as described below:

Highly effective contraceptive methods that are user dependent Failure rate of <1% per year when used consistently and correctly.
i) Combined (estrogen and progestogen containing) hormonal contraception associated with inhibition of ovulation. Oral, Intravaginal. or Transdermal
ii) Progestogen only hormonal contraception associated with inhibition of ovulation: Oral or Injectable

Highly Effective Methods that are User Independent:
iii) Implantable progestogen only hormonal contraception associated with inhibition of ovulation: Intrauterine device (IUD), Intrauterine hormone-releasing system (IUS), or Bilateral tubal occlusion

Vasectomized partner: A vasectomized partner is a highly effective contraception method provided that the partner is the sole male sexual partner of the WOCBP and the absence of sperm has been confirmed. If not, an additional highly effective method of contraception is used.

Sexual abstinence: Sexual abstinence is considered a highly effective method only if defined as refraining from heterosexual intercourse during the entire period of risk associated with the study intervention. The reliability of sexual abstinence is evaluated in relation to the duration of the study and the preferred and usual lifestyle of the participant.

NOTES: Typical use failure rates may differ from those when used consistently and correctly. Use should be consistent with local regulations regarding the use of contraceptive methods for participants participating in clinical studies. Hormonal contraception may be susceptible to interaction with the study intervention, which may reduce the efficacy of the contraceptive method. In this case, two highly effective methods of contraception are utilized during the intervention period and for at least 6 months after the last dose of study intervention. Oral hormonal contraception may be susceptible to interaction with the study intervention, which may reduce the efficacy of the contraceptive method. In this case, if the oral contraceptive cannot be replaced by another highly effective method of contraception with a different route of administration, the hormonal contraception method must be supplemented with a male condom (for partner) during the intervention period and for at least 6 months after the last dose of study intervention.

PREGNANCY TESTING: WOCBP is included only after a confirmed menstrual period and a negative highly sensitive serum pregnancy test. Additional pregnancy testing is performed at the beginning of each treatment cycle during the intervention period and at EOT. Pregnancy testing is performed whenever a menstrual cycle is missed or when pregnancy is otherwise suspected. Pregnancy testing is performed according to local lab procedure. Any female participant who becomes pregnant while participating in the study is to discontinue study intervention and is withdrawn from the study.

Collection of Pregnancy Information:

Male participants with partners who become pregnant—The Investigator attempts to collect pregnancy information on any male participant's female partner who becomes pregnant while the male participant is in this study. This applies only to male participants who receive the cytokine RNA mixture. After obtaining the necessary signed informed consent from the pregnant female partner directly, the Investigator records pregnancy information on the appropriate form and submits it to the Sponsor within 24 hours of learning of the partner's pregnancy. The female partner is also be followed to determine the outcome of the pregnancy. Information on the status of the mother and child is forwarded to the Sponsor. Generally, the follow-up will be no longer than 6 to 8 weeks following the estimated delivery date. Any termination of the pregnancy will be reported regardless of fetal status (presence or absence of anomalies) or indication for the procedure.

Female participants who become pregnant—The Investigator collects pregnancy information on any female participant who becomes pregnant while participating in this study. Information is recorded on the appropriate form and submitted to the Sponsor within 24 hours of learning of a participant's pregnancy. The participant is followed to determine the outcome of the pregnancy. The Investigator will collect follow-up information on the participant and the neonate and the information will be forwarded to the Sponsor. Generally, follow-up is not required for longer than 6 to 8 weeks beyond the estimated delivery date. Any termination of pregnancy is reported, regardless of fetal status (presence or absence of anomalies) or indication for the procedure. Any pregnancy complication or elective termination of a pregnancy is reported as an AE or SAE. A spontaneous abortion is always considered to be an SAE and will be reported as such. Any post-study pregnancy related SAE considered reasonably related to the study intervention by the Investigator is reported to the Sponsor. While the Investigator is not obligated to actively seek this information in former study participants, he or she may learn of an SAE through spontaneous reporting.

Any female participant becoming pregnant while participating in the study discontinues the study intervention and is withdrawn from the study.

### Example 1.13—Recommended Supportive Care or Dose Modification Guidelines for Drug-Related Adverse Events

TABLE 17
Grading System and Mitigation Strategy for Hypersensitivity based on
CTCAE v. 5.0

| | Grade per | | |
|---|---|---|---|
| Severity | CTCAE v. 5.0 | Toxicity | Intervention |
| Mild | “allergic reaction” | Transient flushing or | Systemic intervention |
| Hypersensitivity | Grade 1 | rash, fever <38° C. | not indicated. |
| | | (<100.4° F.) | Continue treatment |
| | | | per Investigator' |
| | | | judgment following |
| | | | close direct |
| | | | monitoring of the |
| | | | participant. |
| | | | Treatment may be |
| | | | stopped at any time if |
| | | | deemed necessary. |
| | | | Intervention not |
| | | | indicated but |
| | | | treatment may be |
| | | | resumed only after |
| | | | participant recovery |
| | | | and with continued |
| | | | close monitoring. |
| Moderate | “allergic reaction” | Moderate | Stop treatment |
| Hypersensitivity | Grade 2 | hypersensitivity, which | prophylactic oral |
| | | responds promptly to | intervention indicated |
| | | symptomatic treatment | for ≤24 hrs (e.g., |
| | | | antihistamines, |
| | | | NSAIDs). |
| | | | Treatment may be |
| | | | resumed only after |
| | | | participant recovery, |
| | | | with close |
| | | | monitoring, and |
| | | | following assessment |
| | | | by the Study |
| | | | Committee. |
| Severe/life- | “allergic reaction” | Grade 3—Symptomatic | Definitive treatment |
| threatening | Grades 3 & 4 | bronchospasm, with or | discontinuation |
| Hypersensitivity | | without urticaria; | (stop treatment). |
| | | parenteral intervention | Urgent intervention |
| | | indicated; allergy- | indicated. |
| | | related | Hospitalization |
| | | edema/angioedema; | indicated for clinical |
| | | hypotension | sequelae. Intravenous |
| | | Grade 4 in addition to | intervention |
| | | Grade 3 symptoms, it | indicated. |
| | | has life-threatening | Give additional |
| | | consequences | medication with |
| | | | diphenhydramine 25 |
| | | | mg IV (or equivalent) |
| | | | and/or |
| | | | methylprednisolone |
| | | | 100 mg IV (or |
| | | | equivalent) and/or |
| | | | epinephrine as |
| | | | needed. |
| Severe/life- | “anaphylaxis” | Symptomatic | Definitive treatment |
| threatening | Grades 3 & 4 | bronchospasm, with or | discontinuation |
| Hypersensitivity | | without urticaria; | (stop treatment). |
| | | parenteral intervention | Urgent intervention |
| | | indicated; allergy- | indicated. |
| | | related | Give additional |
| | | edema/angioedema; | medication with |
| | | hypotension. | diphenhydramine 25 |
| | | Grade 4 has life- | mg IV (or equivalent) |
| | | threatening | and/or |
| | | consequences. | methylprednisolone |
| | | | 100 mg IV (or |
| | | | equivalent) and/or |
| | | | epinephrine as |
| | | | needed. |

Symptoms, Grading, and Management of CRS

Clinical signs and symptoms associated with CRS
Cardiovascular: tachycardia, widened pulse pressure, hypotension, increased cardiac output (early), potentially diminished cardiac output (late)
Coagulation: elevated D-dimer, hypofibrinogenemia±bleeding
Gastrointestinal: nausea, vomiting, diarrhea
General: fever rigors, malaise, fatigue, anorexia, myalgia, arthralgia, headache
Hepatic: transaminitis, hyperbilirubinemia
Neurologic: headache, mental status changes, confusion, delirium, word finding difficulty or frank aphasia, hallucinations, tremor, dysmetria, altered gait, seizures
Renal: azotemia
Respiratory: tachypnea, hypoxemia
Skin: rash

Grading and Management of CRS is provided in Table 18.

TABLE 18
Grading System and Mitigation Strategy for CRS, based on
2014 NCI Consensus guidelines

| Grade | Toxicity | Intervention |
|---|---|---|
| Grade 1 | Fever with or without | Vigilant supportive care. |
| | constitutional symptoms | Assess and treat infection if |
| | (nausea, fatigue, headache, | present. |
| | myalgias, malaise, without | Fluid resuscitation. |
| | life threatening | Provide antipyretics and |
| | complications) | analgesics, if needed |
| Grade 2 | Oxygen requirement <40% | As for Grade 1, but monitor |
| | FiO2 | cardiac and other organ |
| | Hypotension responsive to | function closely |
| | low-dose or single | Consider corticosteroids and/or |
| | vasopressor | anti-IL6 therapy for |
| | Grade 2 organ toxicity | participants with advanced age |
| | | or multiple co-morbidities |
| | | Continuation of study |
| | | intervention should be assessed |
| | | case by case by the Study |
| | | Committee |
| Grade 3 | Oxygen requirement >40% | As for Grade 2, but with the |
| | FiO2 | addition of corticosteroids |
| | Hypotension responsive to | and/or anti-IL6 therapy |
| | high-dose or multiple | Definitive treatment |
| | vasopressors | discontinuation (stop |
| | Grade 3 organ toxicity | treatment) |
| | Grade 4 increase in ALT or | |
| | AST | |
| Grade 4 | Life threatening symptoms | As for Grade 2, but with the |
| | Requirement for mechanical | addition of corticosteroids |
| | ventilation. | and/or anti-IL6 therapy |
| | Grade 4 organ toxicity, | Definitive treatment |
| | excluding increase in ALT | discontinuation (stop |
| | or AST | treatment) |
| Grade 5 | Death | |

Guidance for Other AEs

Table 19A provides guidelines for uveitis management, note that all attempts are made to rule out other causes such as metastatic disease, infection, or other ocular disease (e.g., glaucoma or cataracts).

Table 19B provides guidance and supportive care strategies for the management of adverse events that are attributed to the cytokine RNA mixture.

TABLE 19A
Ophthalmology (uveitis) AE management

| | The cytokine mixture | Cemiplimab dosing | |
|---|---|---|---|
| Uveitis CTCAE v5.1 | dosing management | management | Action and Guidelines |
| Grade 1 | Continue immunotherapy | | Start artificial tears and |
| (mild) | | | refer to |
| | | | ophthalmologist. |
| | | | Treat with topical |
| | | | steroids such as 1% |
| | | | prednisolone acetate |
| | | | suspension. |
| Grade 2 | No change in dose | Delay treatment until | Urgent ophthalmologist |
| (anterior uveitis) | | recovery to Grade 1. | consultation |
| | | Discontinue treatment if | Treatment guided by |
| | | symptoms persist | ophthalmologist to |
| | | despite treatment with | include ophthalmologic |
| | | topical | and systemic |
| | | immunosuppressive | corticosteroid. |
| | | therapy, and do not | |
| | | improve to Grade 1 | |
| | | within the retreatment | |
| | | period, or requires | |
| | | systemic treatment. | |
| Grade ≥3 | Delay or omit dose | Discontinue treatment | Urgent ophthalmologist |
| (Posterior or pan- | until Grade ≤2 | | consultation |
| uveitis) | | | Treatment guided by |
| | | | ophthalmologist to |
| | | | include ophthalmologic |
| | | | and systemic |
| | | | corticosteroid. When |
| | | | symptoms improve to |
| | | | Grade ≤1, steroid taper |
| | | | is started and continued |
| | | | over no less than 4 |
| | | | weeks. |

All attempts are made to rule out other causes such as metastatic disease, infection, or other ocular disease (e.g., glaucoma or cataracts).

TABLE 19B
Guidance of supportive care for adverse events and dose modifications

| | | | Management of |
|---|---|---|---|
| Description | Grading | Supportive Care/Treatment | IMP dosing |
| Injection site | Grade 1-2 | Treatment of symptoms (e.g., pain, | Prevent to inject |
| reactions | | erythema, swelling, superimposed | same lesion if |
| | | bacterial infection). | feasible, |
| | | | otherwise total |
| | | | recovery is |
| | | | needed to inject |
| | | | same lesion. |
| | Grade 3-4 | Operative intervention indicated. | Definitively stop |
| | | | treatment for |
| | | | Grade 3 or 4 |
| | | | events. |
| Dry Eye | Minimization | Baseline Schirmer's test | |
| (lacrimal gland | | Monitor participants at each | |
| atrophy) | | visit for ocular signs and | |
| | | symptoms. | |
| | Grade 2 | Ophthalmologist consultation and | No dose |
| | (Symptomatic) | multi-agent treatment. | modification |
| | Grade 3 | Ophthalmologist consultation and | Omit dose till |
| | | multi-agent treatment. | recovery to |
| | | | Grade ≤1 |
| | | | In case of |
| | | | recurrent |
| | | | Grade 3 event; |
| | | | definitive |
| | | | discontinuation |
| | | | of the |
| | | | cytokine RNA |
| | | | mixture |
| | | | administration |
| Hepatitis | Grade 2 with | Re-check Liver enzyme, | Withhold until |
| | AST or ALT >3 | bilirubin and albumin every | recovely to |
| | to 5 × ULN | 3 days. | Grade <1 |
| | (>3.0-5.0 × | Review potentially linked | |
| | baseline if | medications (statins, | |
| | baseline was | antibiotics, alcohol | |
| | abnormal), or | consumption, etc.). | |
| | total bilirubin | Viral serology. | |
| | >1.5 to 3 × ULN | Consider imaging of | |
| | (>1.5-3.0 × | metastatic disease. | |
| | baseline if | | |
| | baseline was | | |
| | abnormal) | | |
| | Grade 3: AST or | As above but repeat liver | Withhold the |
| | ALT >5.0-20.0 × | enzyme, bilirubin and | treatment |
| | ULN (>5.0- | albumin tests daily. | until |
| | 20.0 × baseline if | Perform USG with doppler. | recovery to |
| | baseline was | | Grade ≤1 or |
| | abnormal) | | baseline |
| | | | value. |
| | | | If confirmed |
| | | | as related to |
| | | | IMP, re- |
| | | | challenge |
| | | | can be |
| | | | discussed by |
| | | | Study |
| | | | Committee. |
| | | | If recurrent |
| | | | G3 event, |
| | | | permanent |
| | | | discontinuation. |
| | Grade 4 AST or | In addition to Grade 3: | Definitively |
| | ALT >20.0 × | Hepatology consultation | discontinue the |
| | ULN (>20.0 × | Consider liver biopsy when | cytokine RNA |
| | baseline if | participant condition is | mixture |
| | baseline was | suitable | administration. |
| | abnormal) | | |
| Acute Kidney | Minimization | Vulnerable group includes | |
| injury | | participants with diabetes mellitus, | |
| Theoretical | | significant coronary or peripheral | |
| risk: | | vascular disease as well as those | |
| Degeneration/ | | receiving nephrotoxic medications. | |
| regeneration of | | Prevention of Acute tubular necrosis | |
| kidney cortical | | includes maintaining euvolemia, | |
| tubules | | avoiding nephrotoxic medications, | |
| | | and supporting blood pressure with | |
| | | vasopressors if necessary. | |
| | | Monitor participants closely | |
| | | for signs and symptoms of | |
| | | kidney injury; special care is | |
| | | needed for vulnerable | |
| | | population (i.e.. participants | |
| | | with diabetes mellitus, | |
| | | significant coronary or | |
| | | peripheral vascular disease | |
| | | as well as those receiving | |
| | | nephrotoxic medications. | |
| | | Monitor chemistry | |
| | | parameters in real time; | |
| | | maintain euvolemia, | |
| | | avoiding nephrotoxic | |
| | | medications, and supporting | |
| | | blood pressure with | |
| | | vasopressors if necessary. | |
| | | Collecting urine samples for | |
| | | exploratory measurement of | |
| | | biomarker Kim-1 | |
| | | retrospectively and in case of | |
| | | increased creatinine or other | |
| | | signs of kidney injury. | |
| Creatinine | Grade 1— | If MDRD calculated GFR >60 | If MDRD |
| Increase: | Creatinine >1- | mL/min; no action. | calculated GFR, |
| | 1.5 × baseline: | If calculated GFR between 40-60 | >60 mL/min; no |
| | >ULN − 1.5 × | mL/min, evaluate 24 hr urine tests | dose |
| | ULN | (GFR, protein, electrolytes). | modification. |
| | | Symptomatic treatment as | If GFR <40 |
| | | mentioned above. | mL/min, delay |
| | | | cycle and |
| | | | consider study |
| | | | intervention |
| | | | discontinuation |
| | | | if the event does |
| | | | not improve |
| | | | with |
| | | | symptomatic |
| | | | treatment. |
| | Grade 2— | Symptomatic treatment as | Delay the |
| | Creatinine 1,5- | mentioned above. | cytokine RNA |
| | 3 × above | | mixture and |
| | baseline; >1.5- | | permanently |
| | 3.0 × ULN | | discontinue if |
| | | | the event |
| | | | persists >7 days |
| | | | or worsens. |
| | Grade 3: | Hospitalization is indicated; | Grade 3-4: |
| | Creatinine >3 × | temporary dialyses can be | Discontinue the |
| | baseline; >3.0- | considered to balance fluid and | cytokine RNA |
| | 6.0 ULN | electrolytes. | mixture. |
| | | Dialysis indicated. | |
| | Grade 4: | If possible, renal biopsy is | |
| | Creatinine >6.0 | recommended to ensure | |
| | ULN | pathogenesis. | |
| | (If Life- | | |
| | threatening | | |
| | consequences | | |
| | report as Grade | | |
| | 4 Acute | | |
| | Kidney Injury) | | |
| Immune- | Minimization: | Exclusion of participants | |
| Mediated | | with underlying autoimmune | |
| Events | | disease. | |
| (Grading | | Monitor participants for | |
| according to | | signs and symptoms. | |
| the NCI | | | |
| CTCAE v5.0 | | | |
| grading of each | Grade 1 | No intervention, symptomatic | Grade 1—No |
| events) | (asymptomatic, | management. | dose |
| Pneumonitis | serologic or | | modification. |
| Hypothyroiditis | other evidence of | | |
| | autoimmune | | |
| | disease) | | |
| | Grade 2 | Medical intervention is indicated. | Grade 2—No |
| | (moderate | | dose |
| | symptoms) | | modification. |
| | Grade 3 | Pulse with methylprednisolone 1-2 | Grade 3—Delay |
| | (severe | mg/kg/day in case of major organ | dose till |
| | symptoms | involvement (e, g. pneumonitis); | symptoms to |
| | medical | additional immunosuppressive | resolve to Grade |
| | intervention | therapy may be indicated | ≤1 event; |
| | and/or | | definitive |
| | hospitalization | | discontinuation |
| | indicated) | | if Grade 3 event |
| | | | leads >1 dose |
| | | | omission. |
| | Grade 4 | | Definitive |
| | (life threatening) | | discontinuation. |

In an attempt to harmonize the reporting of local tumor reactions across clinical sites any local signs of tumor (skin, subcutaneous or lymph node tumors) inflammation in injected and non-injected lesions following cytokine mRNA mixture intratumoral injections will be reported using the CTCAE version 5.0 term: skin and subcutaneous tissue or “injection site reaction.”

### Example 1.14—Response Evaluation Criteria in Solid Tumors Version 1.1

Measurability of Tumor at Baseline

At baseline, tumor lesions/lymph nodes are categorized measurable or non-measurable as follows.

Measurable lesions are accurately measured in at least 1 dimension (longest diameter in the plane of the measurement to be recorded) with a minimum size of:
10 mm by CT scan (CT scan slice thickness no greater than 5 mm).
10 mm caliper measurement by clinical exam (lesions which cannot be accurately measured with calipers should be recorded as non-measurable).
20 mm by chest X-ray.

Malignant lymph nodes: To be considered pathologically enlarged and measurable, a lymph node must be >15 mm in short axis when assessed by CT scan (CT scan slice thickness recommended to be no greater than 5 mm). At baseline and in follow-up, only the short axis is measured and followed.

Non-measurable lesions are all other lesions, including small lesions (longest diameter <10 mm or pathological lymph nodes with ≥10 to <15 mm short axis), as well as non-measurable lesions. Lesions considered non-measurable include; leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

Special Considerations Regarding Lesion Measurability:

Bone Lesions:
Bone scan, positron emission tomography scan or plain films are not considered adequate imaging techniques to measure bone lesions. However, these techniques can be used to confirm the presence or disappearance of bone lesions.
Lytic bone lesions or mixed lytic-blastic lesions, with identifiable soft tissue components, that can be evaluated by cross sectional imaging techniques such as CT or MRI can be considered as measurable lesions if the soft tissue component meets the definition of measurability described above.
Blastic bone lesions are non-measurable.

Cystic Lesions:
Lesions that meet the criteria for radiographically defined simple cysts are not considered as malignant lesions (neither measurable nor non-measurable) since they are, by definition, simple cysts.
‘Cystic lesions’ thought to represent cystic metastases can be considered as measurable lesions, if they meet the definition of measurability described above. However, if non-cystic lesions are present in the same patient, these are preferred for selection as target lesions.

Lesions with Prior Local Treatment:
Tumor lesions situated in a previously irradiated area, or in an area subjected to other loco-regional therapy, are usually not considered measurable unless there has been demonstrated progression in the lesion.

Method of Assessment

All measurements are recorded in metric notation, using calipers if clinically assessed. All baseline evaluations are performed as close as possible to the treatment start and never more than 4 weeks before the beginning of the treatment.

The same method of assessment and the same technique are used to characterize each identified and reported lesion at baseline and during follow-up. Imaging based evaluation is always performed rather than clinical examination unless the lesion(s) being followed cannot be imaged but are assessable by clinical examination.

Clinical lesions: Clinical lesions are only considered measurable when they are superficial and ≥10 mm diameter as assessed using calipers. For the case of skin lesions, documentation by color photography including a ruler to estimate the size of the lesion is suggested. As noted above, when lesions can be evaluated by both clinical exam and imaging, imaging evaluation is undertaken since it is more objective and may be reviewed at the end of the study.

Chest X-ray: Chest CT is preferred over chest X-ray, particularly when progression is an important endpoint, since CT is more sensitive than X-ray, particularly in identifying new lesions. However, lesions on chest X-ray are considered measurable if they are clearly defined and surrounded by aerated lung.

CT, MRI: CT is the best currently available and reproducible method to measure lesions selected for response assessment. Measurability of lesions on CT scan is based on the assumption that CT slice thickness is 5 mm or less. When CT scans have slice thickness greater than 5 mm, the minimum size for a measurable lesion should be twice the slice thickness.

Ultrasound: Ultrasound is not useful in assessment of lesion size and should not be used as a method of measurement. If new lesions are identified by ultrasound in the course of the study, confirmation by CT or MRI is advised.

Endoscopy, laparoscopy: The utilization of these techniques for objective tumor evaluation is not advised.

Tumor markers: Tumor markers alone cannot be used to assess objective tumor response.

Cytology, histology: These techniques can be used to differentiate between PR and CR in rare cases if required by protocol.

FDG PET-CT/CT scans. Performed in lymphoma patients approximately every 12 weeks to confirm CR or PD.

Baseline Documentation of ‘Target’ and ‘Non-Target’ Lesions

When more than 1 measurable lesion is present at baseline all lesions up to a maximum of 5 lesions total (and a maximum of 2 lesions per organ) representative of all involved organs should be identified as target lesions and will be recorded and measured at baseline.

Target lesions are selected based on their size (lesions with the longest diameter), are representative of all involved organs, and lend themselves to reproducible repeated measurements.

Lymph nodes merit special mention since they are normal anatomical structures which may be visible by imaging even if not involved by tumor. Pathological nodes which are defined as measurable and may be identified as target lesions must meet the criterion of a short axis of ≥15 mm by CT scan. Only the short axis of these nodes contributes to the baseline sum. All other pathological nodes (those with short axis ≥10 mm but <15 mm) should not be considered non-target lesions. Nodes that have a short axis <10 mm are considered non-pathological and should not be recorded or followed.

A sum of the diameters (longest for non-nodal lesions, short axis for nodal lesions) for all target lesions is calculated and reported as the baseline sum diameters. The baseline sum diameters are used as reference to further characterize any objective tumor regression in the measurable dimension of the disease.

All other lesions (or sites of disease) including pathological lymph nodes are identified as non-target lesions and are also recorded at baseline. Measurements are not required, and these lesions are followed as “present”, “absent”, or “unequivocal progression”. In addition, it is possible to record multiple non-target lesions involving the same organ as a single item on the case (e.g., “multiple enlarged pelvic lymph nodes” or “multiple liver metastases”).

Response criteria are described in Table 20.

TABLE 20
Response criteria

| Response criteria | Evaluation of target lesions |
|---|---|
| CR | Disappearance of all target lesions. |
| | Any pathological lymph nodes |
| | (whether target or non-target) must have |
| | reduction in short axis to <10 mm. |
| PR | At least a 30% decrease in the sum of |
| | diameters of target lesions, taking as |
| | reference the baseline sum diameters. |
| PD | At least a 20% increase in the sum of |
| | diameters of target lesions, taking as |
| | reference the smallest sum on study |
| | (this includes the baseline sum if |
| | that is the smallest on study). In addition |
| | to the relative increase of 20%, |
| | the sum must also demonstrate an |
| | absolute increase of at least 5 mm. |
| | (Note: the appearance of 1 or more |
| | new lesions is also considered progression). |
| SD | Neither sufficient shrinkage from the |
| | baseline study to qualify for PR nor |
| | sufficient increase to qualify for PD, |
| | taking as reference the smallest |
| | sum diameters while on study. |

Abbreviations:
CR = complete response;
PD = progressive disease;
PR = partial response;
SD = stable disease.

Special Notes on the Assessment of Target Lesions

Lymph nodes identified as target lesions always have the actual short axis measurement recorded and are measured in the same anatomical plane as the baseline examination, even if the nodes regress to below 10 mm on study. This means that when lymph nodes are included as target lesions, the ‘sum’ of lesions may not be zero even if CR criteria are met, since a normal lymph node is defined as having a short axis of <10 mm. For PR, SD and PD, the actual short axis measurement of the nodes is to be included in the sum of target lesions.

Target lesions that become ‘too small to measure’: All lesions (nodal and non-nodal) recorded at baseline should have their actual measurements recorded at each subsequent evaluation, even when very small (e.g., 2 mm). However, sometimes lesions or lymph nodes which are recorded as target lesions at baseline become so faint on CT scan that the radiologist may not feel comfortable assigning an exact measure and may report them as being ‘too small to measure’. When this occurs, it is important that a value is recorded on the CRF. If it is the opinion of the radiologist that the lesion has likely disappeared, the measurement is recorded as 0 mm. If the lesion is believed to be present and is faintly seen but too small to measure, a default value of 5 mm is assigned.

When non-nodal lesions ‘fragment’, the longest diameters of the fragmented portions are added together to calculate the target lesion sum. Similarly, as lesions coalesce, a plane between them is maintained that would aid in obtaining maximal diameter measurements of each individual lesion. If the lesions have truly coalesced such that they are no longer separable, the vector of the longest diameter in this instance is the maximal longest diameter for the “coalesced lesion”.

Evaluation of Non-Target Lesions

While some non-target lesions may be measurable, they need not be measured and instead are assessed only qualitatively at the time points specified in the protocol.

CR: Disappearance of all non-target lesions and normalization of tumor marker level. All lymph nodes must be non-pathological in size (<10 mm short axis).

Non-CR/Non-PD: Persistence of 1 or more non-target lesion(s) and/or maintenance of tumor marker level above the normal limits.

Progressive Disease: Unequivocal progression of existing non-target lesions. (Note: the appearance of 1 or more new lesions is also considered progression).

The concept of progression of non-target disease requires additional explanation as follows:

When the participant also has measurable disease; in this setting, to achieve “unequivocal progression” based on the non-target disease, there must be an overall level of substantial worsening in non-target disease such that, even in presence of SD or PR in target disease, the overall tumor burden has increased sufficiently to merit discontinuation of therapy. A modest “increase” in the size of 1 or more non-target lesions is usually not sufficient to qualify for unequivocal progression status.

When the participant has only non-measurable disease; to achieve ‘unequivocal progression’ based on the non-target disease, there must be an overall level of substantial worsening such that the overall tumor burden has increased sufficiently to merit discontinuation of therapy. A modest ‘increase’ in the size of 1 or more non-target lesions is usually not sufficient to qualify for unequivocal progression status. Because worsening in non-target disease cannot be easily quantified (by definition: if all lesions are truly non-measurable) a useful test that can be applied when assessing patients for unequivocal progression is to consider if the increase in overall disease burden based on the change in non-measurable disease is comparable in magnitude to the increase that would be required to declare PD for measurable disease: i.e., an increase in tumor burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localized to widespread, or may be described in protocols as ‘sufficient to require a change in therapy’. If ‘unequivocal progression’ is seen, the patient is considered to have had overall PD at that point.

New Lesions

The appearance of new malignant lesions denotes disease progression. The finding of a new lesion should be unequivocal: i.e., not attributable to differences in scanning technique, change in imaging modality or findings thought to represent something other than tumor (for example, some ‘new’ bone lesions may be simply healing or flare of pre-existing lesions). This is particularly important when the participant's baseline lesions show PR or CR. For example, necrosis of a liver lesion may be reported on a CT scan report as a ‘new’ cystic lesion, which it is not.

A lesion identified on a follow-up study in an anatomical location that was not scanned at baseline is considered a new lesion and indicates disease progression. An example of this is the patient who has visceral disease at baseline and while on study has a CT or MRI brain ordered which reveals metastases. The participant's brain metastases are considered to be constitute PD even if he/she did not have brain imaging at baseline.

If a new lesion is equivocal, for example because of its small size, continued therapy and follow-up evaluation clarifies if it represents new disease. If repeat scans confirm that there is a new lesion, then progression is declared using the date of the initial scan.

While fluorodeoxyglucose-positron emission tomography (FDG-PET) response assessments need additional study, it is sometimes reasonable to incorporate the use of FDG-PET scanning to complement CT scanning in assessment of progression (particularly possible ‘new’ disease). New lesions based on FDG-PET imaging are identified according to the following algorithm:
A. Negative FDG-PET at baseline, with a positive FDG-PET at follow-up is a sign of PD based on a new lesion.
B. No FDG-PET at baseline and a positive FDG-PET at follow-up:
   If the positive FDG-PET at follow-up corresponds to a new site of disease confirmed by CT, this is PD.
   If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan). If the positive FDG-PET at follow-up corresponds to a pre-existing site of disease on CT that is not progressing on the basis of the anatomic images, this is not PD.

Evaluation of Best Overall Response

Time point response: At each protocol specified time point, a response assessment should occur. Table 21 provides a summary of the overall response status calculation at each time point for patients who have measurable disease at baseline.

TABLE 21
Response in patients with target disease

| Target lesions | Non-target lesions | New lesions | Overall response |
|---|---|---|---|
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | Inevaluable |
| PD | Any | Yes or No | PD |
| Any | PD | Yes or No | PD |
| Any | Any | Yes | PD |

Abbreviations:
CR = complete response;
PD = progressive disease;
PR = partial response;
SD = stable disease.

When patients have non-measurable (therefore non-target) disease only. Table 22 is to be used.

TABLE 22
Response in patients with non-target disease only

| Non-target lesions | New lesions | Overall response |
|---|---|---|
| CR | No | CR |
| Non-CR/non-PD | No | Non-CR/non-PD |
| Not all evaluated | No | Inevaluable |
| Unequivocal PD | Yes or No | PD |
| Any | Yes | PD |

Abbreviations:
CR = complete response;
PD = progressive disease;
PR = partial response;
SD = stable disease.

Missing assessments and inevaluable designation: When no imaging/measurement is done at all at a particular time point, the patient is not evaluable (NE) at that time point.

If only a subset of lesion measurements is made at an assessment, usually the case is also considered NE at that time point, unless a convincing argument can be made that the contribution of the individual missing lesion(s) would not change the assigned time point response. This would be most likely to happen in the case of PD. When no imaging/measurement is done at all at a particular time point, the patient is NE at that time point.

If only a subset of lesion measurements is made at an assessment, usually the case is also considered NE at that time point, unless a convincing argument can be made that the contribution of the individual missing lesion(s) would not change the assigned time point response. This would be most likely to happen in the case of PD.

Special Notes on Response Assessment

When nodal disease is included in the sum of target lesions and the nodes decrease to ‘normal’ size (<10 mm), they may still have a measurement reported on scans. This measurement is recorded even though the nodes are normal in order not to overstate progression should it be based on increase in size of the nodes. As noted earlier, this means that patients with CR may not have a total sum of ‘zero’ on the CRF.

In trials where confirmation of response is required, repeated ‘NE’ time point assessments may complicate best response determination. The analysis plan for the trial must address how missing data/assessments are addressed in determination of response and progression. For example, in most trials it is reasonable to consider a patient with time point responses of PR-NE-PR as a confirmed response.

Patients with a global deterioration of health status requiring discontinuation of treatment without objective evidence of disease progression at that time are reported as ‘symptomatic deterioration’. Every effort should be made to document objective progression even after discontinuation of treatment. Symptomatic deterioration is not a descriptor of an objective response: it is a reason for stopping study therapy.

The objective response status of such patients is determined by evaluation of target and non-target disease. For equivocal findings of progression (e.g., very small and uncertain new lesions; cystic changes or necrosis in existing lesions), treatment may continue until the next scheduled assessment. If at the next scheduled assessment, progression is confirmed, the date of progression is the earlier date when progression was suspected.

Duration of Response

The duration of overall response is measured from the time measurement criteria are first met for CR/PR (whichever is first recorded) until the first date that recurrent or PD is objectively documented (taking as reference for PD the smallest measurements recorded on study).

The duration of overall CR is measured from the time measurement criteria are first met for CR until the first date that recurrent disease is objectively documented.

Stable disease is measured from the start of the treatment until the criteria for progression are met, taking as reference the smallest sum on study (if the baseline sum is the smallest, this is the reference for calculation of PD).

Non-limiting descriptions relating to the RECIST guidelines are provided in Eisenhauer E A, Therasse P. Bogaerts J et al. New response evaluation criteria in solid tumours: Revised RECIST guideline (version 1.1). Eur J Cancer. 2009; 45:228-47, the entire contents of which are incorporated herein by reference.

### Example 1.15—Modified Response Evaluation Criteria in Solid Tumors for Immune-Based Therapeutics

Details are provided in Seymour L, Bogaerts J, Perrone A. Ford R., Schwartz. L H, Mandrekar S, et al. iRECIST: guidelines for response criteria for use in trials testing immunotherapeutics. Lancet Oncol. 2017 March: 18(3):e143-52.

TABLE 23
Comparison of Response Evaluation Criteria in Solid Tumors
(RECIST) 1.1 and modified Response Evaluation Criteria in Solid
Tumors for immune-based therapies (iRECIST)

| | RECIST 1.1 | iRECIST |
|---|---|---|
| Definitions of measurable | Measurable lesions are | No change from RECIST |
| and non-measurable disease | ≥10 mm in diameter | 1.1; however, new lesions are |
| numbers and site of target | (≥15 mm for nodal lesions); | assessed as per RECIST 1.1 |
| disease | maximum of 5 lesions (2 per | but are recorded separately |
| | organ); all other disease is | on the case report form (but |
| | considered non-target (must | not included in the sum of |
| | be ≥10 mm in short axis for | lesions for target lesions |
| | nodal disease) | identified at baseline) |
| Complete response, partial | Cannot have met criteria for | Can have had iUPD (one or |
| response, or stable disease | progression before complete | more instances), but not |
| | response, partial response, or | iCPD, before iCR, iPR, or |
| | stable disease | iSD |
| Confirmation of complete | Only required for non- | As per RECIST 1.1 |
| response or partial response | randomized trials | |
| Confirmation of stable | Not required | As per RECIST 1.1 |
| disease | | |
| New lesions | Result in progression; | Results in iUPD but iCPD is |
| | recorded but not measured | only assigned on the basis of |
| | | this category if at next |
| | | assessment additional new |
| | | lesions appear or an increase |
| | | in size of new lesions is seen |
| | | (≥5 mm for sum of new |
| | | lesion target or any increase |
| | | in new lesion non-target); the |
| | | appearance of new lesions |
| | | when none have previously |
| | | been recorded, can also |
| | | confirm iCPD |
| Independent blinded review | Recommended in some | Collection of scans (but not |
| and central collection of | circumstances -e.g., in some | independent review) |
| scans | trials with progression-based | recommended for all trials |
| | endpoints planned for | |
| | marketing approval | |
| Confirmation of progression | Not required (unless | Required |
| | equivocal) | |
| Consideration of clinical | Not included in assessment | Clinical stability is |
| status | | considered when deciding |
| | | whether treatment is |
| | | continued after iUPD |

“i” indicated immune responses assigned using iRECIST.
Abbreviations:
iCPD = confirrned progression;
iCR = complete response;
iPR = partial response;
iSD = stable disease;
iUPD = unconfirmed progression;
RECIST = Response Evaluation Criteria in Solid Tumors

TABLE 24
Assessment of timepoint response using modified Response Evaluation
Criteria in Solid Tumors for immune-based therapies (iRECIST)

| | | | Timepoint | |
|---|---|---|---|---|
| | | | response | |
| | | | with no | |
| | | | previous | |
| | | | iUPD in | |
| Target | Non-target | New | any | Timepoint response with |
| lesions | lesions | lesions | category | previous iUPD in any category^{a} |
| iCR | iCR | No | iCR | iCR |
| iCR | Non-iCR/non- | No | iPR | iPR |
| | iUPD | | | |
| iPR | Non-iCR/non- | No | iPR | iPR |
| | iUPD | | | |
| iSD | Non-iCR/non- | No | iSD | iSD |
| | iUPD | | | |
| iUPD | iUPD with no | Yes | Not | New lesions confirm iCPD if new lesions |
| with no | change, or | | applicable | were previously identified and they have |
| change, | decrease from | | | increased in size (≥5 mm in sum of |
| or with a | last timepoint | | | measures for new lesion target or any |
| decrease | | | | increase for new lesion non-target) or |
| from last | | | | number; if no change is seen in new lesions |
| timepoint | | | | (size or number) from last timepoint, |
| | | | | assignment remains iUPD |
| iSD, | iUPD | No | iUPD | Remains iUPD unless iCPD is confirmed |
| iPR, iCR | | | | on the basis of a further increase in the size |
| | | | | of non-target disease (does not need to meet |
| | | | | RECIST 1.1 criteria for unequivocal |
| | | | | progression) |
| iUPD | Non-iCR/non- | No | iUPD | Remains iUPD unless iCPD is confirmed |
| | iUPD, or iCR | | | on the basis of a further increase in swn of |
| | | | | measures ≥5 mm; otherwise, assignment |
| | | | | remains iUPD |
| iUPD | iUPD | No | iUPD | Remains iUPD unless iCPD is confirmed |
| | | | | based on a further increase in previously |
| | | | | identified target lesion iUPD in sum of |
| | | | | measures ≥5 mm or non-target lesion iUPD |
| | | | | (previous assessment need not have shown |
| | | | | unequivocal progression) |
| iUPD | iUPD | Yes | iUPD | Remains iUPD unless iCPD is confirmed |
| | | | | on the basis of a further increase in |
| | | | | previously identified target lesion iUPD |
| | | | | sum of measures ≥5 mm, previously |
| | | | | identified non-targe lesion iUPD (does not |
| | | | | need to be unequivocal), or an increase in |
| | | | | the size or number of new lesions |
| | | | | previously identified |
| Non- | Non-iUPD or | Yes | iUPD | Remains iUPD unless iCPD is confirmed |
| iUPD or | progression | | | on the basis of an increase in the size or |
| progression | | | | number of new lesions previously identified |

^{a}Previously identified in assessment immediately before this timepoint.
“i” indicates immune responses assigned using iRECIST Target lesions, non-target lesions, and new lesions defined according to RECIST 1.1 principles; if no pseudoprogression occurs, RECIST 1.1 and iRECIST categories for complete response, partial response, and stable disease would be the same.
Abbreviations:
iCPD = confirmed progression;
iCR = complete response;
iPR = partial response;
iSD = stable disease;
iUPD = unconfirmed progression;
non-iCR/non-iUPD = criteria for neither CR nor PD have been met;
RECIST = Response Evaluation Criteria in Solid Tumors.

TABLE 25
Eastern Cooperative Oncology Group Performance Status Scale

| Performance | |
|---|---|
| Status | Description |
| 0 | Fully active, able to carry on all predisease |
| | performance without restriction. |
| 1 | Restricted in physically strenuous activity but |
| | ambulatory and able to carry out work of a |
| | light or sedentary nature, e.g., light house |
| | work, office work. |
| 2 | Ambulatory and capable of all self-care but |
| | unable to carry out any work activities; |
| | up and about more than 50% of waking hours. |
| 3 | Capable of only limited self-care; confined to |
| | bed or chair more than 50% of waking hours. |
| 4 | Completely disabled; cannot carry on any |
| | self-care; totally confined to bed or chair. |
| 5 | Dead. |

Developed by the Eastern Cooperative Oncology Group, Robert L. Comis, MD, Group Chair.

TABLE 26
Cutaneous melanoma TNM Staging (AJCC Cancer Staging ed 8th)
Melanoma TNM Classification

| T Classification | Thickness | Ulceration Status |
|---|---|---|
| Tis | Not applicable | Not applicable |
| T1 | ≤1.0 mm | a: <0.8 mm w/o |
| | | ulceration |
| | | b: <0.8 mm with |
| | | ulceration 0.8-1.0 mm |
| | | with or without |
| | | ulceration |
| T2 | 1.0-2.0 mm | a: without ulceration |
| | | b: with ulceration |
| T3 | 2.01-4.0 mm | a: without ulceration |
| | | b: with ulceration |
| T4 | >4.0 mm | a: without ulceration |
| | | b: with ulceration |
| | No. of Metastatic | Nodal Metastatic |
| N Classification | Nodes | Mass |
| N1 | 1 node | a: Clinically occult^{a} |
| | | b: Clinically detected^{b} |
| | | c: In-transit |
| | | met(s)/satellites(s) |
| | | without metastatic |
| | | nodes |
| N2 | 2-3 nodes | a: Clinically occult^{a} |
| | | b: Clinically detected^{b} |
| | | c: In-transit |
| | | met(s)/satellites(s) with |
| | | one metastatic node |
| N3 | 4 or more metastatic | |
| | nodes, or matted nodes, | |
| | or in-transit | |
| | met(s)/satellite(s) with | |
| | metastatic nodes(s) | |
| M Classification | Site | Serum LDH |
| M1a (0) | Distant skin, | Normal |
| | subcutaneous, or nodal | |
| | metastases | |
| M1a (1) | | Elevated |
| M1b (0) | Lung metastases | Normal |
| M1b (1) | | Elevated |
| M1c (0) | All other visceral | Normal |
| | metastases | |
| M1c (1) | | Elevated |
| M1d | Metastasis to central | |
| | nervous | |

^{a}Clinically occult are diagnosed after sentinel or elective lymphadenectomy.
^{b}Clinically detected are defined as clinically detectable nodal metastases confirmed by therapeutic lymphadenectomy or when nodal metastasis exhibits gross extracapsular extension.
Source: adapted from Gershenwald JE, Scolyer RA, Hess KR, et al. Melanoma of the skin. In: Amin MB, ed. AJCC Cancer Staging Manual. 8^{th} ed. Chicago, IL: AJCC-Springer; 2017: 563-585.

TABLE 27
Melanoma Stage/Prognostic Groups—Stage IIIB and above
Melanoma Stage/Prognostic Groups (Stage IIIB and above)

| Stage | T | N | M |
|---|---|---|---|
| Stage IIIB | T1-4b | N1a | M0 |
| | T1-4b | N1b | M0 |
| | T1-4a | N1b | M0 |
| | T1-4a | N2c | M0 |
| Stage IIIC | T1-4b | N1b | M0 |
| | T1-4b | N2b | M0 |
| | T1-4b | N2c | M0 |
| | Any T | N3 | M0 |
| Stage IV | Any T | Any N | M1 |

Source: adapted from Gershenwald JE, Scolyer -RA, Hess KR, et al. Melanoma of the skin. In: Amin MB, ed. AJCC Cancer Staging Manual. 8^{th} ed. Chicago, IL: AJCC-Springer; 2017: 563-585.

Disease response will be assessed using the Lugano Classification 2014 (Cheson B D et al. (2014) J. Clinical Oncology 32(27)3059-3068). Response assessments occur at Screening and every 12 weeks (+7 days).

TABLE 28
Disease assessment and Lugano Classification
Reproduced from Lugano Classification 2014, Cheson et al. 2014,
Table 3. Revised Criteria for Response Assessment

| Response and Site | PET-CT-Based Response | CT-Based Response |
|---|---|---|
| | | Complete radiologic response |
| Complete | Complete metabolic response | (all of the following) |
| Lymph nodes and | Score 1, 2, or 3* with or without a | Target nodes/nodal masses must regress to |
| extralymphatic sites | residual mass on 5PS** | ≤1.5 cm in LDi |
| | It is recognized that in Waldeyer's ring | No extralymphatic sites of disease |
| | or extranodal sites with high | |
| | physiologic uptake or with activation | |
| | within spleen or marrow (eg, with | |
| | chemotherapy or myeloid colony- | |
| | stimulating factors), uptake may be | |
| | greater than normal mediastinum | |
| | and/or liver. In this circumstance, | |
| | complete metabolic response may be | |
| | inferred if uptake at sites of initial | |
| | involvement is no greater than | |
| | surrounding normal tissue even if the | |
| | tissue has high physiologic uptake | |
| Nonmeasured lesion | Not applicable | Absent |
| Organ enlargement | Not applicable | Regress to normal |
| New lesions | None | None |
| Bone marrow | No evidence of FDG-avid disease in | Normal by morphology; if indeterminate, |
| | marrow | IHC negative |
| Partial | Partial metabolic response | Partial remission (all of the owing) |
| Lymph nodes and | Score 4 or 5** with reduced uptake | ≥50% decrease in SPD of up to 6 target |
| extralymphatic sites | compared with baseline and residual | measurable nodes and extranodal sites |
| | mass(es) of any size | |
| | At interim, these findings suggest | When a lesion is too small to measure on |
| | responding disease | CT, assign 5 mm × 5 mm as the default |
| | | value |
| | At end of treatment, these findings | When no longer visible, 0 × 0 mm |
| | indicate residual disease | For a node >5 mm × 5 mm, but smaller |
| | | than normal, use actual measurement for |
| | | calculation |
| Nonmeasured lesion | Not applicable | Absent/normal, regressed, but no increase |
| Organ enlargement | Not applicable | Spleen must have regressed by >50% in |
| | | length beyond normal |
| New lesions | None | None |
| Bone marrow | Residual uptake higher than uptake in | Not applicable |
| | normal marrow but reduced compared | |
| | with baseline (diffuse uptake | |
| | compatible with reactive changes from | |
| | chemotherapy allowed). If there are | |
| | persistent focal changes in the marrow | |
| | in the context of a nodal response, | |
| | consideration should be given to | |
| | further evaluation with MRI or biopsy | |
| | or an interval scan | |
| No response | | |
| or stable disease | No metabolic response | Stable disease |
| Target nodes/nodal | Score 4 or 5 with no significant change | <50% decrease from baseline in SPD of up |
| masses, extranodal | in FDG uptake from baseline at interim | to 6 dominant, measurable nodes and |
| lesions | or end of treatment | extranodal sites; no criteria for progressive |
| | | disease are met |
| Nonmeasured lesion | Not applicable | No increase consistent with progression |
| Organ enlargement | Not applicable | No increase consistent with progression |
| New lesions | None | None |
| Bone marrow | No change from baseline | Not applicable |
| | | Progressive disease requires |
| Progressive disease | Progressive metabolic disease | at least 1 of the following |
| Individual target | Score 4 or 5 with an increase in | PPD progression: |
| nodes/nodal masses | intensity of uptake from baseline | |
| | and/or | |
| Extranodal | New FDG-avid foci consistent with | An individual node/lesion must be |
| lesions | lymphoma at interim or end-of- | abnormal with: |
| | treatment assessment | LDi >1.5 cm and |
| | | Increase by ≥50% from PPD nadir and |
| | | An increase in LDi or SDi from nadir |
| | | 0.5 cm for lesions ≤2 cm |
| | | 1.0 cm for lesions >2 cm |
| | | In the setting of splenomegaly, the splenic |
| | | length must increase by >50% of the extent |
| | | of its prior increase beyond baseline (eg, a |
| | | 15-cm spleen must increase to >16 cm). If |
| | | no prior splenomegaly, must increase by at |
| | | least 2 cm from baseline |
| | | New or recurrent splenomegaly |
| Nonmeasured lesion | None | New or clear progression of preexisting |
| | | nonmeasured lesions |
| New lesions | New MG-avid foci consistent with | Regrowth of previously resolved lesions |
| | lymphoma rather than another etiology | A new node >1.5 cm in any axis |
| | (eg, infection, inflammation). If | A new extranodal site >1.0 cm in any axis; |
| | uncertain regarding etiology of new | if <1.0 cm in any axis, its presence must be |
| | lesions, biopsy or interval scan may be | unequivocal and must be attributable to |
| | considered | lymphoma |
| | | Assessable disease of any size |
| | | unequivocally attributable to lymphoma |
| Bone marrow | New or recurrent FDG-avid foci | New or recurrent involvement |

Abbreviations:
5PS, 5-point scale;
CT, computed tomography;
FDG, flurodeoxyglucose;
IHC, immunohistochemistry;
LDi, longest transverse diameter of a lesion;
MRI, magnetic resonance imaging;
PET, positron emission tomography;
PPD, cross product of the LDi and perpendicular diameter,
SDi, shortest axis perpendicular to the LDi;
SPD; sum of the product of the perpendicular diameters for multiple lesions.
*A score of 3 in many patients indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (cg, liver, spleen, kidneys, lungs); GI involvement, cutaneous lesions, or those noted on palpation. Nonmeasured lesions: Any disease not selected as measured, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (eg, GI tract, liver, bone marrow). FDG uptake may be greater than in the mediastinum with complete metabolic response, but should be no higher than surrounding normal physiologic uptake (eg, with marrow activation as a result of chemotherapy or myeloid growth factors).
**PET 5PS: 1, no uptake above background; 2, uptake ≤ mediastinum; 3, uptake > mediastinum but ≤ liver; 4, uptake moderately > liver; 5, uptake markedly higher than liver and/or new lesions; X, new areas of uptake unlikely to be related to lymphoma.

Imaging timing should follow calendar days and should not be adjusted for delays in cycle. For participants who discontinue for reasons other than PD, assessments should continue until the participant has documented PD. The first assessment may be performed earlier than 12 weeks if in the opinion of the Investigator the participant is clinically progressing.

TABLE 29
Abbreviations

| | |
|---|---|
| ADA: | anti-drug antibodies |
| ALT: | alanine aminotransferase |
| ANC: | absolute neutrophil count |
| AST: | aspartate aminotransferase |
| CAR: | chimeric antigen receptor |
| CK: | pancytokeratin |
| CRP: | C-reactive protein |
| DL: | dose level |
| DL1: | starting dose level |
| DLT: | dose limiting toxicity |
| DoR: | duration of response |
| DRE: | disease related event |
| ECOG: | Eastern Cooperative Oncology Group |
| eCRF: | electronic case report form |
| EOT: | end of treatment |
| HBsAg: | hepatitis B surface antigen |
| hCG: | human chorionic gonadotropin |
| HLH: | hemophagocytic lymphohistiocytosis |
| ICF: | Informed Consent Form |
| ICH: | International Council for Harmonisation |
| IP10: | INFγ-induced protein 10 |
| iRECIST: | RECIST for immunotherapies |
| iUPD: | unconfirmed progressive disease |
| IV: | intravenously |
| KIM-1: | kidney injury molecule-1 |
| LDH: | lactate dehydrogenase |
| MRI: | magnetic resonance imaging |
| NCI CTCAE: | National Cancer Institute Commom |
| | Terminology Criteria for Adverse Events |
| NOAEL: | no observed-adverse-effect-level |
| NSAID: | non-steroidal anti-inflammatory drug |
| PDy: | pharmacodynamics |
| PFS: | progression free survival |
| PK: | pharmacokinetics |
| PO: | orally |
| RNAseq: | RNA sequencing |
| SAE: | serious adverse event |
| STD 10: | Severely Toxic Dose in 10% of animals |
| USG: | ultrasonography |
| WOCBP: | women of childbearing potential |

### Example 2—Anti-Tumor Activity in Mice with Acquired Anti PD1-Resistant Tumors

Mouse Model for Acquired Resistance to Anti-PD1 Therapy

A mouse tumor model exhibiting acquired resistance to anti-PD-1 antibody treatment was generated essentially as follows. See, also, Dunn et al. (2002) Nature Immunology 3: 991-998; and Wang X et al. (2017) Cancer Res 77(4): 839-850. Female C57BL6/J mice (Jackson Laboratory. Bar Harbor, Me., USA) bearing MC38 tumors were treated with an anti-PD-1 antibody (clone RMP1-14; as first described in Yamaz.aki et al. (2005) J Immunol 175(3): 1586-1592 at methods), growing tumors were excised, and cells were cultured ex vivo in RPMI-1640 with L-glutamine (Life Technologies) supplemented with 10% FBS. Female C57BL6/J mice aged 6 to 8 weeks were housed in a temperature controlled environment on 12 hour light cycle with free access to food and sterile water. All mice were acclimated for at least 3 days prior to experimentation. Body weight and tumor volume, if measured, were measured twice weekly until the experimental endpoints. Tumor volume is expressed as the product of the perpendicular diameters using the following formula: a²*b/2, where a<b.

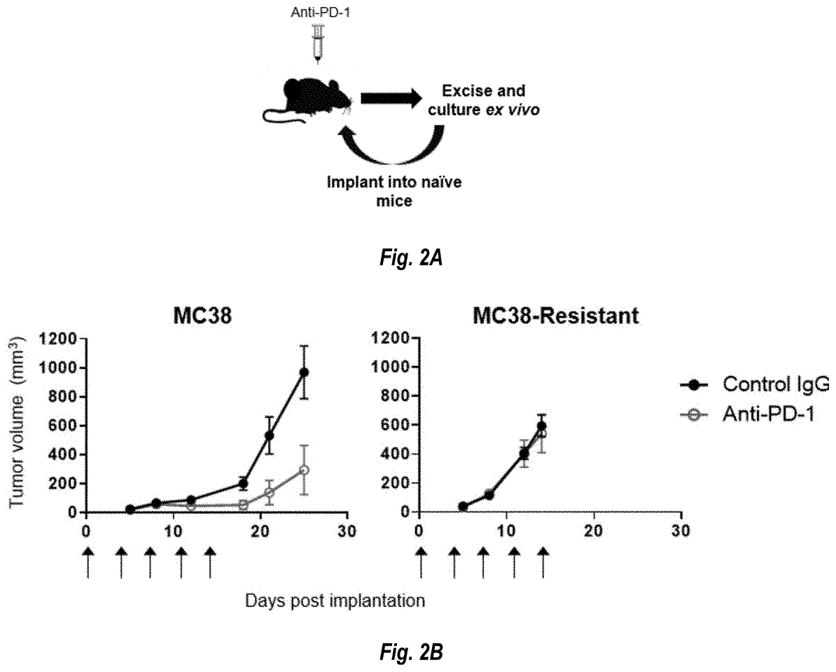

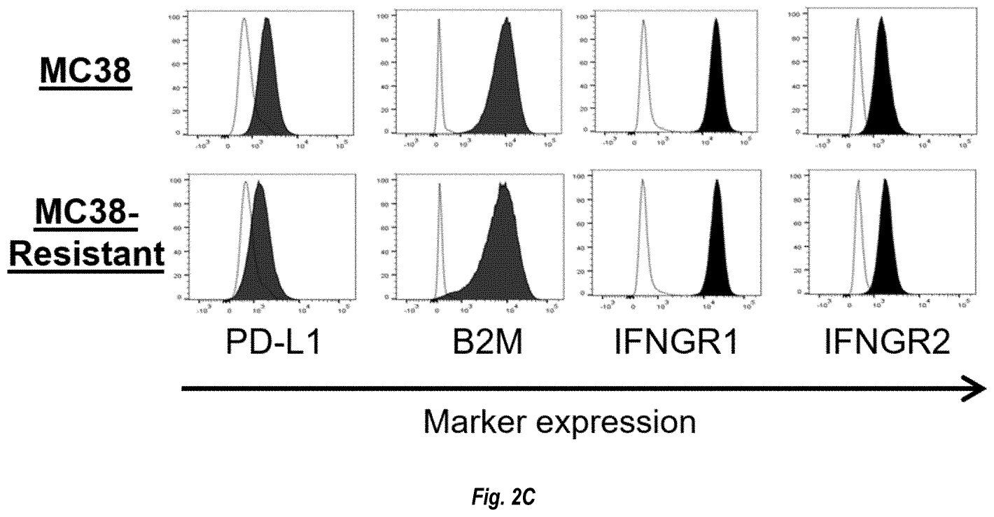

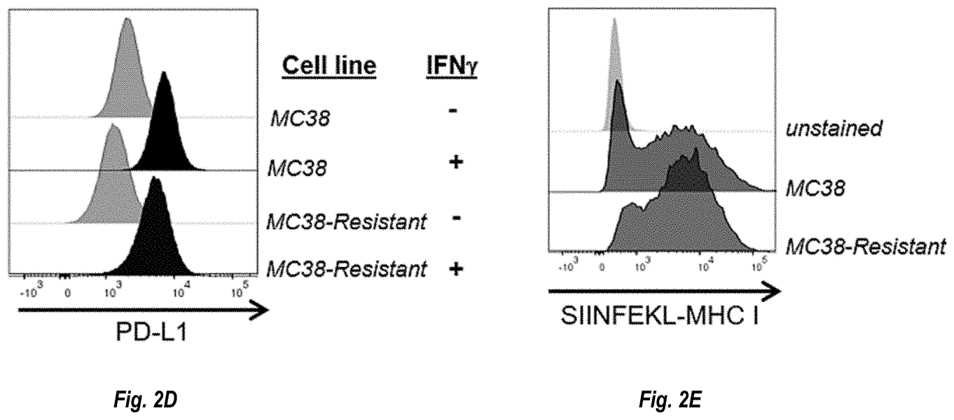

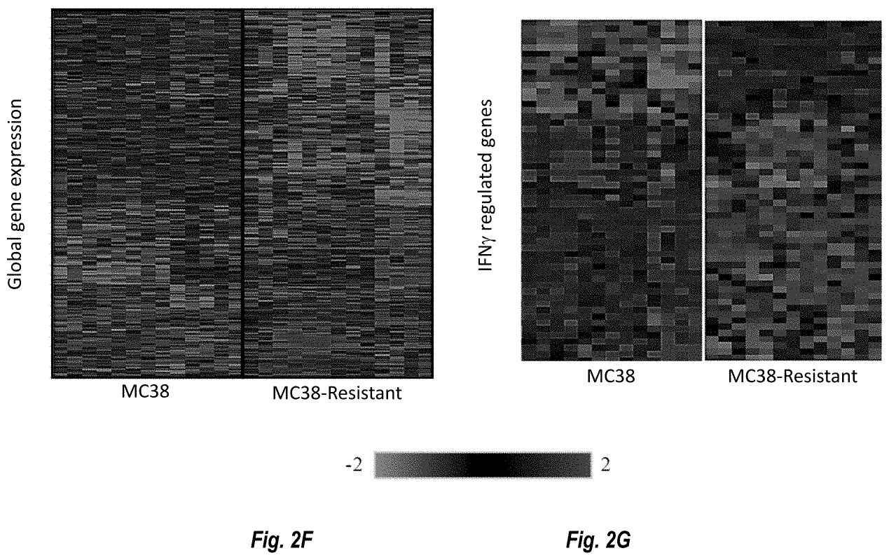

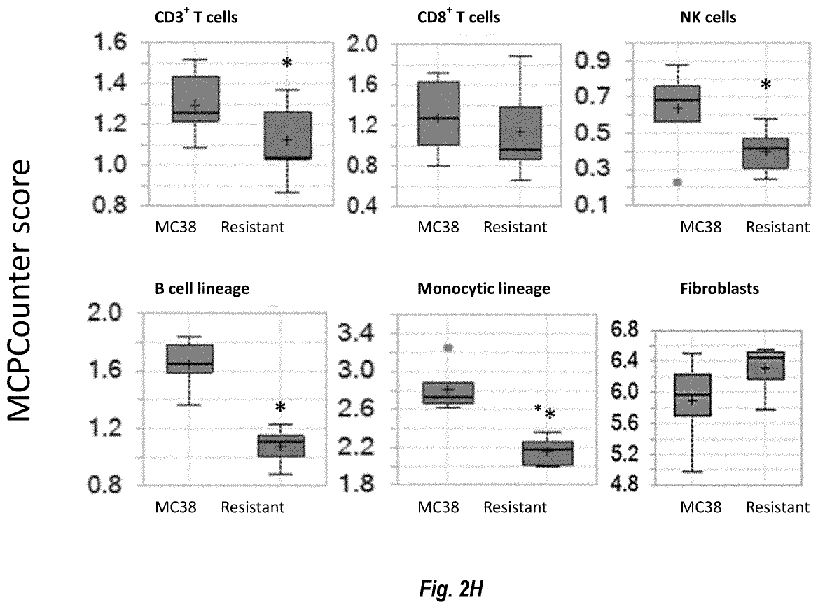

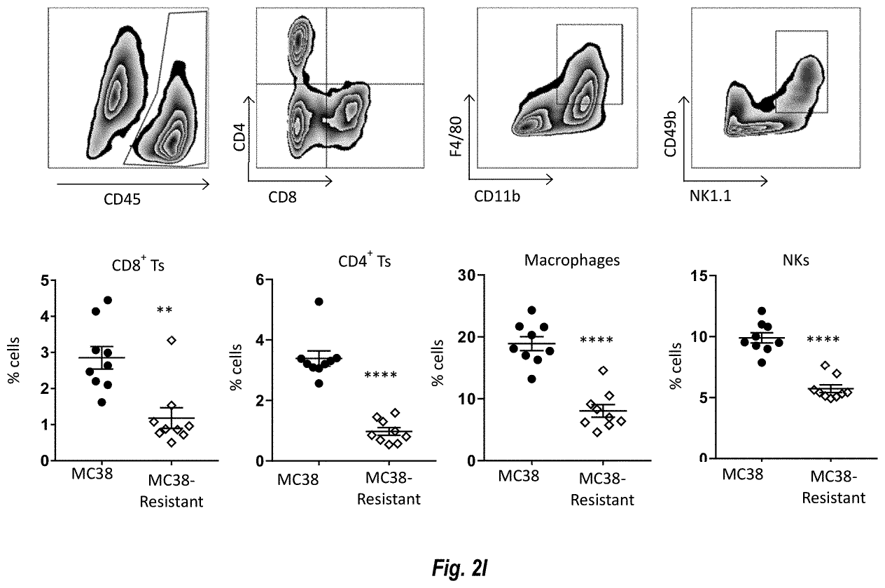

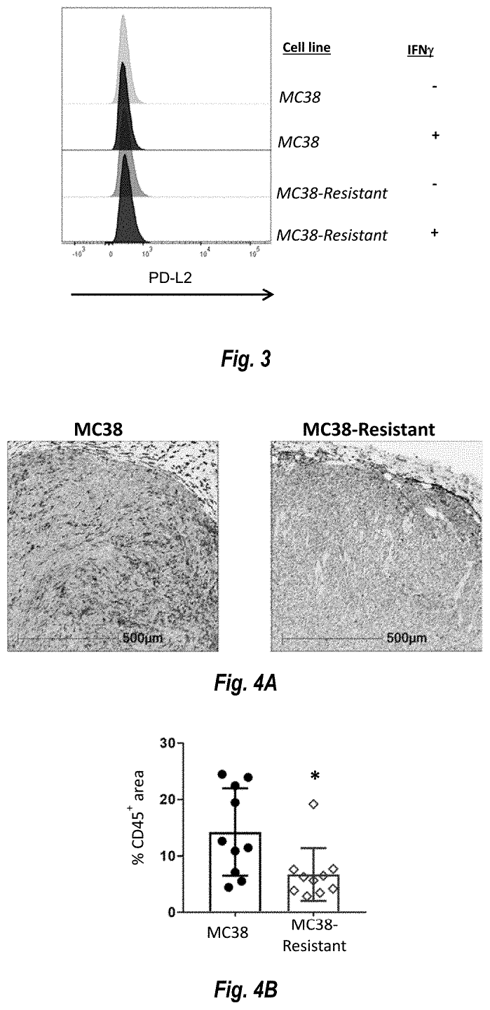

For FIGS. 2A-3, one million MC38 or MC38-resistant cells were suspended in 200 pI DPBS and injected subcutaneously into the right flank of each mouse.

In Vivo Drug Administration

Four doses of mouse cytokine mRNA mixture or control mRNA encoding luciferase (Luc mRNA) were administered every four days (Q4D) by intratumoral (IT) injection at 40 μg in 50 μl per tumor starting when tumors reached an average of 60 mm3. Mouse body weight and tumor volume were measured twice weekly until the experimental endpoints. Tumor volume was expressed as the product of perpendicular diameters using the following formula: a²*b/2, where a<b. All procedures were approved by an Institutional Animal Care and Use Committee and were conducted in accordance with the NIH Guide for the Care and Use of Laboratory Animals.

Preparation of mRNA

Synthetic DNA fragments coding for the gene of interest were cloned into a common starting vector, comprising a 5′-untranslated region (UTR) and 3′ UTR, a 3′ UTR, and a poly(A)-tail of 110 nucleotides in total. Linearization of plasmid DNA was performed downstream of the poly(dA:dT) with a classIIS restriction enzyme to generate a template with no additional nucleotides beyond poly(dA:dT) (See, e.g., Holtkamp et al. (2006) Blood December 15; 108(13):4009-17). Linearized plasmid DNA was subjected to in vitro transcription with T7 RNA polymerase (Thermo Fisher, Waltham Mass., USA) as described by Grudzien-Nogalska et al (2013) Methods Mol Bio. 969:55-72, in the presence of 7.5 mM ATP, CTP, GTP, and N1-methyl-pseudouridinetriphosphate. RNA was purified using magnetic particles (Berensmeier S. (2006) Applied Microbiology and Biotechnology 73(3):495-504) and subsequently a Cap1 structure was introduced using the Vaccinia Capping system (New England Biolabs, Ipswich, Mass., USA) and 2′-O-methylation of the mRNA cap. The RNA was further purified using cellulose-based chromatography to remove double-stranded RNA (dsRNA) impurities (see Day P R et al (1977) Phytopathology 67:1393; Morris T J et al. (1979)Phytopathology 69:854-858; and Castillo A et al. (2011) Virol J. 8:38). RNA concentration and quality were assessed using spectrophotometry and capillary gel electrophoresis systems. Presence of dsRNA was assessed in a Northwestern dot-blot assay using dsRNA-specific J2 mAb (English & Scientific Consulting, KFt. Szirák, Hungary) as described by Karikó et al (2011) Nucleic Acids Res. November: 39(21): e142.

Results

Several mechanisms of innate and acquired resistance to checkpoint blockade have been defined and include mutations of MHC I and IFNγ signaling pathways. See, for example, Sharma et al. (2017) Cell 168(4):707-723; Sade-Feldman M et al. (2017) Nat Commun 8(1):1136; Zaretsky J M et al. (2016) N Engl J Med 375(9): 819-29; Gettinger S. et al. (2017) 7(12): 1420-1435; Rodig S J et al. (2018) Sci Transl Med 10(450). However, such mutations occur in a low frequency of patients and additional mechanisms have yet to be defined. In an effort to better understand acquired resistance to checkpoint blockade, we generated a mouse tumor model exhibiting in vivo resistance to anti-PD-1 antibody treatment. MC38 tumors acquired resistance to PD-1 blockade following serial in vivo passaging (FIG. 2A, B). Lack of sensitivity to PD-1 blockade was not attributed to dysregulation of PD-L1 or B2M expression, as both were expressed at similar levels in parental and resistant cells (FIG. 2C). Similarly, IFNγ signaling and antigen processing and presentation pathways were functional in both parental and resistant cell lines (FIG. 2D, 2E). Unbiased gene expression analysis was used to further characterize potential resistance mechanisms. RNA-sequencing revealed substantial differences in global gene expression with PD-1 resistant tumors displaying a marked reduction in expression of immune-related genes relative to parental MC38 tumors (FIGS. 2F, 2G). Indeed, PD-1 resistant tumors exhibit reduced immune infiltration across multiple cell types, including T and NK cells (FIGS. 2H, 2I).

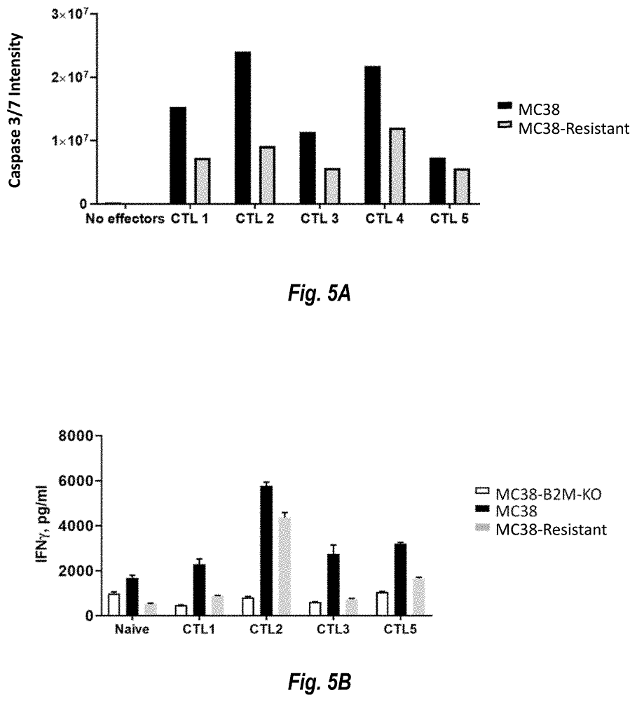

Further validation of the model was performed and the results are shown in FIGS. 3-5. Briefly, MC38-resistant cells were shown to not express PD-L2, and expression was not induced following IFNγ treatment (FIG. 3). Immunohistochemical staining showed reduced frequency of immune cells in resistant tumors (FIG. 4A). Paraffin embedded MC38 and MC38-resistant tumors were analyzed by immunohistochemical staining for infiltration of CD45′ cells (dark color). Results are representative of two independent experiments; n=10 tumors per group. FIG. 4A shows representative images. FIG. 4B shows quantification. FIGS. 5A-5B show reduced immunogenicity of resistant tumors. In short, cytotoxic T lymphocyte (CTL) cultures were generated from 5 individual C57BL6 mice bearing parental MC38 tumors that exhibited complete regression in response to PD-1 blockade. CTLs were co-cultured with MC38 and resistant tumor cells, and killing (FIG. 5A) and IFNγ release (FIG. 5B) were measured.

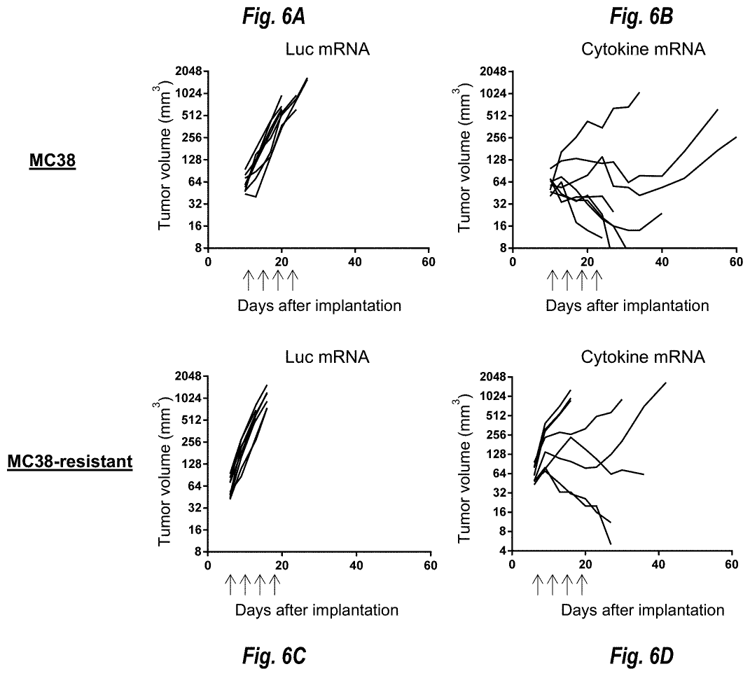

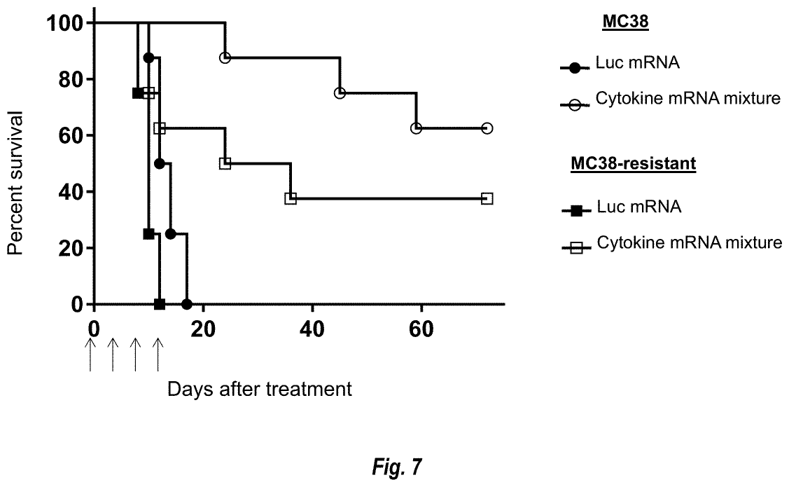

Using this validated model, cytokine RNA mixture was administered intratumorally as monotherapy. Monotherapy with murine cytokine RNA mixture inhibited the growth of both MC38 and MC38-resistant tumors as compared to control. See. FIGS. 6A-6D. Monotherapy with murine cytokine RNA mixture also significantly prolonged the survival of mice bearing MC38 and MC38-resistant tumors. See, FIG. 7. Five out of eight (62.5%) mice bearing MC38 tumors (FIG. 6B) and three out of eight (37.5%) mice bearing MC38-resistant tumors (FIG. 6D) exhibited complete tumor remission and were tumor-free at the end of the experiment. See also, FIG. 7.

### Example 3—Anti-Tumor Activity in Mice with Anti PD1-Resistant Tumors

Mouse Model for Resistance to anti-PD1 Therapy

MC38 cells, a gift from Dr. S. A. Rosenberg (National Institute of Health, Bethesda, Md., USA), were cultured in RPMI-1640 with L-glutamine (Life Technologies) supplemented with 10% FBS. In general, for the single flank tumor model, MC38 cells were suspended in DPBS and 1×10⁶ cells in 200 μl were implanted SC into the right flank of C57BL/6J mice. In general, for the dual flank tumor MC38 model, 1×10⁶ cells on the right side and 0.5×10⁶ cells on the left side were implanted SC on day 0.

To generate an in vivo tumor model of resistance to anti-PD-1 therapy, MC38-B2M-knockout cells were generated using CRISPR using the sgRNA 5′-GGCGTATGTATCAGTCTCAG-3′ (SEQ ID NO: 31). MC38 cells were transiently transfected (Lipofectamine™ CRISPRMAX™; ThermoFisher Scientific, Waltham, Mass., USA) with pre-complexed Cas9 and sgRNA (GeneArt™ Platinum™ Cas9 Nuclease V.2: ThermoFisher Scientific) according to the manufacturer's instructions. B2M^{-/-} cells were enriched using MACS technology (Miltenyi Biotec. Bergisch Gladbach, Germany), then single cell colonies were isolated and knockout confirmed by flow cytometry.

In Vivo Drug Administration

Cytokine RNA mixture was administered by intratumoral injection. Mice were anesthetized with isoflurane and 80 μg in 50 μl mRNA in saline solution injected intratumorally (IT) into the right tumor every 4 days for four doses total unless detailed otherwise. Antibodies were obtained from BioXCell (West Lebanon, N.H., USA) unless otherwise noted and administered by IP injection. Control (MOPC-21) and anti-PD-1 (RMP1-14) were administered at a dose of 5 mg/kg every three days (Q3D).

Results

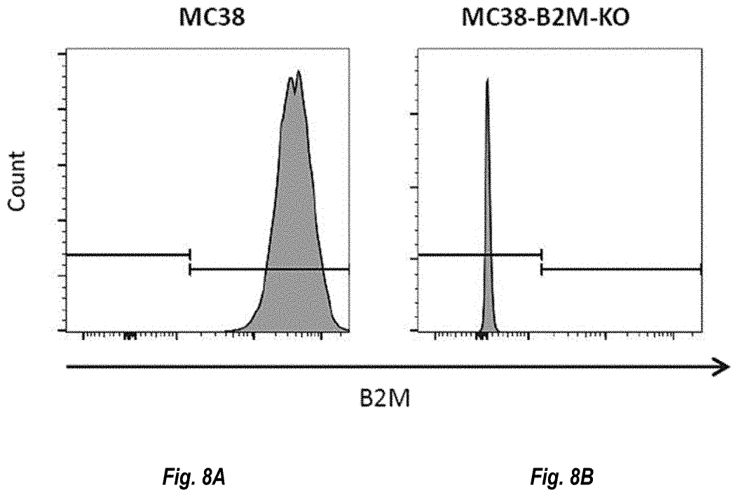

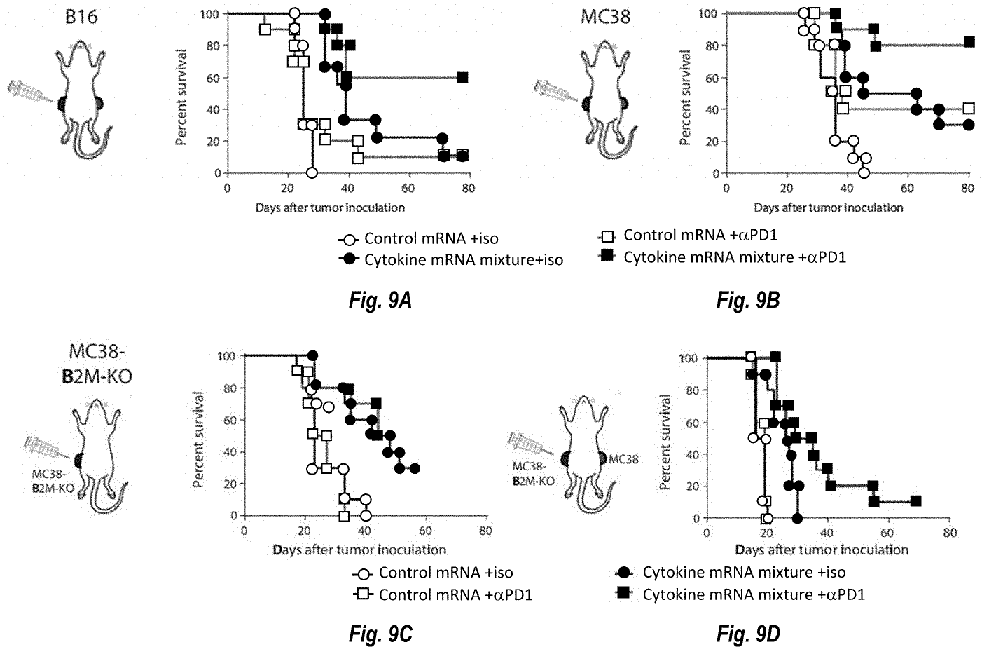

To investigate therapeutic efficacy of cytokine mRNA treatment in a checkpoint resistant setting, B2M was genetically deleted in MC38 cells (FIG. 8) which led to in vivo resistance to anti-PD-1 treatment (FIG. 9C), whereas mice bearing parental tumors remained partially responsive to anti-PD-1 treatment (FIG. 9B). Cytokine RNA mixture treatment alone conferred prolonged survival in animals bearing B2M knockout tumors, however, no additional increase in survival was observed by combining cytokine mRNA with anti-PD-1 checkpoint blockade (FIG. 9C).

To model the intertumoral heterogeneity often observed in human malignancies, a dual flank setting was established with MC38-B2M knockout on one side and the MC38-WT tumors on the contralateral flank (FIG. 9D). The MC38-B2M knockout tumors were injected with cytokine RNA mixture while the contralateral MC38-WT tumors were left untreated. Treatment with anti PD-1 therapy alone had no effect on the survival of tumor-bearing mice, whereas cytokine RNA mixture alone prolonged survival, although all mice eventually succumbed to tumor burden (FIG. 9D). Combination treatment further increased overall survival in this setting, indicating that combination treatment with cytokine RNA mixture and anti-PD-1 antibody has an abscopal effect even when the treated lesion is resistant to T cell-mediated killing due to lack of MHC I expression (FIG. 9D).

### Example 4—Anti-Tumor Activity in Additional Murine Models

Materials and Methods

Twelve syngenic cell lines were maintained in vitro with different medium (shown below) at 37° C. in an atmosphere of 5% CO₂ in air. The tumor cells will be routinely subcultured twice weekly. The cells in an exponential growth phase were harvested and counted for tumor inoculation. Each mouse was inoculated subcutaneously with tumor cells in 0.1 mL of PBS for tumor development. After tumor cells inoculation, the animals were checked daily for morbidity and mortality. During routine monitoring, the animals were checked for any effects of tumor growth and treatments on behavior such as mobility, food and water consumption, body weight gain/loss (Body weights would be measured twice per week after randomization), eye/hair matting and any other abnormalities. Mortality and observed clinical signs were recorded for individual animals in detail. Tumor volumes were measured twice per week after randomization in two dimensions using a caliper, and the volume was expressed in mm³ using the formula: V=(L×W×W)/2, where V is tumor volume. L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were measured by using StudyDirector™ software (version 3.1.399.19).

TABLE 30
Medium and Cell Line information

| | | | Cell | |
|---|---|---|---|---|
| Cell line | Vendor of cell line | Medium | amount/mouse | Inoculation site |
| CT26 | SIBS, Shanghai | RPMI1640 + 10% FBS | 5 × 10e5 | right lower flank |
| | Institutes for | | | |
| | Biological Sciences | | | |
| Pan02 | NIH | RPMI1640 + 10% FBS | 1 × 10e6 | right front flank |
| H22 | CCTCC, China | RPMI1640 + 10% FBS | 3 × 10e6 | right front flank |
| | Center for Type | | | |
| | Culture Collection | | | |
| MC38 | FDCC, Fudan Cell | DMEM + 10% FBS | 1 × 10e6 | right lower flank |
| | Center | | | |
| A20 | ATCC | RPMI1640 + 10% FBS | 5 × 10e5 | right lower flank |
| B16BL6 | Nanjing Keygen | RPMI1640 + 10% FBS | 2 × 10e5 | right lower flank |
| | biotech | | | |
| Renca | ATCC | DMEM + 10% FBS | 1 × 10e6 | right lower flank |
| LL/2 | SIBS, Shanghai | DMEM + 10% FBS | 5 × 10e5 | right lower flank |
| | Institutes for | | | |
| | Biological Sciences | | | |
| EMT-6 | ATCC | DMEM + 10% FBS | 3 × 10e5 | right lower flank |
| RM-1 | SIBS, Shanghai | RPMI1640 + 10% FBS | 1 × 10e6 | right lower flank |
| | Institutes for | | | |
| | Biological Sciences | | | |
| B16F10 | SIBS, Shanghai | DMEM + 10% FBS | 2 × 10e5 | right lower flank |
| | Institutes for | | | |
| | Biological Sciences | | | |
| Hepa 1-6 | SIBS, Shanghai | DMEM + 10% FBS | 5 × 106e | right front flank |
| | Institutes for | | | |
| | Biological Sciences | | | |

TABLE 31
Study Design

| | | | Dosing | Dosing | | Dosing |
|---|---|---|---|---|---|---|
| | | Dose level | Solution | Volume | | Frequency & |
| N | Treatment | (mg/kg) | (mg/ml) | (μL/g) | ROA | Duration* |
| 10 | Cytokine mRNA | 40 μg/ | 40 μg/50 μl | 50 μl/tumor | intratumoral | Q4D × 4 (day |
| | Mixture | tumor | | | | 1, 5, 9, 13) |
| 10 | Anti-PD-1 | 10 | 1 | 10 | i.p. | BIW × 3 |
| | | | | | | weeks (day 1, |
| | | | | | | 4,8. 11, 15, |
| | | | | | | 18) |
| 10 | Anti-PD-1 | 10 | 1 | 10 | i.p. | BIW × 3 |
| | + | | | | | weeks (day 1, |
| | | | | | | 4, 8, 11, 15, |
| | | | | | | 18) |
| | Cytokine mRNA | 40 μg/ | 40 μg/50 μl | 50 μl/tumor | intratumoral | Q4D × 4 (day |
| | Mixture | tumor | | | | 1, 5, 9, 13) |

Results

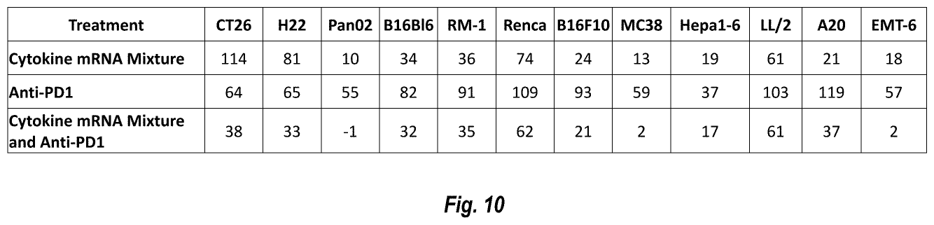

To further understand the influence of tumor heterogeneity, twelve muring models were tested for sensitivity towards cytokine mRNA mixture or combinatorial treatment with an anti-PD-1 antibody. In contrast to the anti-PD1 antibody alone, most tumor types were sensitive to single-agent mRNA therapy. Furthermore, all models showed tumor growth deceleration upon combined treatment with cytokine mRNA and anti-PD1 (FIG. 10), further highlighting the versatility of cytokine-encoding, local mRNA therapy.

1. A method of treating a subject having a solid tumor cancer, comprising administering an effective amount of RNAs comprising RNA encoding an IL-12sc protein, RNA encoding an IL-15 sushi protein, RNA encoding an IFNα protein, and RNA encoding a GM-CSF protein, wherein the subject has failed, or become intolerant, resistant, or refractory to an anti-programmed cell death 1 (PD-1) or anti-programmed cell death 1 ligand 1 (PD-L1) therapy.

2-7. (canceled)

8. The method of claim 1,
i) wherein the subject has anti-PD-1 and/or anti-PD-L1 resistant solid tumor cancer; or
ii) wherein the subject has a solid tumor cancer with acquired resistance or innate resistance to anti-PD-1 and/or anti-PD-L1 therapy: or
iii) wherein the subject has a solid tumor cancer with innate resistance to anti-PD-1 and/or anti-PD-L1 therapy; or
iv) wherein the subject has an advanced-stage, unresectable, or metastatic solid tumor ganger; or
v) wherein the refractory or resistant cancer is one that does not respond to a specified treatment; or
vi) wherein the refraction occurs from the very beginning of treatment; or
vii) wherein the refraction occurs during treatment; or
viii) wherein the cancer is resistant before treatment begins; or
ix) wherein the subject has a cancer that does not respond to the anti-programmed cell death 1 (PD-1) and/or anti-programmed cell death 1 ligand 1 (PD-L1) therapy; or
x) wherein the subject has a cancer that is becoming refractory or resistant to a specified treatment, wherein the specified treatment is an anti-PD1 or anti-PD-L1 therapy; or
xi) wherein the subject has become less responsive to the therapy since first receiving it; or
xii) wherein the subject has not received the therapy, but has a type of cancer that does not typically respond to the therapy.

9-20. (canceled)

21. The method of claim 1, wherein the method further comprises selecting a subject that has failed, or become intolerant, resistant, or refractory to an anti-programmed cell death 1 (PD-1) or anti-programmed cell death 1 ligand 1 (PD-L1) therapy.

22. The method of claim 1, wherein the subject is human.

23. The method of claim 1, wherein one or more of the following is met:
wherein the subject has a metastatic solid tumor; or
wherein the subject has an unresectable solid tumor; or
wherein the subject has not been treated previously with an anti-PD-1 or anti-PD-L1 therapy; or
wherein the subject is without other treatment options; or
wherein the subject has two or three tumor lesions; or
wherein the subject has measurable disease according to the Response Evaluation Criteria in Solid Tumors (RECIST) 1.1 criteria; or
wherein the subject has a life expectancy of more than 3 months; or
wherein the subject is at least 18 years of age.

24. (canceled)

25. The method of claim 1, wherein the subject has a cancer cell comprising a partial or total loss of beta-2-microglobulin (B2M) function.

26. The method of claim 25, wherein the cancer cell has a partial loss of B2M function or a total loss of B2M function.

27. (canceled)

28. (canceled)

29. The method of claim 25, wherein the subject comprises a cell comprising a mutation in the B2M gene; or
wherein the mutation is a substitution, insertion, or deletion; or
wherein the mutation is a frameshift mutation, wherein the frameshift mutation is in exon 1 of B2M; or
wherein the frameshift mutation comprises p.Leu13fs and/or p.Ser14fs.

30. (canceled)

31. The method of claim 25, wherein the B2M gene comprises a loss of heterozygosity (LOH).

32-34. (canceled)

35. The method of claim 25, wherein the subject has a reduced level of B2M protein as compared to a subject without a partial or total loss of B2M function.

36. The method of claim 1, wherein the subject has a reduced level of surface expressed major histocompatibility complex class I (MHC I) as compared to a control, optionally wherein the control is a non-cancerous sample from the same subject.

37. The method of claim 1, wherein the solid tumor cancer is an epithelial tumor, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, melanoma tumor, small cell lung tumor, non-small cell lung cancer, neuroblastoma tumor, testicular tumor, carcinoma tumor, adenocarcinoma tumor, seminoma tumor, retinoblastoma, cutaneous squamous cell carcinoma (CSCC), squamous cell carcinoma for the head and neck (HNSCC), head and neck cancer, osteosarcoma tumor, kidney tumor, thyroid tumor, anaplastic thyroid cancer (ATC), liver tumor, colon tumor, or other solid tumors amenable to intratumoral injection.

38. The method of claim 1,
(a) wherein the solid tumor cancer is lymphoma;
(b) wherein the solid tumor cancer is melanoma; or
(c) wherein the solid tumor cancer is uveal melanoma or mucosal melanoma; or
(d) wherein the solid tumor cancer is melanoma comprising superficial, subcutaneous and/or lymph node metastases amenable for intratumoral injection; or
(e) wherein the solid tumor cancer is HNSCC and/or mucosal melanoma with only mucosal sites; or
(f) wherein the solid tumor cancer is not melanoma.

39. The method of claim 38, wherein the lymphoma is Non-Hodgkin lymphoma or Hodgkin lymphoma.

40-46. (canceled)

47. The method of claim 1, wherein the RNAs are administered as monotherapy.

48. The method of claim 1, wherein the subject has more than one solid tumor.

49. The method of claim 48, wherein at least one tumor is resistant, refractory, or intolerant to an anti-PD-1 or anti-PD-L1 therapy and at least one tumor is not.

50. The method of claim 49, wherein both resistant and non-resistant tumors are successfully treated.

51. The method of claim 1, wherein one or more of the following (1)-(10) is met:
(1) the solid tumor cancer is stage III, subsets of stage III, stage IV, or subsets of stage IV;
(2) the solid tumor cancer is advanced-stage and unresectable;
(3) the solid tumor cancer has spread from its origin to another site in the subject;
(4) the solid tumor cancer has one or more cutaneous or subcutaneous lesions, wherein the cancer is not a skin cancer;
(5) the solid tumor cancer is stage IIIB, stage IIIC, or stage IV melanoma;
(6) the solid tumor cancer is one in which an anti-PD-1 or anti-PD-L1 therapy is not routinely used;
(7) the solid tumor cancer is not melanoma, non-small cell lung cancer, kidney cancer, head and neck cancer, breast cancer, or CSCC;
(8) the solid tumor cancer is one for which an anti-PD1 or anti-PD-L1 therapy is routinely used, but which has not been treated with the therapy yet;
(9) the solid tumor cancer is stage IIIB, IIIC, or unresectable stage IV melanoma that is resistant and/or refractory to anti-PD-1 or anti-PD-L1 therapy; or
(10) the solid tumor cancer comprises superficial or subcutaneous lesions and/or metastases.

52-59. (canceled)

60. The method of claim 1, wherein
i. the solid tumor cancer is not melanoma, CSCC, or HNSCC; and
ii. an anti-PD-1 or anti-PD-L1 therapy is not routinely used; and
iii. there are no other suitable treatment options.

61-67. (canceled)

68. A method for treating an advanced-stage melanoma comprising administering to a subject having an advanced-stage melanoma an effective amount of RNAs comprising RNA encoding an IL-12sc protein, RNA encoding an IL-15 sushi protein, RNA encoding an IFNα protein, and RNA encoding a GM-CSF protein, wherein
i. the subject is at least 18 years of age;
ii. the subject has failed prior anti-PD1 or anti-PD-L1 therapies;
iii. the subject has a minimum of 2 lesions; and
iv. the melanoma comprises a tumor that is suitable for direct intratumoral injection.

69. The method of claim 1, wherein
i. the RNA encoding an IL-12sc protein comprises the nucleotide sequence of SEQ ID NO: 17 or 18, or a nucleotide sequence having at least 80% identity to the nucleotide sequence of SEQ ID NO: 17 or 18; and/or
ii. the IL-12sc protein comprises the amino acid sequence of SEQ ID NO: 14, or an amino acid sequence having at least 80% identity to the amino acid sequence of SEQ ID NO:14; and/or
iii. the RNA encoding an IL-12sc protein comprises a nucleotide sequence having at least 80% identity to the p40 portion of IL-12sc (nucleotides 1-984 of SEQ ID NO: 17 or 18) and at least 80% identity to the p30 portion of IL-12sc (nucleotides 1027-1623 of SEQ ID NO: 17 or 18) and further comprises nucleotides between the p40 and p35 portions encoding a linker polypeptide.

70. The method of claim 1, wherein
i. the RNA encoding an IL-15 sushi protein comprises the nucleotide sequence of SEQ ID NO: 26, or a nucleotide sequence having at least 80% identity to the nucleotide sequence of SEQ ID NO: 26; and/or
ii. the IL-15 sushi protein comprises the amino acid sequence of SEQ ID NO: 24, or an amino acid sequence having at least 80% identity to the amino acid sequence of SEQ ID NO: 24; and/or
iii. the RNA encoding an IL-15 sushi protein comprises a nucleotide sequence having at least 80% identity to the sushi domain of IL-15 receptor alpha (nucleotides 1-321 of SEQ ID NO: 26) and at least 80% identity to mature IL-15 (nucleotides 382-729 of SEQ ID NO: 26) and optionally further comprises nucleotides between the sushi domain of IL-15 and the mature IL-15 encoding a linker polypeptide.

71. The method of claim 1, wherein
i. the RNA encoding an IFNα protein comprises the nucleotide sequence of SEQ ID NO: 22 or 23, or a nucleotide sequence having at least 80% identity to the nucleotide sequence of SEQ ID NO: 22 or 23 and/or
ii. the IFNα protein comprises the amino acid sequence of SEQ ID NO: 19, or an amino acid sequence having at least 80% identity to the amino acid sequence of SEQ ID NO: 19.

72. The method of claim 1, wherein
i. the RNA encoding a GM-CSF protein comprises the nucleotide sequence of SEQ ID NO: 29, or a nucleotide sequence having at least 80% identity to the nucleotide sequence of SEQ ID NO: 29 and/or
ii. the GM-CSF protein comprises the amino acid sequence of SEQ ID NO: 27, or an amino acid sequence having at least 80% identity to the amino acid sequence of SEQ ID NO: 27.

73. The method of claim 1, wherein at least one RNA comprises a modified nucleoside in place of at least one uridine, wherein the modified nucleoside is independently selected from pseudouridine (ψ), N1-methyl-pseudouridine (m¹ψ), and 5-methyl-uridine (m⁵U).

74. (canceled)

75. The method of claim 1, wherein each RNA comprises a modified nucleoside in place of at least one uridine, wherein the modified nucleoside is independently selected from pseudouridine (ψ), N1-methyl-pseudouridine (m¹ψ), and 5-methyl-uridine (m⁵U).

76-79. (canceled)

80. The method of claim 1, wherein at least one RNA comprises the 5′ cap m₂^{7,3′-O}Gppp(m₁^{2′-O})ApG or 3′-O-Me-m⁷G(5′)ppp(5′)G.

81. (canceled)

82. The method of claim 1, wherein at least one RNA comprises a 5′ UTR comprising a nucleotide sequence selected from the group consisting of SEQ ID NOs: 4 and 6, or a nucleotide sequence having at least 80% identity to a nucleotide sequence selected from the group consisting of SEQ ID NOs: 4 and 6.

83. (canceled)

84. The method of claim 1, wherein at least one RNA comprises a 3′ UTR comprising the nucleotide sequence of SEQ ID NO: 8, or a nucleotide sequence having at least 80% identity to the nucleotide sequence of SEQ ID NO: 8.

85. (canceled)

86. The method of claim 1, wherein at least one RNA comprises a poly-A tail of at least 100 nucleotides or a poly-A tail shown in SEO ID NO: 30.

87-89. (canceled)

90. The method of claim 1, wherein one or more RNA comprises:
i. a 5′ cap comprising m₂^{7,3′-O}Gppp(m₁^{2′-O})ApG or 3′-O-Me-m⁷G(5′)ppp(5′)G;
ii. a 5′ UTR comprising (i) a nucleotide sequence selected from the group consisting of SEQ ID NOs: 4 and 6, or (ii) a nucleotide sequence having at least 80% identity to a nucleotide sequence selected from the group consisting of SEQ ID NOs: 4 and 6;
iii. a 3′ UTR comprising (i) the nucleotide sequence of SEQ ID NO: 8, or (ii) a nucleotide sequence having at least 80% identity to the nucleotide sequence of SEQ ID NO:8; and
iv. a poly-A tail comprising at least 100 nucleotides.

91. (canceled)

92. The method of claim 1, wherein treating the solid tumor cancer comprises reducing the size of a tumor or preventing cancer metastasis in a subject.

93. The method of claim 1, wherein the RNAs are administered at the same time.

94. The method of claim 1, wherein the RNAs are administered via injection.

95. The method of claim 94, wherein the RNAs are mixed together in liquid solution prior to injection.

96. The method of claim 1, wherein the RNAs are administered in a neoadjuvant setting.


SEQUENCE LISTING (31 sequences)

SEQ ID NO: 1-2 - skipped (2 entries)

SEQ ID NO: 3 - DNA, 145 bp, Artificial Sequence
  Synthetic: 5 UTR (DNA)
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagcc

SEQ ID NO: 4 - RNA, 145 nt, Artificial Sequence
  Synthetic: 5 UTR (RNA)
ggaauaaacuagucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagcc

SEQ ID NO: 5 - DNA, 52 bp, Artificial Sequence
  Synthetic: Alternative Mod 5 UTR (DNA)
agacgaactagtattcttctggtccccacagactcagagagaacccgccacc

SEQ ID NO: 6 - RNA, 52 nt, Artificial Sequence
  Synthetic: Alternative Mod 5 UTR (RNA)
agacgaacuaguauucuucugguccccacagacucagagagaacccgccacc

SEQ ID NO: 7 - DNA, 317 bp, Artificial Sequence
  Synthetic: 3 UTR (DNA)
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 8 - RNA, 317 nt, Artificial Sequence
  Synthetic: 3 UTR (RNA)
cucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcu

SEQ ID NO: 9-13 - skipped (5 entries)

SEQ ID NO: 14 - protein, 539 aa, Homo sapiens
  misc_feature (1)..(539): Human IL-12sc (amino acid)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSSGGGGSPGGGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 15 - DNA, 1623 bp, Homo sapiens
  misc_feature (1)..(1623): Human non-optimized IL-12sc (CDS DNA)
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagagggtcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggctctagcggagggggaggctctcctggcgggggatctagcagaaacctccccgtggccactccagacccaggaatgttcccatgccttcaccactcccaaaacctgctgagggccgtcagcaacatgctccagaaggccagacaaactctagaattttacccttgcacttctgaggaaattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctgatga

SEQ ID NO: 16 - DNA, 1623 bp, Homo sapiens
  misc_feature (1)..(1623): Human optimized IL-12sc (CDS DNA)
atgtgtcaccagcagctggtgatctcatggttctccctggtatttctggcatctcctcttgtcgcaatctgggaactgaagaaagacgtgtatgtcgttgagctcgactggtatccggatgcgcctggcgagatggtggtgctgacctgtgacaccccagaggaggatgggatcacttggacccttgatcaatcctccgaagtgctcgggtctggcaagactctgaccatacaagtgaaagagtttggcgatgccgggcagtacacttgccataagggcggagaagttctgtcccactcactgctgctgctgcacaagaaagaggacggaatttggagtaccgatatcctgaaagatcagaaagagcccaagaacaaaaccttcttgcggtgcgaagccaagaactactcagggagatttacttgttggtggctgacgacgatcagcaccgatctgactttctccgtgaaatcaagtaggggatcatctgaccctcaaggagtcacatgtggagcggctactctgagcgctgaacgcgtaagaggggacaataaggagtacgagtatagcgttgagtgccaagaggatagcgcatgccccgccgccgaagaatcattgcccattgaagtgatggtggatgctgtacacaagctgaagtatgagaactacacaagctccttcttcatccgtgacatcatcaaaccagatcctcctaagaacctccagcttaaacctctgaagaactctagacaggtggaagtgtcttgggagtatcccgacacctggtctacaccacattcctacttcagtctcacattctgcgttcaggtacagggcaagtccaaaagggagaagaaggatcgggtctttacagataaaacaagtgccaccgttatatgccggaagaatgcctctatttctgtgcgtgcgcaggacagatactatagcagctcttggagtgaatgggccagtgtcccatgttcagggtcatccggtggtggcggcagccccggaggcggtagctccagaaatctccctgtggctacacctgatccaggcatgtttccctgtttgcaccatagccaaaacctcctgagagcagtcagcaacatgctccagaaagctagacaaacactggaattctacccatgcacctccgaggaaatagatcacgaggatatcactaaggacaaaacaagcactgtcgaagcatgccttcccttggaactgacaaagaacgagagttgccttaattcaagagaaacatctttcattacaaacggtagctgcttggcaagcagaaaaacatcttttatgatggccctttgtctgagcagtatttatgaggatctcaaaatgtaccaggtggagtttaagaccatgaatgccaagctgctgatggacccaaagagacagattttcctcgatcagaatatgctggctgtgattgatgaactgatgcaggccttgaatttcaacagcgaaaccgttccccagaaaagcagtcttgaagaacctgacttttataagaccaagatcaaactgtgtattctcctgcatgcctttagaatcagagcagtcactatagatagagtgatgtcctacctgaatgcttcctgatga

SEQ ID NO: 17 - RNA, 1623 nt, Homo sapiens
  misc_feature (1)..(1623): Human non-optimized IL-12sc (RNA encoding CDS)
augugucaccagcaguuggucaucucuugguuuucccugguuuuucuggcaucuccccucguggccauaugggaacugaagaaagauguuuaugucguagaauuggauugguauccggaugccccuggagaaauggugguccucaccugugacaccccugaagaagaugguaucaccuggaccuuggaccagagcagugaggucuuaggcucuggcaaaacccugaccauccaagucaaagaguuuggagaugcuggccaguacaccugucacaaaggaggcgagguucuaagccauucgcuccugcugcuucacaaaaaggaagauggaauuugguccacugauauuuuaaaggaccagaaagaacccaaaaauaagaccuuucuaagaugcgaggccaagaauuauucuggacguuucaccugcugguggcugacgacaaucaguacugauuugacauucagugucaaaagcagcagagggucuucugacccccaaggggugacgugcggagcugcuacacucucugcagagagagucagaggggacaacaaggaguaugaguacucaguggagugccaggaggacagugccugcccagcugcugaggagagucugcccauugaggucaugguggaugccguucacaagcucaaguaugaaaacuacaccagcagcuucuucaucagggacaucaucaaaccugacccacccaagaacuugcagcugaagccauuaaagaauucucggcagguggaggucagcugggaguacccugacaccuggaguacuccacauuccuacuucucccugacauucugcguucagguccagggcaagagcaagagagaaaagaaagauagagucuucacggacaagaccucagccacggucaucugccgcaaaaaugccagcauuagcgugcgggcccaggaccgcuacuauagcucaucuuggagcgaaugggcaucugugcccugcaguggcucuagcggagggggaggcucuccuggcgggggaucuagcagaaaccuccccguggccacuccagacccaggaauguucccaugccuucaccacucccaaaaccugcugagggccgucagcaacaugcuccagaaggccagacaaacucuagaauuuuacccuugcacuucugaggaaauugaucaugaagauaucacaaaagauaaaaccagcacaguggaggccuguuuaccauuggaauuaaccaagaaugagaguugccuaaauuccagagagaccucuuucauaacuaaugggaguugccuggccuccagaaagaccucuuuuaugauggcccugugccuuaguaguauuuaugaagacuugaagauguaccagguggaguucaagaccaugaaugcaaagcuucugauggauccuaagaggcagaucuuucuagaucaaaacaugcuggcaguuauugaugagcugaugcaggcccugaauuucaacagugagacugugccacaaaaauccucccuugaagaaccggauuuuuauaaaacuaaaaucaagcucugcauacuucuucaugcuuucagaauucgggcagugacuauugauagagugaugagcuaucugaaugcuuccugauga

SEQ ID NO: 18 - RNA, 1623 nt, Homo sapiens
  misc_feature (1)..(1623): Human optimized IL-12sc (RNA encoding CDS)
augugucaccagcagcuggugaucucaugguucucccugguauuucuggcaucuccucuugucgcaaucugggaacugaagaaagacguguaugucguugagcucgacugguauccggaugcgccuggcgagaugguggugcugaccugugacaccccagaggaggaugggaucacuuggacccuugaucaauccuccgaagugcucgggucuggcaagacucugaccauacaagugaaagaguuuggcgaugccgggcaguacacuugccauaagggcggagaaguucugucccacucacugcugcugcugcacaagaaagaggacggaauuuggaguaccgauauccugaaagaucagaaagagcccaagaacaaaaccuucuugcggugcgaagccaagaacuacucagggagauuuacuuguugguggcugacgacgaucagcaccgaucugacuuucuccgugaaaucaaguaggggaucaucugacccucaaggagucacauguggagcggcuacucugagcgcugaacgcguaagaggggacaauaaggaguacgaguauagcguugagugccaagaggauagcgcaugccccgccgccgaagaaucauugcccauugaagugaugguggaugcuguacacaagcugaaguaugagaacuacacaagcuccuucuucauccgugacaucaucaaaccagauccuccuaagaaccuccagcuuaaaccucugaagaacucuagacagguggaagugucuugggaguaucccgacaccuggucuacaccacauuccuacuucagucucacauucugcguucagguacagggcaaguccaaaagggagaagaaggaucgggucuuuacagauaaaacaagugccaccguuauaugccggaagaaugccucuauuucugugcgugcgcaggacagauacuauagcagcucuuggagugaaugggccagugucccauguucagggucauccggugguggcggcagccccggaggcgguagcuccagaaaucucccuguggcuacaccugauccaggcauguuucccuguuugcaccauagccaaaaccuccugagagcagucagcaacaugcuccagaaagcuagacaaacacuggaauucuacccaugcaccuccgaggaaauagaucacgaggauaucacuaaggacaaaacaagcacugucgaagcaugccuucccuuggaacugacaaagaacgagaguugccuuaauucaagagaaacaucuuucauuacaaacgguagcugcuuggcaagcagaaaaacaucuuuuaugauggcccuuugucugagcaguauuuaugaggaucucaaaauguaccagguggaguuuaagaccaugaaugccaagcugcugauggacccaaagagacagauuuuccucgaucagaauaugcuggcugugauugaugaacugaugcaggccuugaauuucaacagcgaaaccguuccccagaaaagcagucuugaagaaccugacuuuuauaagaccaagaucaaacuguguauucuccugcaugccuuuagaaucagagcagucacuauagauagagugauguccuaccugaaugcuuccugauga

SEQ ID NO: 19 - protein, 188 aa, Homo sapiens
  misc_feature (1)..(188): Human IFN-alpha-2b (amino acid)
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 20 - DNA, 570 bp, Homo sapiens
  misc_feature (1)..(570): Human non-optimized IFN-alpha-2b (CDS DNA)
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaaccttttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgatga

SEQ ID NO: 21 - DNA, 570 bp, Homo sapiens
  misc_feature (1)..(570): Human optimized IFN-alpha-2b (CDS DNA)
atggccctgacttttgcccttctcgtggctttgttggtgctgagttgcaaatcttcctgtagtgtcggatgtgatctgcctcaaacccacagtctgggatctaggagaacactgatgctgttggcacagatgaggagaattagcctcttttcctgcctgaaggatagacatgacttcggctttccccaagaggagtttggcaatcagttccagaaagcggaaacgattcccgttctgcacgagatgatccagcagatcttcaacctcttttcaaccaaagacagctcagcagcctgggatgagacactgctggacaaattctacacagaactgtatcagcagcttaacgatctggaggcatgcgtgatccaaggggttggtgtgactgaaactccgcttatgaaggaggactccattctggctgtacggaagtacttccagagaataaccctctatctgaaggagaagaagtactcaccatgtgcttgggaagtcgtgagagccgaaatcatgagatccttcagccttagcaccaatctccaggaatctctgagaagcaaagagtgatga

SEQ ID NO: 22 - RNA, 570 nt, Homo sapiens
  misc_feature (1)..(570): Human non-optimized IFN-alpha-2b (RNA encoding CDS)
auggccuugaccuuugcuuuacugguggcccuccuggugcucagcugcaagucaagcugcucugugggcugugaucugccucaaacccacagccuggguagcaggaggaccuugaugcuccuggcacagaugaggagaaucucucuuuucuccugcuugaaggacagacaugacuuuggauuuccccaggaggaguuuggcaaccaguuccaaaaggcugaaaccaucccuguccuccaugagaugauccagcagaucuucaaccuuuucagcacaaaggacucaucugcugcuugggaugagacccuccuagacaaauucuacacugaacucuaccagcagcugaaugaccuggaagccugugugauacagggggugggggugacagagacuccccugaugaaggaggacuccauucuggcugugaggaaauacuuccaaagaaucacucucuaucugaaagagaagaaauacagcccuugugccugggagguugucagagcagaaaucaugagaucuuuuucuuugucaacaaacuugcaagaaaguuuaagaaguaaggaaugauga

SEQ ID NO: 23 - RNA, 570 nt, Homo sapiens
  misc_feature (1)..(570): Human optimized IFN-alpha-2b (RNA encoding CDS)
auggcccugacuuuugcccuucucguggcuuuguuggugcugaguugcaaaucuuccuguagugucggaugugaucugccucaaacccacagucugggaucuaggagaacacugaugcuguuggcacagaugaggagaauuagccucuuuuccugccugaaggauagacaugacuucggcuuuccccaagaggaguuuggcaaucaguuccagaaagcggaaacgauucccguucugcacgagaugauccagcagaucuucaaccucuuuucaaccaaagacagcucagcagccugggaugagacacugcuggacaaauucuacacagaacuguaucagcagcuuaacgaucuggaggcaugcgugauccaagggguuggugugacugaaacuccgcuuaugaaggaggacuccauucuggcuguacggaaguacuuccagagaauaacccucuaucugaaggagaagaaguacucaccaugugcuugggaagucgugagagccgaaaucaugagauccuucagccuuagcaccaaucuccaggaaucucugagaagcaaagagugauga

SEQ ID NO: 24 - protein, 241 aa, Homo sapiens
  misc_feature (1)..(241): Human IL-15 sushi (amino acid)
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 25 - DNA, 729 bp, Homo sapiens
  misc_feature (1)..(729): Human IL-15 sushi (CDS DNA)
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccgggggaggatctggcggcggtgggtctggcgggggatctggcggaggaggaagcttacagaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatga

SEQ ID NO: 26 - RNA, 729 nt, Homo sapiens
  misc_feature (1)..(729): Human IL-15 sushi (RNA encoding CDS)
auggccccgcggcgggcgcgcggcugccggacccucggucucccggcgcugcuacugcugcugcugcuccggccgccggcgacgcggggcaucacgugcccuccccccauguccguggaacacgcagacaucugggucaagagcuacagcuuguacuccagggagcgguacauuuguaacucugguuucaagcguaaagccggcacguccagccugacggagugcguguugaacaaggccacgaaugucgcccacuggacaacccccagucucaaaugcauuagagacccugcccugguucaccaaaggccagcgccacccgggggaggaucuggcggcggugggucuggcgggggaucuggcggaggaggaagcuuacagaacugggugaauguaauaagugauuugaaaaaaauugaagaucuuauucaaucuaugcauauugaugcuacuuuauauacggaaagugauguucaccccaguugcaaaguaacagcaaugaagugcuuucucuuggaguuacaaguuauuucacuugaguccggagaugcaaguauucaugauacaguagaaaaucugaucauccuagcaaacaacaguuugucuucuaaugggaauguaacagaaucuggaugcaaagaaugugaggaacuggaggaaaaaaauauuaaagaauuuuugcagaguuuuguacauauuguccaaauguucaucaacacuucuugauga

SEQ ID NO: 27 - protein, 144 aa, Homo sapiens
  misc_feature (1)..(144): Human GM-CSF (amino acid)
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 28 - DNA, 438 bp, Homo sapiens
  misc_feature (1)..(438): Human GM-CSF (CDS DNA)
atgtggctccagagcctgctgctcttgggcactgtggcctgctccatctctgcacccgcccgctcgcccagccccagcacgcagccctgggagcatgtgaatgccatccaggaggcccggcgtctgctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgatga

SEQ ID NO: 29 - RNA, 438 nt, Homo sapiens
  misc_feature (1)..(438): Human GM-CSF (RNA encoding CDS)
auguggcuccagagccugcugcucuugggcacuguggccugcuccaucucugcacccgcccgcucgcccagccccagcacgcagcccugggagcaugugaaugccauccaggaggcccggcgucugcugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaagcagcacugcccuccaaccccggaaacuuccugugcaacccagauuaucaccuuugaaaguuucaaagagaaccugaaggacuuucugcuugucauccccuuugacugcugggagccaguccaggagugauga

SEQ ID NO: 30 - RNA, 110 nt, Artificial Sequence
  Synthetic: Exemplary Poly-A
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 31 - DNA, 20 bp, Artificial Sequence
  Synthetic: sgRNA
ggcgtatgtatcagtctcag